SEQUENCE LISTING (4 sequences)

SEQ ID NO: 1 - DNA, 24 bp, synthetic construct
  misc_feature 1..24: note = Description of sequence: Component of compound NSC-617145
  source 1..24: mol_type = other DNA
gcactggccgtcgttttacggtcg

SEQ ID NO: 2 - DNA, 23 bp, synthetic construct
  misc_feature 1..23: note = Description of sequence: Component of compound NSC-617145
  source 1..23: mol_type = other DNA
tccaagtaaaacgacggccagtg

SEQ ID NO: 3 - DNA, 2208 bp, Homo sapiens
  misc_feature 1..2208: note = Description of sequence: Gene sequence of WRN-BV08
  source 1..2208: mol_type = genomic DNA
atgaacgagggcgaagaagacgacgacaaggacttcctgtggcctgcccctaacgaagaacaagtgacatgcctgaagatgtacttcggacacagtagcttcaagcctgtgcaatggaaggtcatccactccgtgctggaagaaagaagggacaacgtggctgtgatggctaccggatacggtaagtccctgtgcttccagtaccctcccgtgtacgtgggcaagatcggtctggtgatctcccctctgatctctctgatggaggaccaggtgctgcaattgaagatgtccaacatccccgcttgcttcctgggttccgctcaaagtgagaacgtgctgacagacatcaagctgggcaagtaccgcatcgtgtacgtgacccctgagtactgctccggtaacatgggtctgctgcaacagctggaggctgacatcggaatcaccctgatcgctgtggacgaggctcactgcatctccgagtggggacacgacttccgcgactccttccgtaagctgggatccttgaagaccgctctccctatggtgcctatcgtggccctgaccgccactgcttcctcctccatccgcgaggacatcgtgcgttgcctgaacctgcgcaaccctcagatcacttgcaccggtttcgaccgccctaacttgtacctcgaggtgcgtcgcaagaccggtaacatcctccaggacctgcagcctttcctggtcaagacctcctcccactgggaatttgagggccctaccatcatctactgcccttcccgcaagatgacccagcaagtcaccggcgagctgcgcaagctcaacctctcctgcggtacctaccacgctggtatgtccttctccacccgcaaggacatccaccaccgcttcgtccgtgacgaaatccaatgcgtcatcgctaccatcgctttcggaatgggcatcaacaaggctgacatccgccaggtgatccactacggcgcccccaaggacatggaatcctactaccaggaaatcggtcgcgccggtcgcgacggtctgcagtcttcctgtcacgtgctgtgggcccccgctgacatcaacctgaaccgccacctgctgaccgaaatccgcaacgagaagttccgcctgtacaagctcaagatgatggctaagatggagaagtacctgcactcctcccgctgtcgccgtcagatcatcctctcccacttcgaggacaagcaagtgcaaaaggctagcctgggtatcatgggcaccgaaaagtgttgtgacaactgccgctcccgcctcgaccactgctactccatggacgacagcgaggacacctcctgggacttcggtcctcaagctttcaagctcttgtccgctgtggacatcctgggcgagaagttcggtatcggtctccccatcctcttcctgcgtggtagcaactcccaacgcctggctgaccagtaccgccgccactccctcttcggtaccggtaaggaccagaccgagtcctggtggaaggctttctctcgccaactgatcaccgaaggtttcctggtggaggtgtcccgctacaacaagttcatgaagatctgcgctctcactaagaagggaaggaactggctgcacaaggctaacactgagtcccaatccctcatcctgcaggctaacgaggagctgtgccctaagaagttcctgctgccttcctccaagaccgtgtcctccggaacaaaggaacactgctacaaccaagtccctgtggagctctccaccgagaagaagtccaacctggagaagctgtacagctacaagccttgcgacaagatcagctccggttccaacatcagcaagaagtccatcatggtgcaatcccctgaaaaggcctactccagctcccaacctgtcatctccgctcaagagcaagagacccagatcgtgctgtacggtaagctggtcgaagcccgccaaaagcacgctaacaagatggacgtccctcccgctatcctcgccaccaacaagatcctcgtggatatggctaagatgcgccccaccaccgtcgagaacgtgaagcgcatcgacggtgtctccgagggtaaggccgctatgctggctcctctgctggaagtgatcaagcacttctgccagaccaactccgtgcagaccgacctgttcagtagtgagaacctgtacttccaaggccaccatcatcatcatcatcaccactaa

SEQ ID NO: 4 - protein, 735 aa, Homo sapiens
  REGION 1..735: note = Description of sequence: Protein sequence of WRN-BV08
MNEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKFLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSENLYFQGHHHHHHHH

(19) United States
(12) Patent Application Publication
(10) Pub. No.: US 2026/0297070 A1
(43) Pub. Date: Oct. 1, 2026

(54) CYCLIC VINYL SULFONE WRN INHIBITORS

(71) Applicant: Nimbus Wadjet, Inc., Boston, MA (US)

(72) Inventors: Florian Bartels, Berlin (DE); Jennifer Borthwick, Letchworth Garden City (GB); Sebastien Campos, Hoddesdon (GB); Justin Caravella, Cambridge, MA (US); Leela Dodda, Boston, MA (US); Robert Lee Dow, Groton, CT (US); Scott D. Edmondson, Concord, MA (US); Nathan E. Genung, Boston, MA (US); Silvana Marcel Leit de Moradei, Burlington, MA (US); Derun Li, West Roxbury, MA (US); Angela V. West, Franklin, MA (US)

(21) Appl. No.: 19/474,462

(22) PCT Filed: Apr. 11, 2024
(86) PCT No.: PCT/US2024/024124

§ 371 Date: Oct. 10, 2025

Related U.S. Application Data
(60) Provisional application No. 63/495,440, filed on Apr. 11, 2023.

(51) Int. Cl. C07D 409/14; A61K 31/4025; A61K 31/4155; A61K 31/416; A61K 31/422; A61K 31/4245; A61K 31/427; A61K 31/428; A61K 31/429; A61K 31/435; A61K 31/4355; A61K 31/437; A61K 31/4436; A61K 31/4439; A61K 31/444; A61K 31/4545; A61K 31/4709; A61K 31/4725; A61K 31/496; A61K 31/498; A61K 31/501; A61K 31/506; A61K 31/513; A61K 31/5377; A61K 31/5415; A61K 31/55; A61K 31/69; A61K 31/695; C07D 409/12; C07D 413/12; C07D 417/12; C07D 417/14; C07D 471/04; C07D 487/04; C07D 491/048; C07D 495/04; C07D 513/04; C07F 5/02; C07F 7/08

(52) U.S. Cl. CPC C07D 409/14; A61K 31/4025; A61K 31/4155; A61K 31/416; A61K 31/422; A61K 31/4245; A61K 31/427; A61K 31/428; A61K 31/429; A61K 31/435; A61K 31/4355; A61K 31/437; A61K 31/4436; A61K 31/4439; A61K 31/444; A61K 31/4545; A61K 31/4709; A61K 31/4725; A61K 31/496; A61K 31/498; A61K 31/501; A61K 31/506; A61K 31/513; A61K 31/5377; A61K 31/5415; A61K 31/55; A61K 31/69; A61K 31/695; C07D 409/12; C07D 413/12; C07D 417/12; C07D 417/14; C07D 471/04; C07D 487/04; C07D 491/048; C07D 495/04; C07D 513/04; C07F 5/025; C07F 7/0812

(57) ABSTRACT

The present disclosure is directed to cyclic sulfone-containing compounds, including compounds of Formula I: (I) pharmaceutically acceptable salts thereof, and compositions thereof, as well as methods of treatment of cancers such as those involving WRN protein.

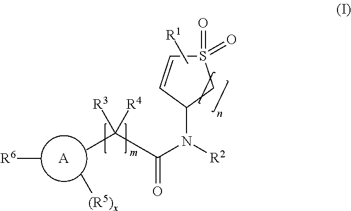

### CROSS-REFERENCE TO RELATED APPLICATIONS

This application claims the benefit of and priority to U.S. Provisional Patent Application Ser. No. 63/495,440, filed Apr. 11, 2023; the contents of which are hereby incorporated by reference in their entirety.

### SEQUENCE LISTING

This application contains a Sequence Listing which has been submitted in .xml format via EFS and is hereby incorporated by reference. The ST.26 copy, created on Mar. 30, 2023, is named 407274-075USP1_ST26.xml and is 8,751 bytes in size.

### FIELD OF INVENTION

The invention provides bicyclic compounds and compositions, the use thereof and methods using the compounds, for inhibiting Werner Syndrome RecQ DNA helicase (WRN) and methods of treating disease using said compounds, in particular the use in treating cancer, and in particular the treatment of cancer characterized as microsatellite instability-high (MSI-H) or mismatch repair deficient (dMMR), including colorectal, gastric and endometrial cancer. The invention also provides the use of said compounds as research chemicals, intermediate compounds, combinations, processes and formulations.

### BACKGROUND

Loss of DNA mismatch repair is a common initiating event in cancer development occurring in 10-30% of colorectal, endometrial, ovarian and gastric cancers (Aaltonen, L. A. et al. Clues to the pathogenesis of familial colorectal cancer, Science 260, 812-816 (1993), Bonneville R et al., Landscape of Microsatellite Instability Across 39 Cancer Types. JCO Precis Oncol. 1: PO.17.00073 (2017)). Cancers that are deficient in mismatch repair (dMMR) have a high mutational burden, and frequent deletion and insertion events in repetitive DNA tracts, a phenotype known as microsatellite instability (MSI). While progress has been made in the treatment of microsatellite instability high (MSI-H) cancers, and the demonstration that pembrolizumab (anti-PD1) treatment led to significantly longer progression-free survival than chemotherapy when received as first-line therapy for MSI-H-dMMR metastatic colorectal cancer (CRC) which resulted in the recent approval of pembrolizumab as first-line treatment of these cancers, there is still a significant unmet medical need in CRC and other MSI-H indications (Andre T., et al. Pembrolizumab in Microsatellite-Instability-High Advanced Colorectal Cancer. N Engl J Med 383(23):22072218 (2020)). Several large-scale functional genomics screens across large panels of cell lines, including Novartis with 398 cell lines from the Cancer Cell Line Encyclopedia (CCLE) (McDonald E. R. et al., Project DRIVE: A Compendium of Cancer Dependencies and Synthetic Lethal Relationships Uncovered by Large-Scale, Deep RNAi Screening. Cell 170(3):577-592 (2017)), have identified the Werner Syndrome RecQ helicase (WRN) as being selectively required for the survival of cell lines with defective mismatch repair that have become MSI-H (Behan, F. M. et al. Prioritization of cancer therapeutic targets using CRISPR-Cas9 screens. Nature 568, 511-516 (2019), Chan, E. M. et al. WRN helicase is a synthetic lethal target in microsatellite unstable cancers. Nature 568, 551-556 (2019). Kategaya, L., Perumal, S. K., Hager, J. H. & Belmont, L. D. Werner syndrome helicase is required for the survival of cancer cells with microsatellite instability. iScience 13, 488-497 (2019), Lieb, S. et al. Werner syndrome helicase is a selective vulnerability of microsatellite instability-high tumor cells. eLife 8, e43333 (2019)). WRN is synthetically lethal with MSI cancers. Depletion of WRN leads to anti-proliferative effects and results in activation of multiple DNA damage signaling markers, induction of cell cycle arrest and apoptosis in MSI-H cancer models but not cancer cells with an intact MMR pathway (otherwise known as microsatellite stable or MSS). The anti-proliferative effects of WRN depletion could not be rescued with a helicase deficient WRN construct, demonstrating that helicase activity of WRN is required for MSI-H viability. These findings indicate that WRN helicase provides a DNA repair and maintenance function that is essential for cell survival in MSI cancers. Recently, the mechanism of WRN dependence has been elucidated. It has been shown that dinucleotide TA repeats are selectively unstable in MSI cells and undergo large scale expansions. These expanded TA repeats form secondary DNA structures that require the WRN helicase for unwinding (van Wietmarschen, N. et al. Repeat expansions confer WRN dependence in microsatellite-unstable cancers. Nature 586, 292-298, 2020). In the absence of WRN (or upon WRN helicase inhibition), expanded TA repeats in MSI cells are subject to nuclease cleavage and chromosome breakage. Thus, inhibiting the WRN helicase is an attractive strategy for the treatment of MSI-H cancers.

### SUMMARY

There remains a need for new treatments and therapies for the treatment of cancer, and in particular cancers characterized as microsatellite instability-high (MSI-H) or mismatch repair deficient (dMMR), including colorectal, gastric or endometrial cancer. The invention provides compounds, pharmaceutically acceptable salts thereof, pharmaceutical compositions thereof and combinations thereof, said compounds being inhibitors of Werner Syndrome RecQ DNA Helicase (WRN). The invention further provides methods of treating, preventing, or ameliorating a disease or condition, comprising administering to a subject in need thereof an effective amount of a WRN inhibitor. The invention also provides compounds, pharmaceutically acceptable salts thereof, pharmaceutical compositions thereof and combinations thereof, said compounds being useful for the treatment of cancer, in particular cancers characterized as microsatellite instability-high (MSI-H) or mismatch repair deficient (dMMR). Also provided are compounds that bind to, and/or inhibit WRN, and are therefore useful as research chemicals, e.g. as a chemical probe, and as tool compounds. Various embodiments of the invention are described herein.

In one aspect, the disclosure provides a compound, or pharmaceutically acceptable salt thereof, according to the Formula I:

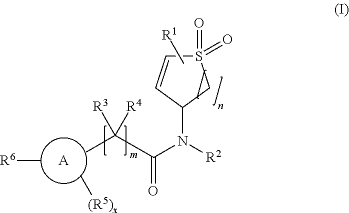

or a pharmaceutically acceptable salt thereof, wherein R¹, R², R³, R⁴, R⁵, R⁶, m, n, x, and Ring A are as described and defined herein.

In another aspect, the invention provides a pharmaceutical composition comprising a compound of Formula I of the present invention and one or more pharmaceutically acceptable carriers.

In another aspect, the invention provides a combination, in particular a pharmaceutical combination, comprising a compound of Formula I of the present invention and one or more therapeutically active agents.

In another aspect, the invention provides a compound of Formula I of the present invention for use as a medicament, in particular for the treatment of a disorder or disease which can be treated by WRN inhibition.

In another aspect, the invention provides a compound of Formula I of the present invention for use in the treatment of cancer, particularly wherein the cancer is characterized as microsatellite instability-high (MSI-H) or mismatch repair deficient (dMMR).

In another aspect, the invention provides a method of treating a disorder or disease which can be treated by WRN inhibition in a subject, comprising administering to the subject a therapeutically effective amount of a compound of Formula I of the present invention.

In another aspect, the invention provides a method of treating cancer in a subject, more particularly wherein the cancer is characterized as microsatellite instability-high (MSI-H) or mismatch repair deficient (dMMR), comprising administering to the subject a therapeutically effective amount of a compound of Formula I of the present invention.

In another aspect, the invention provides the use of a compound of Formula I of the present invention in the manufacture of a medicament for the treatment of a disorder or disease which can be treated by WRN inhibition.

In another aspect, the invention provides a compound of Formula I of the present invention for use as a research chemical, for example as a chemical probe or as a tool compound.

In another aspect, the invention provides a solid form, process or intermediate as described herein.

### DETAILED DESCRIPTION

### 1. General Description of Certain Embodiments of the Invention

In one aspect, the disclosure provides a compound, or pharmaceutically acceptable salt thereof, according to the Formula I:

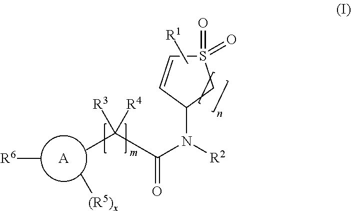

wherein
Ring A is a bivalent cyclic group selected from:
   a 3-8 membered saturated or partially unsaturated monocyclic carbocyclylene,
   phenylene,
   a 4-8 membered saturated or partially unsaturated monocyclic heterocyclylene having 1-2 heteroatoms independently selected from nitrogen, oxygen, and sulfur,
   a 5-6 membered monocyclic heteroarylene having 1-4 heteroatoms independently selected from nitrogen, oxygen, and sulfur,
   an 8-10 membered bicyclic heteroarylene ring having 1-5 heteroatoms independently selected from nitrogen, oxygen, and sulfur,
   a 7-12 membered saturated or partially unsaturated bicyclic heterocyclylene having 1-4 heteroatoms independently selected from nitrogen, oxygen, and sulfur,
   a 5-12 membered saturated or partially unsaturated bicyclic carbocyclylene that is optionally bridged or spirocyclic, and
   a 5-12 membered saturated or partially unsaturated bicyclic heterocyclylene that is optionally bridged or spirocyclic having 1-4 heteroatoms independently selected from nitrogen, oxygen, and sulfur;
R¹ is hydrogen, halogen, —CN, or an optionally substituted C₁₋₆ aliphatic group;
R² is a C₁₋₆ aliphatic group, a C₁₋₆ aliphatic-Cy′ group, or a cyclic group selected from:
   a 3-8 membered saturated or partially unsaturated monocyclic carbocyclic ring,
   phenyl,
   a 3-8 membered saturated or partially unsaturated monocyclic heterocyclic ring having 1-2 heteroatoms independently selected from nitrogen, oxygen, and sulfur,
   a 5-6 membered monocyclic heteroaromatic ring having 1-4 heteroatoms independently selected from nitrogen, oxygen, and sulfur,
   an 8-10 membered bicyclic heteroaromatic ring having 1-5 heteroatoms independently selected from nitrogen, oxygen, and sulfur,
   a 7-12 membered saturated or partially unsaturated bicyclic heterocyclic ring having 1-4 heteroatoms independently selected from nitrogen, oxygen, and sulfur,
   a 5-12 membered saturated or partially unsaturated bicyclic carbocyclic ring that is optionally bridged or spirocyclic, and
   a 5-12 membered saturated or partially unsaturated bicyclic heterocyclic ring that is optionally bridged or spirocyclic having 1-4 heteroatoms independently selected from nitrogen, oxygen, and sulfur,
      wherein R² is substituted with y instances of R^{A};
R³ and R⁴ are each independently selected at each occurrence from hydrogen, halogen, —CN, —NO₂, —OR, —SR, —NR₂, —NRC(O)R—S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group optionally substituted with —C(O)NR₂ or NR₂, a C₁₋₆ aliphatic-Cy″ group, or Cy″; wherein when m is 2 or 3, two adjacent instances of CR³R⁴ are optionally combined to form a carbon-carbon double bond of the formula —C(R⁴)═C(R⁴)—;
each instance of R⁵ is independently selected from hydrogen, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group substituted with z instances of R^{B}, a C₁₋₆ aliphatic-Cy′″ group substituted with z instances of R^{B}, and Cy′″ substituted with z instances of R^{B}; or two instances of R⁵ on the same atom together form oxo; or two instances of R⁵ on adjacent atoms of Ring A, taken together with said adjacent atoms, form a phenyl ring fused to Ring A, wherein said phenyl ring is substituted with 0-4 independently selected halogens;
R⁶ is hydrogen, halogen, a C₁₋₆ aliphatic group, —SF₅, —OR, —SR, —S(O)₂NR₂, —NR₂, —S(O)₂R, —SiR₃, or a cyclic group selected from:
   a 3-8 membered saturated or partially unsaturated monocyclic carbocyclic ring,
   phenyl,
   a 3-8 membered saturated or partially unsaturated monocyclic heterocyclic ring having 1-2 heteroatoms independently selected from nitrogen, oxygen, and sulfur,
   a 5-6 membered monocyclic heteroaromatic ring having 1-4 heteroatoms independently selected from nitrogen, oxygen, and sulfur,
   an 8-10 membered bicyclic heteroaromatic ring having 1-5 heteroatoms independently selected from nitrogen, oxygen, and sulfur,
   a 5-12 membered saturated or partially unsaturated bicyclic carbocyclic ring that is optionally bridged or spirocyclic,
   a 5-12 membered saturated or partially unsaturated bicyclic heterocyclic ring that is optionally bridged or spirocyclic having 1-4 heteroatoms independently selected from nitrogen, oxygen, and sulfur,
      wherein the C₁₋₆ aliphatic group and cyclic group are substituted with z instances of R^{B};
each instance of R^{A} is independently selected from hydrogen; a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂;
—N(R)C(NR)NR₂; —N(R)S(O)₂NR₂; —N(R)S(O)₂R; two instances of R^{A} on the same atom together form oxo; and a cyclic group selected from:
   3-8 membered saturated or partially unsaturated monocyclic carbocyclic ring;
   a 5-6 membered monocyclic heteroaromatic ring having 1-4 heteroatoms independently selected from nitrogen, oxygen, and sulfur; and
   a 3-8 membered saturated or partially unsaturated monocyclic heterocyclic ring having 1-2 heteroatoms independently selected from nitrogen, oxygen, and sulfur, wherein the cyclic group is optionally substituted with 1, 2, or 3 groups independently selected from —OR, —CR₂OR, and halogen;
each instance of R^{B} is independently selected from hydrogen; a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; —N(R)S(O)₂R; phenyl; and two instances of R^{B} on the same atom together form oxo;
each of Cy′, Cy″, and Cy′″ is independently an optionally substituted cyclic group selected from:
   a 3-8 membered saturated or partially unsaturated monocyclic carbocyclic ring,
   phenyl,
   a 3-8 membered saturated or partially unsaturated monocyclic heterocyclic ring having 1-2 heteroatoms independently selected from nitrogen, oxygen, and sulfur,
   a 5-6 membered monocyclic heteroaromatic ring having 1-4 heteroatoms independently selected from nitrogen, oxygen, and sulfur,
   an 8-10 membered bicyclic heteroaromatic ring having 1-5 heteroatoms independently selected from nitrogen, oxygen, and sulfur,
   a 5-12 membered saturated or partially unsaturated bicyclic carbocyclic ring that is optionally bridged or spirocyclic,
   a 5-12 membered saturated or partially unsaturated bicyclic heterocyclic ring that is optionally bridged or spirocyclic having 1-4 heteroatoms independently selected from nitrogen, oxygen, and sulfur, and
   an 8-10 membered bicyclic aromatic carbocyclic ring;
each R is independently hydrogen, —C(O)N(CH₃)₂, —C(O)₂CH₃, —C(O)₂C(CH₃)₃, —C(O)₂CH(CH₃)₂, —S(O)₂CH₃, an optionally substituted C₁₋₆ aliphatic group, an optionally substituted cyclic group selected from:
   phenyl,
   an optionally substituted 3-7 membered saturated or partially unsaturated carbocyclic ring,
   an optionally substituted 3-7 membered saturated or partially unsaturated heterocyclic ring having 1-2 heteroatoms independently selected from nitrogen, oxygen, and sulfur,
   or an optionally substituted 5-6 membered heteroaryl ring having 1-4 heteroatoms independently selected from nitrogen, oxygen, and sulfur; and/or
      two R groups on the same atom are taken together with the same atom to form a cyclic group selected from:
an optionally substituted 4-7 membered saturated or partially unsaturated carbocycyl,
an optionally substituted 4-7 membered saturated or partially unsaturated heterocyclyl having 1-3 heteroatoms independently selected from nitrogen, oxygen, and sulfur, and
an optionally substituted 5-6 membered heteroaryl ring having 1-3 heteroatoms independently selected from nitrogen, oxygen, and sulfur;
x is 0, 1, 2, 3, or 4;
m is 0, 1, 2, or 3;
n is 1 or 2;
y is 0, 1, 2, or 3; and
z is 0, 1, 2, or 3.

In another aspect, the invention provides a method of treating a disorder or disease which can be treated by WRN inhibition in a subject, comprising administering to the subject a therapeutically effective amount of a compound of Formula I of the present invention.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 5-12 ring members and 0-4 heteroatoms independently selected from nitrogen, oxygen, or sulfur unless otherwise specified, a bridged bicyclic group is optionally substituted with one or more substituents as set forth for aliphatic groups. Additionally or alternatively, any substitutable nitrogen of a bridged bicyclic group is optionally substituted. The term “alkyl” refers to a C₁₋₁₂ straight or branched saturated aliphatic group. In certain instances, alkyl refers to a C₁₋₈ straight or branched saturated aliphatic group or a C₁₋₆ straight or branched saturated aliphatic group. The term “lower alkyl” refers to a C₁₋₄ straight or branched alkyl group.

Exemplary lower alkyl groups are methyl, ethyl, propyl, isopropyl (also referred to interchangeably herein as 2-propyl, iPr, ⁱPr and i-Pr), butyl, isobutyl (also referred to interchangeably herein as 2-butyl, iBu, ⁱBu and i-Bu) and tert-butyl (also referred to interchangeably herein as 2-methyl-2-butyl, tBu, ^{t}Bu and t-Bu).

The term “alkenyl” refers to a C₂₋₁₂ straight or branched partially unsaturated aliphatic group comprising at least one unsaturated carbon carbon double bond. In certain instances, alkenyl refers to a C₂₋₈ or a C₂₋₆ straight or branched partially unsaturated aliphatic group comprising at least one unsaturated carbon carbon double bond. The term “lower alkenyl” refers to a C₂₋₄ straight or branched partially unsaturated aliphatic group comprising at least one unsaturated carbon carbon double bond. Alkenyl groups include both cis (Z) and trans (E) regioisomers. Exemplary lower alkenyl groups are vinyl, allyl, 2-propenyl, and butenyl isomers (—CH₂CH₂CH═CH₂, —CH₂CH═CHCH₃ and —CH═CHCH₂CH₃).

The term “alkynyl” refers to a C₂₋₁₂ straight or branched partially unsaturated aliphatic group comprising at least one unsaturated carbon carbon triple bond. In certain instances, alkynyl refers to a C₂₋₈ or a C₂₋₆ straight or branched partially unsaturated aliphatic group comprising at least one unsaturated carbon carbon triple bond. The term “lower alkynyl” refers to a C₂₋₄ straight or branched partially unsaturated aliphatic group comprising at least one unsaturated carbon carbon triple bond. Exemplary lower alkynyl groups are ethynyl, 1-propynyl, 2-propynyl, 1-butynyl, 2-butynyl, and 3-butynyl.

The term “haloalkyl” refers to a straight or branched alkyl group that is substituted with one or more halogen atoms. The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain,” refers to bivalent alkylene, alkenylene, and alkynylene chains that are straight or branched as defined herein.

As used herein, the term “bivalent,” to describe a cyclic (and noncyclic) group refers to, for example, bivalent carbocyclylene, phenylene, heterocyclylene, and heteroarylene that are bivalent cyclic moieties of carbocycles, phenyls, heterocycles, and heteroaryls described herein. Non-limiting examples include

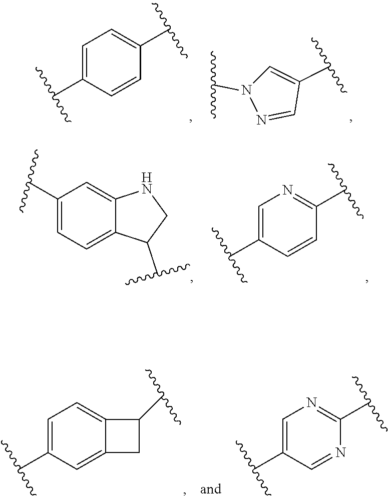 [SMILES: *c1ccc(*)cc1 ; *c1cnn(*)c1 ; *c1ccc2c(c1)NCC2* ; *c1ccc(*)nc1 ; *c1ccc2c(c1)CC2* ; *c1cnc(*)nc1]

“Carbocyclylene” as used herein refers to a carbocyclic or cycloalkyl moiety that is bivalent as described above (i.e., attached at two different points to the rest of the compound).

A carbocyclylene may be multi-cyclic, for example, bicyclic or tricyclic. Such multi-cyclic carbocyclylene systems may be saturated or partially unsaturated (while one ring of the bicyclic system may be aromatic it is to be understood that multi-cyclic ring systems that are not in their entirety aromatic may also fall under the definition of carbocyclylene). The rings may form bridged, fused, or spiro systems.

“Heterocyclylene” as used herein refers to a heterocyclic or heterocyclyl moiety that is bivalent as described above (i.e., attached at two different points to the rest of the compound) and may also be saturated or partially unsaturated. Non-limiting examples include those shown below. Heterocyclylene is understood to include bicyclic heterocyclylene systems. Non-limiting examples of bicyclic heterocyclylene moieties are also shown below and said bicyclic systems may be spirocyclic, fused, or bridged and may be saturated or partially unsaturated.

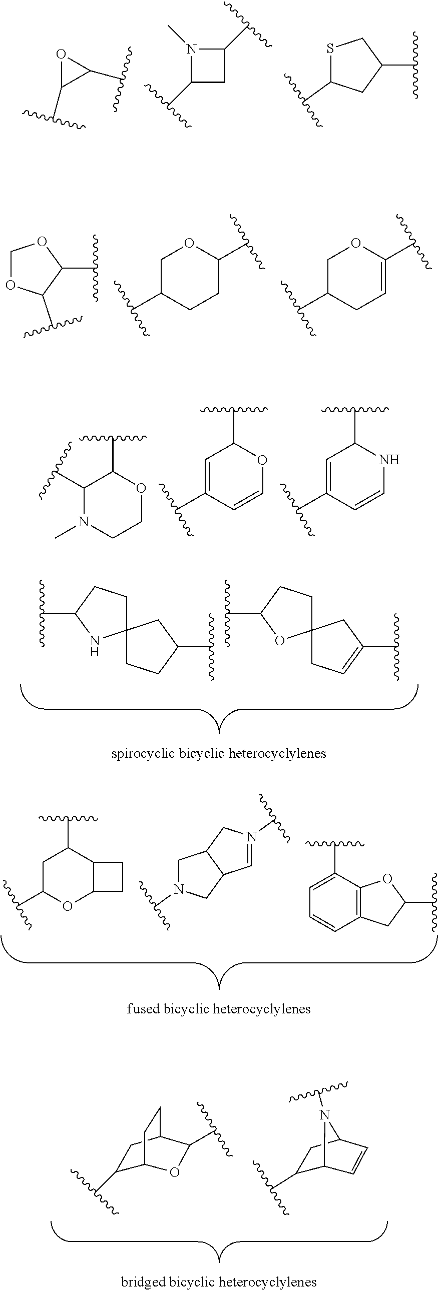

“Phenylene” as used herein refers to a phenyl moiety that is bivalent as described above (i.e., attached at two different points to the rest of the compound). Examples of phenylene include attachment points that are arranged ortho, meta, and para. at

“Arylene” as used herein refers to an mono or multi-cyclic aryl (i.e., phenyl or a multi-cyclic aryl) moiety that is bivalent as described above (i.e., attached at two different points to the rest of the compound), wherein the arylene group contains no heteroatoms.

“Heteroarylene,” as used herein refers to a mono or multi-cyclic aryl ring system that contains at least one heteroatom wherein the ring system is bivalent as described above (i.e., attached at two different points to the rest of the compound).

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, 9 or 10 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyrazinyl, pyrazolyl, pyridazinyl, pyridyl, pyrimidinyl, triazinyl, thienyl, furanyl, pyrrolyl, imidazolyl, pyrazolyl, triazolyl (i.e., 1,2,3-triazolyl), 1,2,4-triazolyl, 1,2,5-triazolyl, 1,3,4-triazolyl, tetrazolyl, oxazolyl, isoxazolyl, oxadiazolyl, 1,2,3-oxadiazolyl, 1,2,4-oxadiazolyl, 1,2,5-oxadiazolyl, 1,3,4-oxadiazolyl, thiazolyl, isothiazolyl, thiadiazolyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, indolizinyl, isoindolin-1-only, 1,2-dihydro-3H-pyrrolo[3,4-c]pyridin-3-onyl, 2,3-dihydro-1H-pyrrolo[3,4-c]pyridin-1-only, imidazo[1,2-a]pyridyl, imidazo[1,5-a]pyridyl, pyrazolo[1,5-a]pyridyl, pyrrolo[1,2-b]pyridazinyl, pyrrolo[1,2-a]pyrimidinyl, imidazo[1,2-b]pyridazinyl, imidazo[1,2-a]pyrimidinyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, oxetanyl, azetidinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

“Arylene” or “heteroarylene,” as used herein (i.e., phenylene), refers to any bivalent aryl or heterocyclyl described herein, that is a bisradical substituted at each of two substitutable positions of the ring system.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

[symbol]

As used herein, the phrase “the left-hand is attached to R⁶” is intended to refer to Ring A,

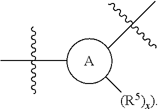

This phrase indicates that the [symbol] marked with an arrow in

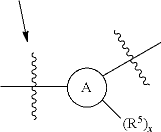

attaches to R⁶ as shown in Formula I with the same arrow at the same site of attachment to R⁶

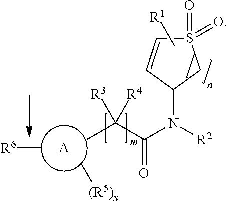

“Left-hand” as it refers to

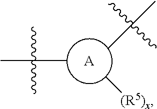

or any specific drawings of said generic variable such as

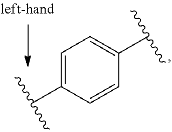 [SMILES: *c1ccc(*)cc1]

refers to the left-side of the page as viewed by the reader.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄B(ORº)₂; —(CH₂)₀₋₄Rº; —(CH₂)₀₋₄ORº; —O(CH₂)₀₋₄Rº; —O—(CH₂)₀₋₄C(O)ORº; —(CH₂)₀₋₄CH(ORº)₂; —(CH₂)₀₋₄SRº; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; (CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —N(Rº)C(NRº)N(Rº)₂: —(CH₂)₀₋₄C(O)Rº; —C(S)Rº; —(CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄SRº; —(CH₂)₀₋₄SC(O)Rº; —(CH₂)₀₋₄C(O)NRº₂; —C(S)NRº₂; —C(S)SRº; —SC(S)SRº, —(CH₂)₀₋₄₀C(O)NRº₂; —C(O)N(ORº)Rº; —C(O)C(O)Rº; —C(O)CH₂C(O)Rº; —C(NORº)Rº; —(CH₂)₀₋₄SSRº; —(CH₂)₀₋₄S(O)₂Rº; —(CH₂)₀₋₄S(O)₂ORº; —(CH₂)₀₋₄OS(O)₂Rº; —S(O)₂NRº₂; —(CH₂)₀₋₄S(O)Rº; —N(Rº)S(O)₂NRº₂; —N(Rº)S(O)₂Rº; —N(ORº)Rº; —C(NH)NRº₂; —(CH₂)₀₋₄P(O)₂Rº; —(CH₂)₀₋₄P(O)Rº₂; —(CH₂)₀₋₄OP(O)Rº₂; —(CH₂)₀₋₄OP(O)(ORº)₂; —SiRº₃; —(C₁₋₄ straight or branched alkylene)O—N(Rº)₂; or —(C₁₋₄ straight or branched alkylene)C(O)O—N(Rº)₂, wherein each Rº may be substituted as defined below and is independently hydrogen, C₁₋₆ aliphatic, —SO₂—C₁₋₄ aliphatic (e.g., —SO₂CH₃)—CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of Rº, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on Rº (or the ring formed by taking two independent occurrences of Rº together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}. —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of Rº include ═O and ═S.

Suitable divalent substituents on a saturated carbon atom of an “optionally substituted” group, which includes instances of Rº (or the ring formed by taking two independent occurrences of Rº together with their intervening atoms), include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{●}₂)₂₋₃O—, wherein each independent occurrence of R^{●} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, Z and E conformational isomers and Rₐ (or M) and Sₐ (or P) atropisomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety of a provided compound comprises one or more deuterium atoms. In certain embodiments, Ring A of a provided compound may be substituted with one or more deuterium atoms.

### 3. Description of Exemplary Embodiments

In one aspect, the disclosure provides a compound, or pharmaceutically acceptable salt thereof, according to the Formula I:

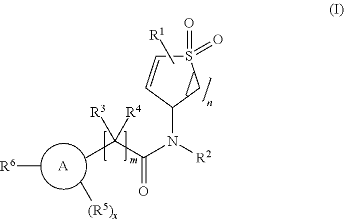

wherein
Ring A is a bivalent cyclic group selected from:
   a 3-8 membered saturated or partially unsaturated monocyclic carbocyclylene,
   phenylene,
   a 4-8 membered saturated or partially unsaturated monocyclic heterocyclylene having 1-2 heteroatoms independently selected from nitrogen, oxygen, and sulfur,
   a 5-6 membered monocyclic heteroarylene having 1-4 heteroatoms independently selected from nitrogen, oxygen, and sulfur,
   an 8-10 membered bicyclic heteroarylene ring having 1-5 heteroatoms independently selected from nitrogen, oxygen, and sulfur,
   a 7-12 membered saturated or partially unsaturated bicyclic heterocyclylene having 1-4 heteroatoms independently selected from nitrogen, oxygen, and sulfur,
   a 5-12 membered saturated or partially unsaturated bicyclic carbocyclylene that is optionally bridged or spirocyclic, and
   a 5-12 membered saturated or partially unsaturated bicyclic heterocyclylene that is optionally bridged or spirocyclic having 1-4 heteroatoms independently selected from nitrogen, oxygen, and sulfur;
R¹ is hydrogen, halogen, —CN, or an optionally substituted C₁₋₆ aliphatic group;
R² is a C₁₋₆ aliphatic group, a C₁₋₆ aliphatic-Cy′ group, or a cyclic group selected from:
   a 3-8 membered saturated or partially unsaturated monocyclic carbocyclic ring,
   phenyl,
   a 3-8 membered saturated or partially unsaturated monocyclic heterocyclic ring having 1-2 heteroatoms independently selected from nitrogen, oxygen, and sulfur,
   a 5-6 membered monocyclic heteroaromatic ring having 1-4 heteroatoms independently selected from nitrogen, oxygen, and sulfur,
   an 8-10 membered bicyclic heteroaromatic ring having 1-5 heteroatoms independently selected from nitrogen, oxygen, and sulfur,
   a 7-12 membered saturated or partially unsaturated bicyclic heterocyclic ring having 1-4 heteroatoms independently selected from nitrogen, oxygen, and sulfur,
   a 5-12 membered saturated or partially unsaturated bicyclic carbocyclic ring that is optionally bridged or spirocyclic, and
   a 5-12 membered saturated or partially unsaturated bicyclic heterocyclic ring that is optionally bridged or spirocyclic having 1-4 heteroatoms independently selected from nitrogen, oxygen, and sulfur,
      wherein R² is substituted with y instances of R^{A};
R³ and R⁴ are each independently selected at each occurrence from hydrogen, halogen, —CN, —NO₂, —OR, —SR, —NR₂, —NRC(O)R—S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group optionally substituted with —C(O)NR₂ or NR₂, a C₁₋₆ aliphatic-Cy″ group, or Cy″; wherein when m is 2 or 3, two adjacent instances of CR³R⁴ are optionally combined to form a carbon-carbon double bond of the formula —C(R⁴)═C(R⁴)—;
each instance of R⁵ is independently selected from hydrogen, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group substituted with z instances of R^{B}, a C₁₋₆ aliphatic-Cy′″ group substituted with z instances of R^{B}, and Cy′″ substituted with z instances of R^{B}; or two instances of R⁵ on the same atom together form oxo; or two instances of R⁵ on adjacent atoms of Ring A, taken together with said adjacent atoms, form a phenyl ring fused to Ring A, wherein said phenyl ring is substituted with 0-4 independently selected halogens;
R⁶ is hydrogen, halogen, a C₁₋₆ aliphatic group, —SF₅, —OR, —SR, —S(O)₂NR₂, —NR₂, —S(O)₂R, —SiR₃, or a cyclic group selected from:
   a 3-8 membered saturated or partially unsaturated monocyclic carbocyclic ring,
   phenyl,
   a 3-8 membered saturated or partially unsaturated monocyclic heterocyclic ring having 1-2 heteroatoms independently selected from nitrogen, oxygen, and sulfur,
   a 5-6 membered monocyclic heteroaromatic ring having 1-4 heteroatoms independently selected from nitrogen, oxygen, and sulfur,
   an 8-10 membered bicyclic heteroaromatic ring having 1-5 heteroatoms independently selected from nitrogen, oxygen, and sulfur,
   a 5-12 membered saturated or partially unsaturated bicyclic carbocyclic ring that is optionally bridged or spirocyclic,
   a 5-12 membered saturated or partially unsaturated bicyclic heterocyclic ring that is optionally bridged or spirocyclic having 1-4 heteroatoms independently selected from nitrogen, oxygen, and sulfur,
      wherein the C₁₋₆ aliphatic group and cyclic group are substituted with z instances of R^{B};
each instance of R^{A} is independently selected from hydrogen; a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; —N(R)S(O)₂R; two instances of R^{A} on the same atom together form oxo; and a cyclic group selected from:
   3-8 membered saturated or partially unsaturated monocyclic carbocyclic ring;
   a 5-6 membered monocyclic heteroaromatic ring having 1-4 heteroatoms independently selected from nitrogen, oxygen, and sulfur; and
   a 3-8 membered saturated or partially unsaturated monocyclic heterocyclic ring having 1-2 heteroatoms independently selected from nitrogen, oxygen, and sulfur,
      wherein the cyclic group is optionally substituted with 1, 2, or 3 groups independently selected from —OR, —CR₂OR, and halogen;
each instance of R^{B} is independently selected from hydrogen; a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; —N(R)S(O)₂R; phenyl; and two instances of R^{B} on the same atom together form oxo;
each of Cy′, Cy″, and Cy′″ is independently an optionally substituted cyclic group selected from:
   a 3-8 membered saturated or partially unsaturated monocyclic carbocyclic ring,
   phenyl,
   a 3-8 membered saturated or partially unsaturated monocyclic heterocyclic ring having 1-2 heteroatoms independently selected from nitrogen, oxygen, and sulfur,
   a 5-6 membered monocyclic heteroaromatic ring having 1-4 heteroatoms independently selected from nitrogen, oxygen, and sulfur,
   an 8-10 membered bicyclic heteroaromatic ring having 1-5 heteroatoms independently selected from nitrogen, oxygen, and sulfur,
   a 5-12 membered saturated or partially unsaturated bicyclic carbocyclic ring that is optionally bridged or spirocyclic,
   a 5-12 membered saturated or partially unsaturated bicyclic heterocyclic ring that is optionally bridged or spirocyclic having 1-4 heteroatoms independently selected from nitrogen, oxygen, and sulfur, and
   an 8-10 membered bicyclic aromatic carbocyclic ring;
each R is independently hydrogen, —C(O)N(CH₃)₂, —C(O)₂CH₃, —C(O)₂C(CH₃)₃, —C(O)₂CH(CH₃)₂, —S(O)₂CH₃, an optionally substituted C₁₋₆ aliphatic group, an optionally substituted cyclic group selected from:
   phenyl,
   an optionally substituted 3-7 membered saturated or partially unsaturated carbocyclic ring,
   an optionally substituted 3-7 membered saturated or partially unsaturated heterocyclic ring having 1-2 heteroatoms independently selected from nitrogen, oxygen, and sulfur,
   or an optionally substituted 5-6 membered heteroaryl ring having 1-4 heteroatoms independently selected from nitrogen, oxygen, and sulfur; and/or
      two R groups on the same atom are taken together with the same atom to form a cyclic group selected from:
an optionally substituted 4-7 membered saturated or partially unsaturated carbocycyl,
an optionally substituted 4-7 membered saturated or partially unsaturated heterocyclyl having 1-3 heteroatoms independently selected from nitrogen, oxygen, and sulfur, and
an optionally substituted 5-6 membered heteroaryl ring having 1-3 heteroatoms independently selected from nitrogen, oxygen, and sulfur;
x is 0, 1, 2, 3, or 4;
m is 0, 1, 2, or 3;
n is 1 or 2;
y is 0, 1, 2, or 3; and
z is 0, 1, 2, or 3.

As described generally above, Ring A is a bivalent cyclic group selected from:
a 3-8 membered saturated or partially unsaturated monocyclic carbocyclylene,
phenylene,
a 4-8 membered saturated or partially unsaturated monocyclic heterocyclylene having 1-2 heteroatoms independently selected from nitrogen, oxygen, and sulfur,
a 5-6 membered monocyclic heteroarylene having 1-4 heteroatoms independently selected from nitrogen, oxygen, and sulfur,
an 8-10 membered bicyclic heteroarylene ring having 1-5 heteroatoms independently selected from nitrogen, oxygen, and sulfur,
a 7-12 membered saturated or partially unsaturated bicyclic heterocyclylene having 1-4 heteroatoms independently selected from nitrogen, oxygen, and sulfur,
a 5-12 membered saturated or partially unsaturated bicyclic carbocyclylene that is optionally bridged or spirocyclic, and
a 5-12 membered saturated or partially unsaturated bicyclic heterocyclylene that is optionally bridged or spirocyclic having 1-4 heteroatoms independently selected from nitrogen, oxygen, and sulfur.

In some embodiments, Ring A is an optionally substituted bivalent phenylene substituted with x instances of R⁵. In some embodiments, Ring A is an optionally substituted bivalent cyclic group selected from:
a 3-8 membered saturated or partially unsaturated monocyclic carbocyclylene,
phenylene,
a 4-8 membered saturated or partially unsaturated monocyclic heterocyclylene having 1-2 heteroatoms independently selected from nitrogen, oxygen, and sulfur,
a 5-6 membered monocyclic heteroarylene having 1-4 heteroatoms independently selected from nitrogen, oxygen, and sulfur, an 8-10 membered bicyclic heteroarylene ring having 1-5 heteroatoms independently selected from nitrogen, oxygen, and sulfur,
a 7-12 membered saturated or partially unsaturated bicyclic heterocyclylene having 1-4 heteroatoms independently selected from nitrogen, oxygen, and sulfur,
a 5-12 membered saturated or partially unsaturated bicyclic carbocyclylene that is optionally bridged or spirocyclic,
a 5-12 membered saturated or partially unsaturated bicyclic heterocyclylene that is optionally bridged or spirocyclic having 1-4 heteroatoms independently selected from nitrogen, oxygen, and sulfur;
   wherein Ring A is substituted with x instances of R⁵.

In some embodiments, Ring A is an optionally substituted bivalent 3-8 membered saturated or partially unsaturated monocyclic carbocyclylene substituted with x instances of R⁵. In some embodiments, Ring A is an optionally substituted bivalent 4-8 membered saturated or partially unsaturated monocyclic heterocyclylene having 1-2 heteroatoms independently selected from nitrogen, oxygen, and sulfur substituted with x instances of R⁵. In some embodiments, Ring A is an optionally substituted bivalent 5-6 membered monocyclic heteroarylene having 1-4 heteroatoms independently selected from nitrogen, oxygen, and sulfur substituted with x instances of R⁵. In some embodiments, Ring A is an optionally substituted bivalent 8-10 membered bicyclic heteroarylene ring having 1-5 heteroatoms independently selected from nitrogen, oxygen, and sulfur substituted with x instances of R⁵. In some embodiments, Ring A is an optionally substituted bivalent 7-12 membered saturated or partially unsaturated bicyclic heterocyclylene having 1-4 heteroatoms independently selected from nitrogen, oxygen, and sulfur substituted with x instances of R⁵. In some embodiments, Ring A is an optionally substituted bivalent 5-12 membered saturated or partially unsaturated bicyclic carbocyclylene that is optionally bridged or spirocyclic substituted with x instances of R⁵. In some embodiments, Ring A is an optionally substituted bivalent 5-12 membered saturated or partially unsaturated bicyclic heterocyclylene that is optionally bridged or spirocyclic having 1-4 heteroatoms independently selected from nitrogen, oxygen, and sulfur substituted with x instances of R⁵.

In some embodiments, Ring A is bivalent phenylene. In some embodiments, Ring A is an optionally substituted bivalent phenylene substituted with 1 instance of R⁵. In some embodiments, Ring A is an optionally substituted bivalent phenylene substituted with 2 instances of R⁵. In some embodiments, Ring A is an optionally substituted bivalent phenylene substituted with 3 instances of R⁵. In some embodiments, Ring A is an optionally substituted bivalent phenylene substituted with 4 instances of R⁵.

In some embodiments, Ring A and its x R⁵ substituents:

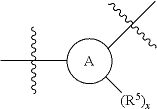

is

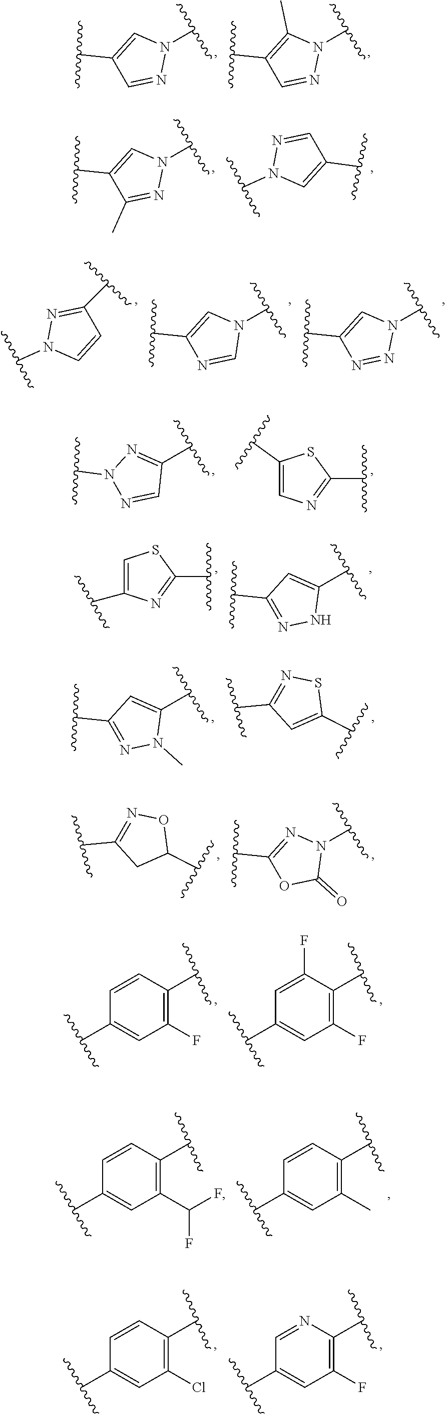

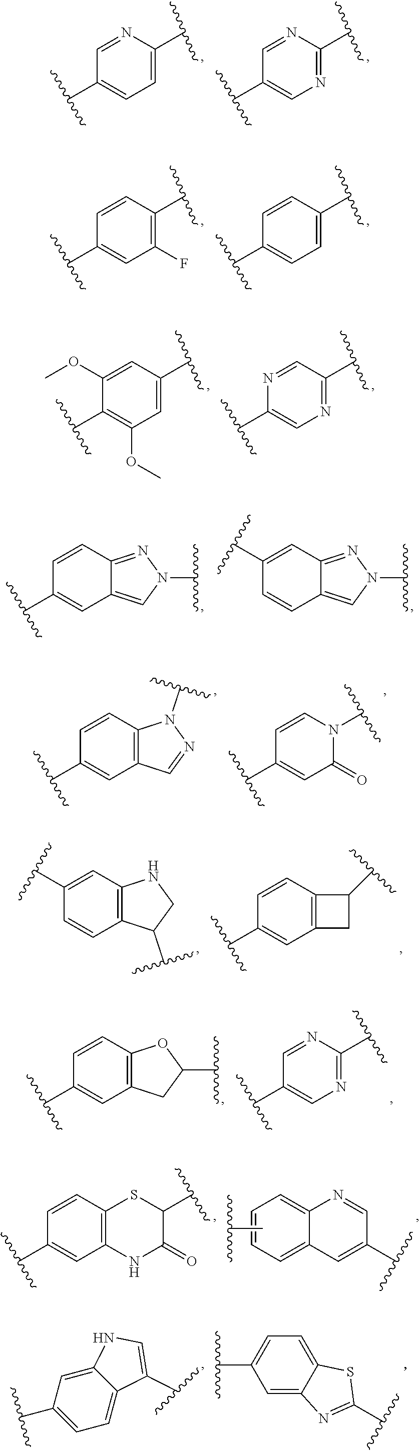

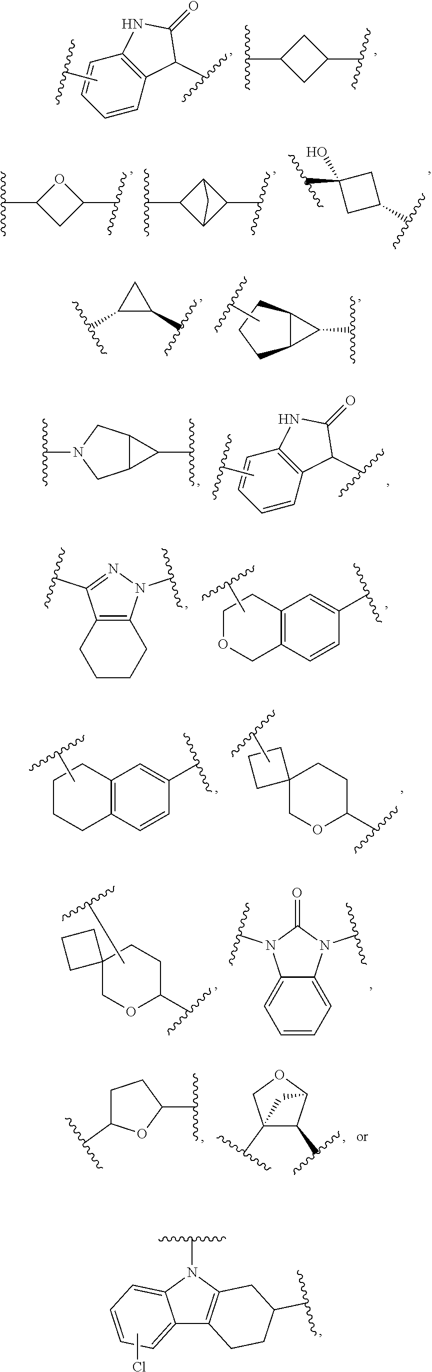

wherein the left-hand [symbol] is attached to R⁶.

In some embodiments, Ring A s as selected from one of the substituents of Table 1.

As described generally above, R¹ is hydrogen, halogen, —CN, or an optionally substituted C₁₋₆ aliphatic. In some embodiments, R¹ is hydrogen or a C₁₋₆ aliphatic. In some embodiments, R¹ is hydrogen or halogen. In some embodiments, R¹ is a C₁₋₆ aliphatic or halogen.

In some embodiments, R¹ is hydrogen. In some embodiments, R¹ is halogen. In some embodiments, R¹ is a C₁₋₆ aliphatic. In some embodiments, R¹ is —CH₂OH.

In some embodiments, R¹ is fluoro. In some embodiments, R¹ is chloro. In some embodiments, R¹ is —CH₃. In some embodiments, R¹ is as selected from one of the substituents of Table 1.

As described generally above, R² is a C₁₋₆ aliphatic group, a C₁₋₆ aliphatic-Cy′ group, or a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 7-12 membered saturated or partially unsaturated bicyclic heterocyclic ring having 1-4 heteroatoms independently selected from nitrogen, oxygen, and sulfur,
a 5-12 membered saturated or partially unsaturated bicyclic carbocyclic ring that is optionally bridged or spirocyclic, and
a 5-12 membered saturated or partially unsaturated bicyclic heterocyclic ring that is optionally bridged or spirocyclic having 1-4 heteroatoms independently selected from nitrogen, oxygen, and sulfur,
   wherein R² is substituted with y instances of R^{A}.

In some embodiments, R² is an optionally substituted cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 7-12 membered saturated or partially unsaturated bicyclic heterocyclic ring having 1-4 heteroatoms independently selected from nitrogen, oxygen, and sulfur,
a 5-12 membered saturated or partially unsaturated bicyclic carbocyclic ring that is optionally bridged or spirocyclic, and
a 5-12 membered saturated or partially unsaturated bicyclic heterocyclic ring that is optionally bridged or spirocyclic having 1-4 heteroatoms independently selected from nitrogen, oxygen, and sulfur,
   wherein R² is substituted with y instances of R^{A}.

In some embodiments, R² is optionally substituted phenyl.

In some embodiments, R² is a C₁₋₆ aliphatic-Cy′ group substituted with y instances of R^{A}. In some embodiments, R² is a —CH₂—Cy′ group. In some embodiments, R² is

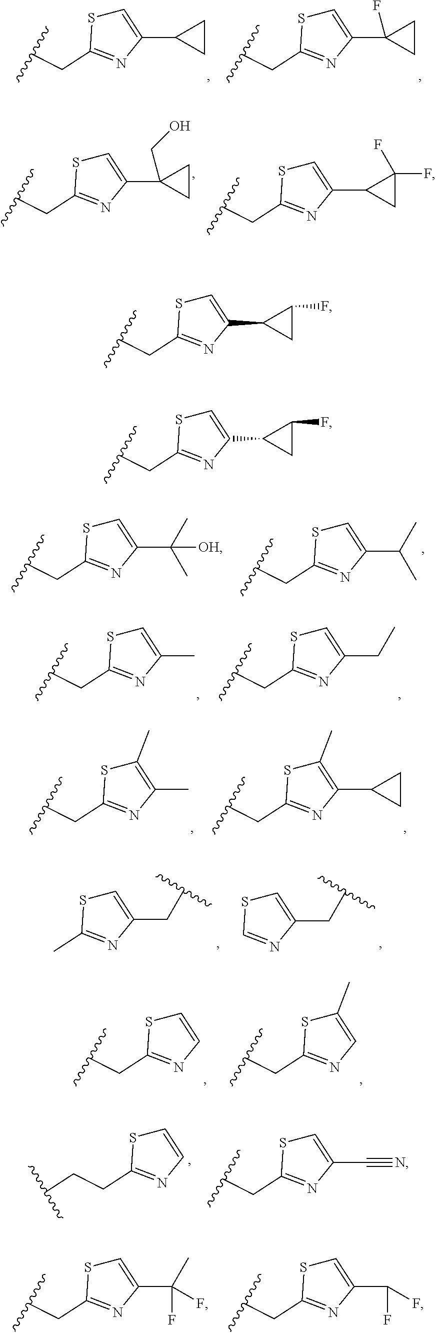

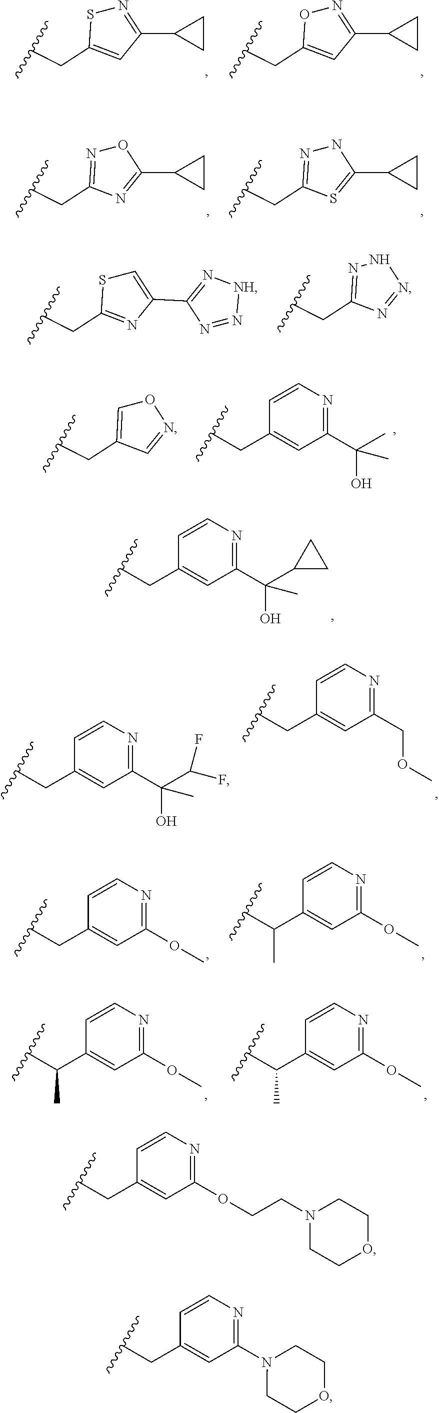

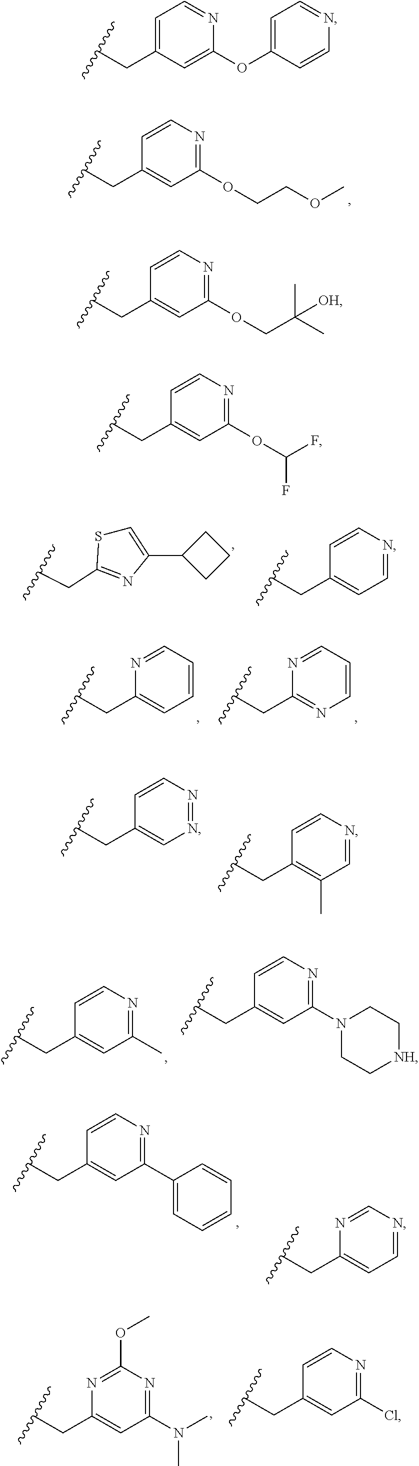

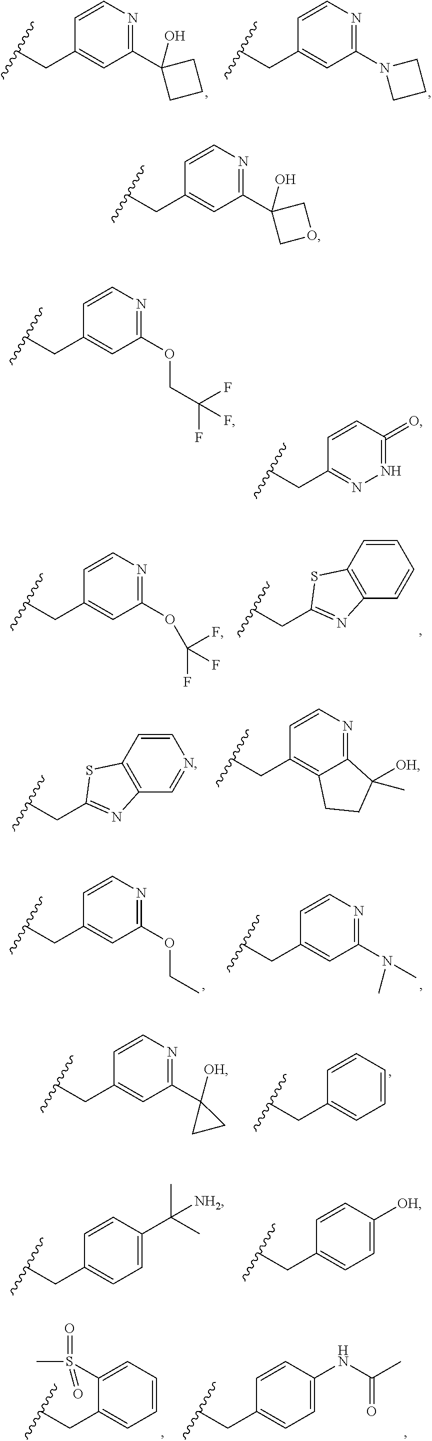

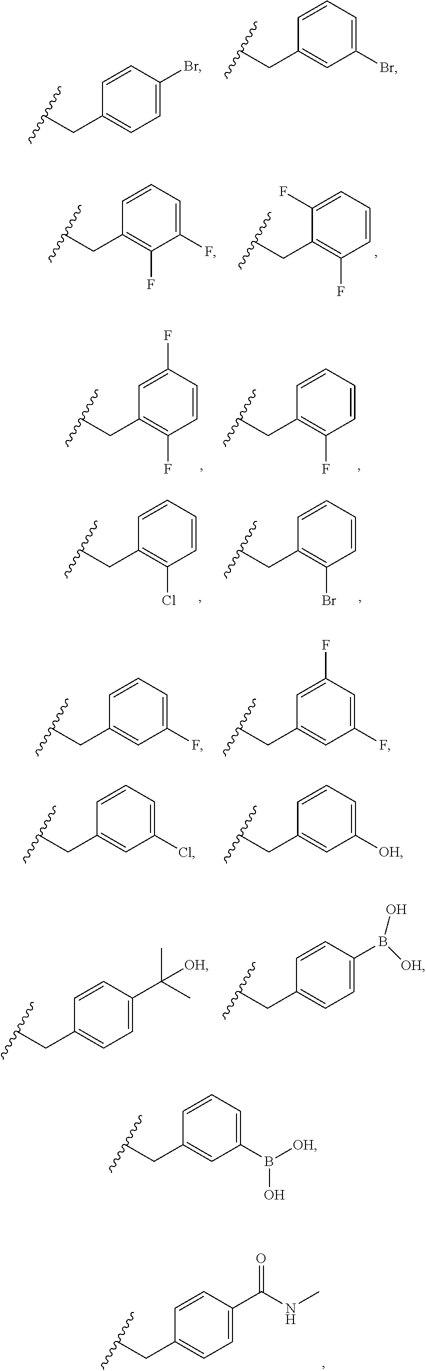

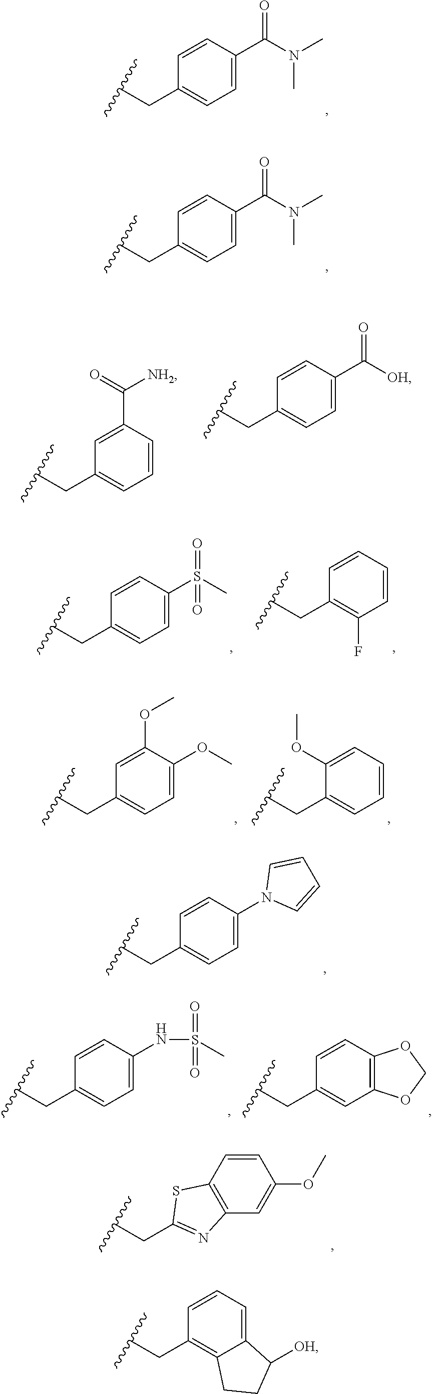

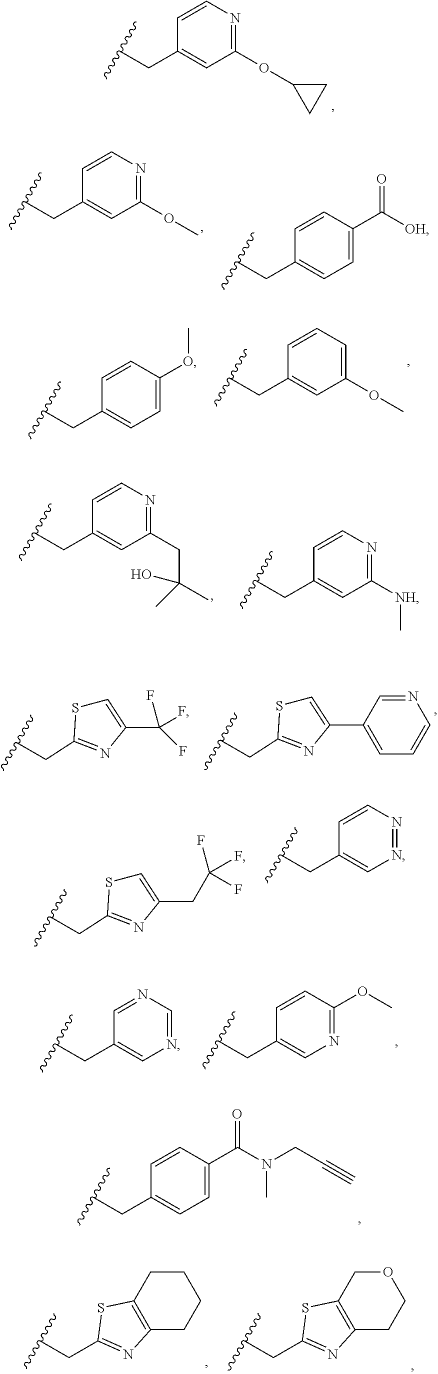

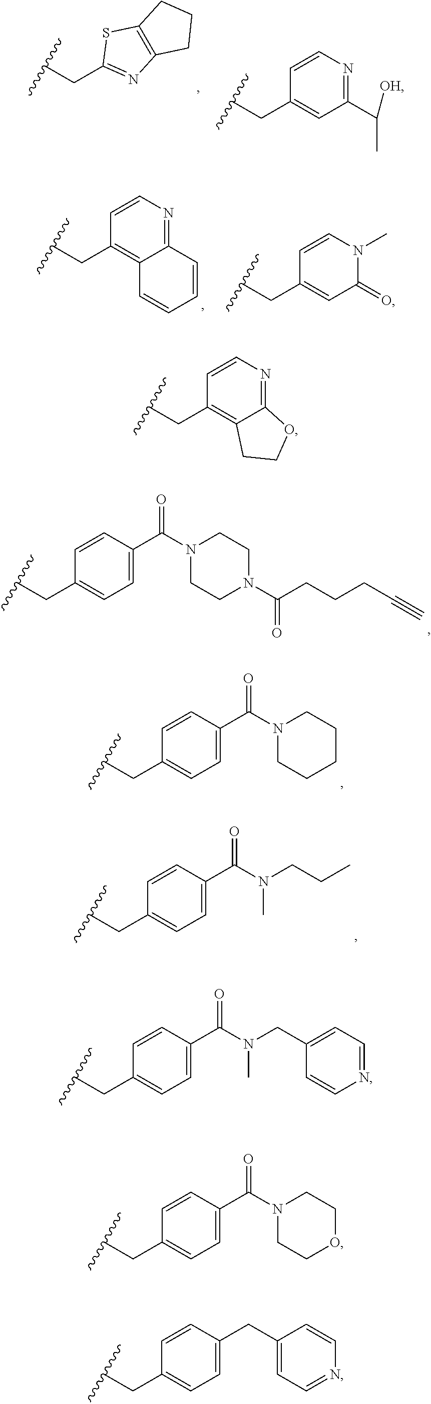

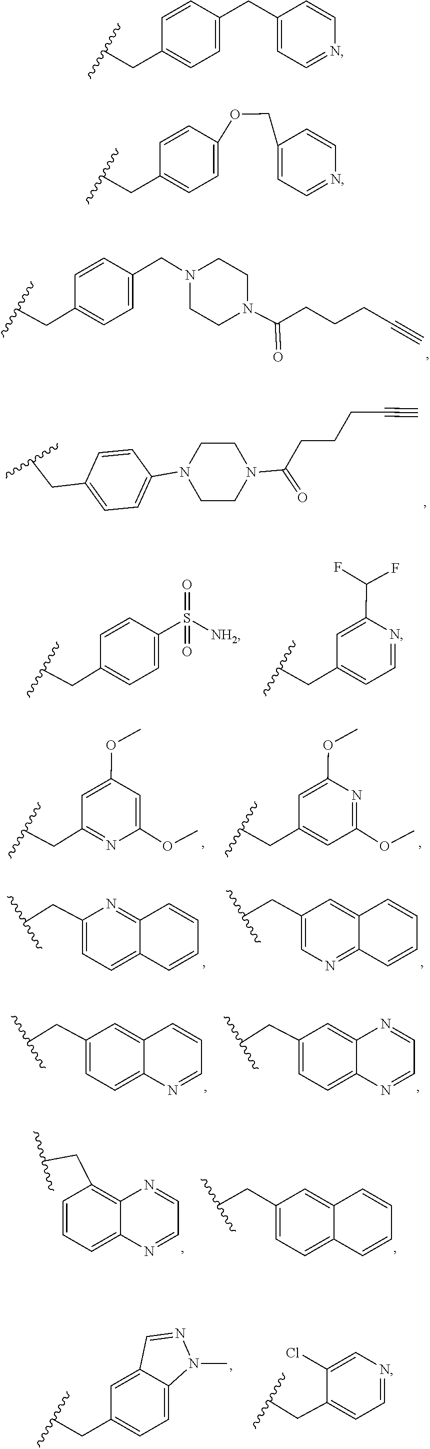

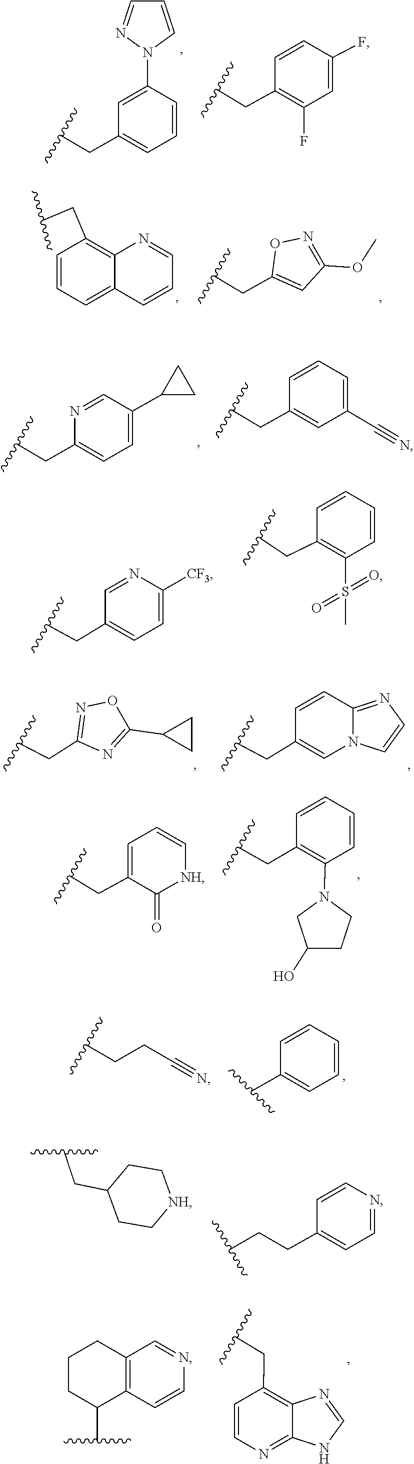

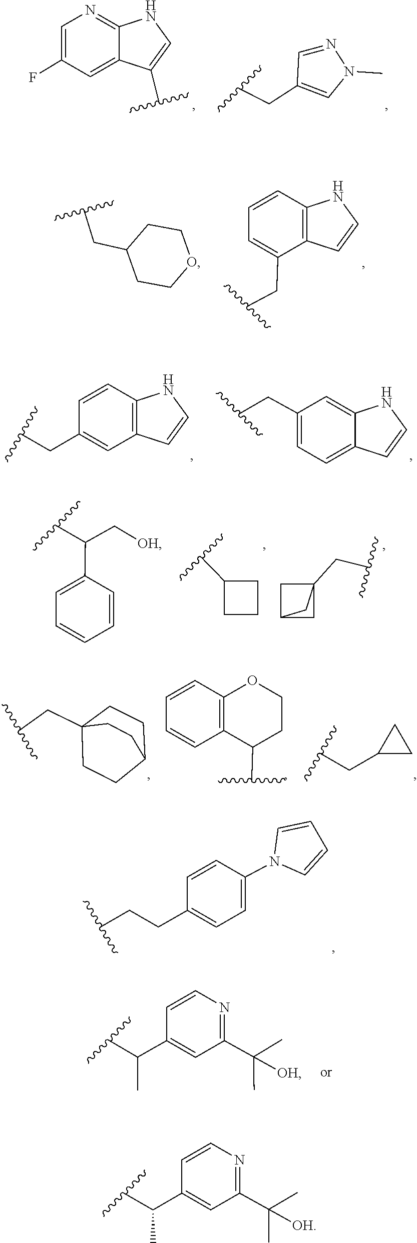

In some embodiments, R² is as selected from one of the substituents of Table 1.

As described generally above, R³ is independently selected at each occurrence from hydrogen, halogen, —CN, —NO₂, —OR, —SR, —NR₂, —NRC(O)R—S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group optionally substituted with —C(O)NR₂ or NR₂, a C₁₋₆ aliphatic-Cy″ group, or Cy″.

In some embodiments, R³ is hydrogen. In some embodiments, R³ is independently selected at each occurrence from halogen, —CN, —NO₂, —OR, —SR, —NR₂, —NRC(O)R—S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group optionally substituted with —C(O)NR₂ or NR₂, a C₁₋₆ aliphatic-Cy″ group, or Cy″. In some embodiments, R³ is independently selected at each occurrence from halogen; a C₁₋₆ aliphatic group optionally substituted with —C(O)NR₂ or NR₂, —OR, and Cy″.

In some embodiments, R³ is independently selected at each occurrence from halogen, a C₁₋₆ aliphatic group, —C(O)NR₂, —NR₂, or —OR. In some embodiments, R³ is independently selected at each occurrence from —CH₃, fluoro, —C(O)N(CH₃)₂, —C(O)N(CH₂CH₃)₂,

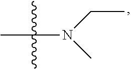 [SMILES: *N(C)CC]

—OCH₃, —OCH₂CH₃, —OCF₂CH₃, —OCH₂CH₂CH₃, or

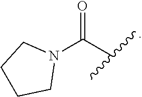 [SMILES: *C(=O)N1CCCC1]

In some embodiments, m is 1, R⁴ is hydrogen, and R³ is halogen, a C₁₋₆ aliphatic group, —C(O)NR₂, —NR₂, or —OR. In some embodiments, m is 1, R⁴ is hydrogen, and R³ is —CH₃, fluoro, —C(O)N(CH₃)₂, —C(O)N(CH₂CH₃)₂,

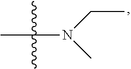 [SMILES: *N(C)CC]

—OCH₃, —OCH₂CH₃, —OCF₂CH₃, —OCH₂CH₂CH₃, or

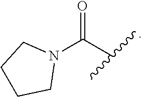 [SMILES: *C(=O)N1CCCC1]

In some embodiments, R³ is as selected from one of the substituents of Table 1.

As described generally above, R⁴ is independently selected at each occurrence from hydrogen, halogen, —CN, —NO₂, —OR, —SR, —NR₂, —NRC(O)R—S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group optionally substituted with —C(O)NR₂ or NR₂, a C₁₋₆ aliphatic-Cy″ group, or Cy″.

In some embodiments, R⁴ is hydrogen. In some embodiments, R⁴ is independently selected at each occurrence from hydrogen and halogen. In some embodiments, R⁴ is independently selected at each occurrence from halogen, —CN, —NO₂, —OR, —SR, —NR₂, —NRC(O)R—S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group optionally substituted with —C(O)NR₂ or NR₂, a C₁₋₆ aliphatic-Cy″ group, or Cy″. In some embodiments, R⁴ is independently selected at each occurrence from halogen, —CN, —NO₂, —OR, —SR, —NR₂, —NRC(O)R—S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, and a C₁₋₆ aliphatic group optionally substituted with —C(O)NR₂ or NR₂. In some embodiments, R⁴ is as selected from one of the substituents of Table 1.

In some embodiments, m is 2 or 3 and two adjacent instances of CR³R⁴ are optionally combined to form a carbon carbon double bond of the formula —C(R⁴)═C(R⁴)—. In some embodiments, m is 2 and the two adjacent instances of CR³R⁴ are optionally combined to form a carbon carbon double bond of the formula R⁴ or

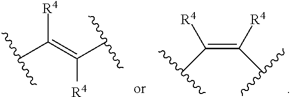

In some embodiments, m is 2 and the two adjacent instances of CR³R⁴ are combined to form a carbon carbon double bond of the formula

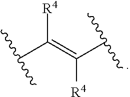

In some embodiments, m is 2 and the two adjacent instances of CR³R⁴ are combined to form a carbon carbon double bond of the formula

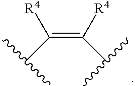

In some embodiments, m is 3 and the three instances of CR³R⁴ are combined to form

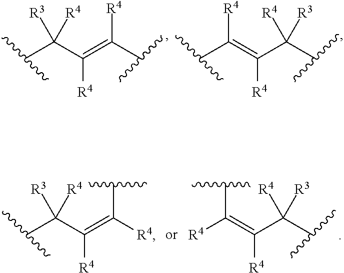

In some embodiments, m is 3 and the three instances of CR³R⁴ are combined to form

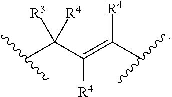

In some embodiments, m is 3 and the three instances of CR³R⁴ are combined to form

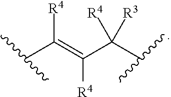

In some embodiments, m is 3 and the three instances of CR³R⁴ are combined to form

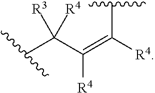

In some embodiments, m is 3 and the three instances of CR³R⁴ are combined to form

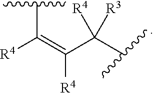

In some embodiments, R⁴ is hydrogen and R³ is independently selected at each occurrence from halogen, a C₁₋₆ aliphatic group, —C(O)NR₂, —NR₂, or —OR. In some embodiments, R⁴ is hydrogen and R³ is independently selected at each occurrence from —CH₃, fluoro, —C(O)N(CH₃)₂, —C(O)N(CH₂CH₃)₂,

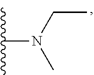 [SMILES: *N(C)CC]

—OCH₃, —OCH₂CH₃, —OCF₂CH₃, —OCH₂CH₂CH₃, or

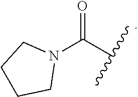 [SMILES: *C(=O)N1CCCC1]

In some embodiments, R⁴ is hydrogen, m is 1, and R³ is halogen, a C₁₋₆ aliphatic group, —C(O)NR₂, —NR₂, or —OR. In some embodiments, R⁴ is hydrogen, m is 1, and R³ is —CH₃, fluoro, —C(O)N(CH₃)₂, —C(O)NH(CH₃), —C(O)N(CH₂CH₃)₂,

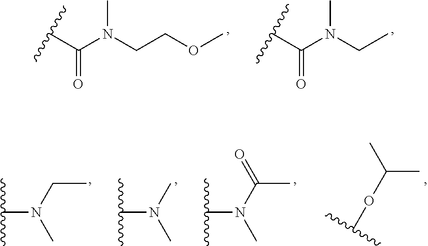

—OCH₃, —SCH₃, —CH₂OCH₃, —OCH₂CH₃, —OCF₂CH₃, —OCH₂CH₂CH₃,

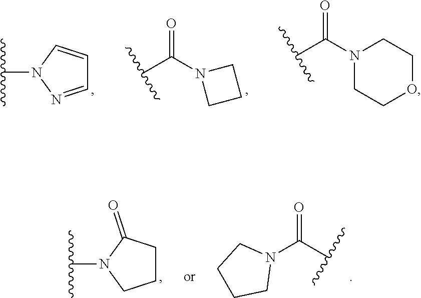 [SMILES: *n1cccn1 ; *C(=O)N1CCC1 ; *C(=O)N1CCOCC1 ; *N1CCCC1=O ; *C(=O)N1CCCC1]

In some embodiments, R⁴ and R³ are each independently selected from a halogen. In some embodiments, R⁴ and R³ are each fluoro.

As described generally above, each instance of R⁵ is independently selected from hydrogen, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group substituted with z instances of R^{B}, a C₁₋₆ aliphatic-Cy′″ group substituted with z instances of R^{B}, and Cy′″ substituted with z instances of R^{B}; or two instances of R⁵ on the same atom together form oxo; or two instances of R⁵ on adjacent atoms of Ring A, taken together with said adjacent atoms, form a phenyl ring fused to Ring A, wherein said phenyl ring is substituted with 0-4 independently selected halogens.

In some embodiments, R⁵ is hydrogen.

In some embodiments, each instance of R⁵ is independently halogen —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group substituted with z instances of R^{B}, a C₁₋₆ aliphatic-Cy′″ group substituted with z instances of R^{B}, or Cy′″ substituted with z instances of R^{B}. In some embodiments, each instance of R⁵ is independently halogen, —CN, —NO₂, —OR, —SR, —NR₂, —S(O)₂R, —S(O)₂NR₂, —S(O)R, —S(O)NR₂, —C(O)R, —C(O)OR, or —C(O)NR₂. In some embodiments, each instance of R⁵ is independently —N(R)S(O)₂NR₂ or —N(R)S(O)₂R. In some embodiments, R⁵ is —N(R)S(O)₂NR₂. In some embodiments, R⁵ is —N(R)S(O)₂R.

In some embodiments, R⁵ is halogen. In some embodiments, R⁵ is halogen and x is 1. In some embodiments, R⁵ is halogen and x is 2. In some embodiments, R⁵ is fluoro. In some embodiments, R⁵ is fluoro and x is 1. In some embodiments, R⁵ is fluoro and x is 2.

In some embodiments, R⁵ is as selected from one of the substituents of Table 1.

In some embodiments, Ring A is phenylene and R⁵ is halogen. In some embodiments, Ring A is phenylene, R⁵ is halogen, and x is 1. In some embodiments, Ring A is phenylene, R⁵ is halogen, and x is 2. In some embodiments, Ring A is phenylene, R⁵ is fluoro. In some embodiments, Ring A is phenylene, R⁵ is fluoro and x is 1. In some embodiments, Ring A is phenylene, R⁵ is fluoro, and x is 2.

As described generally above, R⁶ is hydrogen, halogen, a C₁₋₆ aliphatic group, —SF₅, —OR, —SR, —S(O)₂NR₂, —NR₂, —S(O)₂R, —SiR₃ or a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic, and
a 5-12 membered saturated or partially unsaturated bicyclic heterocyclic ring that is optionally bridged or spirocyclic having 1-4 heteroatoms independently selected from nitrogen, oxygen, and sulfur, wherein the C₁₋₆ aliphatic group and cyclic group are substituted with z instances of R.

In some embodiments, R⁶ is halogen, a C₁₋₆ aliphatic group, —SF₅, —OR, —SR, —S(O)₂NR₂, —NR₂, —S(O)₂R, —SiR₃ or a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic, and
a 5-12 membered saturated or partially unsaturated bicyclic heterocyclic ring that is optionally bridged or spirocyclic having 1-4 heteroatoms independently selected from nitrogen, oxygen, and sulfur,
   wherein the C₁₋₆ aliphatic group and cyclic group are substituted with z instances of R^{B}.

In some embodiments, R⁶ is halogen. In some embodiments, R⁶ is a C₁₋₆ aliphatic group, —SF₅, —OR, —SR, —S(O)₂NR₂, —S(O)₂R, or a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic, and
a 5-12 membered saturated or partially unsaturated bicyclic heterocyclic ring that is optionally bridged or spirocyclic having 1-4 heteroatoms independently selected from nitrogen, oxygen, and sulfur,
   wherein the C₁₋₆ aliphatic group and cyclic group are substituted with z instances of R^{B}.

In some embodiments, R⁶ is a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur, and
a 5-6 membered monocyclic heteroaromatic ring having 1-4 heteroatoms independently selected from nitrogen, oxygen, and sulfur,
   wherein the cyclic group is substituted with z instances of R^{B}.

In some embodiments, R⁶ is a 3-8 membered saturated or partially unsaturated monocyclic carbocyclic ring substituted with z instances of R^{B}. In some embodiments, R⁶ is a phenyl substituted with z instances of R^{B}. In some embodiments, R⁶ is a 3-8 membered saturated or partially unsaturated monocyclic heterocyclic ring having 1-2 heteroatoms independently selected from nitrogen, oxygen, and sulfur substituted with z instances of R^{B}. In some embodiments, R⁶ is a 5-6 membered monocyclic heteroaromatic ring having 1-4 heteroatoms independently selected from nitrogen, oxygen, and sulfur substituted with z instances of R^{B}.

In some embodiments, R⁶ is phenyl substituted with z instances of R^{B}. In some embodiments, R⁶ is fluoro substituted phenyl. In some embodiments, R⁶ is —SF₅. In some embodiments, Ring A is phenylene substituted with x instances of R⁵ and R⁶ is phenyl substituted with z instances of R^{B}. In some embodiments, Ring A is phenylene substituted with x instances of R⁵ and R⁶ is fluoro substituted phenyl. In some embodiments, Ring A is phenylene substituted with x instances of R⁵ and R⁶ is —SF₅.

In some embodiments, R⁶ is a 5-membered heteroaryl substituted with z instances of R^{B}. In some embodiments, R⁶ pyrrole substituted with z instances of R^{B}. In some embodiments, R⁶ pyrrole substituted with —CH₃.

In some embodiments, R⁶ is isopropyl, sec-butyl, —CF₃, —SF₅, —CH₂CF₃, or —OCF₃. In some embodiments, R⁶ is —SF₅. In some embodiments, R⁶ is —CF₃.

In some embodiments, Ring A is bicyclic, m is greater than 0, and R⁶ is hydrogen. In some embodiments, R⁶ is not hydrogen.

In some embodiments, R⁶ is —F, —Br, —Cl, —SF₅, —CF₃, —CH₃, —CH₂CF₃, —OCF₃, —OCH₃, —SCH₃,

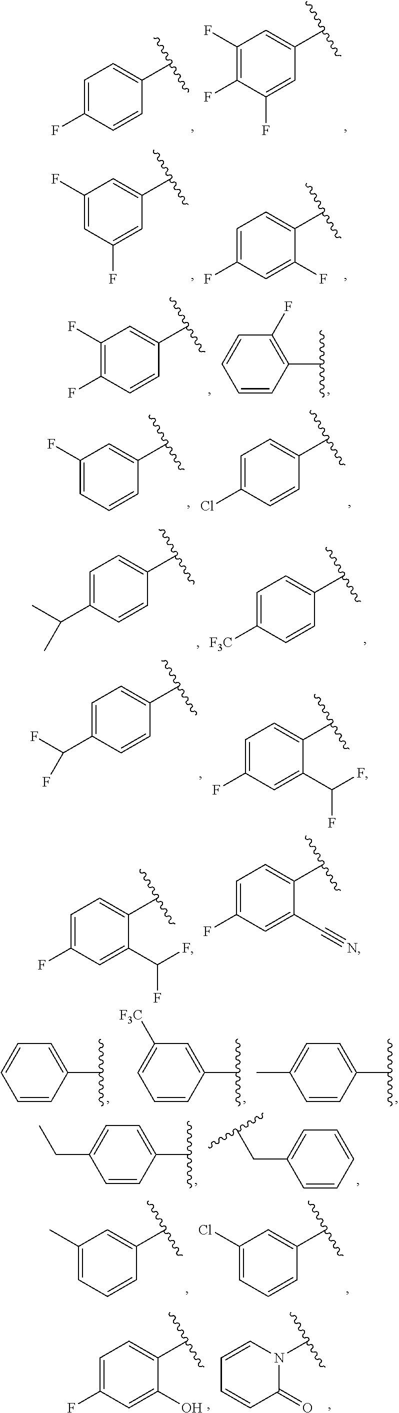

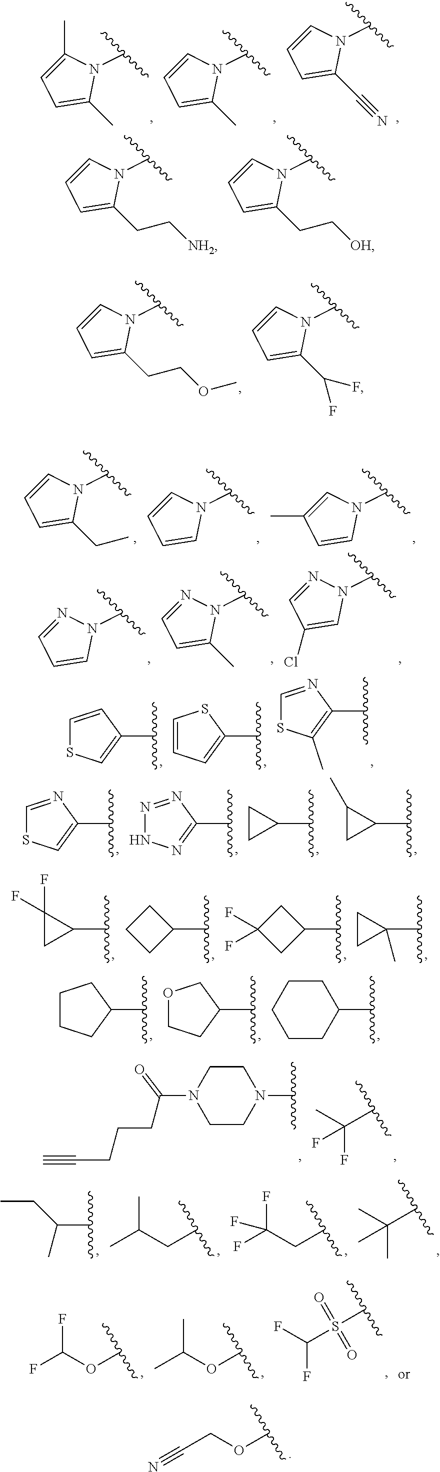

In some embodiments, R⁶ is as selected from one of the substituents of Table 1.

As described generally above, each instance of R^{A} is independently selected from hydrogen; a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; —N(R)S(O)₂R; two instances of R^{A} on the same atom together form oxo; and a cyclic group selected from:
3-8 membered saturated or partially unsaturated monocyclic carbocyclic ring;
a 5-6 membered monocyclic heteroaromatic ring having 1-4 heteroatoms independently selected from nitrogen, oxygen, and sulfur; and
a 3-8 membered saturated or partially unsaturated monocyclic heterocyclic ring having 1-2 heteroatoms independently selected from nitrogen, oxygen, and sulfur, wherein the cyclic group is optionally substituted with 1, 2, or 3 groups independently selected from —OR, —CR₂OR, and halogen.

In some embodiments, R^{A} is hydrogen. In some embodiments, each instance of R^{A} is independently selected from a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; —N(R)S(O)₂R, two instances of R^{A} on the same atom together form oxo; and a cyclic group selected from:
3-8 membered saturated or partially unsaturated monocyclic carbocyclic ring;
a 5-6 membered monocyclic heteroaromatic ring having 1-4 heteroatoms independently selected from nitrogen, oxygen, and sulfur; and
a 3-8 membered saturated or partially unsaturated monocyclic heterocyclic ring having 1-2 heteroatoms independently selected from nitrogen, oxygen, and sulfur,
   wherein the cyclic group is optionally substituted with 1, 2, or 3 groups independently selected from —OR, —CR₂OR, and halogen.

In some embodiments, each instance of R^{A} is independently selected from a C₁₋₆ aliphatic group optionally substituted with 1, 2, or 3 groups independently selected from halogen, —CN, —NR₂, and —OR. In some embodiments, each instance of R^{A} is independently selected from halogen. In some embodiments, each instance of R^{A} is independently selected from a cyclic group selected from:
3-8 membered saturated or partially unsaturated monocyclic carbocyclic ring;
a 5-6 membered monocyclic heteroaromatic ring having 1-4 heteroatoms independently selected from nitrogen, oxygen, and sulfur; and
a 3-8 membered saturated or partially unsaturated monocyclic heterocyclic ring having 1-2 heteroatoms independently selected from nitrogen, oxygen, and sulfur,
   wherein the cyclic group is optionally substituted with 1, 2, or 3 groups independently selected from —OR, —CR₂OR, and halogen.

In some embodiments, R^{A} is as selected from one of the substituents of Table 1.

As described generally above, each instance of R^{B} is independently selected from hydrogen; a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; —N(R)S(O)₂R; phenyl; and two instances of R^{B} on the same atom together form oxo.

In some embodiments, R^{B} is hydrogen. In some embodiments, each instance of R^{B} is independently selected a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂, —N(R)C(NR)NR₂; —N(R)S(O)₂NR₂; —N(R)S(O)₂R; phenyl; and two instances of R^{B} on the same atom together form oxo. In some embodiments, each instance of R^{B} is independently selected a C₁₋₆ aliphatic group optionally substituted with 1, 2, or 3 groups independently selected from halogen, —CN, —NR₂, and —OR. In some embodiments, each instance of R^{B} is independently a halogen.

In some embodiments, R^{B} is as selected from one of the substituents of Table 1.

In some embodiments, R⁶ is a cyclic group substituted with three instances of R^{B}, wherein R^{B} is fluoro. In some embodiments, R⁶ is a cyclic group substituted with two instances of R^{B}, wherein R^{B} is fluoro. In some embodiments, R⁶ is a cyclic group substituted with one instance of R^{B}, wherein R^{B} is fluoro.

In some embodiments, R⁶ is a cyclic group substituted with three instances of R^{B}, wherein R^{B} is chloro. In some embodiments, R⁶ is a cyclic group substituted with two instances of R^{B}, wherein R^{B} is chloro. In some embodiments, R⁶ is a cyclic group substituted with one instance of R^{B}, wherein R^{B} is chloro.

In some embodiments, R⁶ is a cyclic group substituted with two instances of R^{B}, wherein R^{B} is —CH₃. In some embodiments, R⁶ is a cyclic group substituted with one instance of R^{B}, wherein R^{B} is —CH₃. In some embodiments, R⁶ is a cyclic group substituted with two instances of R^{B}, wherein R^{B} is —CH₂CH₃. In some embodiments, R⁶ is a cyclic group substituted with one instance of R^{B}, wherein R^{B} is —CH₂CH₃.

In some embodiments, R⁶ is a cyclic group substituted with two instances of R^{B}, wherein one R^{B} is chloro and the other R^{B} is —OH.

In some embodiments, R⁴ is hydrogen and R¹ is independently selected at each occurrence from halogen; a C₁₋₆ aliphatic group optionally substituted with 1, 2, or 3 groups independently selected from halogen, —CN, —NR₂, and —OR; —CN; —OH; —SR; and —OR. In some embodiments, R⁴ is hydrogen and R^{B} is independently selected at each occurrence from —CH₃, —CH₂CH₃, isopropyl, cyclopropyl, trifluoromethyl, fluoro, chloro, OH, —CN, —OR, SR,

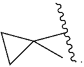 [SMILES: *C1(C)CC1]

In some embodiments, R⁴ is hydrogen, m is 1, and R^{B} is halogen; a C₁₋₆ aliphatic group optionally substituted with 1, 2, or 3 groups independently selected from halogen, —CN, —NR₂, and —OR; —CN; —OH; —SR; and —OR. In some embodiments, R⁴ is hydrogen, m is 1, and R^{B} is —CH₃, —CH₂CH₃, isopropyl, cyclopropyl, trifluoromethyl, fluoro, chloro, OH, —CN, OR, SR, or

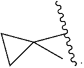 [SMILES: *C1(C)CC1]

As described generally above, Cy′ is an optionally substituted cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic,
a 5-12 membered saturated or partially unsaturated bicyclic heterocyclic ring that is optionally bridged or spirocyclic having 1-4 heteroatoms independently selected from nitrogen, oxygen, and sulfur, and
an 8-10 membered bicyclic aromatic carbocyclic ring.

In some embodiments, Cy′ is an optionally substituted 3-8 membered saturated or partially unsaturated monocyclic carbocyclic ring. In some embodiments, Cy′ is an optionally substituted phenyl. In some embodiments, Cy′ is an optionally substituted 3-8 membered saturated or partially unsaturated monocyclic heterocyclic ring having 1-2 heteroatoms independently selected from nitrogen, oxygen, and sulfur. In some embodiments, Cy′ is an optionally substituted 5-6 membered monocyclic heteroaromatic ring having 1-4 heteroatoms independently selected from nitrogen, oxygen, and sulfur. In some embodiments, Cy′ is an optionally substituted 8-10 membered bicyclic heteroaromatic ring having 1-5 heteroatoms independently selected from nitrogen, oxygen, and sulfur. In some embodiments, Cy′ is an optionally substituted 5-12 membered saturated or partially unsaturated bicyclic carbocyclic ring that is optionally bridged or spirocyclic. In some embodiments, Cy′ is an optionally substituted 5-12 membered saturated or partially unsaturated bicyclic heterocyclic ring that is optionally bridged or spirocyclic having 1-4 heteroatoms independently selected from nitrogen, oxygen, and sulfur. In some embodiments, Cy′ is an optionally substituted 8-10 membered bicyclic aromatic carbocyclic ring.

In some embodiments, Cy′ is an optionally substituted 5 membered monocyclic heteroaromatic ring having 1-4 heteroatoms independently selected from nitrogen, oxygen, and sulfur. In some embodiments, Cy′ is an optionally substituted 5 membered monocyclic heteroaromatic ring having a sulfur atom and a nitrogen atom. In some embodiments, Cy′ is optionally substituted thiazolyl. In some embodiments, Cy′ is thiazolyl optionally substituted with 3-5 membered carbocyclyl, —CH₃, hydroxyl substituted C₁₋₄ aliphatic, pyridinyl, pyrrolyl, trifluoromethyl, —CH₂CF₃, or the thiazolyl is substituted at two adjacent carbon atoms with two independent occurrences of Rº that are taken together with their intervening atoms to form a 5-6 membered saturated, partially unsaturated, or aryl ring fused to the thiazolyl, wherein the 5-6 membered ring comprises 0-2 oxygen atoms.

In some embodiments, Cy′ is an optionally substituted 6 membered monocyclic heteroaromatic ring having 1-4 heteroatoms independently selected from nitrogen, oxygen, and sulfur. In some embodiments, Cy′ is an optionally substituted pyridinyl. In some embodiments, Cy′ is a pyridinyl substituted with hydroxyl substituted C₁₋₄ aliphatic, hydroxyl substituted 3-6 membered carbocyclyl, —OCH₃, —OCH₂CF₃, —OCH₂CH₃, —N(CH₃)₂; or the pyridinyl is substituted at two adjacent carbon atoms with two independent occurrences of Rº that are taken together with their intervening atoms to form a 5-6 membered saturated or partially unsaturated ring fused to the pyridinyl, wherein the 5-6 membered saturated or partially unsaturated ring is substituted with hydroxyl.

In some embodiments, Cy′ is as selected from one of the substituents of Table 1.

As described generally above, Cy″ is an optionally substituted cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic,
a 5-12 membered saturated or partially unsaturated bicyclic heterocyclic ring that is optionally bridged or spirocyclic having 1-4 heteroatoms independently selected from nitrogen, oxygen, and sulfur, and
an 8-10 membered bicyclic aromatic carbocyclic ring.

In some embodiments, Cy″ is optionally substituted 3-8 membered saturated or partially unsaturated monocyclic carbocyclic ring. In some embodiments, Cy″ is optionally substituted phenyl. In some embodiments, Cy″ is optionally substituted 3-8 membered saturated or partially unsaturated monocyclic heterocyclic ring having 1-2 heteroatoms independently selected from nitrogen, oxygen, and sulfur. In some embodiments, Cy″ is an optionally substituted 5-6 membered monocyclic heteroaromatic ring having 1-4 heteroatoms independently selected from nitrogen, oxygen, and sulfur. In some embodiments, Cy″ is an optionally substituted 8-10 membered bicyclic heteroaromatic ring having 1-5 heteroatoms independently selected from nitrogen, oxygen, and sulfur. In some embodiments, Cy″ is an optionally substituted 5-12 membered saturated or partially unsaturated bicyclic carbocyclic ring that is optionally bridged or spirocyclic. In some embodiments, Cy″ is an optionally substituted 5-12 membered saturated or partially unsaturated bicyclic heterocyclic ring that is optionally bridged or spirocyclic having 1-4 heteroatoms independently selected from nitrogen, oxygen, and sulfur. In some embodiments, Cy″ is an optionally substituted 8-10 membered bicyclic aromatic carbocyclic ring.

In some embodiments, Cy″ is as selected from one of the substituents of Table 1.

As described generally above, Cy′″ is an optionally substituted cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic,
a 5-12 membered saturated or partially unsaturated bicyclic heterocyclic ring that is optionally bridged or spirocyclic having 1-4 heteroatoms independently selected from nitrogen, oxygen, and sulfur, and
an 8-10 membered bicyclic aromatic carbocyclic ring.

In some embodiments, Cy′″ is an optionally substituted 3-8 membered saturated or partially unsaturated monocyclic carbocyclic ring. In some embodiments, Cy′″ is optionally substituted phenyl. In some embodiments, Cy′″ is an optionally substituted 3-8 membered saturated or partially unsaturated monocyclic heterocyclic ring having 1-2 heteroatoms independently selected from nitrogen, oxygen, and sulfur. In some embodiments, Cy′″ is an optionally substituted 5-6 membered monocyclic heteroaromatic ring having 1-4 heteroatoms independently selected from nitrogen, oxygen, and sulfur. In some embodiments, Cy′″ is an optionally substituted 8-10 membered bicyclic heteroaromatic ring having 1-5 heteroatoms independently selected from nitrogen, oxygen, and sulfur. In some embodiments, Cy′″ is an optionally substituted 5-12 membered saturated or partially unsaturated bicyclic carbocyclic ring that is optionally bridged or spirocyclic. In some embodiments, Cy′″ is an optionally substituted 5-12 membered saturated or partially unsaturated bicyclic heterocyclic ring that is optionally bridged or spirocyclic having 1-4 heteroatoms independently selected from nitrogen, oxygen, and sulfur. In some embodiments, Cy′″ is an optionally substituted 8-10 membered bicyclic aromatic carbocyclic ring.

In some embodiments, Cy′″ is as selected from one of the substituents of Table 1.

As described generally above, each R is independently hydrogen, —C(O)N(CH₃)₂, —C(O)₂CH₃, —C(O)₂C(CH₃)₃, —C(O)₂CH(CH₃)₂, —S(O)₂CH₃, an optionally substituted C₁₋₆ aliphatic group, an optionally substituted cyclic group selected from:
phenyl,
an optionally substituted 3-7 membered saturated or partially unsaturated carbocyclic ring,
an optionally substituted 3-7 membered saturated or partially unsaturated heterocyclic ring having 1-2 heteroatoms independently selected from nitrogen, oxygen, and sulfur,
or an optionally substituted 5-6 membered heteroaryl ring having 1-4 heteroatoms independently selected from nitrogen, oxygen, and sulfur; and/or
   two R groups on the same atom are taken together with the same atom to form a cyclic group selected from:
an optionally substituted 4-7 membered saturated or partially unsaturated carbocycyl,
an optionally substituted 4-7 membered saturated or partially unsaturated heterocyclyl having 1-3 heteroatoms independently selected from nitrogen, oxygen, and sulfur, and
an optionally substituted 5-6 membered heteroaryl ring having 1-3 heteroatoms independently selected from nitrogen, oxygen, and sulfur.

In some embodiments, each R is hydrogen. In some embodiments each R is independently —C(O)N(CH₃)₂, —C(O)₂CH₃, —C(O)₂C(CH₃)₃, —C(O)₂CH(CH₃)₂, or —S(O)₂CH₃. In some embodiments each R is independently an optionally substituted C₁₋₆ aliphatic group or an optionally substituted cyclic group selected from:
an optionally substituted phenyl,
an optionally substituted 3-7 membered saturated or partially unsaturated carbocyclic ring,
an optionally substituted 3-7 membered saturated or partially unsaturated heterocyclic ring having 1-2 heteroatoms independently selected from nitrogen, oxygen, and sulfur, or
an optionally substituted 5-6 membered heteroaryl ring having 1-4 heteroatoms independently selected from nitrogen, oxygen, and sulfur.

In some embodiments, two R groups on the same atom are taken together with the same atom to form a cyclic group selected from:
an optionally substituted 4-7 membered saturated or partially unsaturated carbocycyl,
an optionally substituted 4-7 membered saturated or partially unsaturated heterocyclyl having 1-3 heteroatoms independently selected from nitrogen, oxygen, and sulfur, and
an optionally substituted 5-6 membered heteroaryl ring having 1-3 heteroatoms independently selected from nitrogen, oxygen, and sulfur.

In some embodiments, each R is as selected from the substituents of Table 1.

As described generally above, x is 0, 1, 2, 3, or 4. In some embodiments, x is 0. In some embodiments, x is 1. In some embodiments, x is 2. In some embodiments, x is 3. In some embodiments, x is 4. In some embodiments, x is as selected from one of the substituents of Table 1.

As described generally above, m is 0, 1, 2, or 3. In some embodiments, m is 0. In some embodiments, m is 1. In some embodiments, m is 2. In some embodiments, m is 3. In some embodiments, m is as selected from one of the substituents of Table 1.

As described generally above, n is 1 or 2. In some embodiments, n is 1. In some embodiments, n is 2. In some embodiments, n is as selected from one of the substituents of Table 1.

In some embodiments, n is 1 and m is 0. In some embodiments, n is 1 and m is 1. In some embodiments, n is 1, m is 0, and x is 0. In some embodiments, n is 1, m is 1, and x is 0. In some embodiments, n is 1, m is 0, and x is 1. In some embodiments, n is 1, m is 1, and x is 1.

As described generally above, y is 0, 1, 2, or 3. In some embodiments, y is 0. In some embodiments, y is 1. In some embodiments, y is 2. In some embodiments, y is 3.

In some embodiments, y is as selected from one of the substituents of Table 1.

As described generally above, z is 0, 1, 2, or 3. In some embodiments, z is 0. In some embodiments, z is 1. In some embodiments, z is 2. In some embodiments, z is 3.

In some embodiments, z is as selected from one of the substituents of Table 1.

In some embodiments, the compound of Formula I is a compound of Formula II:

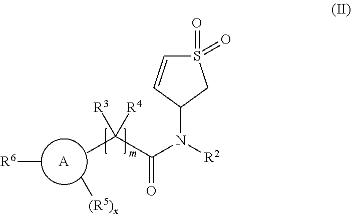

wherein Ring A, R², R³, R⁴, R⁵, R⁶, m, and x are as defined herein, both singly and in combination.

In some embodiments, the compound of Formula I is a compound of Formula IIIa or IIIb:

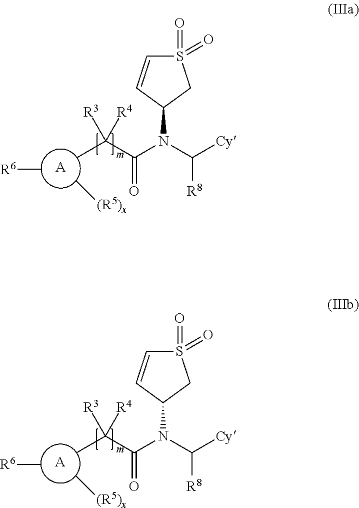

wherein Ring A, Cy′, R³, R⁴, R⁵, R⁶, m, and x are as defined herein, both singly and in combination, and wherein R⁸ is hydrogen, optionally substituted C₁₋₆ aliphatic group, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, or —N(R)S(O)₂R. In certain embodiments, the compound of Formula IIIa or IIIb includes compounds in which R⁸ is hydrogen, —CH₃ or —CH₂CH₃.

In some embodiments, the compound of Formula I is a compound of Formula IVa or IVb:

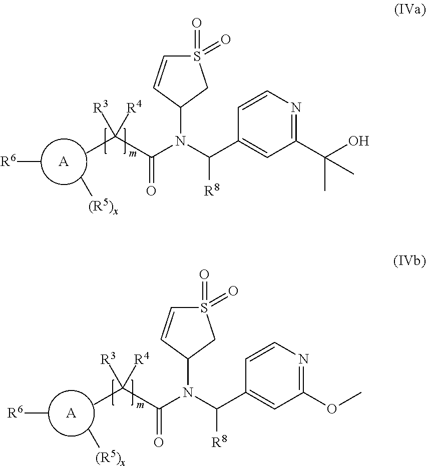

wherein Ring A, R³, R⁴, R⁵, R⁶, m, and x are as defined herein, both singly and in combination, and wherein R¹ is hydrogen, optionally substituted C₁₋₆ aliphatic group, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, or —N(R)S(O)₂R. In certain embodiments, the compound of Formula IVa or IVb includes compounds in which R⁸ is hydrogen, —CH₃ or —CH₂CH₃.

In some embodiments, the compound of Formula I is a compound of Formula Va or Vb:

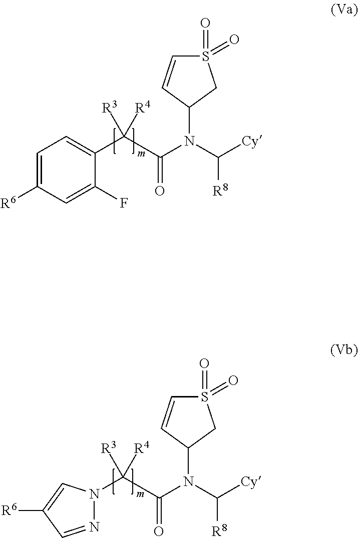

wherein Cy′, R³, R⁴, R⁶, and m are as defined herein, both singly and in combination, and wherein R⁸ is hydrogen, optionally substituted C₁₋₆ aliphatic group, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, or —N(R)S(O)₂R. In certain embodiments, the compound of Formula Va or Vb includes compounds in which R⁸ is hydrogen, —CH₃ or —CH₂CH₃. In some embodiments, the compound is of Formula Va or Vb and m is 0. In some embodiments, the compound is of Formula Va or Vb and m is 1.

In some embodiments, the compound of Formula I is selected from Table 1. Table 1 identifies compounds by their IUPAC name and Table 2 lists the same compounds and shows their chemical structure. In the event of any discrepancy between Table 1's name for a compound and Table 2's structure for that same compound, Table 2's compound structures will identify the compound corresponding to each respective compound number (I-#) in Table 1. Each stereocenter in the IUPAC names below marked with “*” or “*” refers to “or1” or “or2,” respectively, as described below in the description of Table 2.

TABLE 1

| Cmpd. No. | IUPAC Name |
|---|---|
| I-1 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[4-(3,4,5- |
| | trifluorophenyl)-1H-pyrazol-1-yl]acetamide |
| I-2 | (2R*)-N-[(3R)-1,1-dioxo-2,3-dihydro-1lambda6-thiophen-3-yl]- |
| | N-{[2-(2-hydroxypropan-2-yl)pyridin-4-yl]methyl}-2-methoxy-2- |
| | {3,3′,4′-trifluoro-[1,1′-biphenyl]-4-yl}acetamide |
| I-3 | N-[(3R)-1,1-dioxo-2,3-dihydro-1lambda6-thiophen-3-yl]-N-{[2- |
| | (2-hydroxypropan-2-yl)pyridin-4-yl]methyl}-N′-methyl-2- |
| | {3,3′,4′-trifluoro-[1,1′-biphenyl]-4-yl}propanediamide |
| I-4 | rel-N-[(4-cyclopropyl-1,3-thiazol-2-yl)methyl]-N-[(3R)-1,1- |
| | dioxo-2,3-dihydro-1lambda6-thiophen-3-yl]-2-[4-(pentafluoro- |
| | lambda6-sulfanyl)phenyl]acetamide |
| I-5 | rel-N-[(4-cyclopropyl-1,3-thiazol-2-yl)methyl]-2-[4-(3,4- |
| | difluorophenyl)-1H-pyrazol-1-yl]-N-[(3R)-1,1-dioxo-2,3- |
| | dihydro-1lambda6-thiophen-3-yl]acetamide |
| I-6 | 2-[4-(3,4-difluorophenyl)-1H-pyrazol-1-yl]-N-[(3R)-1,1-dioxo- |
| | 2,3-dihydro-1lambda6-thiophen-3-yl]-N-{[2-(2-hydroxypropan- |
| | 2-yl)pyridin-4-yl]methyl}acetamide |
| I-7 | N-[(3R)-1,1-dioxo-2,3-dihydro-1lambda6-thiophen-3-yl]-N-{[2- |
| | (2-hydroxypropan-2-yl)pyridin-4-yl]methyl}-2-[5-(pentafluoro- |
| | lambda6-sulfanyl)-2H-indazol-2-yl]acetamide |
| I-8 | (2R**)-N-[(3R*)-1,1-dioxo-2,3-dihydro-1lambda6-thiophen-3- |
| | yl]-2-{4′-fluoro-[1,1′-biphenyl]-4-yl}-N-{[2-(2-hydroxypropan-2- |
| | yl)pyridin-4-yl]methyl}-N′,N′-dimethylpropanediamide |
| I-9 | (2S)-2-{3,4′-difluoro-[1,1′-biphenyl]-4-yl}-N-[(3R)-1,1-dioxo- |
| | 2,3-dihydro-1lambda6-thiophen-3-yl]-N-[(2-methoxypyridin-4- |
| | yl)methyl]-N′,N′-dimethylpropanediamide |
| I-10 | (2R*)-2-{3,4′-difluoro-[1,1′-biphenyl]-4-yl}-N-[(3R**)-1,1- |
| | dioxo-2,3-dihydro-1lambda6-thiophen-3-yl]-N-{[2-(2- |
| | hydroxypropan-2-yl)pyridin-4-yl]methyl}-2-methoxyacetamide |
| I-11 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-{3,3′,4′- |
| | trifluoro-[1,1′-biphenyl]-4-yl}acetamide |
| I-12 | (2R**)-2-{3,4′-difluoro-[1,1′-biphenyl]-4-yl}-N-[(3R*)-1,1- |
| | dioxo-2,3-dihydro-1lambda6-thiophen-3-yl]-N-{[2-(2- |
| | hydroxypropan-2-yl)pyridin-4-yl]methyl}-N′,N′- |
| | dimethylpropanediamide |
| I-13 | 2-[4-(3,4-difluorophenyl)-2-oxo-1,2-dihydropyridin-1-yl]-N- |
| | [(3R)-1,1-dioxo-2,3-dihydro-1lambda6-thiophen-3-yl]-N-{[2-(2- |
| | hydroxypropan-2-yl)pyridin-4-yl]methyl}acetamide |
| I-14 | N-[(4-cyclobutyl-1,3-thiazol-2-yl)methyl]-N-(1,1-dioxo-2,3- |
| | dihydro-1lambda6-thiophen-3-yl)-2-[4-(pentafluoro-lambda6- |
| | sulfanyl)phenyl]acetamide |
| I-15 | N-[(3R)-1,1-dioxo-2,3-dihydro-1lambda6-thiophen-3-yl]-2-[4-(4- |
| | fluorophenyl)-1H-pyrazol-1-yl]-N-{[2-(2-hydroxypropan-2- |
| | yl)pyridin-4-yl]methyl}acetamide |
| I-16 | N-[(3R)-1,1-dioxo-2,3-dihydro-1lambda6-thiophen-3-yl]-N-{[2- |
| | (2-hydroxypropan-2-yl)pyridin-4-yl]methyl}-2-[4-(pentafluoro- |
| | lambda6-sulfanyl)phenyl]acetamide |
| I-17 | N-[(3R)-1,1-dioxo-2,3-dihydro-1lambda6-thiophen-3-yl]-N-[(2- |
| | methoxypyridin-4-yl)methyl]-2-[4-(pentafluoro-lambda6- |
| | sulfanyl)phenyl]acetamide |
| I-18 | N-(1,1-dioxo-2,3-dihydro-1lambda6-thiophen-3-yl)-N-{[2-(2- |
| | hydroxypropan-2-yl)pyridin-4-yl]methyl}-2-methoxy-2-{3,3′,4′- |
| | trifluoro-[1,1′-biphenyl]-4-yl}acetamide |
| I-19 | N-(1,1-dioxo-2,3-dihydro-1lambda6-thiophen-3-yl)-N-{[2-(2- |
| | hydroxypropan-2-yl)pyridin-4-yl]methyl}-2-{3,3′,4′,5′- |
| | tetrafluoro-[1,1′-biphenyl]-4-yl}acetamide |
| I-20 | N′-[(3R)-1,1-dioxo-2,3-dihydro-1lambda6-thiophen-3-yl]-N′-{[2- |
| | (2-hydroxypropan-2-yl)pyridin-4-yl]methyl}-N-(2- |
| | methoxyethyl)-N-methyl-2-{3,3′,4′-trifluoro-[1,1′-biphenyl]-4- |
| | yl}propanediamide |
| I-21 | 2-{3,4′-difluoro-[1,1′-biphenyl]-4-yl}-N-(1,1-dioxo-2,3-dihydro- |
| | 1lambda6-thiophen-3-yl)-N-{[2-(2-hydroxypropan-2-yl)pyridin- |
| | 4-yl]methyl}-N′,N′-dimethylpropanediamide |
| I-22 | N-(1,1-dioxo-2,3-dihydro-1lambda6-thiophen-3-yl)-2-[2-fluoro- |
| | 4-(pentafluoro-lambda6-sulfanyl)phenyl]-N-[(2-methoxypyridin- |
| | 4-yl)methyl]acetamide |
| I-23 | N′-[(4-cyclopropyl-1,3-thiazol-2-yl)methyl]-2-{3,4′-difluoro- |
| | [1,1′-biphenyl]-4-yl}-N′-(1,1-dioxo-2,3-dihydro-1lambda6- |
| | thiophen-3-yl)-N,N-dimethylpropanediamide |
| I-24 | N′-(1,1-dioxo-2,3-dihydro-1lambda6-thiophen-3-yl)-N′-{[2-(2- |
| | hydroxypropan-2-yl)pyridin-4-yl]methyl}-N,N-dimethyl-2- |
| | {3,3′,4′-trifluoro-[1,1′-biphenyl]-4-yl}propanediamide |
| I-25 | N′-[(4-cyclopropyl-1,3-thiazol-2-yl)methyl]-N′-(1,1-dioxo-2,3- |
| | dihydro-1lambda6-thiophen-3-yl)-N,N-dimethyl-2-{3,3′,4′- |
| | trifluoro-[1,1′-biphenyl]-4-yl}propanediamide |
| I-26 | N-[(3R)-1,1-dioxo-2,3-dihydro-1lambda6-thiophen-3-yl]-2-[4-(4- |
| | fluorophenyl)-1H-pyrazol-1-yl]-N-[(2-methoxypyridin-4- |
| | yl)methyl]acetamide |
| I-27 | (2R**)-N′-[(3R*)-1,1-dioxo-2,3-dihydro-1lambda6-thiophen-3- |
| | yl]-2-{4′-fluoro-[1,1′-biphenyl]-4-yl}-N,N-dimethyl-N′-[(pyridin- |
| | 4-yl)methyl]propanediamide |
| I-28 | 2-{3,4′-difluoro-[1,1′-biphenyl]-4-yl}-N-(1,1-dioxo-2,3-dihydro- |
| | 1lambda6-thiophen-3-yl)-N-{[2-(2-hydroxypropan-2-yl)pyridin- |
| | 4-yl]methyl}-2-methoxyacetamide |
| I-29 | N-(1,1-dioxo-2,3-dihydro-1lambda6-thiophen-3-yl)-N-[(2- |
| | methoxypyridin-4-yl)methyl]-2-[4-(pentafluoro-lambda6- |
| | sulfanyl)phenyl]acetamide |
| I-30 | N-[(3R)-1,1-dioxo-2,3-dihydro-1lambda6-thiophen-3-yl]-N-{[2- |
| | (2-hydroxypropan-2-yl)pyridin-4-yl]methyl}-2-(2- |
| | methoxyethoxy)-2-{3,3′,4′-trifluoro-[1,1′-biphenyl]-4- |
| | yl}acetamide |
| I-31 | rel-N-[(3R)-1,1-dioxo-2,3-dihydro-1lambda6-thiophen-3-yl]-2- |
| | [2-fluoro-4-(2-methyl-1H-pyrrol-1-yl)phenyl]-N-[(2- |
| | methoxypyridin-4-yl)methyl]acetamide |
| I-32 | N-(1,1-dioxo-2,3-dihydro-1lambda6-thiophen-3-yl)-N-{[2-(2- |
| | hydroxypropan-2-yl)pyridin-4-yl]methyl}-2-{3,3′,4′-trifluoro- |
| | [1,1′-biphenyl]-4-yl}acetamide |
| I-33 | (2R*)-2-{3,4′-difluoro-[1,1′-biphenyl]-4-yl}-N-[(3R**)-1,1- |
| | dioxo-2,3-dihydro-1lambda6-thiophen-3-yl]-2-methoxy-N-[(2- |
| | methoxypyridin-4-yl)methyl]acetamide |
| I-34 | N-(1,1-dioxo-2,3-dihydro-1lambda6-thiophen-3-yl)-2-{4′-fluoro- |
| | [1,1′-biphenyl]-4-yl}-N-{[2-(2-hydroxypropan-2-yl)pyridin-4- |
| | yl]methyl}-N′,N′-dimethylpropanediamide |
| I-35 | rel-N-[(3R)-1,1-dioxo-2,3-dihydro-1lambda6-thiophen-3-yl]-N- |
| | [(2-methoxypyridin-4-yl)methyl]-2-{3,3′,4′-trifluoro-[1,1′- |
| | biphenyl]-4-yl}acetamide |
| I-36 | N-(1,1-dioxo-2,3-dihydro-1lambda6-thiophen-3-yl)-N-{[2-(2- |
| | hydroxypropan-2-yl)pyridin-4-yl]methyl}-2-(methylsulfanyl)-2- |
| | {3,3′,4′-trifluoro-[1,1′-biphenyl]-4-yl}acetamide |
| I-37 | N′-(1,1-dioxo-2,3-dihydro-1lambda6-thiophen-3-yl)-N′-[(2- |
| | methoxypyridin-4-yl)methyl]-N,N-dimethyl-2-{3,3′,4′-trifluoro- |
| | [1,1′-biphenyl]-4-yl}propanediamide |
| I-38 | N-[(3R)-1,1-dioxo-2,3-dihydro-1lambda6-thiophen-3-yl]-2,2- |
| | difluoro-N-{[2-(2-hydroxypropan-2-yl)pyridin-4-yl]methyl}-2- |
| | {3,3′,4′-trifluoro-[1,1′-biphenyl]-4-yl}acetamide |
| I-39 | N-(1,1-dioxo-2,3-dihydro-1lambda6-thiophen-3-yl)-N-{[2-(2- |
| | hydroxypropan-2-yl)pyridin-4-yl]methyl}-2-[4-(pentafluoro- |
| | lambda6-sulfanyl)phenyl]acetamide |
| I-40 | rel-N-[(3R)-1,1-dioxo-2,3-dihydro-1lambda6-thiophen-3-yl]-2- |
| | [2-fluoro-4-(1H-pyrrol-1-yl)phenyl]-N-{[2-(2-hydroxypropan-2- |
| | yl)pyridin-4-yl]methyl}acetamide |
| I-41 | N-[(4-cyclopropyl-1,3-thiazol-2-yl)methyl]-N-(1,1-dioxo-2,3- |
| | dihydro-1lambda6-thiophen-3-yl)-2-[4-(pentafluoro-lambda6- |
| | sulfanyl)phenyl]acetamide |
| I-42 | N-(1,1-dioxo-2,3-dihydro-1lambda6-thiophen-3-yl)-2-[4-(4- |
| | fluorophenyl)-1H-pyrazol-1-yl]-N-{[2-(2-hydroxypropan-2- |
| | yl)pyridin-4-yl]methyl}acetamide |
| I-43 | N-[(3R)-1,1-dioxo-2,3-dihydro-1lambda6-thiophen-3-yl]-N-{[2- |
| | (2-hydroxypropan-2-yl)pyridin-4-yl]methyl}-2-(4-phenyl-1H- |
| | pyrazol-1-yl)acetamide |
| I-44 | N-(1,1-dioxo-2,3-dihydro-1lambda6-thiophen-3-yl)-2-[2-fluoro- |
| | 4-(pentafluoro-lambda6-sulfanyl)phenyl]-N-{[2-(2- |
| | hydroxypropan-2-yl)pyridin-4-yl]methyl}acetamide |
| I-45 | N-(1,1-dioxo-2,3-dihydro-1lambda6-thiophen-3-yl)-2-methoxy- |
| | N-[(2-methoxypyridin-4-yl)methyl]-2-{3,3′,4′-trifluoro-[1,1′- |
| | biphenyl]-4-yl}acetamide |
| I-46 | 2-{3,4′-difluoro-[1,1′-biphenyl]-4-yl}-N-(1,1-dioxo-2,3-dihydro- |
| | 1lambda6-thiophen-3-yl)-N-[(2-methoxypyridin-4-yl)methyl]- |
| | N′,N′-dimethylpropanediamide |
| I-47 | N-[(4-cyclopropyl-1,3-thiazol-2-yl)methyl]-N-(1,1-dioxo-2,3- |
| | dihydro-1lambda6-thiophen-3-yl)-2-methoxy-2-{3,3′,4′-trifluoro- |
| | [1,1′-biphenyl]-4-yl}acetamide |
| I-48 | N-(1,1-dioxo-2,3-dihydro-1lambda6-thiophen-3-yl)-2-(1H- |
| | pyrazol-1-yl)-N-[(pyridin-4-yl)methyl]-2-{3,3′,4′-trifluoro-[1,1′- |
| | biphenyl]-4-yl}acetamide |
| I-49 | N-[(4-cyclopropyl-1,3-thiazol-2-yl)methyl]-N-(1,1-dioxo-2,3- |
| | dihydro-1lambda6-thiophen-3-yl)-2-[4-(4-fluorophenyl)-1H- |
| | pyrazol-1-yl]acetamide |
| I-50 | rel-2-[5-(3,4-difluorophenyl)-3-fluoropyridin-2-yl]-N-[(3R)-1,1- |
| | dioxo-2,3-dihydro-1lambda6-thiophen-3-yl]-N-{[2-(2- |
| | hydroxypropan-2-yl)pyridin-4-yl]methyl}acetamide |
| I-51 | rel-N-[(3R)-1,1-dioxo-2,3-dihydro-1lambda6-thiophen-3-yl]-2- |
| | [4-(pentafluoro-lambda6-sulfanyl)phenyl]-N-[(pyridin-4- |
| | yl)methyl]acetamide |
| I-52 | 2-{3,4′-difluoro-[1,1′-biphenyl]-4-yl}-N-(1,1-dioxo-2,3-dihydro- |
| | 1lambda6-thiophen-3-yl)-N-{[2-(2-hydroxypropan-2-yl)pyridin- |
| | 4-yl]methyl}acetamide |
| I-53 | N-[(3R)-1,1-dioxo-2,3-dihydro-1lambda6-thiophen-3-yl]-6-(4- |
| | fluorophenyl)-N-[(2-methoxypyridin-4-yl)methyl]-2,3-dihydro- |
| | 1H-indole-3-carboxamide |
| I-54 | N-(1,1-dioxo-2,3-dihydro-1lambda6-thiophen-3-yl)-2-[4-(3- |
| | fluorophenyl)-1H-pyrazol-1-yl]-N-[(2-methoxypyridin-4- |
| | yl)methyl]acetamide |
| I-55 | N-(1,1-dioxo-2,3-dihydro-1lambda6-thiophen-3-yl)-2-{4′-fluoro- |
| | [1,1′-biphenyl]-4-yl}-N-[(2-methoxypyridin-4-yl)methyl]-N′,N′- |
| | dimethylpropanediamide |
| I-56 | N-[(3R)-1,1-dioxo-2,3-dihydro-1lambda6-thiophen-3-yl]-2-[2- |
| | fluoro-4-(2-methyl-1H-pyrrol-1-yl)phenyl]-N-[(2- |
| | methoxypyridin-4-yl)methyl]acetamide |
| I-57 | rel-N-[(3R)-1,1-dioxo-2,3-dihydro-1lambda6-thiophen-3-yl]-N- |
| | [(2-methoxypyridin-4-yl)methyl]-2-[4- |
| | (trifluoromethyl)phenyl]acetamide |
| I-58 | 2-[4-(4-chlorophenyl)-1H-pyrazol-1-yl]-N-(1,1-dioxo-2,3- |
| | dihydro-1lambda6-thiophen-3-yl)-N-{[2-(2-hydroxypropan-2- |
| | yl)pyridin-4-yl]methyl}acetamide |
| I-59 | N-(1,1-dioxo-2,3-dihydro-1lambda6-thiophen-3-yl)-2-[4-(3- |
| | fluorophenyl)-1H-pyrazol-1-yl]-N-{[2-(2-hydroxypropan-2- |
| | yl)pyridin-4-yl]methyl}acetamide |
| I-60 | N-[(4-cyclopropyl-1,3-thiazol-2-yl)methyl]-N-(1,1-dioxo-2,3- |
| | dihydro-1lambda6-thiophen-3-yl)-2-[2-fluoro-4-(2-methyl-1H- |
| | pyrrol-1-yl)phenyl]acetamide |
| I-61 | N-(1,1-dioxo-2,3-dihydro-1lambda6-thiophen-3-yl)-2-[2-fluoro- |
| | 4-(1H-pyrrol-1-yl)phenyl]-N-{[2-(2-hydroxypropan-2-yl)pyridin- |
| | 4-yl]methyl}acetamide |
| I-62 | N′-(1,1-dioxo-2,3-dihydro-1lambda6-thiophen-3-yl)-N,N-diethyl- |
| | N′-{[2-(2-hydroxypropan-2-yl)pyridin-4-yl]methyl}-2-{3,3′,4′- |
| | trifluoro-[1,1′-biphenyl]-4-yl}propanediamide |
| I-63 | N-(1,1-dioxo-2,3-dihydro-1lambda6-thiophen-3-yl)-2-[4- |
| | (pentafluoro-lambda6-sulfanyl)phenyl]-N-[(pyridin-4- |
| | yl)methyl]acetamide |
| I-64 | N-(1,1-dioxo-2,3-dihydro-1lambda6-thiophen-3-yl)-N-[(2- |
| | methoxypyridin-4-yl)methyl]-2-{3,3′,4′-trifluoro-[1,1′-biphenyl]- |
| | 4-yl}acetamide |
| I-65 | N-(1,1-dioxo-2,3-dihydro-1lambda6-thiophen-3-yl)-2-[2-fluoro- |
| | 4-(2-methyl-1H-pyrrol-1-yl)phenyl]-N-{[2-(1- |
| | hydroxycyclobutyl)pyridin-4-yl]methyl}acetamide |
| I-66 | N-(1,1-dioxo-2,3-dihydro-1lambda6-thiophen-3-yl)-2-[2-fluoro- |
| | 4-(2-methyl-1H-pyrrol-1-yl)phenyl]-N-{[2-(2-hydroxypropan-2- |
| | yl)pyridin-4-yl]methyl}acetamide |
| I-67 | N-(1,1-dioxo-2,3-dihydro-1lambda6-thiophen-3-yl)-2-[2-fluoro- |
| | 4-(2-methyl-1H-pyrrol-1-yl)phenyl]-N-{[2-(2,2,2- |
| | trifluoroethoxy)pyridin-4-yl]methyl}acetamide |
| I-68 | N-(1,1-dioxo-2,3-dihydro-1lambda6-thiophen-3-yl)-2-[4-(4- |
| | fluorophenyl)-1H-pyrazol-1-yl]-N-[(2-methoxypyridin-4- |
| | yl)methyl]acetamide |
| I-69 | N-(1,1-dioxo-2,3-dihydro-1lambda6-thiophen-3-yl)-2-[2-fluoro- |
| | 4-(2-methyl-1H-pyrrol-1-yl)phenyl]-N-[(2-methoxypyridin-4- |
| | yl)methyl]acetamide |
| I-70 | 2-{3′-chloro-3-fluoro-[1,1′-biphenyl]-4-yl}-N-(1,1-dioxo-2,3- |
| | dihydro-1lambda6-thiophen-3-yl)-N-{[2-(2-hydroxypropan-2- |
| | yl)pyridin-4-yl]methyl}acetamide |
| I-71 | 2-{3,4′-difluoro-[1,1′-biphenyl]-4-yl}-N-(1,1-dioxo-2,3-dihydro- |
| | 1lambda6-thiophen-3-yl)-2-methoxy-N-[(2-methoxypyridin-4- |
| | yl)methyl]acetamide |
| I-72 | (2R*)-N-[(3R**)-1,1-dioxo-2,3-dihydro-1lambda6-thiophen-3- |
| | yl]-2-{4′-fluoro-[1,1′-biphenyl]-4-yl}-2-methoxy-N-[(pyridin-4- |
| | yl)methyl]acetamide |
| I-73 | N-[(1,3-benzothiazol-2-yl)methyl]-N-(1,1-dioxo-2,3-dihydro- |
| | 1lambda6-thiophen-3-yl)-2-[2-fluoro-4-(2-methyl-1H-pyrrol-1- |
| | yl)phenyl]acetamide |
| I-74 | N-[(4-cyclopropyl-1,3-thiazol-2-yl)methyl]-N-(1,1-dioxo-2,3- |
| | dihydro-1lambda6-thiophen-3-yl)-2-methoxy-2-[4-(pentafluoro- |
| | lambda6-sulfanyl)phenyl]acetamide |
| I-75 | N′-(1,1-dioxo-2,3-dihydro-1lambda6-thiophen-3-yl)-N-ethyl-N′- |
| | {[2-(2-hydroxypropan-2-yl)pyridin-4-yl]methyl}-N-methyl-2- |
| | {3,3′,4′-trifluoro-[1,1′-biphenyl]-4-yl}propanediamide |
| I-76 | N-(1,1-dioxo-2,3-dihydro-1lambda6-thiophen-3-yl)-N-{[2-(2- |
| | hydroxypropan-2-yl)pyridin-4-yl]methyl}-2-(4-phenyl-1H- |
| | pyrazol-1-yl)acetamide |
| I-77 | N-(1,1-dioxo-2,3-dihydro-1lambda6-thiophen-3-yl)-2-fluoro-N- |
| | {[2-(2-hydroxypropan-2-yl)pyridin-4-yl]methyl}-2-{3,3′,4′- |
| | trifluoro-[1,1′-biphenyl]-4-yl}acetamide |
| I-78 | N-(1,1-dioxo-2,3-dihydro-1lambda6-thiophen-3-yl)-2-[2-fluoro- |
| | 4-(2-methyl-1H-pyrrol-1-yl)phenyl]-N-({7-hydroxy-7-methyl- |
| | 5H,6H,7H-cyclopenta[b]pyridin-4-yl}methyl)acetamide |
| I-79 | 2-{3,4′-difluoro-[1,1′-biphenyl]-4-yl}-N-(1,1-dioxo-2,3-dihydro- |
| | 1lambda6-thiophen-3-yl)-N-{[4-(2-hydroxypropan-2-yl)-1,3- |
| | thiazol-2-yl]methyl}acetamide |
| I-80 | N-[(4-cyclopropyl-1,3-thiazol-2-yl)methyl]-2-{3,4′-difluoro-[1,1′- |
| | biphenyl]-4-yl}-N-(1,1-dioxo-2,3-dihydro-1lambda6-thiophen-3- |
| | yl)acetamide |
| I-81 | N-(1,1-dioxo-2,3-dihydro-1lambda6-thiophen-3-yl)-N-{[2-(2- |
| | hydroxypropan-2-yl)pyridin-4-yl]methyl}-2-methoxy-2-[4- |
| | (pentafluoro-lambda6-sulfanyl)phenyl]acetamide |
| I-82 | 2-[5-(3,4-difluorophenyl)-3-fluoropyridin-2-yl]-N-(1,1-dioxo-2,3- |
| | dihydro-1lambda6-thiophen-3-yl)-N-{[2-(2-hydroxypropan-2- |
| | yl)pyridin-4-yl]methyl}acetamide |
| I-83 | N-[(4-cyclopropyl-1,3-thiazol-2-yl)methyl]-N-[(3R)-1,1-dioxo- |
| | 2,3-dihydro-1lambda6-thiophen-3-yl]-2-[4-(1H-pyrrol-1- |
| | yl)phenyl]acetamide |
| I-84 | 2-{3,4′-difluoro-[1,1′-biphenyl]-4-yl}-N-(1,1-dioxo-2,3-dihydro- |
| | 1lambda6-thiophen-3-yl)-N-[(2-ethoxypyridin-4- |
| | yl)methyl]acetamide |
| I-85 | 2-[4-(4-chlorophenyl)-1H-pyrazol-1-yl]-N-(1,1-dioxo-2,3- |
| | dihydro-1lambda6-thiophen-3-yl)-N-[(2-methoxypyridin-4- |
| | yl)methyl]acetamide |
| I-86 | N-(1,1-dioxo-2,3-dihydro-1lambda6-thiophen-3-yl)-2-{4′-fluoro- |
| | [1,1′-biphenyl]-4-yl}-2-methoxy-N-[(2-methoxypyridin-4- |
| | yl)methyl]acetamide |
| I-87 | 2-{3,4′-difluoro-[1,1′-biphenyl]-4-yl}-N-(1,1-dioxo-2,3-dihydro- |
| | 1lambda6-thiophen-3-yl)-N-[(2-methoxypyridin-4- |
| | yl)methyl]acetamide |
| I-88 | N-[(4-cyclopropyl-1,3-thiazol-2-yl)methyl]-N-(1,1-dioxo-2,3- |
| | dihydro-1lambda6-thiophen-3-yl)-2-{4′-fluoro-[1,1′-biphenyl]-4- |
| | yl}acetamide |
| I-89 | N-{[2-(dimethylamino)pyridin-4-yl]methyl}-N-(1,1-dioxo-2,3- |
| | dihydro-1lambda6-thiophen-3-yl)-2-[4-(pentafluoro-lambda6- |
| | sulfanyl)phenyl]acetamide |
| I-90 | N-(1,1-dioxo-2,3-dihydro-1lambda6-thiophen-3-yl)-2-[2-fluoro- |
| | 4-(2-methyl-1H-pyrrol-1-yl)phenyl]-N-{[2-(1- |
| | hydroxycyclopropyl)pyridin-4-yl]methyl}acetamide |
| I-91 | 2-{3,3′-difluoro-[1,1′-biphenyl]-4-yl}-N-(1,1-dioxo-2,3-dihydro- |
| | 1lambda6-thiophen-3-yl)-N-[(2-methoxypyridin-4- |
| | yl)methyl]acetamide |
| I-92 | N-(1,1-dioxo-2,3-dihydro-1lambda6-thiophen-3-yl)-2-methoxy- |
| | N-[(2-methoxypyridin-4-yl)methyl]-2-[4-(pentafluoro-lambda6- |
| | sulfanyl)phenyl]acetamide |
| I-93 | N-(1,1-dioxo-2,3-dihydro-1lambda6-thiophen-3-yl)-N-[(2- |
| | methoxypyridin-4-yl)methyl]-3-(1H-pyrrol-1- |
| | yl)bicyclo[4.2.0]octa-1,3,5-triene-7-carboxamide |
| I-94 | N-(1,1-dioxo-2,3-dihydro-1lambda6-thiophen-3-yl)-N-[(2- |
| | methoxypyridin-4-yl)methyl]-2-(4-phenyl-1H-pyrazol-1- |
| | yl)acetamide |
| I-95 | N-(1,1-dioxo-2,3-dihydro-1lambda6-thiophen-3-yl)-2-[2-fluoro- |
| | 4-(1H-pyrrol-1-yl)phenyl]-N-[(2-methoxypyridin-4- |
| | yl)methyl]acetamide |
| I-96 | N-{[2-(dimethylamino)pyridin-4-yl]methyl}-N-(1,1-dioxo-2,3- |
| | dihydro-1lambda6-thiophen-3-yl)-2-[2-fluoro-4-(2-methyl-1H- |
| | pyrrol-1-yl)phenyl]acetamide |
| I-97 | N-(1,1-dioxo-2,3-dihydro-1lambda6-thiophen-3-yl)-2-[2-fluoro- |
| | 4-(1-methylcyclopropyl)phenyl]-N-[(2-methoxypyridin-4- |
| | yl)methyl]acetamide |
| I-98 | N-(1,1-dioxo-2,3-dihydro-1lambda6-thiophen-3-yl)-6-(4- |
| | fluorophenyl)-N-{[2-(2-hydroxypropan-2-yl)pyridin-4- |
| | yl]methyl}-2,3-dihydro-1H-indole-3-carboxamide |
| I-99 | N-{[2-(dimethylamino)pyridin-4-yl]methyl}-N-(1,1-dioxo-2,3- |
| | dihydro-1lambda6-thiophen-3-yl)-2-{3,3′,4′-trifluoro-[1,1′- |
| | biphenyl]-4-yl}acetamide |
| I-100 | N-[(4-bromophenyl)methyl]-N-(1,1-dioxo-2,3-dihydro- |
| | 1lambda6-thiophen-3-yl)-2-[2-fluoro-4-(2-methyl-1H-pyrrol-1- |
| | yl)phenyl]acetamide |
| I-101 | 2-{3-chloro-4′-fluoro-[1,1′-biphenyl]-4-yl}-N-(1,1-dioxo-2,3- |
| | dihydro-1lambda6-thiophen-3-yl)-N-{[2-(2-hydroxypropan-2- |
| | yl)pyridin-4-yl]methyl}acetamide |
| I-102 | N-(1,1-dioxo-2,3-dihydro-1lambda6-thiophen-3-yl)-N-[(5- |
| | methoxy-1,3-benzothiazol-2-yl)methyl]-2-[4-(1H-pyrrol-1- |
| | yl)phenyl]acetamide |
| I-103 | N-(1,1-dioxo-2,3-dihydro-1lambda6-thiophen-3-yl)-2-[2-fluoro- |
| | 4-(2-methyl-1H-pyrrol-1-yl)phenyl]-N-({7-hydroxy-5H,6H,7H- |
| | cyclopenta[b]pyridin-4-yl}methyl)acetamide |
| I-104 | N′-(1,1-dioxo-2,3-dihydro-1lambda6-thiophen-3-yl)-2-{4′-fluoro- |
| | [1,1′-biphenyl]-4-yl}-N,N-dimethyl-N′-[(pyridin-4- |
| | yl)methyl]propanediamide |
| I-105 | N-(1,1-dioxo-2,3-dihydro-1lambda6-thiophen-3-yl)-N-[(2- |
| | methoxypyridin-4-yl)methyl]-2-[4- |
| | (trifluoromethyl)phenyl]acetamide |
| I-106 | N-[(3R)-1,1-dioxo-2,3-dihydro-1lambda6-thiophen-3-yl]-2-[4-(4- |
| | fluorophenyl)-1H-pyrazol-1-yl]-N-{[2-(2-hydroxypropan-2- |
| | yl)pyridin-4-yl]methyl}propanamide |
| I-107 | N′-[(4-cyclopropyl-1,3-thiazol-2-yl)methyl]-N′-(1,1-dioxo-2,3- |
| | dihydro-1lambda6-thiophen-3-yl)-N,N-dimethyl-2-[4- |
| | (pentafluoro-lambda6-sulfanyl)phenyl]propanediamide |
| I-108 | N-(1,1-dioxo-2,3-dihydro-1lambda6-thiophen-3-yl)-2-[4-(2- |
| | fluorophenyl)-1H-pyrazol-1-yl]-N-{[2-(2-hydroxypropan-2- |
| | yl)pyridin-4-yl]methyl}acetamide |
| I-109 | N-(1,1-dioxo-2,3-dihydro-1lambda6-thiophen-3-yl)-N-[(2- |
| | methoxypyridin-4-yl)methyl]-5-(2-methyl-1H-pyrrol-1-yl)-2,3- |
| | dihydro-1-benzofuran-2-carboxamide |
| I-110 | N-[(2-cyclopropoxypyridin-4-yl)methyl]-2-{3,4′-difluoro-[1,1′- |
| | biphenyl]-4-yl}-N-(1,1-dioxo-2,3-dihydro-1lambda6-thiophen-3- |
| | yl)acetamide |
| I-111 | N-[(4-cyclopropyl-1,3-thiazol-2-yl)methyl]-N-(1,1-dioxo-2,3- |
| | dihydro-1lambda6-thiophen-3-yl)-2-[4-(1H-pyrrol-1- |
| | yl)phenyl]acetamide |
| I-112 | N-(1,1-dioxo-2,3-dihydro-1lambda6-thiophen-3-yl)-2-[2-fluoro- |
| | 4-(2-methyl-1H-pyrrol-1-yl)phenyl]-N-[(2-methoxypyrimidin-4- |
| | yl)methyl]acetamide |
| I-113 | N-(1,1-dioxo-2,3-dihydro-1lambda6-thiophen-3-yl)-2-ethoxy-2- |
| | {4′-fluoro-[1,1′-biphenyl]-4-yl}-N-[(2-methoxypyridin-4- |
| | yl)methyl]acetamide |
| I-114 | N-[(3R)-1,1-dioxo-2,3-dihydro-1lambda6-thiophen-3-yl]-N-[(2- |
| | methoxypyridin-4-yl)methyl]-5-(trifluoromethyl)-2,3-dihydro-1- |
| | benzofuran-2-carboxamide |
| I-115 | 2-{3,4′-difluoro-[1,1′-biphenyl]-4-yl}-N-(1,1-dioxo-2,3-dihydro- |
| | 1lambda6-thiophen-3-yl)-N-[(pyridin-4-yl)methyl]acetamide |
| I-116 | 2-[5-(3,4-difluorophenyl)pyrimidin-2-yl]-N-(1,1-dioxo-2,3- |
| | dihydro-1lambda6-thiophen-3-yl)-N-{[2-(2-hydroxypropan-2- |
| | yl)pyridin-4-yl]methyl}acetamide |
| I-117 | 2-[4-(1,1-difluoroethyl)phenyl]-N-(1,1-dioxo-2,3-dihydro- |
| | 1lambda6-thiophen-3-yl)-N-[(2-methoxypyridin-4- |
| | yl)methyl]acetamide |
| I-118 | N-(1,1-dioxo-2,3-dihydro-1lambda6-thiophen-3-yl)-2-[2-fluoro- |
| | 4-(2-methyl-1H-pyrrol-1-yl)phenyl]-N-{[2- |
| | (methoxymethyl)pyridin-4-yl]methyl}acetamide |
| I-119 | N-(1,1-dioxo-2,3-dihydro-1lambda6-thiophen-3-yl)-N-[(2- |
| | methoxypyridin-4-yl)methyl]-2-[4-(2-methyl-1H-pyrrol-1- |
| | yl)phenyl]acetamide |
| I-120 | 2-{3,3′-difluoro-[1,1′-biphenyl]-4-yl}-N-(1,1-dioxo-2,3-dihydro- |
| | 1lambda6-thiophen-3-yl)-N-[(pyridin-4-yl)methyl]acetamide |
| I-121 | N-(1,1-dioxo-2,3-dihydro-1lambda6-thiophen-3-yl)-6-(4- |
| | fluorophenyl)-N-[(2-methoxypyridin-4-yl)methyl]-2,3-dihydro- |
| | 1H-indole-3-carboxamide |
| I-122 | N-[(4-bromophenyl)methyl]-N-(1,1-dioxo-2,3-dihydro- |
| | 1lambda6-thiophen-3-yl)-2-[2-fluoro-4-(1H-pyrrol-1- |
| | yl)phenyl]acetamide |
| I-123 | N-(1,1-dioxo-2,3-dihydro-1lambda6-thiophen-3-yl)-N-{[2-(2- |
| | hydroxypropan-2-yl)pyridin-4-yl]methyl}-2-[4-(propan-2- |
| | yl)phenyl]acetamide |
| I-124 | N-(1,1-dioxo-2,3-dihydro-1lambda6-thiophen-3-yl)-6-(4- |
| | fluorophenyl)-N-[(pyridin-4-yl)methyl]-2,3-dihydro-1H-indole-3- |
| | carboxamide |
| I-125 | N-(1,1-dioxo-2,3-dihydro-1lambda6-thiophen-3-yl)-2-{4′-fluoro- |
| | [1,1′-biphenyl]-4-yl}-3-oxo-N-[(pyridin-4-yl)methyl]-3- |
| | (pyrrolidin-1-yl)propanamide |
| I-126 | N-(1,1-dioxo-2,3-dihydro-1lambda6-thiophen-3-yl)-N-[(2- |
| | methoxypyridin-4-yl)methyl]-2-[4-(pentafluoro-lambda6- |
| | sulfanyl)phenyl]propanamide |
| I-127 | rel-N-[(3R)-1,1-dioxo-2,3-dihydro-1lambda6-thiophen-3-yl]-2- |
| | [2-fluoro-4-(1H-pyrrol-1-yl)phenyl]-N-[(pyridin-4- |
| | yl)methyl]acetamide |
| I-128 | N-(1,1-dioxo-2,3-dihydro-1lambda6-thiophen-3-yl)-2-[2-fluoro- |
| | 4-(trifluoromethyl)phenyl]-N-{[2-(2-hydroxypropan-2-yl)pyridin- |
| | 4-yl]methyl}acetamide |
| I-129 | 2-[5-(3,4-difluorophenyl)-3-fluoropyridin-2-yl]-N-(1,1-dioxo-2,3- |
| | dihydro-1lambda6-thiophen-3-yl)-N-[(2-methoxypyridin-4- |
| | yl)methyl]acetamide |
| I-130 | rel-N-[(3R)-1,1-dioxo-2,3-dihydro-1lambda6-thiophen-3-yl]-2- |
| | [6-(4-fluoro-2-hydroxyphenyl)pyridin-3-yl]-N-[(2- |
| | methoxypyridin-4-yl)methyl]acetamide |
| I-131 | N-(1,1-dioxo-2,3-dihydro-1lambda6-thiophen-3-yl)-N-{[2-(2- |
| | hydroxypropan-2-yl)pyridin-4-yl]methyl}-5-(trifluoromethyl)- |
| | 2,3-dihydro-1-benzofuran-2-carboxamide |
| I-132 | N-(1,1-dioxo-2,3-dihydro-1lambda6-thiophen-3-yl)-N-[(2- |
| | methoxypyridin-4-yl)methyl]-3-(2-methyl-1H-pyrrol-1- |
| | yl)bicyclo[4.2.0]octa-1,3,5-triene-7-carboxamide |
| I-133 | rel-N-[(3R)-1,1-dioxo-2,3-dihydro-1lambda6-thiophen-3-yl]-2- |
| | (4-phenyl-1H-pyrazol-1-yl)-N-[(pyridin-4-yl)methyl]acetamide |
| I-134 | N-(1,1-dioxo-2,3-dihydro-1lambda6-thiophen-3-yl)-2-[4-(2- |
| | fluorophenyl)-1H-pyrazol-1-yl]-N-[(2-methoxypyridin-4- |
| | yl)methyl]acetamide |
| I-135 | N-(1,1-dioxo-2,3-dihydro-1lambda6-thiophen-3-yl)-N-{[2-(2- |
| | hydroxypropan-2-yl)pyridin-4-yl]methyl}-2-[4- |
| | (trifluoromethyl)phenyl]acetamide |
| I-136 | 2-{3-chloro-4′-fluoro-[1,1′-biphenyl]-4-yl}-N-(1,1-dioxo-2,3- |
| | dihydro-1lambda6-thiophen-3-yl)-N-[(2-methoxypyridin-4- |
| | yl)methyl]acetamide |
| I-137 | N-[(3R)-1,1-dioxo-2,3-dihydro-1lambda6-thiophen-3-yl]-2-[4-(4- |
| | fluorophenyl)-1H-pyrazol-1-yl]-N-[(2-methoxypyridin-4- |
| | yl)methyl]propanamide |
| I-138 | 4-{[N-(1,1-dioxo-2,3-dihydro-1lambda6-thiophen-3-yl)-2-[2- |
| | fluoro-4-(2-methyl-1H-pyrrol-1- |
| | yl)phenyl]acetamido]methyl}benzoic acid |
| I-139 | N-(1,1-dioxo-2,3-dihydro-1lambda6-thiophen-3-yl)-2-[2-fluoro- |
| | 4-(2-methyl-1H-pyrrol-1-yl)phenyl]-N-{[2-(2-hydroxy-2- |
| | methylpropyl)pyridin-4-yl]methyl}acetamide |
| I-140 | 3-(azetidin-1-yl)-N-(1,1-dioxo-2,3-dihydro-1lambda6-thiophen-3- |
| | yl)-2-{4′-fluoro-[1,1′-biphenyl]-4-yl}-3-oxo-N-[(pyridin-4- |
| | yl)methyl]propanamide |
| I-141 | N-(1,1-dioxo-2,3-dihydro-1lambda6-thiophen-3-yl)-N-[(2- |
| | ethoxypyridin-4-yl)methyl]-2-[4-(1H-pyrrol-1- |
| | yl)phenyl]acetamide |
| I-142 | N-(1,1-dioxo-2,3-dihydro-1lambda6-thiophen-3-yl)-2-[2-fluoro- |
| | 4-(2-methyl-1H-pyrrol-1-yl)phenyl]-N-{[2- |
| | (methylamino)pyridin-4-yl]methyl}acetamide |
| I-143 | 2-{3′,4′-difluoro-[1,1′-biphenyl]-4-yl}-N-(1,1-dioxo-2,3-dihydro- |
| | 1lambda6-thiophen-3-yl)-N-[(pyridin-4-yl)methyl]acetamide |
| I-144 | N-(1,1-dioxo-2,3-dihydro-1lambda6-thiophen-3-yl)-2-{4′-fluoro- |
| | [1,1′-biphenyl]-4-yl}-2-methoxy-N-[(pyridin-4- |
| | yl)methyl]acetamide |
| I-145 | N-(1,1-dioxo-2,3-dihydro-1lambda6-thiophen-3-yl)-2-[4-(1H- |
| | pyrrol-1-yl)phenyl]-N-{[4-(trifluoromethyl)-1,3-thiazol-2- |
| | yl]methyl}acetamide |
| I-146 | N-(1,1-dioxo-2,3-dihydro-1lambda6-thiophen-3-yl)-N-{[4- |
| | (pyridin-3-yl)-1,3-thiazol-2-yl]methyl}-2-[4-(1H-pyrrol-1- |
| | yl)phenyl]acetamide |
| I-147 | N-(1,1-dioxo-2,3-dihydro-1lambda6-thiophen-3-yl)-2-[4-(4- |
| | fluorophenyl)-2-oxo-1,2-dihydropyridin-1-yl]-N-[(pyridin-4- |
| | yl)methyl]acetamide |
| I-148 | N-(1,1-dioxo-2,3-dihydro-1lambda6-thiophen-3-yl)-2-[4-(1H- |
| | pyrrol-1-yl)phenyl]-N-{[4-(2,2,2-trifluoroethyl)-1,3-thiazol-2- |
| | yl]methyl}acetamide |
| I-149 | N-[(1,3-benzothiazol-2-yl)methyl]-N-(1,1-dioxo-2,3-dihydro- |
| | 1lambda6-thiophen-3-yl)-2-[4-(1H-pyrrol-1-yl)phenyl]acetamide |
| I-150 | N-(1,1-dioxo-2,3-dihydro-1lambda6-thiophen-3-yl)-2-[2-fluoro- |
| | 4-(2-methyl-1H-pyrrol-1-yl)phenyl]-N-[(pyridazin-4- |
| | yl)methyl]acetamide |
| I-151 | N-(1,1-dioxo-2,3-dihydro-1lambda6-thiophen-3-yl)-2-[2-fluoro- |
| | 4-(propan-2-yl)phenyl]-N-[(pyridin-4-yl)methyl]acetamide |
| I-152 | N-(1,1-dioxo-2,3-dihydro-1lambda6-thiophen-3-yl)-N-{[2-(2- |
| | hydroxypropan-2-yl)pyridin-4-yl]methyl}-2-[4-(1H-pyrrol-1- |
| | yl)phenyl]acetamide |
| I-153 | 4-{[N-(1,1-dioxo-2,3-dihydro-1lambda6-thiophen-3-yl)-2-[2- |
| | fluoro-4-(1H-pyrrol-1-yl)phenyl]acetamido]methyl}-N-methyl- |
| | N-(prop-2-yn-1-yl)benzamide |
| I-154 | N-(1,1-dioxo-2,3-dihydro-1lambda6-thiophen-3-yl)-2-[4-(1H- |
| | pyrrol-1-yl)phenyl]-N-[(4,5,6,7-tetrahydro-1,3-benzothiazol-2- |
| | yl)methyl]acetamide |
| I-155 | N-({4H,5H,6H-cyclopenta[d][1,3]thiazol-2-yl}methyl)-N-(1,1- |
| | dioxo-2,3-dihydro-1lambda6-thiophen-3-yl)-2-[4-(1H-pyrrol-1- |
| | yl)phenyl]acetamide |
| I-156 | N-(1,1-dioxo-2,3-dihydro-1lambda6-thiophen-3-yl)-N-[(2- |
| | methoxypyridin-4-yl)methyl]-2-[4-(1H-pyrrol-1- |
| | yl)phenyl]acetamide |
| I-157 | N-[(3-bromophenyl)methyl]-N-(1,1-dioxo-2,3-dihydro- |
| | 1lambda6-thiophen-3-yl)-2-[4-(1H-pyrrol-1-yl)phenyl]acetamide |
| I-158 | N-(1,1-dioxo-2,3-dihydro-1lambda6-thiophen-3-yl)-2-{4′-fluoro- |
| | [1,1′-biphenyl]-4-yl}-N-[(2-methoxypyridin-4- |
| | yl)methyl]acetamide |
| I-159 | N′-(1,1-dioxo-2,3-dihydro-1lambda6-thiophen-3-yl)-N′-[(2- |
| | methoxypyridin-4-yl)methyl]-N,N-dimethyl-2-[4-(pentafluoro- |
| | lambda6-sulfanyl)phenyl]propanediamide |
| I-160 | N-[(2-chloropyridin-4-yl)methyl]-N-(1,1-dioxo-2,3-dihydro- |
| | 1lambda6-thiophen-3-yl)-2-[4-(1H-pyrrol-1-yl)phenyl]acetamide |
| I-161 | N-(1,1-dioxo-2,3-dihydro-1lambda6-thiophen-3-yl)-2-[2-fluoro- |
| | 4-(1H-pyrrol-1-yl)phenyl]-N-[(pyridin-4-yl)methyl]acetamide |
| I-162 | 2-(4-cyclopropylphenyl)-N-(1,1-dioxo-2,3-dihydro-1lambda6- |
| | thiophen-3-yl)-N-[(2-methoxypyridin-4-yl)methyl]acetamide |
| I-163 | N-[(4-bromophenyl)methyl]-N-(1,1-dioxo-2,3-dihydro- |
| | 1lambda6-thiophen-3-yl)-2-[4-(1H-pyrrol-1-yl)phenyl]acetamide |
| I-164 | 2-(4-cyclohexylphenyl)-N-(1,1-dioxo-2,3-dihydro-1lambda6- |
| | thiophen-3-yl)-N-[(pyridin-4-yl)methyl]acetamide |
| I-165 | N-(1,1-dioxo-2,3-dihydro-1lambda6-thiophen-3-yl)-2-[4-(1H- |
| | pyrrol-1-yl)phenyl]-N-({[1,3]thiazolo[4,5-c]pyridin-2- |
| | yl}methyl)acetamide |
| I-166 | 2-{3′,5′-difluoro-[1,1′-biphenyl]-4-yl}-N-(1,1-dioxo-2,3-dihydro- |
| | 1lambda6-thiophen-3-yl)-N-[(pyridin-4-yl)methyl]acetamide |
| I-167 | N-(1,1-dioxo-2,3-dihydro-1lambda6-thiophen-3-yl)-N-{[2-(2- |
| | hydroxypropan-2-yl)pyridin-4-yl]methyl}-2-(propan-2-yloxy)-2- |
| | {3,3′,4′-trifluoro-[1,1′-biphenyl]-4-yl}acetamide |
| I-168 | N-(1,1-dioxo-2,3-dihydro-1lambda6-thiophen-3-yl)-N-[(2- |
| | methoxypyridin-4-yl)methyl]-2-{3,4′,5-trifluoro-[1,1′-biphenyl]- |
| | 4-yl}acetamide |
| I-169 | N-(1,1-dioxo-2,3-dihydro-1lambda6-thiophen-3-yl)-N-{[2-(1- |
| | hydroxyethyl)pyridin-4-yl]methyl}-2-[4-(1H-pyrrol-1- |
| | yl)phenyl]acetamide |
| I-170 | N-(1,1-dioxo-2,3-dihydro-1lambda6-thiophen-3-yl)-2-[4-(1H- |
| | pyrrol-1-yl)phenyl]-N-[(quinolin-4-yl)methyl]acetamide |
| I-171 | 2-[4-(2,5-dimethyl-1H-pyrrol-1-yl)-2-fluorophenyl]-N-(1,1- |
| | dioxo-2,3-dihydro-1lambda6-thiophen-3-yl)-N-[(2- |
| | methoxypyridin-4-yl)methyl]acetamide |
| I-172 | N-(1,1-dioxo-2,3-dihydro-1lambda6-thiophen-3-yl)-2-[3-fluoro- |
| | 5-(4-fluorophenyl)pyridin-2-yl]-N-[(2-methoxypyridin-4- |
| | yl)methyl]acetamide |
| I-173 | N-(1,1-dioxo-2,3-dihydro-1lambda6-thiophen-3-yl)-2-[2-fluoro- |
| | 4-(2-methyl-1H-pyrrol-1-yl)phenyl]-N-[(1-methyl-2-oxo-1,2- |
| | dihydropyridin-4-yl)methyl]acetamide |
| I-174 | N-(1,1-dioxo-2,3-dihydro-1lambda6-thiophen-3-yl)-2-[2-fluoro- |
| | 4-(2-methyl-1H-pyrrol-1-yl)phenyl]-N-{[2-(2-hydroxypropan-2- |
| | yl)pyridin-4-yl]methyl}-N′,N′-dimethylpropanediamide |
| I-175 | N-(1,1-dioxo-2,3-dihydro-1lambda6-thiophen-3-yl)-2-[5-(4- |
| | fluorophenyl)pyridin-2-yl]-N-[(2-methoxypyridin-4- |
| | yl)methyl]acetamide |
| I-176 | N-(1,1-dioxo-2,3-dihydro-1lambda6-thiophen-3-yl)-3-(4- |
| | fluorophenyl)-N-[(2-methoxypyridin-4- |
| | yl)methyl]bicyclo[4.2.0]octa-1,3,5-triene-7-carboxamide |
| I-177 | 2-(4-cyclobutylphenyl)-N-(1,1-dioxo-2,3-dihydro-1lambda6- |
| | thiophen-3-yl)-N-[(pyridin-4-yl)methyl]acetamide |
| I-178 | N-(1,1-dioxo-2,3-dihydro-1lambda6-thiophen-3-yl)-N- |
| | ({4H,6H,7H-pyrano[4,3-d][1,3]thiazol-2-yl}methyl)-2-[4-(1H- |
| | pyrrol-1-yl)phenyl]acetamide |
| I-179 | N-(1,1-dioxo-2,3-dihydro-1lambda6-thiophen-3-yl)-2-[2-fluoro- |
| | 4-(2-methyl-1H-pyrrol-1-yl)phenyl]-N-({2H,3H-furo[2,3- |
| | b]pyridin-4-yl}methyl)acetamide |
| I-180 | 2-{4′-chloro-3-fluoro-[1,1′-biphenyl]-4-yl}-N-(1,1-dioxo-2,3- |
| | dihydro-1lambda6-thiophen-3-yl)-N-{[2-(2-hydroxypropan-2- |
| | yl)pyridin-4-yl]methyl}acetamide |
| I-181 | N-(1,1-dioxo-2,3-dihydro-1lambda6-thiophen-3-yl)-2-[2-fluoro- |
| | 4-(2-methyl-1H-pyrrol-1-yl)phenyl]-N-[(pyrimidin-4- |
| | yl)methyl]acetamide |
| I-182 | N-(1,1-dioxo-2,3-dihydro-1lambda6-thiophen-3-yl)-2-[2-fluoro- |
| | 4-(2-methyl-1H-pyrrol-1-yl)phenyl]-N-({4-[4-(hex-5- |
| | ynoyl)piperazine-1-carbonyl]phenyl}methyl)acetamide |
| I-183 | N-(1,1-dioxo-2,3-dihydro-1lambda6-thiophen-3-yl)-N-[(pyridin- |
| | 4-yl)methyl]-2-[6-(trifluoromethyl)-2H-indazol-2-yl]acetamide |
| I-184 | N-(1,1-dioxo-2,3-dihydro-1lambda6-thiophen-3-yl)-2-[4-(1H- |
| | pyrrol-1-yl)phenyl]-N-[(4-sulfamoylphenyl)methyl]acetamide |
| I-185 | 4-{[N-(1,1-dioxo-2,3-dihydro-1lambda6-thiophen-3-yl)-2-[2- |
| | fluoro-4-(1H-pyrrol-1-yl)phenyl]acetamido]methyl}benzoic acid |
| I-186 | N-(1,1-dioxo-2,3-dihydro-1lambda6-thiophen-3-yl)-2-(4-phenyl- |
| | 1H-pyrazol-1-yl)-N-[(pyridin-4-yl)methyl]acetamide |
| I-187 | N-{[2-(difluoromethyl)pyridin-4-yl]methyl}-N-(1,1-dioxo-2,3- |
| | dihydro-1lambda6-thiophen-3-yl)-2-[4-(1H-pyrrol-1- |
| | yl)phenyl]acetamide |
| I-188 | N-(1,1-dioxo-2,3-dihydro-1lambda6-thiophen-3-yl)-2-{3′-fluoro- |
| | [1,1′-biphenyl]-4-yl}-N-[(pyridin-4-yl)methyl]acetamide |
| I-189 | N-(1,1-dioxo-2,3-dihydro-1lambda6-thiophen-3-yl)-N-[(4- |
| | methyl-1,3-thiazol-2-yl)methyl]-2-[4-(1H-pyrrol-1- |
| | yl)phenyl]acetamide |
| I-190 | N-[(4-cyano-1,3-thiazol-2-yl)methyl]-N-(1,1-dioxo-2,3-dihydro- |
| | 1lambda6-thiophen-3-yl)-2-[4-(1H-pyrrol-1-yl)phenyl]acetamide |
| I-191 | 2-[4-(1,1-difluoroethyl)phenyl]-N-(1,1-dioxo-2,3-dihydro- |
| | 1lambda6-thiophen-3-yl)-N-[(pyridin-4-yl)methyl]acetamide |
| I-192 | N′-(1,1-dioxo-2,3-dihydro-1lambda6-thiophen-3-yl)-N′-{[2-(2- |
| | hydroxypropan-2-yl)pyridin-4-yl]methyl}-N,N-dimethyl-2-[4- |
| | (pentafluoro-lambda6-sulfanyl)phenyl]propanediamide |
| I-193 | rel-N-[(3R)-1,1-dioxo-2,3-dihydro-1lambda6-thiophen-3-yl]-N- |
| | [(pyridin-4-yl)methyl]-2-[4-(1H-pyrrol-1-yl)phenyl]acetamide |
| I-194 | N-(1,1-dioxo-2,3-dihydro-1lambda6-thiophen-3-yl)-2-[6-(4- |
| | fluoro-2-hydroxyphenyl)pyridin-3-yl]-N-[(2-methoxypyridin-4- |
| | yl)methyl]acetamide |
| I-195 | N-(1,1-dioxo-2,3-dihydro-1lambda6-thiophen-3-yl)-N-{[2-(2- |
| | hydroxypropan-2-yl)pyridin-4-yl]methyl}-3-methoxy-2-{3,3′,4′- |
| | trifluoro-[1,1′-biphenyl]-4-yl}propanamide |
| I-196 | 2-[3-(difluoromethyl)-4′-fluoro-[1,1′-biphenyl]-4-yl]-N-(1,1- |
| | dioxo-2,3-dihydro-1lambda6-thiophen-3-yl)-N-[(pyridin-4- |
| | yl)methyl]acetamide |
| I-197 | N-(1,1-dioxo-2,3-dihydro-1lambda6-thiophen-3-yl)-2-[4-(propan- |
| | 2-yl)phenyl]-N-[(pyridin-4-yl)methyl]acetamide |
| I-198 | 2-[5-(3,4-difluorophenyl)pyrimidin-2-yl]-N-(1,1-dioxo-2,3- |
| | dihydro-1lambda6-thiophen-3-yl)-N-[(2-methoxypyridin-4- |
| | yl)methyl]acetamide |
| I-199 | (2R*)-2-{3,4′-difluoro-[1,1′-biphenyl]-4-yl}-N-[(3R**)-1,1- |
| | dioxo-2,3-dihydro-1lambda6-thiophen-3-yl]-N-{[2-(2- |
| | hydroxypropan-2-yl)pyridin-4-yl]methyl}-2-methoxyacetamide |
| I-200 | N-(1,1-dioxo-2,3-dihydro-1lambda6-thiophen-3-yl)-2-{4′-fluoro- |
| | [1,1′-biphenyl]-4-yl}-N-[(pyridin-4-yl)methyl]acetamide |
| I-201 | N-[(3-chlorophenyl)methyl]-N-(1,1-dioxo-2,3-dihydro-1lambda6- |
| | thiophen-3-yl)-2-[4-(1H-pyrrol-1-yl)phenyl]acetamide |
| I-202 | N-[(4,6-dimethoxypyridin-2-yl)methyl]-N-(1,1-dioxo-2,3- |
| | dihydro-1lambda6-thiophen-3-yl)-2-[2-fluoro-4-(2-methyl-1H- |
| | pyrrol-1-yl)phenyl]acetamide |
| I-203 | 2-{2′,4′-difluoro-[1,1′-biphenyl]-4-yl}-N-(1,1-dioxo-2,3-dihydro- |
| | 1lambda6-thiophen-3-yl)-N-[(pyridin-4-yl)methyl]acetamide |
| I-204 | N-(1,1-dioxo-2,3-dihydro-1lambda6-thiophen-3-yl)-2-[4-(2-ethyl- |
| | 1H-pyrrol-1-yl)-2-fluorophenyl]-N-[(2-methoxypyridin-4- |
| | yl)methyl]acetamide |
| I-205 | N-(1,1-dioxo-2,3-dihydro-1lambda6-thiophen-3-yl)-2-[3-fluoro- |
| | 5-(4-fluorophenyl)pyridin-2-yl]-N-[(pyridin-4- |
| | yl)methyl]acetamide |
| I-206 | N-(1,1-dioxo-2,3-dihydro-1lambda6-thiophen-3-yl)-2-[4-(2- |
| | methyl-1H-pyrrol-1-yl)phenyl]-N-[(pyridin-4- |
| | yl)methyl]acetamide |
| I-207 | N-(1,1-dioxo-2,3-dihydro-1lambda6-thiophen-3-yl)-N-[(2- |
| | methylpyridin-4-yl)methyl]-2-[4-(1H-pyrrol-1- |
| | yl)phenyl]acetamide |
| I-208 | 2-[2-chloro-4-(1H-pyrrol-1-yl)phenyl]-N-(1,1-dioxo-2,3-dihydro- |
| | 1lambda6-thiophen-3-yl)-N-[(pyridin-4-yl)methyl]acetamide |
| I-209 | N-(1,1-dioxo-2,3-dihydro-1lambda6-thiophen-3-yl)-N-{[2-(2- |
| | hydroxypropan-2-yl)pyridin-4-yl]methyl}-2-methoxy-2-[4- |
| | (trifluoromethyl)phenyl]acetamide |
| I-210 | N-(1,1-dioxo-2,3-dihydro-1lambda6-thiophen-3-yl)-2-[4-(1H- |
| | pyrrol-1-yl)phenyl]-N-[(quinolin-2-yl)methyl]acetamide |
| I-211 | N-(1,1-dioxo-2,3-dihydro-1lambda6-thiophen-3-yl)-2-[2-fluoro- |
| | 4-(1H-pyrrol-1-yl)phenyl]-N-[(4-{[4-(hex-5-ynoyl)piperazin-1- |
| | yl]methyl}phenyl)methyl]acetamide |
| I-212 | N-(1,1-dioxo-2,3-dihydro-1lambda6-thiophen-3-yl)-N-[(2- |
| | methoxypyridin-4-yl)methyl]-2-[4-(1H-pyrrol-1- |
| | yl)phenyl]propanamide |
| I-213 | N-(1,1-dioxo-2,3-dihydro-1lambda6-thiophen-3-yl)-2-[2-fluoro- |
| | 4-(1H-pyrrol-1-yl)phenyl]-N-({4-[4-(hex-5-ynoyl)piperazin-1- |
| | yl]phenyl}methyl)acetamide |
| I-214 | 2-{4-[2-(difluoromethyl)-1H-pyrrol-1-yl]-2-fluorophenyl}-N- |
| | (1,1-dioxo-2,3-dihydro-1lambda6-thiophen-3-yl)-N-[(2- |
| | methoxypyridin-4-yl)methyl]acetamide |
| I-215 | N-{[2-(difluoromethoxy)pyridin-4-yl]methyl}-N-(1,1-dioxo-2,3- |
| | dihydro-1lambda6-thiophen-3-yl)-2-[4-(1H-pyrrol-1- |
| | yl)phenyl]acetamide |
| I-216 | 2-[4-(butan-2-yl)phenyl]-N-(1,1-dioxo-2,3-dihydro-1lambda6- |
| | thiophen-3-yl)-N-[(pyridin-4-yl)methyl]acetamide |
| I-217 | 2-(4-cyclopentylphenyl)-N-(1,1-dioxo-2,3-dihydro-1lambda6- |
| | thiophen-3-yl)-N-[(pyridin-4-yl)methyl]acetamide |
| I-218 | N-(1,1-dioxo-2,3-dihydro-1lambda6-thiophen-3-yl)-2-{4′-fluoro- |
| | [1,1′-biphenyl]-4-yl}-3-(morpholin-4-yl)-3-oxo-N-[(pyridin-4- |
| | yl)methyl]propanamide |
| I-219 | N-(1,1-dioxo-2,3-dihydro-1lambda6-thiophen-3-yl)-N-[(4- |
| | methoxyphenyl)methyl]-2-[4-(1H-pyrrol-1-yl)phenyl]acetamide |
| I-220 | 2-(6-bromonaphthalen-2-yl)-N-(1,1-dioxo-2,3-dihydro-1lambda6- |
| | thiophen-3-yl)-N-[(pyridin-4-yl)methyl]acetamide |
| I-221 | N-[(2,3-difluorophenyl)methyl]-N-(1,1-dioxo-2,3-dihydro- |
| | 1lambda6-thiophen-3-yl)-2-[4-(1H-pyrrol-1-yl)phenyl]acetamide |
| I-222 | N-(1,1-dioxo-2,3-dihydro-1lambda6-thiophen-3-yl)-2-[4-(1H- |
| | pyrrol-1-yl)phenyl]-N-[(quinolin-6-yl)methyl]acetamide |
| I-223 | N-(1,1-dioxo-2,3-dihydro-1lambda6-thiophen-3-yl)-3-(4- |
| | fluorophenyl)-N-{[2-(2-hydroxypropan-2-yl)pyridin-4- |
| | yl]methyl}bicyclo[4.2.0]octa-1,3,5-triene-7-carboxamide |
| I-224 | N-(1,1-dioxo-2,3-dihydro-1lambda6-thiophen-3-yl)-2-[2-fluoro- |
| | 4-(2-methyl-1H-pyrrol-1-yl)phenyl]-N-[(pyridazin-3- |
| | yl)methyl]acetamide |
| I-225 | N-[(2H-1,3-benzodioxol-5-yl)methyl]-N-(1,1-dioxo-2,3-dihydro- |
| | 1lambda6-thiophen-3-yl)-2-[4-(1H-pyrrol-1-yl)phenyl]acetamide |
| I-226 | N-(1,1-dioxo-2,3-dihydro-1lambda6-thiophen-3-yl)-N-[(1- |
| | methyl-1H-indazol-5-yl)methyl]-2-[4-(1H-pyrrol-1- |
| | yl)phenyl]acetamide |
| I-227 | N-(1,1-dioxo-2,3-dihydro-1lambda6-thiophen-3-yl)-2-{4′-fluoro- |
| | 3-methyl-[1,1′-biphenyl]-4-yl}-N-[(pyridin-4- |
| | yl)methyl]acetamide |
| I-228 | N-[(3-chloropyridin-4-yl)methyl]-N-(1,1-dioxo-2,3-dihydro- |
| | 1lambda6-thiophen-3-yl)-2-[4-(1H-pyrrol-1-yl)phenyl]acetamide |
| I-229 | N-(1,1-dioxo-2,3-dihydro-1lambda6-thiophen-3-yl)-2-[2-fluoro- |
| | 4-(1H-pyrrol-1-yl)phenyl]-N-({4-[4-(hex-5-ynoyl)piperazine-1- |
| | carbonyl]phenyl}methyl)acetamide |
| I-230 | N-[(3S)-1,1-dioxo-2,3-dihydro-1lambda6-thiophen-3-yl]-N-{[2- |
| | (2-hydroxypropan-2-yl)pyridin-4-yl]methyl}-2-[4-(pentafluoro- |
| | lambda6-sulfanyl)phenyl]acetamide |
| I-231 | N-(1,1-dioxo-2,3-dihydro-1lambda6-thiophen-3-yl)-2-[3-oxo-6- |
| | (trifluoromethyl)-3,4-dihydro-2H-1,4-benzothiazin-2-yl]-N- |
| | [(pyridin-4-yl)methyl]acetamide |
| I-232 | N-(1,1-dioxo-2,3-dihydro-1lambda6-thiophen-3-yl)-N-{[3-(1H- |
| | pyrazol-1-yl)phenyl]methyl}-2-[4-(1H-pyrrol-1- |
| | yl)phenyl]acetamide |
| I-233 | N-(1,1-dioxo-2,3-dihydro-1lambda6-thiophen-3-yl)-N-[(pyridin- |
| | 4-yl)methyl]-3-(1H-pyrrol-1-yl)bicyclo[4.2.0]octa-1,3,5-triene-7- |
| | carboxamide |
| I-234 | N-(1,1-dioxo-2,3-dihydro-1lambda6-thiophen-3-yl)-N-[(3- |
| | methoxyphenyl)methyl]-2-[4-(1H-pyrrol-1-yl)phenyl]acetamide |
| I-235 | (2R**)-2-{3,4′-difluoro-[1,1′-biphenyl]-4-yl}-N-[(3R*)-1,1- |
| | dioxo-2,3-dihydro-1lambda6-thiophen-3-yl]-N-[(2- |
| | methoxypyridin-4-yl)methyl]-N′,N′-dimethylpropanediamide |
| I-236 | N-(1,1-dioxo-2,3-dihydro-1lambda6-thiophen-3-yl)-2-[3-fluoro- |
| | 4-(trifluoromethyl)phenyl]-N-[(pyridin-4-yl)methyl]acetamide |
| I-237 | (2R**)-2-{3,4′-difluoro-[1,1′-biphenyl]-4-yl}-N-[(3R*)-1,1- |
| | dioxo-2,3-dihydro-1lambda6-thiophen-3-yl]-N-{[2-(2- |
| | hydroxypropan-2-yl)pyridin-4-yl]methyl}-N′,N′- |
| | dimethylpropanediamide |
| I-238 | N-(1,1-dioxo-2,3-dihydro-1lambda6-thiophen-3-yl)-N-({4-[4- |
| | (hex-5-ynoyl)piperazin-1-yl]phenyl}methyl)-2-[4-(1H-pyrrol-1- |
| | yl)phenyl]acetamide |
| I-239 | N-(1,1-dioxo-2,3-dihydro-1lambda6-thiophen-3-yl)-N-[(pyridin- |
| | 4-yl)methyl]-2-[4-(1H-pyrrol-1-yl)phenyl]acetamide |
| I-240 | 3-(6-chloro-1H-indol-3-yl)-N-(1,1-dioxo-2,3-dihydro-1lambda6- |
| | thiophen-3-yl)-N-[(2-methoxypyridin-4-yl)methyl]propanamide |
| I-241 | 2-[2,6-difluoro-4-(2-methyl-1H-pyrrol-1-yl)phenyl]-N-(1,1- |
| | dioxo-2,3-dihydro-1lambda6-thiophen-3-yl)-N-[(2- |
| | methoxypyridin-4-yl)methyl]acetamide |
| I-242 | N-[(2,4-difluorophenyl)methyl]-N-(1,1-dioxo-2,3-dihydro- |
| | 1lambda6-thiophen-3-yl)-2-[4-(1H-pyrrol-1-yl)phenyl]acetamide |
| I-243 | N-(1,1-dioxo-2,3-dihydro-1lambda6-thiophen-3-yl)-2-[4-(1H- |
| | pyrrol-1-yl)phenyl]-N-[(quinolin-8-yl)methyl]acetamide |
| I-244 | N-(1,1-dioxo-2,3-dihydro-1lambda6-thiophen-3-yl)-N-[(pyridin- |
| | 4-yl)methyl]-5-(1H-pyrrol-1-yl)-2,3-dihydro-1-benzofuran-2- |
| | carboxamide |
| I-245 | N-(1,1-dioxo-2,3-dihydro-1lambda6-thiophen-3-yl)-2-[4-(2- |
| | methylcyclopropyl)phenyl]-N-[(pyridin-4-yl)methyl]acetamide |
| I-246 | N-(1,1-dioxo-2,3-dihydro-1lambda6-thiophen-3-yl)-2-methoxy- |
| | N-[(2-methoxypyridin-4-yl)methyl]-2-[4- |
| | (trifluoromethyl)phenyl]acetamide |
| I-247 | N-(1,1-dioxo-2,3-dihydro-1lambda6-thiophen-3-yl)-2-{4′-fluoro- |
| | [1,1′-biphenyl]-4-yl}-N-[(3-methoxy-1,2-oxazol-5- |
| | yl)methyl]acetamide |
| I-248 | N-(1,1-dioxo-2,3-dihydro-1lambda6-thiophen-3-yl)-N-[(pyridin- |
| | 4-yl)methyl]-2-[4-(thiophen-3-yl)phenyl]acetamide |
| I-249 | N-(1,1-dioxo-2,3-dihydro-1lambda6-thiophen-3-yl)-3-(4- |
| | fluorophenyl)-N-[(pyridin-4-yl)methyl]bicyclo[4.2.0]octa- |
| | 1(6),2,4-triene-7-carboxamide |
| I-250 | N-(1,1-dioxo-2,3-dihydro-1lambda6-thiophen-3-yl)-N-[(2- |
| | methoxypyridin-4-yl)methyl]-2-[5-(2-methyl-1H-pyrrol-1- |
| | yl)pyridin-2-yl]acetamide |
| I-251 | N-(1,1-dioxo-2,3-dihydro-1lambda6-thiophen-3-yl)-2-[4-(1H- |
| | pyrrol-1-yl)phenyl]-N-[(quinoxalin-6-yl)methyl]acetamide |
| I-252 | N-(1,1-dioxo-2,3-dihydro-1lambda6-thiophen-3-yl)-N-[(pyridin- |
| | 4-yl)methyl]-2-[4-(trifluoromethoxy)phenyl]acetamide |
| I-253 | rel-N-[(4-cyclopropyl-1,3-thiazol-2-yl)methyl]-N-[(3R)-1,1- |
| | dioxo-2,3-dihydro-1lambda6-thiophen-3-yl]-2-[4-(pentafluoro- |
| | lambda6-sulfanyl)phenyl]acetamide |
| I-254 | N-(1,1-dioxo-2,3-dihydro-1lambda6-thiophen-3-yl)-N-[(pyridin- |
| | 4-yl)methyl]-2-[4-(trifluoromethyl)phenyl]acetamide |
| I-255 | 4-{[N-(1,1-dioxo-2,3-dihydro-1lambda6-thiophen-3-yl)-2-[4- |
| | (1H-pyrrol-1-yl)phenyl]acetamido]methyl}-N-methylbenzamide |
| I-256 | N-(1,1-dioxo-2,3-dihydro-1lambda6-thiophen-3-yl)-N-[(4- |
| | methanesulfonylphenyl)methyl]-2-[4-(1H-pyrrol-1- |
| | yl)phenyl]acetamide |
| I-257 | N-(1,1-dioxo-2,3-dihydro-1lambda6-thiophen-3-yl)-N-[(2- |
| | fluorophenyl)methyl]-2-[4-(1H-pyrrol-1-yl)phenyl]acetamide |
| I-258 | (2R**)-2-{3,4′-difluoro-[1,1′-biphenyl]-4-yl}-N-[(3R*)-1,1- |
| | dioxo-2,3-dihydro-1lambda6-thiophen-3-yl]-N-{[2-(2- |
| | hydroxypropan-2-yl)pyridin-4-yl]methyl}-N′,N′- |
| | dimethylpropanediamide |
| I-259 | 4-{[N-(1,1-dioxo-2,3-dihydro-1lambda6-thiophen-3-yl)-2-[4- |
| | (1H-pyrrol-1-yl)phenyl]acetamido]methyl}-N,N- |
| | dimethylbenzamide |
| I-260 | N-(1,1-dioxo-2,3-dihydro-1lambda6-thiophen-3-yl)-2-[2-fluoro- |
| | 4-(trifluoromethyl)phenyl]-N-[(pyridin-4-yl)methyl]acetamide |
| I-261 | 2-{2′,3′-difluoro-[1,1′-biphenyl]-4-yl}-N-(1,1-dioxo-2,3-dihydro- |
| | 1lambda6-thiophen-3-yl)-N-[(pyridin-4-yl)methyl]acetamide |
| I-262 | rel-N-benzyl-N-[(3R)-1,1-dioxo-2,3-dihydro-1lambda6-thiophen- |
| | 3-yl]-2-[4-(1H-pyrrol-1-yl)phenyl]acetamide |
| I-263 | N-(1,1-dioxo-2,3-dihydro-1lambda6-thiophen-3-yl)-N-[(pyridin- |
| | 4-yl)methyl]-2-[4-(2,2,2-trifluoroethyl)phenyl]acetamide |
| I-264 | (2R**)-N-[(3R*)-1,1-dioxo-2,3-dihydro-1lambda6-thiophen-3- |
| | yl]-2-{4′-fluoro-[1,1′-biphenyl]-4-yl}-N-{[2-(2-hydroxypropan-2- |
| | yl)pyridin-4-yl]methyl}-N′,N′-dimethylpropanediamide |
| I-265 | N-(1,1-dioxo-2,3-dihydro-1lambda6-thiophen-3-yl)-N-[(pyridin- |
| | 4-yl)methyl]-2-[4-(1H-pyrrol-1-yl)phenyl]propanamide |
| I-266 | N-(1,1-dioxo-2,3-dihydro-1lambda6-thiophen-3-yl)-2-{4′-fluoro- |
| | [1,1′-biphenyl]-4-yl}-N-[(pyridin-4-yl)methyl]propanamide |
| I-267 | (2R**)-2-{3,4′-difluoro-[1,1′-biphenyl]-4-yl}-N-[(3R*)-1,1- |
| | dioxo-2,3-dihydro-1lambda6-thiophen-3-yl]-N-[(2- |
| | methoxypyridin-4-yl)methyl]-N′,N′-dimethylpropanediamide |
| I-268 | 2-{4-[2-(2-aminoethyl)-1H-pyrrol-1-yl]phenyl}-N-benzyl-N-(1,1- |
| | dioxo-2,3-dihydro-1lambda6-thiophen-3-yl)acetamide |
| I-269 | N-benzyl-N-(5-bromo-1,1-dioxo-2,3-dihydro-1lambda6- |
| | thiophen-3-yl)-2-[4-(1H-pyrrol-1-yl)phenyl]acetamide |
| I-270 | N-(1,1-dioxo-2,3-dihydro-1lambda6-thiophen-3-yl)-N-[(3- |
| | fluorophenyl)methyl]-2-[4-(1H-pyrrol-1-yl)phenyl]acetamide |
| I-271 | (2R**)-N-[(3R*)-1,1-dioxo-2,3-dihydro-1lambda6-thiophen-3- |
| | yl]-2-{4′-fluoro-[1,1′-biphenyl]-4-yl}-N-{[2-(2-hydroxypropan-2- |
| | yl)pyridin-4-yl]methyl}-N′,N′-dimethylpropanediamide |
| I-272 | 2-[4-(2-cyano-1H-pyrrol-1-yl)phenyl]-N-(1,1-dioxo-2,3-dihydro- |
| | 1lambda6-thiophen-3-yl)-N-[(pyridin-4-yl)methyl]acetamide |
| I-273 | 2-[4-(2,2-difluorocyclopropyl)phenyl]-N-(1,1-dioxo-2,3-dihydro- |
| | 1lambda6-thiophen-3-yl)-N-[(pyridin-4-yl)methyl]acetamide |
| I-274 | N-(1,1-dioxo-2,3-dihydro-1lambda6-thiophen-3-yl)-N-[(5- |
| | methyl-1,3-thiazol-2-yl)methyl]-2-[4-(1H-pyrrol-1- |
| | yl)phenyl]acetamide |
| I-275 | 4-{[N-(1,1-dioxo-2,3-dihydro-1lambda6-thiophen-3-yl)-2-[4- |
| | (1H-pyrrol-1-yl)phenyl]acetamido]methyl}benzoic acid |
| I-276 | N-(1,1-dioxo-2,3-dihydro-1lambda6-thiophen-3-yl)-2-[4-(1H- |
| | pyrrol-1-yl)phenyl]-N-[(1,3-thiazol-2-yl)methyl]acetamide |
| I-277 | N-(1,1-dioxo-2,3-dihydro-1lambda6-thiophen-3-yl)-N′,N′- |
| | dimethyl-2-{4′-methyl-[1,1′-biphenyl]-4-yl}-N-[(pyridin-4- |
| | yl)methyl]propanediamide |
| I-278 | N-[(2,5-difluorophenyl)methyl]-N-(1,1-dioxo-2,3-dihydro- |
| | 1lambda6-thiophen-3-yl)-2-[4-(1H-pyrrol-1-yl)phenyl]acetamide |
| I-279 | N-(1,1-dioxo-2,3-dihydro-1lambda6-thiophen-3-yl)-N-[(pyridin- |
| | 4-yl)methyl]-2-[5-(1H-pyrrol-1-yl)-1,3-thiazol-2-yl]acetamide |
| I-280 | N-[(3,4-dimethoxyphenyl)methyl]-N-(1,1-dioxo-2,3-dihydro- |
| | 1lambda6-thiophen-3-yl)-2-[4-(1H-pyrrol-1-yl)phenyl]acetamide |
| I-281 | rel-N-[(3R)-1,1-dioxo-2,3-dihydro-1lambda6-thiophen-3-yl]-2- |
| | (4-phenyl-1H-pyrazol-1-yl)-N-[(pyridin-4-yl)methyl]acetamide |
| I-282 | rel-(2R)-N-(1,1-dioxo-2,3-dihydro-1lambda6-thiophen-3-yl)-2- |
| | {4′-fluoro-[1,1′-biphenyl]-4-yl}-2-(N-methylacetamido)-N- |
| | [(pyridin-4-yl)methyl]acetamide |
| I-283 | N-(1,1-dioxo-2,3-dihydro-1lambda6-thiophen-3-yl)-2-[4-(2- |
| | methylpropyl)phenyl]-N-[(pyridin-4-yl)methyl]acetamide |
| I-284 | N-(1,1-dioxo-2,3-dihydro-1lambda6-thiophen-3-yl)-N-[(4- |
| | methanesulfonamidophenyl)methyl]-2-[4-(1H-pyrrol-1- |
| | yl)phenyl]acetamide |
| I-285 | N-[(5-cyclopropylpyridin-2-yl)methyl]-N-(1,1-dioxo-2,3- |
| | dihydro-1lambda6-thiophen-3-yl)-2-[4-(1H-pyrrol-1- |
| | yl)phenyl]acetamide |
| I-286 | N-[(3-cyanophenyl)methyl]-N-(1,1-dioxo-2,3-dihydro-1lambda6- |
| | thiophen-3-yl)-2-[4-(1H-pyrrol-1-yl)phenyl]acetamide |
| I-287 | 2-(4-cyclopropylphenyl)-N-(1,1-dioxo-2,3-dihydro-1lambda6- |
| | thiophen-3-yl)-N-[(pyridin-4-yl)methyl]acetamide |
| I-288 | N-(1,1-dioxo-2,3-dihydro-1lambda6-thiophen-3-yl)-2-{3′-methyl- |
| | [1,1′-biphenyl]-4-yl}-N-[(pyridin-4-yl)methyl]acetamide |
| I-289 | N-(1,1-dioxo-2,3-dihydro-1lambda6-thiophen-3-yl)-2-(3-phenyl- |
| | 1H-pyrazol-5-yl)-N-[(pyridin-4-yl)methyl]acetamide |
| I-290 | N-benzyl-N-(1,1-dioxo-2,3-dihydro-1lambda6-thiophen-3-yl)-2- |
| | [4-(1H-pyrrol-1-yl)phenyl]acetamide |
| I-291 | N-(1,1-dioxo-2,3-dihydro-1lambda6-thiophen-3-yl)-2-(4-phenyl- |
| | 2H-1,2,3-triazol-2-yl)-N-[(pyridin-4-yl)methyl]acetamide |
| I-292 | N-(1,1-dioxo-2,3-dihydro-1lambda6-thiophen-3-yl)-N-[(3- |
| | hydroxyphenyl)methyl]-2-[4-(1H-pyrrol-1-yl)phenyl]acetamide |
| I-293 | 2-[2-bromo-4-(trifluoromethyl)phenyl]-N-(1,1-dioxo-2,3- |
| | dihydro-1lambda6-thiophen-3-yl)-N-[(pyridin-4- |
| | yl)methyl]acetamide |
| I-294 | N-[(2,6-difluorophenyl)methyl]-N-(1,1-dioxo-2,3-dihydro- |
| | 1lambda6-thiophen-3-yl)-2-[4-(1H-pyrrol-1-yl)phenyl]acetamide |
| I-295 | N-(1,1-dioxo-2,3-dihydro-1lambda6-thiophen-3-yl)-2-[4-(1H- |
| | pyrrol-1-yl)phenyl]-N-{[6-(trifluoromethyl)pyridin-3- |
| | yl]methyl}acetamide |
| I-296 | N-(1,1-dioxo-2,3-dihydro-1lambda6-thiophen-3-yl)-2-[4-(1H- |
| | pyrrol-1-yl)phenyl]-N-[(quinolin-3-yl)methyl]acetamide |
| I-297 | N-(1,1-dioxo-2,3-dihydro-1lambda6-thiophen-3-yl)-N-[(2- |
| | phenylpyridin-4-yl)methyl]-2-[4-(1H-pyrrol-1- |
| | yl)phenyl]acetamide |
| I-298 | N-(1,1-dioxo-2,3-dihydro-1lambda6-thiophen-3-yl)-5-(4- |
| | fluorophenyl)-N-{[2-(2-hydroxypropan-2-yl)pyridin-4- |
| | yl]methyl}-2,3-dihydro-1-benzofuran-2-carboxamide |
| I-299 | 2-[3-chloro-4-(trifluoromethyl)phenyl]-N-(1,1-dioxo-2,3-dihydro- |
| | 1lambda6-thiophen-3-yl)-N-[(pyridin-4-yl)methyl]acetamide |
| I-300 | N-(1,1-dioxo-2,3-dihydro-1lambda6-thiophen-3-yl)-2-(5-phenyl- |
| | 1,3-thiazol-2-yl)-N-[(pyridin-4-yl)methyl]acetamide |
| I-301 | 2-(1-benzothiophen-2-yl)-N-(1,1-dioxo-2,3-dihydro-1lambda6- |
| | thiophen-3-yl)-N-[(pyridin-4-yl)methyl]acetamide |
| I-302 | N-(1,1-dioxo-2,3-dihydro-1lambda6-thiophen-3-yl)-N-[(4- |
| | acetamidophenyl)methyl]-2-[4-(1H-pyrrol-1-yl)phenyl]acetamide |
| I-303 | N-(1,1-dioxo-2,3-dihydro-1lambda6-thiophen-3-yl)-N-[(4- |
| | hydroxyphenyl)methyl]-2-[4-(1H-pyrrol-1-yl)phenyl]acetamide |
| I-304 | 3-(6-bromo-1H-indol-3-yl)-N-(1,1-dioxo-2,3-dihydro-1lambda6- |
| | thiophen-3-yl)-N-[(pyridin-4-yl)methyl]propanamide |
| I-305 | N-(1,1-dioxo-2,3-dihydro-1lambda6-thiophen-3-yl)-2-[4-(1H- |
| | indol-1-yl)phenyl]-N-[(pyridin-4-yl)methyl]acetamide |
| I-306 | 3-(6-chloro-1H-indol-3-yl)-N-(1,1-dioxo-2,3-dihydro-1lambda6- |
| | thiophen-3-yl)-N-[(pyridin-4-yl)methyl]propanamide |
| I-307 | 2-(dimethylamino)-N-(1,1-dioxo-2,3-dihydro-1lambda6- |
| | thiophen-3-yl)-N-{[2-(2-hydroxypropan-2-yl)pyridin-4- |
| | yl]methyl}-2-{3,3′,4′-trifluoro-[1,1′-biphenyl]-4-yl}acetamide |
| I-308 | N-(1,1-dioxo-2,3-dihydro-1lambda6-thiophen-3-yl)-2-(4- |
| | ethylphenyl)-N-[(pyridin-4-yl)methyl]acetamide |
| I-309 | (2R**)-N-[(3R*)-1,1-dioxo-2,3-dihydro-1lambda6-thiophen-3- |
| | yl]-2-{4′-fluoro-[1,1′-biphenyl]-4-yl}-N-{[2-(2-hydroxypropan-2- |
| | yl)pyridin-4-yl]methyl}-N′,N′-dimethylpropanediamide |
| I-310 | 2-(4-tert-butylphenyl)-N-(1,1-dioxo-2,3-dihydro-1lambda6- |
| | thiophen-3-yl)-N-[(pyridin-4-yl)methyl]acetamide |
| I-311 | N-[(3S)-1,1-dioxo-2,3-dihydro-1lambda6-thiophen-3-yl]-N-[(2- |
| | methoxypyridin-4-yl)methyl]-2-[4-(pentafluoro-lambda6- |
| | sulfanyl)phenyl]acetamide |
| I-312 | (2R**)-2-{3,4′-difluoro-[1,1′-biphenyl]-4-yl}-N-[(3R*)-1,1- |
| | dioxo-2,3-dihydro-1lambda6-thiophen-3-yl]-N-[(2- |
| | methoxypyridin-4-yl)methyl]-N′,N′-dimethylpropanediamide |
| I-313 | N-(1,1-dioxo-2,3-dihydro-1lambda6-thiophen-3-yl)-N-[(3- |
| | methylpyridin-4-yl)methyl]-2-[4-(1H-pyrrol-1- |
| | yl)phenyl]acetamide |
| I-314 | (3-{[N-(1,1-dioxo-2,3-dihydro-1lambda6-thiophen-3-yl)-2-[4- |
| | (1H-pyrrol-1-yl)phenyl]acetamido]methyl}phenyl)boronic acid |
| I-315 | N-(1,1-dioxo-2,3-dihydro-1lambda6-thiophen-3-yl)-2- |
| | (naphthalen-2-yl)-N-[(pyridin-4-yl)methyl]acetamide |
| I-316 | N-(1,1-dioxo-2,3-dihydro-1lambda6-thiophen-3-yl)-2-[4-(1H- |
| | pyrrol-1-yl)phenyl]-N-[(quinoxalin-5-yl)methyl]acetamide |
| I-317 | 2-[4-(difluoromethyl)phenyl]-N-(1,1-dioxo-2,3-dihydro- |
| | 1lambda6-thiophen-3-yl)-N-[(pyridin-4-yl)methyl]acetamide |
| I-318 | 3-{[N-(1,1-dioxo-2,3-dihydro-1lambda6-thiophen-3-yl)-2-[4- |
| | (1H-pyrrol-1-yl)phenyl]acetamido]methyl}benzamide |
| I-319 | N-(1,1-dioxo-2,3-dihydro-1lambda6-thiophen-3-yl)-N-[(pyridin- |
| | 4-yl)methyl]-2-[5-(1H-pyrrol-1-yl)pyridin-2-yl]acetamide |
| I-320 | N-(1,1-dioxo-2,3-dihydro-1lambda6-thiophen-3-yl)-2-[2-fluoro- |
| | 4-(5-methyl-1,3-thiazol-4-yl)phenyl]-N-[(pyridin-4- |
| | yl)methyl]acetamide |
| I-321 | (4-{[N-(1,1-dioxo-2,3-dihydro-1lambda6-thiophen-3-yl)-2-[4- |
| | (1H-pyrrol-1-yl)phenyl]acetamido]methyl}phenyl)boronic acid |
| I-322 | N-(1,1-dioxo-2,3-dihydro-1lambda6-thiophen-3-yl)-N-[(2- |
| | methanesulfonylphenyl)methyl]-2-[4-(1H-pyrrol-1- |
| | yl)phenyl]acetamide |
| I-323 | 2-{[1,1′-biphenyl]-4-yl}-N-(1,1-dioxo-2,3-dihydro-1lambda6- |
| | thiophen-3-yl)-N-[(pyridin-4-yl)methyl]acetamide |
| I-324 | N-(1,1-dioxo-2,3-dihydro-1lambda6-thiophen-3-yl)-N-{[2-(2- |
| | hydroxypropan-2-yl)pyridin-4-yl]methyl}-3-methoxy-2-(4- |
| | phenyl-1H-pyrazol-1-yl)propanamide |
| I-325 | N-(1,1-dioxo-2,3-dihydro-1lambda6-thiophen-3-yl)-2-(6-oxo-4- |
| | phenyl-1,6-dihydropyrimidin-1-yl)-N-[(pyridin-4- |
| | yl)methyl]acetamide |
| I-326 | N-(1,1-dioxo-2,3-dihydro-1lambda6-thiophen-3-yl)-2-(6-oxo-3- |
| | phenyl-1,6-dihydropyridazin-1-yl)-N-[(pyridin-4- |
| | yl)methyl]acetamide |
| I-327 | N-(1,1-dioxo-2,3-dihydro-1lambda6-thiophen-3-yl)-2-[3-fluoro- |
| | 4-(1H-pyrrol-1-yl)phenyl]-N-[(pyridin-4-yl)methyl]acetamide |
| I-328 | N-(1,1-dioxo-2,3-dihydro-1lambda6-thiophen-3-yl)-2-{4′-fluoro- |
| | [1,1′-biphenyl]-4-yl}-2-(2-oxopyrrolidin-1-yl)-N-[(pyridin-4- |
| | yl)methyl]acetamide |
| I-329 | rel-N-[(3R)-1,1-dioxo-2,3-dihydro-1lambda6-thiophen-3-yl]-2- |
| | [2-fluoro-4-(2-methyl-1H-pyrrol-1-yl)phenyl]-N-[(2- |
| | methoxypyridin-4-yl)methyl]acetamide |
| I-330 | (2R*)-N-[(3R**)-1,1-dioxo-2,3-dihydro-1lambda6-thiophen-3- |
| | yl]-2-{4′-fluoro-[1,1′-biphenyl]-4-yl}-2-methoxy-N-[(pyridin-4- |
| | yl)methyl]acetamide |
| I-331 | 2-[4-(difluoromethoxy)phenyl]-N-(1,1-dioxo-2,3-dihydro- |
| | 1lambda6-thiophen-3-yl)-N-[(pyridin-4-yl)methyl]acetamide |
| I-332 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-{3,3′,4′- |
| | trifluoro-[1,1′-biphenyl]-4-yl}acetamide |
| I-333 | N-(1,1-dioxo-2,3-dihydro-1lambda6-thiophen-3-yl)-2-[2-(3- |
| | methylphenyl)-1,3-thiazol-4-yl]-N-[(pyridin-4- |
| | yl)methyl]acetamide |
| I-334 | 2-[2′-(difluoromethyl)-4′-fluoro-[1,1′-biphenyl]-4-yl]-N-(1,1- |
| | dioxo-2,3-dihydro-1lambda6-thiophen-3-yl)-N-[(pyridin-4- |
| | yl)methyl]acetamide |
| I-335 | N-(1,1-dioxo-2,3-dihydro-1lambda6-thiophen-3-yl)-N-[(2- |
| | methyl-1,3-thiazol-4-yl)methyl]-2-[4-(1H-pyrrol-1- |
| | yl)phenyl]acetamide |
| I-336 | rel-N-[(3R)-1,1-dioxo-2,3-dihydro-1lambda6-thiophen-3-yl]-2- |
| | [4-(pentafluoro-lambda6-sulfanyl)phenyl]-N-[(pyridin-4- |
| | yl)methyl]acetamide |
| I-337 | N-benzyl-N-(1,1-dioxo-2,3-dihydro-1lambda6-thiophen-3-yl)-2- |
| | methoxy-2-[4-(1H-pyrrol-1-yl)phenyl]acetamide |
| I-338 | N-(1,1-dioxo-2,3-dihydro-1lambda6-thiophen-3-yl)-N-[(pyridin- |
| | 4-yl)methyl]-2-[4-(thiophen-2-yl)phenyl]acetamide |
| I-339 | N-(1,1-dioxo-2,3-dihydro-1lambda6-thiophen-3-yl)-2-[4- |
| | (methylsulfanyl)phenyl]-N-[(pyridin-4-yl)methyl]acetamide |
| I-340 | (2R**)-2-{3,4′-difluoro-[1,1′-biphenyl]-4-yl}-N-[(3R*)-1,1- |
| | dioxo-2,3-dihydro-1lambda6-thiophen-3-yl]-N-{[2-(2- |
| | hydroxypropan-2-yl)pyridin-4-yl]methyl}-N′,N′- |
| | dimethylpropanediamide |
| I-341 | N-benzyl-N-(1,1-dioxo-2,3-dihydro-1lambda6-thiophen-3-yl)-2- |
| | (5-phenylpyridin-2-yl)acetamide |
| I-342 | N-[(5-cyclopropyl-1,2,4-oxadiazol-3-yl)methyl]-N-(1,1-dioxo- |
| | 2,3-dihydro-1lambda6-thiophen-3-yl)-2-[4-(1H-pyrrol-1- |
| | yl)phenyl]acetamide |
| I-343 | N-(1,1-dioxo-2,3-dihydro-1lambda6-thiophen-3-yl)-N-[(2- |
| | methoxypyridin-4-yl)methyl]-3-[4-(pentafluoro-lambda6- |
| | sulfanyl)phenyl]propanamide |
| I-344 | N-(1,1-dioxo-2,3-dihydro-1lambda6-thiophen-3-yl)-N-[(3- |
| | methanesulfonamidophenyl)methyl]-2-[4-(1H-pyrrol-1- |
| | yl)phenyl]acetamide |
| I-345 | N-(1,1-dioxo-2,3-dihydro-1lambda6-thiophen-3-yl)-2-{4′-fluoro- |
| | 3-methoxy-[1,1′-biphenyl]-4-yl}-N-[(pyridin-4- |
| | yl)methyl]acetamide |
| I-346 | (1s,3s)-N-(1,1-dioxo-2,3-dihydro-1lambda6-thiophen-3-yl)-N- |
| | [(pyridin-4-yl)methyl]-3-[4-(trifluoromethyl)phenyl]cyclobutane- |
| | 1-carboxamide |
| I-347 | 2-(4-{bicyclo[1.1.1]pentan-1-yl}phenyl)-N-(1,1-dioxo-2,3- |
| | dihydro-1lambda6-thiophen-3-yl)-N-[(pyridin-4- |
| | yl)methyl]acetamide |
| I-348 | 2-{2′-cyano-4′-fluoro-[1,1′-biphenyl]-4-yl}-N-(1,1-dioxo-2,3- |
| | dihydro-1lambda6-thiophen-3-yl)-N-[(pyridin-4- |
| | yl)methyl]acetamide |
| I-349 | N-(1,1-dioxo-2,3-dihydro-1lambda6-thiophen-3-yl)-2-[5-(4- |
| | fluorophenyl)-2-oxo-2,3-dihydro-1,3,4-oxadiazol-3-yl]-N- |
| | [(pyridin-4-yl)methyl]acetamide |
| I-350 | N-(1,1-dioxo-2,3-dihydro-1lambda6-thiophen-3-yl)-2-[4′-fluoro- |
| | 3-(hydroxymethyl)-[1,1′-biphenyl]-4-yl]-N-[(pyridin-4- |
| | yl)methyl]acetamide |
| I-351 | 2-(4-difluoromethanesulfonylphenyl)-N-(1,1-dioxo-2,3-dihydro- |
| | 1lambda6-thiophen-3-yl)-N-[(pyridin-4-yl)methyl]acetamide |
| I-352 | 3-{[N-(1,1-dioxo-2,3-dihydro-1lambda6-thiophen-3-yl)-2-[4- |
| | (1H-pyrrol-1-yl)phenyl]acetamido]methyl}benzoic acid |
| I-353 | rel-N-[(3R)-1,1-dioxo-2,3-dihydro-1lambda6-thiophen-3-yl]-2- |
| | [6-(4-fluoro-2-hydroxyphenyl)pyridin-3-yl]-N-[(2- |
| | methoxypyridin-4-yl)methyl]acetamide |
| I-354 | N-(1,1-dioxo-2,3-dihydro-1lambda6-thiophen-3-yl)-2-(5-phenyl- |
| | 1,2-oxazol-3-yl)-N-[(pyridin-4-yl)methyl]acetamide |
| I-355 | N-(1,1-dioxo-2,3-dihydro-1lambda6-thiophen-3-yl)-2-[4-(3- |
| | methyl-1H-pyrrol-1-yl)phenyl]-N-[(pyridin-4- |
| | yl)methyl]acetamide |
| I-356 | N-(1,1-dioxo-2,3-dihydro-1lambda6-thiophen-3-yl)-2-methoxy-2- |
| | {4′-methyl-[1,1′-biphenyl]-4-yl}-N-[(pyridin-4- |
| | yl)methyl]acetamide |
| I-357 | N-(1,1-dioxo-2,3-dihydro-1lambda6-thiophen-3-yl)-3-methoxy- |
| | N-[(2-methoxypyridin-4-yl)methyl]-2-(4-phenyl-1H-pyrazol-1- |
| | yl)propanamide |
| I-358 | N-(1,1-dioxo-2,3-dihydro-1lambda6-thiophen-3-yl)-N-[(pyridin- |
| | 3-yl)methyl]-2-[4-(1H-pyrrol-1-yl)phenyl]acetamide |
| I-359 | 2-[4-(3,3-difluorocyclobutyl)phenyl]-N-(1,1-dioxo-2,3-dihydro- |
| | 1lambda6-thiophen-3-yl)-N-[(pyridin-4-yl)methyl]acetamide |
| I-360 | N-(1,1-dioxo-2,3-dihydro-1lambda6-thiophen-3-yl)-N-{[2- |
| | (hydroxymethyl)phenyl]methyl}-2-[4-(1H-pyrrol-1- |
| | yl)phenyl]acetamide |
| I-361 | N-(1,1-dioxo-2,3-dihydro-1lambda6-thiophen-3-yl)-N- |
| | ({imidazo[1,2-a]pyridin-6-yl}methyl)-2-[4-(1H-pyrrol-1- |
| | yl)phenyl]acetamide |
| I-362 | (2R**)-N′-[(3R*)-1,1-dioxo-2,3-dihydro-1lambda6-thiophen-3- |
| | yl]-2-{4′-fluoro-[1,1′-biphenyl]-4-yl}-N,N-dimethyl-N′-[(pyridin- |
| | 4-yl)methyl]propanediamide |
| I-363 | N-(1,1-dioxo-2,3-dihydro-1lambda6-thiophen-3-yl)-2-[2-fluoro- |
| | 4-(1,3-thiazol-4-yl)phenyl]-N-[(2-methoxypyridin-4- |
| | yl)methyl]acetamide |
| I-364 | N-(1,1-dioxo-2,3-dihydro-1lambda6-thiophen-3-yl)-2-[2-fluoro- |
| | 4-(2-methyl-1H-pyrrol-1-yl)phenyl]-N-[(pyrimidin-5- |
| | yl)methyl]acetamide |
| I-365 | N-(1,1-dioxo-2,3-dihydro-1lambda6-thiophen-3-yl)-2-[4-(propan- |
| | 2-yloxy)phenyl]-N-[(pyridin-4-yl)methyl]acetamide |
| I-366 | (2R*)-2-{3,4′-difluoro-[1,1′-biphenyl]-4-yl}-N-[(3R**)-1,1- |
| | dioxo-2,3-dihydro-1lambda6-thiophen-3-yl]-2-methoxy-N-[(2- |
| | methoxypyridin-4-yl)methyl]acetamide |
| I-367 | N-(1,1-dioxo-2,3-dihydro-1lambda6-thiophen-3-yl)-N-[(pyridin- |
| | 4-yl)methyl]-2-[6-(trifluoromethyl)pyridin-3-yl]acetamide |
| I-368 | N-(1,1-dioxo-2,3-dihydro-1lambda6-thiophen-3-yl)-3-(6-fluoro- |
| | 1H-indol-3-yl)-N-[(pyridin-4-yl)methyl]propanamide |
| I-369 | N-(1,1-dioxo-2,3-dihydro-1lambda6-thiophen-3-yl)-N-[(2-oxo- |
| | 1,2-dihydropyridin-3-yl)methyl]-2-[4-(1H-pyrrol-1- |
| | yl)phenyl]acetamide |
| I-370 | N-(1,1-dioxo-2,3-dihydro-1lambda6-thiophen-3-yl)-N-[(pyridin- |
| | 4-yl)methyl]-2-[4-(2H-1,2,3,4-tetrazol-5-yl)phenyl]acetamide |
| I-371 | N-(1,1-dioxo-2,3-dihydro-1lambda6-thiophen-3-yl)-N-[(pyridin- |
| | 4-yl)methyl]-2-[4-(trifluoromethyl)-1,3-thiazol-2-yl]acetamide |
| I-372 | N-(1,1-dioxo-2,3-dihydro-1lambda6-thiophen-3-yl)-N-{[2-(3- |
| | hydroxypyrrolidin-1-yl)phenyl]methyl}-2-[4-(1H-pyrrol-1- |
| | yl)phenyl]acetamide |
| I-373 | N-benzyl-N-[5-(hydroxymethyl)-1,1-dioxo-2,3-dihydro- |
| | 1lambda6-thiophen-3-yl]-2-[4-(1H-pyrrol-1-yl)phenyl]acetamide |
| I-374 | (2R**)-N′-[(3R*)-1,1-dioxo-2,3-dihydro-1lambda6-thiophen-3- |
| | yl]-2-{4′-fluoro-[1,1′-biphenyl]-4-yl}-N,N-dimethyl-N′-[(pyridin- |
| | 4-yl)methyl]propanediamide |
| I-375 | (2R**)-N′-[(3R*)-1,1-dioxo-2,3-dihydro-1lambda6-thiophen-3- |
| | yl]-2-{4′-fluoro-[1,1′-biphenyl]-4-yl}-N,N-dimethyl-N′-[(pyridin- |
| | 4-yl)methyl]propanediamide |
| I-376 | N-(1,1-dioxo-2,3-dihydro-1lambda6-thiophen-3-yl)-2-[4′-fluoro- |
| | 3-(methoxymethyl)-[1,1′-biphenyl]-4-yl]-N-[(pyridin-4- |
| | yl)methyl]acetamide |
| I-377 | N-(1,1-dioxo-2,3-dihydro-1lambda6-thiophen-3-yl)-2-(N- |
| | methylacetamido)-N-[(pyridin-4-yl)methyl]-2-[4-(1H-pyrrol-1- |
| | yl)phenyl]acetamide |
| I-378 | rel-(2R)-N-(1,1-dioxo-2,3-dihydro-1lambda6-thiophen-3-yl)-2- |
| | {4′-fluoro-[1,1′-biphenyl]-4-yl}-2-(N-methylacetamido)-N- |
| | [(pyridin-4-yl)methyl]acetamide |
| I-379 | 2-[1-(4-chlorophenyl)-1H-pyrazol-4-yl]-N-(1,1-dioxo-2,3- |
| | dihydro-1lambda6-thiophen-3-yl)-N-[(2-methoxypyridin-4- |
| | yl)methyl]acetamide |
| I-380 | 2-[1-(4-chlorophenyl)-1H-pyrazol-3-yl]-N-(1,1-dioxo-2,3- |
| | dihydro-1lambda6-thiophen-3-yl)-N-[(2-methoxypyridin-4- |
| | yl)methyl]acetamide |
| I-381 | N-(1,1-dioxo-2,3-dihydro-1lambda6-thiophen-3-yl)-2-[2-fluoro- |
| | 4-(2-oxo-1,2-dihydropyridin-1-yl)phenyl]-N-[(2-methoxypyridin- |
| | 4-yl)methyl]acetamide |
| I-382 | rel-N-[(3R)-1,1-dioxo-2,3-dihydro-1lambda6-thiophen-3-yl]-N- |
| | [(2-methoxypyridin-4-yl)methyl]-2-[4- |
| | (trifluoromethyl)phenyl]acetamide |
| I-383 | N-benzyl-N-(1,1-dioxo-2,3-dihydro-1lambda6-thiophen-3-yl)-2- |
| | {4-[2-(2-hydroxyethyl)-1H-pyrrol-1-yl]phenyl}acetamide |
| I-384 | N-benzyl-N-(1,1-dioxo-2,3-dihydro-1lambda6-thiophen-3-yl)-2- |
| | {4-[2-(2-methoxyethyl)-1H-pyrrol-1-yl]phenyl}acetamide |
| I-385 | N-(1,1-dioxo-2,3-dihydro-1lambda6-thiophen-3-yl)-6-(4- |
| | fluorophenyl)-N-[(2-methoxypyridin-4-yl)methyl]-1-methyl-2,3- |
| | dihydro-1H-indole-3-carboxamide |
| I-386 | N-(2-cyanoethyl)-N-(1,1-dioxo-2,3-dihydro-1lambda6-thiophen- |
| | 3-yl)-2-{4′-fluoro-[1,1′-biphenyl]-4-yl}acetamide |
| I-387 | N-(1,1-dioxo-2,3-dihydro-1lambda6-thiophen-3-yl)-N-[(pyridin- |
| | 4-yl)methyl]-2-[4-(trifluoromethyl)phenyl]cyclopropane-1- |
| | carboxamide |
| I-388 | 2-[4-(dimethylsulfamoyl)phenyl]-N-(1,1-dioxo-2,3-dihydro- |
| | 1lambda6-thiophen-3-yl)-N-[(pyridin-4-yl)methyl]acetamide |
| I-389 | N-(1,1-dioxo-2,3-dihydro-1lambda6-thiophen-3-yl)-N-[(pyridin- |
| | 4-yl)methyl]-2-[5-(trifluoromethyl)pyridin-2-yl]acetamide |
| I-390 | N-(1,1-dioxo-2,3-dihydro-1lambda6-thiophen-3-yl)-2-(1-phenyl- |
| | 1H-pyrazol-3-yl)-N-[(pyridin-4-yl)methyl]acetamide |
| I-391 | 3-(5-chloro-1,3-benzothiazol-2-yl)-N-(1,1-dioxo-2,3-dihydro- |
| | 1lambda6-thiophen-3-yl)-N-[(pyridin-4-yl)methyl]propanamide |
| I-392 | N-(1,1-dioxo-2,3-dihydro-1lambda6-thiophen-3-yl)-3- |
| | (naphthalen-2-yl)-N-[(pyridin-4-yl)methyl]propanamide |
| I-393 | N-(1,1-dioxo-2,3-dihydro-1lambda6-thiophen-3-yl)-N-[(pyridin- |
| | 4-yl)methyl]-2-[3-(2,2,2-trifluoroethyl)phenyl]acetamide |
| I-394 | N-(1,1-dioxo-2,3-dihydro-1lambda6-thiophen-3-yl)-2-[1-(4- |
| | fluorophenyl)-1H-pyrazol-3-yl]-N-[(pyridin-4- |
| | yl)methyl]acetamide |
| I-395 | 3-benzyl-N-(1,1-dioxo-2,3-dihydro-1lambda6-thiophen-3-yl)-N- |
| | [(pyridin-4-yl)methyl]cyclobutane-1-carboxamide |
| I-396 | N-(1,1-dioxo-2,3-dihydro-1lambda6-thiophen-3-yl)-3-(1H-indol- |
| | 2-yl)-N-[(pyridin-4-yl)methyl]propanamide |
| I-397 | (2R)-2-(cyclopropylformamido)-N-(1,1-dioxo-2,3-dihydro- |
| | 1lambda6-thiophen-3-yl)-3-(1H-indol-3-yl)-N-[(pyridin-4- |
| | yl)methyl]propanamide |
| I-398 | (3s)-N-(1,1-dioxo-2,3-dihydro-1lambda6-thiophen-3-yl)-3-[(3- |
| | fluorophenyl)methyl]-3-hydroxy-N-[(pyridin-4- |
| | yl)methyl]cyclobutane-1-carboxamide |
| I-399 | N-(1,1-dioxo-2,3-dihydro-1lambda6-thiophen-3-yl)-5-(4- |
| | fluorophenyl)-N-[(2-methoxypyridin-4-yl)methyl]-2,3-dihydro-1- |
| | benzofuran-2-carboxamide |
| I-400 | rel-N-[(3R)-1,1-dioxo-2,3-dihydro-1lambda6-thiophen-3-yl]-N- |
| | [(2-methoxypyridin-4-yl)methyl]-2-{3,3′,4′-trifluoro-[1,1′- |
| | biphenyl]-4-yl}acetamide |
| I-401 | 2-[2-(difluoromethyl)-4′-fluoro-[1,1′-biphenyl]-4-yl]-N-(1,1- |
| | dioxo-2,3-dihydro-1lambda6-thiophen-3-yl)-N-[(pyridin-4- |
| | yl)methyl]acetamide |
| I-402 | N-[(4-cyclopropyl-1,3-thiazol-2-yl)methyl]-N-[(3S)-1,1-dioxo- |
| | 2,3-dihydro-1lambda6-thiophen-3-yl]-2-[4-(1H-pyrrol-1- |
| | yl)phenyl]acetamide |
| I-403 | N-(1,1-dioxo-2,3-dihydro-1lambda6-thiophen-3-yl)-2-(4-phenyl- |
| | 1H-1,2,3-triazol-1-yl)-N-[(pyridin-4-yl)methyl]acetamide |
| I-404 | (2R*)-2-{3,4′-difluoro-[1,1′-biphenyl]-4-yl}-N-[(3R**)-1,1- |
| | dioxo-2,3-dihydro-1lambda6-thiophen-3-yl]-2-methoxy-N-[(2- |
| | methoxypyridin-4-yl)methyl]acetamide |
| I-405 | (2R*)-2-{3,4′-difluoro-[1,1′-biphenyl]-4-yl}-N-[(3R**)-1,1- |
| | dioxo-2,3-dihydro-1lambda6-thiophen-3-yl]-2-methoxy-N-[(2- |
| | methoxypyridin-4-yl)methyl]acetamide |
| I-406 | N-(1,1-dioxo-2,3-dihydro-1lambda6-thiophen-3-yl)-2-(2-phenyl- |
| | 2H-1,2,3-triazol-4-yl)-N-[(pyridin-4-yl)methyl]acetamide |
| I-407 | N-(1,1-dioxo-2,3-dihydro-1lambda6-thiophen-3-yl)-2-[4-(oxolan- |
| | 3-yl)-1H-pyrazol-1-yl]-N-[(pyridin-4-yl)methyl]acetamide |
| I-408 | 2-(4-bromo-1H-pyrazol-1-yl)-N-(1,1-dioxo-2,3-dihydro- |
| | 1lambda6-thiophen-3-yl)-N-[(pyridin-4-yl)methyl]acetamide |
| I-409 | N-(1,1-dioxo-2,3-dihydro-1lambda6-thiophen-3-yl)-2-(1-phenyl- |
| | 1H-pyrazol-4-yl)-N-[(pyridin-4-yl)methyl]acetamide |
| I-410 | 2-[2-(3,4-difluorophenyl)-1,3-oxazol-4-yl]-N-(1,1-dioxo-2,3- |
| | dihydro-1lambda6-thiophen-3-yl)-N-[(pyridin-4- |
| | yl)methyl]acetamide |
| I-411 | rac-(1R,2R)-N-(1,1-dioxo-2,3-dihydro-1lambda6-thiophen-3-yl)- |
| | 2-(naphthalen-2-yl)-N-[(pyridin-4-yl)methyl]cyclopropane-1- |
| | carboxamide |
| I-412 | N-(1,1-dioxo-2,3-dihydro-1lambda6-thiophen-3-yl)-2-(5- |
| | methoxy-1H-indol-2-yl)-N-[(pyridin-4-yl)methyl]acetamide |
| I-413 | N-(1,1-dioxo-2,3-dihydro-1lambda6-thiophen-3-yl)-3-(2H- |
| | indazol-3-yl)-N-[(pyridin-4-yl)methyl]propanamide |
| I-414 | N-(1,1-dioxo-2,3-dihydro-1lambda6-thiophen-3-yl)-2-(2H- |
| | indazol-6-yl)-N-[(pyridin-4-yl)methyl]acetamide |
| I-415 | N-(1,1-dioxo-2,3-dihydro-1lambda6-thiophen-3-yl)-3-phenyl-N- |
| | [(pyridin-4-yl)methyl]bicyclo[1.1.1]pentane-1-carboxamide |
| I-416 | N-(1,1-dioxo-2,3-dihydro-1lambda6-thiophen-3-yl)-2-(4- |
| | phenylcyclohexyl)-N-[(pyridin-4-yl)methyl]acetamide |
| I-417 | 2-(6-chloro-1H-indol-3-yl)-N-(1,1-dioxo-2,3-dihydro-1lambda6- |
| | thiophen-3-yl)-N-[(pyridin-4-yl)methyl]acetamide |
| I-418 | N-(1,1-dioxo-2,3-dihydro-1lambda6-thiophen-3-yl)-2-(4-methyl- |
| | 2-oxo-1,2-dihydroquinolin-3-yl)-N-[(pyridin-4- |
| | yl)methyl]acetamide |
| I-419 | N-(1,1-dioxo-2,3-dihydro-1lambda6-thiophen-3-yl)-2-[4-(1H- |
| | pyrazol-1-yl)phenyl]-N-[(pyridin-4-yl)methyl]acetamide |
| I-420 | N-(1,1-dioxo-2,3-dihydro-1lambda6-thiophen-3-yl)-2-(3-phenyl- |
| | 1H-1,2,4-triazol-1-yl)-N-[(pyridin-4-yl)methyl]acetamide |
| I-421 | N-(1,1-dioxo-2,3-dihydro-1lambda6-thiophen-3-yl)-N-phenyl-2- |
| | [4-(1H-pyrrol-1-yl)phenyl]acetamide |
| I-422 | N-(1,1-dioxo-2,3-dihydro-1lambda6-thiophen-3-yl)-N-[(pyridin- |
| | 4-yl)methyl]-5-(1H-pyrrol-1-yl)-2,3-dihydro-1H-indene-2- |
| | carboxamide |
| I-423 | N-(1,1-dioxo-2,3-dihydro-1lambda6-thiophen-3-yl)-6-(4- |
| | fluorophenyl)-N-[(pyridin-4-yl)methyl]-1H-indole-3- |
| | carboxamide |
| I-424 | N-(1,1-dioxo-2,3-dihydro-1lambda6-thiophen-3-yl)-6-(4- |
| | fluorophenyl)-N-{[2-(2-hydroxypropan-2-yl)pyridin-4- |
| | yl]methyl}-1H-indole-3-carboxamide |
| I-425 | N-[(3S)-1,1-dioxo-2,3-dihydro-1lambda6-thiophen-3-yl]-2-[2- |
| | fluoro-4-(2-methyl-1H-pyrrol-1-yl)phenyl]-N-[(2- |
| | methoxypyridin-4-yl)methyl]acetamide |
| I-426 | N-(1,1-dioxo-2,3-dihydro-1lambda6-thiophen-3-yl)-2-(4-phenyl- |
| | 1H-pyrazol-1-yl)-N-[(pyridin-4-yl)methyl]cyclobutane-1- |
| | carboxamide |
| I-427 | 2-(4-chloro-1H-pyrazol-1-yl)-N-(1,1-dioxo-2,3-dihydro- |
| | 1lambda6-thiophen-3-yl)-N-[(pyridin-4-yl)methyl]acetamide |
| I-428 | N-(1,1-dioxo-2,3-dihydro-1lambda6-thiophen-3-yl)-2-{8- |
| | oxatricyclo[7.4.0.0\hat{ }{2,7}]trideca-1(13),2,4,6,9,11-hexaen-4-yl}- |
| | N-[(pyridin-4-yl)methyl]acetamide |
| I-429 | 2-(4-cyclopropyl-1H-pyrazol-1-yl)-N-(1,1-dioxo-2,3-dihydro- |
| | 1lambda6-thiophen-3-yl)-N-[(pyridin-4-yl)methyl]acetamide |
| I-430 | N-(1,1-dioxo-2,3-dihydro-1lambda6-thiophen-3-yl)-N-phenyl-2- |
| | (4-phenyl-1,2,3,6-tetrahydropyridin-1-yl)acetamide |
| I-431 | N-(1,1-dioxo-2,3-dihydro-1lambda6-thiophen-3-yl)-2- |
| | (naphthalen-1-yl)-N-[(pyridin-4-yl)methyl]acetamide |
| I-432 | 2-[4-(cyanomethoxy)phenyl]-N-(1,1-dioxo-2,3-dihydro- |
| | 1lambda6-thiophen-3-yl)-N-[(pyridin-4-yl)methyl]acetamide |
| I-433 | N-(1,1-dioxo-2,3-dihydro-1lambda6-thiophen-3-yl)-2-(2H- |
| | indazol-2-yl)-N-[(pyridin-4-yl)methyl]acetamide |
| I-434 | N-(1,1-dioxo-2,3-dihydro-1lambda6-thiophen-3-yl)-2-(5-methyl- |
| | 1-phenyl-1H-1,2,3-triazol-4-yl)-N-[(pyridin-4- |
| | yl)methyl]acetamide |
| I-435 | 2-cyclopropyl-N-(1,1-dioxo-2,3-dihydro-1lambda6-thiophen-3- |
| | yl)-2-(4-phenyl-1H-pyrazol-1-yl)-N-[(pyridin-4- |
| | yl)methyl]acetamide |
| I-436 | N-(1,1-dioxo-2,3-dihydro-1lambda6-thiophen-3-yl)-2-(4-methyl- |
| | 1H-pyrazol-1-yl)-N-[(pyridin-4-yl)methyl]acetamide |
| I-437 | 2-(3-cyclopropylphenyl)-N-(1,1-dioxo-2,3-dihydro-1lambda6- |
| | thiophen-3-yl)-N-[(pyridin-4-yl)methyl]acetamide |
| I-438 | 2-(2-cyclopropyl-1,3-thiazol-4-yl)-N-(1,1-dioxo-2,3-dihydro- |
| | 1lambda6-thiophen-3-yl)-N-[(pyridin-4-yl)methyl]acetamide |
| I-439 | N-(1,1-dioxo-2,3-dihydro-1lambda6-thiophen-3-yl)-2-{3- |
| | phenylbicyclo[1.1.1]pentan-1-yl}-N-[(pyridin-4- |
| | yl)methyl]acetamide |
| I-440 | 2-(4-cyclopentyl-1,3-thiazol-2-yl)-N-(1,1-dioxo-2,3-dihydro- |
| | 1lambda6-thiophen-3-yl)-N-[(pyridin-4-yl)methyl]acetamide |
| I-441 | N-(1,1-dioxo-2,3-dihydro-1lambda6-thiophen-3-yl)-2-[4-(5- |
| | methyl-1H-pyrazol-1-yl)phenyl]-N-[(pyridin-4- |
| | yl)methyl]acetamide |
| I-442 | N-(1,1-dioxo-2,3-dihydro-1lambda6-thiophen-3-yl)-2-(5-phenyl- |
| | 2H-1,2,3,4-tetrazol-2-yl)-N-[(pyridin-4-yl)methyl]acetamide |
| I-443 | N-(1,1-dioxo-2,3-dihydro-1lambda6-thiophen-3-yl)-2-(4-methyl- |
| | 5-phenyl-4H-1,2,4-triazol-3-yl)-N-[(pyridin-4- |
| | yl)methyl]acetamide |
| I-444 | N-(1,1-dioxo-2,3-dihydro-1lambda6-thiophen-3-yl)-2-[4-(2- |
| | oxopyrrolidin-1-yl)phenyl]-N-[(pyridin-4-yl)methyl]acetamide |
| I-445 | 2-(2-cyclopentyl-1,3-thiazol-4-yl)-N-(1,1-dioxo-2,3-dihydro- |
| | 1lambda6-thiophen-3-yl)-N-[(pyridin-4-yl)methyl]acetamide |
| I-446 | N-(1,1-dioxo-2,3-dihydro-1lambda6-thiophen-3-yl)-2-(3-methyl- |
| | 4-phenyl-1H-pyrazol-1-yl)-N-[(pyridin-4-yl)methyl]acetamide |
| I-447 | N-(1,1-dioxo-2,3-dihydro-1lambda6-thiophen-3-yl)-2-(2-oxo-5- |
| | phenyl-2,3-dihydro-1,3,4-oxadiazol-3-yl)-N-[(pyridin-4- |
| | yl)methyl]acetamide |
| I-448 | N-(4-{[(1,1-dioxo-2,3-dihydro-1lambda6-thiophen-3- |
| | yl)[(pyridin-4- |
| | yl)methyl]carbamoyl]methyl}phenyl)cyclopropanecarboxamide |
| I-449 | N-(1,1-dioxo-2,3-dihydro-1lambda6-thiophen-3-yl)-2-[4-(2- |
| | hydroxycyclopentyl)phenyl]-N-[(pyridin-4-yl)methyl]acetamide |
| I-450 | N-(1,1-dioxo-2,3-dihydro-1lambda6-thiophen-3-yl)-2-(3-phenyl- |
| | 1H-pyrazol-1-yl)-N-[(pyridin-4-yl)methyl]acetamide |
| I-451 | N-(1,1-dioxo-2,3-dihydro-1lambda6-thiophen-3-yl)-2-[4-(5- |
| | fluoropyridin-2-yl)-3-[(4-methoxyphenyl)methoxy]phenyl]-N- |
| | [(2-methoxypyridin-4-yl)methyl]acetamide |
| I-452 | N-(1,1-dioxo-2,3-dihydro-1lambda6-thiophen-3-yl)-2-{4′-fluoro- |
| | [1,1′-biphenyl]-4-yl}-3-(N-methylacetamido)-N-[(pyridin-4- |
| | yl)methyl]propanamide |
| I-453 | N-(1,1-dioxo-2,3-dihydro-1lambda6-thiophen-3-yl)-2-[2-fluoro- |
| | 4-(4-fluoro-2-oxo-1,2-dihydropyridin-1-yl)phenyl]-N-[(2- |
| | methoxypyridin-4-yl)methyl]acetamide |
| I-454 | N-(1,1-dioxo-2,3-dihydro-1lambda6-thiophen-3-yl)-2-(1H- |
| | indazol-1-yl)-N-[(pyridin-4-yl)methyl]acetamide |
| I-455 | N-(1,1-dioxo-2,3-dihydro-1lambda6-thiophen-3-yl)-N-[(pyridin- |
| | 4-yl)methyl]-2-[6-(trifluoromethyl)-1H-indazol-1-yl]acetamide |
| I-456 | N-benzyl-N-(1,1-dioxo-2,3-dihydro-1lambda6-thiophen-3-yl)-2- |
| | (4-{5-methyl-1H,4H,5H,6H,7H-pyrrolo[3,2-c]pyridin-1- |
| | yl}phenyl)acetamide |
| I-457 | N-(1,1-dioxo-2,3-dihydro-1lambda6-thiophen-3-yl)-2-[4-(4- |
| | fluorophenyl)-2-oxopiperidin-1-yl]-N-[(pyridin-4- |
| | yl)methyl]acetamide |
| I-458 | N-(1,1-dioxo-2,3-dihydro-1lambda6-thiophen-3-yl)-2-{4′-fluoro- |
| | [1,1′-biphenyl]-4-yl}-3-(N-methylmethanesulfonamido)-N- |
| | [(pyridin-4-yl)methyl]propanamide |
| I-459 | 2-(6-chloro-1H-indol-3-yl)-N-(1,1-dioxo-2,3-dihydro-1lambda6- |
| | thiophen-3-yl)-N-[(pyridin-4-yl)methyl]cyclopropane-1- |
| | carboxamide |
| I-460 | N-(1,1-dioxo-2,3-dihydro-1lambda6-thiophen-3-yl)-N-{[2-(2- |
| | hydroxypropan-2-yl)pyridin-4-yl]methyl}-N′,N′-dimethyl-2-[4- |
| | (pentafluoro-lambda6-sulfanyl)phenyl]butanediamide |
| I-461 | 2-[4-(azetidin-1-yl)phenyl]-N-(1,1-dioxo-2,3-dihydro-1lambda6- |
| | thiophen-3-yl)-N-[(pyridin-4-yl)methyl]acetamide |
| I-462 | N-(1,1-dioxo-2,3-dihydro-1lambda6-thiophen-3-yl)-N-[2- |
| | (pyridin-4-yl)ethyl]-2-[4-(1H-pyrrol-1-yl)phenyl]acetamide |
| I-463 | 2-(4-cyclobutoxyphenyl)-N-(1,1-dioxo-2,3-dihydro-1lambda6- |
| | thiophen-3-yl)-N-[(pyridin-4-yl)methyl]acetamide |
| I-464 | N-(1,1-dioxo-2,3-dihydro-1lambda6-thiophen-3-yl)-N-[(pyridin- |
| | 4-yl)methyl]-2-[4-(pyrrolidin-1-yl)phenyl]acetamide |
| I-465 | N-(1,1-dioxo-2,3-dihydro-1lambda6-thiophen-3-yl)-2-[4′-fluoro- |
| | 3-(trifluoromethyl)-[1,1′-biphenyl]-4-yl]-N-[(pyridin-4- |
| | yl)methyl]acetamide |
| I-466 | N-(1,1-dioxo-2,3-dihydro-1lambda6-thiophen-3-yl)-N-[(pyridin- |
| | 4-yl)methyl]-2-[4-(1,3-thiazol-2-yl)phenyl]acetamide |
| I-467 | N-(1,1-dioxo-2,3-dihydro-1lambda6-thiophen-3-yl)-N-[(pyridin- |
| | 2-yl)methyl]-2-[4-(1H-pyrrol-1-yl)phenyl]acetamide |
| I-468 | 2-[2-cyano-4-(1H-pyrrol-1-yl)phenyl]-N-(1,1-dioxo-2,3-dihydro- |
| | 1lambda6-thiophen-3-yl)-N-[(pyridin-4-yl)methyl]acetamide |
| I-469 | rel-N-[(3R)-1,1-dioxo-2,3-dihydro-1lambda6-thiophen-3-yl]-2- |
| | [2-fluoro-4-(1H-pyrrol-1-yl)phenyl]-N-{[2-(2-hydroxypropan-2- |
| | yl)pyridin-4-yl]methyl}acetamide |
| I-470 | N-(1,1-dioxo-2,3-dihydro-1lambda6-thiophen-3-yl)-2-{4′-methyl- |
| | [1,1′-biphenyl]-4-yl}-N-[(pyridin-4-yl)methyl]acetamide |
| I-471 | N-(1,1-dioxo-2,3-dihydro-1lambda6-thiophen-3-yl)-2-[4-(1H- |
| | pyrrol-1-yl)phenyl]-N-(5,6,7,8-tetrahydroisoquinolin-5- |
| | yl)acetamide |
| I-472 | N-(1,1-dioxo-2,3-dihydro-1lambda6-thiophen-3-yl)-N-({3H- |
| | imidazo[4,5-b]pyridin-7-yl}methyl)-2-[4-(1H-pyrrol-1- |
| | yl)phenyl]acetamide |
| I-473 | 2-{4-[2-(aminomethyl)-1H-pyrrol-1-yl]phenyl}-N-benzyl-N-(1,1- |
| | dioxo-2,3-dihydro-1lambda6-thiophen-3-yl)acetamide |
| I-474 | 2-(4-bromophenyl)-N′-(1,1-dioxo-2,3-dihydro-1lambda6- |
| | thiophen-3-yl)-N,N-dimethyl-N′-[(pyridin-4- |
| | yl)methyl]propanediamide |
| I-475 | N-(1,1-dioxo-2,3-dihydro-1lambda6-thiophen-3-yl)-2-{4′-fluoro- |
| | [1,1′-biphenyl]-4-yl}-N′,N′-dimethyl-N-[(pyridin-4- |
| | yl)methyl]butanediamide |
| I-476 | (2R*)-N-[(3R**)-1,1-dioxo-2,3-dihydro-1lambda6-thiophen-3- |
| | yl]-2-{4′-fluoro-[1,1′-biphenyl]-4-yl}-2-methoxy-N-[(pyridin-4- |
| | yl)methyl]acetamide |
| I-477 | N-(1,1-dioxo-2,3-dihydro-1lambda6-thiophen-3-yl)-N-[(pyridin- |
| | 4-yl)methyl]-5-(1H-pyrrol-1-yl)-2,3-dihydro-1H-indene-1- |
| | carboxamide |
| I-478 | N-(1,1-dioxo-2,3-dihydro-1lambda6-thiophen-3-yl)-2-(4-phenyl- |
| | 1,2,3,6-tetrahydropyridin-1-yl)-N-[(pyridin-4- |
| | yl)methyl]acetamide |
| I-479 | N-(1,1-dioxo-2,3-dihydro-1lambda6-thiophen-3-yl)-N-{5-fluoro- |
| | 1H-pyrrolo[2,3-b]pyridin-3-yl}-2-[4-(1H-pyrrol-1- |
| | yl)phenyl]acetamide |
| I-480 | N-(1,1-dioxo-2,3-dihydro-1lambda6-thiophen-3-yl)-2-(4- |
| | ethynylphenyl)-N-[(pyridin-4-yl)methyl]acetamide |
| I-481 | N-(1,1-dioxo-2,3-dihydro-1lambda6-thiophen-3-yl)-N-[(1- |
| | methyl-1H-pyrazol-4-yl)methyl]-2-[4-(1H-pyrrol-1- |
| | yl)phenyl]acetamide |
| I-482 | 2-[4-(3,3-difluoroazetidin-1-yl)phenyl]-N-(1,1-dioxo-2,3- |
| | dihydro-1lambda6-thiophen-3-yl)-N-[(pyridin-4- |
| | yl)methyl]acetamide |
| I-483 | N-(1,1-dioxo-2,3-dihydro-1lambda6-thiophen-3-yl)-N- |
| | [(pyrimidin-2-yl)methyl]-2-[4-(1H-pyrrol-1-yl)phenyl]acetamide |
| I-484 | N-(1,1-dioxo-2,3-dihydro-1lambda6-thiophen-3-yl)-N-[(pyridin- |
| | 4-yl)methyl]-2-[4-(1H-pyrrol-1-yl)phenyl]butanamide |
| I-485 | N-(1,1-dioxo-2,3-dihydro-1lambda6-thiophen-3-yl)-N-[(oxan-4- |
| | yl)methyl]-2-[4-(1H-pyrrol-1-yl)phenyl]acetamide |
| I-486 | N-benzyl-N-(1,1-dioxo-2,3-dihydro-1lambda6-thiophen-3-yl)-4- |
| | (1H-pyrrol-1-yl)benzamide |
| I-487 | N-benzyl-N-(1,1-dioxo-2,3-dihydro-1lambda6-thiophen-3-yl)-5- |
| | phenylpyridine-2-carboxamide |
| I-488 | rel-N-benzyl-N-[(3R)-1,1-dioxo-2,3-dihydro-1lambda6-thiophen- |
| | 3-yl]-2-[4-(1H-pyrrol-1-yl)phenyl]acetamide |
| I-489 | N-(1,1-dioxo-2,3-dihydro-1lambda6-thiophen-3-yl)-N-methyl-2- |
| | [4-(1H-pyrrol-1-yl)phenyl]acetamide |
| I-490 | rel-N-[(3R)-1,1-dioxo-2,3-dihydro-1lambda6-thiophen-3-yl]-N- |
| | [(pyridin-4-yl)methyl]-2-[4-(1H-pyrrol-1-yl)phenyl]acetamide |
| I-491 | N-(1,1-dioxo-2,3-dihydro-1lambda6-thiophen-3-yl)-2-phenyl-N- |
| | [(pyridin-4-yl)methyl]acetamide |
| I-492 | 2-(1H-1,2,3-benzotriazol-1-yl)-N-(1,1-dioxo-2,3-dihydro- |
| | 1lambda6-thiophen-3-yl)-N-[(pyridin-4-yl)methyl]acetamide |
| I-493 | N-(1,1-dioxo-2,3-dihydro-1lambda6-thiophen-3-yl)-2-(1H-indol- |
| | 3-yl)-N-[(pyridin-4-yl)methyl]acetamide |
| I-494 | 2-(2H-1,3-benzodioxol-5-yl)-N-(1,1-dioxo-2,3-dihydro- |
| | 1lambda6-thiophen-3-yl)-N-[(pyridin-4-yl)methyl]acetamide |
| I-495 | 3-(1-benzothiophen-2-yl)-N-(1,1-dioxo-2,3-dihydro-1lambda6- |
| | thiophen-3-yl)-N-[(pyridin-4-yl)methyl]propanamide |
| I-496 | N-(1,1-dioxo-2,3-dihydro-1lambda6-thiophen-3-yl)-3-[3-(2- |
| | fluorophenyl)-1,2,4-oxadiazol-5-yl]-N-[(pyridin-4- |
| | yl)methyl]propanamide |
| I-497 | N-(1,1-dioxo-2,3-dihydro-1lambda6-thiophen-3-yl)-N-[(1H- |
| | indol-4-yl)methyl]-2-[4-(1H-pyrrol-1-yl)phenyl]acetamide |
| I-498 | N-(1,1-dioxo-2,3-dihydro-1lambda6-thiophen-3-yl)-N-[(1H- |
| | indol-5-yl)methyl]-2-[4-(1H-pyrrol-1-yl)phenyl]acetamide |
| I-499 | N-(1,1-dioxo-2,3-dihydro-1lambda6-thiophen-3-yl)-N-[(1H- |
| | indol-6-yl)methyl]-2-[4-(1H-pyrrol-1-yl)phenyl]acetamide |
| I-500 | N-(1,1-dioxo-2,3-dihydro-1lambda6-thiophen-3-yl)-4-(1H-indol- |
| | 3-yl)-N-[(pyridin-4-yl)methyl]butanamide |
| I-501 | 2-[4-(benzyloxy)phenyl]-N-(1,1-dioxo-2,3-dihydro-1lambda6- |
| | thiophen-3-yl)-N-[(pyridin-4-yl)methyl]acetamide |
| I-502 | N-(1,1-dioxo-2,3-dihydro-1lambda6-thiophen-3-yl)-3-(1H-indol- |
| | 1-yl)-N-[(pyridin-4-yl)methyl]propanamide |
| I-503 | N-(1,1-dioxo-2,3-dihydro-1lambda6-thiophen-3-yl)-2-(4- |
| | phenoxyphenyl)-N-[(pyridin-4-yl)methyl]acetamide |
| I-504 | (2S)-N-[(3R*)-1,1-dioxo-2,3-dihydro-1lambda6-thiophen-3-yl]- |
| | 2-acetamido-3-phenyl-N-[(pyridin-4-yl)methyl]propanamide |
| I-505 | (2S)-N-[(3R*)-1,1-dioxo-2,3-dihydro-1lambda6-thiophen-3-yl]- |
| | 2-acetamido-3-phenyl-N-[(pyridin-4-yl)methyl]propanamide |
| I-506 | N-(1,1-dioxo-2,3-dihydro-1lambda6-thiophen-3-yl)-2-(1H-indol- |
| | 6-yl)-N-[(pyridin-4-yl)methyl]acetamide |
| I-507 | N-(1,1-dioxo-2,3-dihydro-1lambda6-thiophen-3-yl)-N-[(pyridin- |
| | 4-yl)methyl]-3-[4-(trifluoromethyl)phenyl]propanamide |
| I-508 | N-(1,1-dioxo-2,3-dihydro-1lambda6-thiophen-3-yl)-N-[(pyridin- |
| | 4-yl)methyl]-3-[3-(trifluoromethoxy)phenyl]propanamide |
| I-509 | 4-benzyl-N-(1,1-dioxo-2,3-dihydro-1lambda6-thiophen-3-yl)-N- |
| | [(pyridin-4-yl)methyl]morpholine-2-carboxamide |
| I-510 | N-(1,1-dioxo-2,3-dihydro-1lambda6-thiophen-3-yl)-2-(3- |
| | phenoxyphenyl)-N-[(pyridin-4-yl)methyl]acetamide |
| I-511 | N-(1,1-dioxo-2,3-dihydro-1lambda6-thiophen-3-yl)-2-(6- |
| | methoxynaphthalen-2-yl)-N-[(pyridin-4-yl)methyl]acetamide |
| I-512 | N-(1,1-dioxo-2,3-dihydro-1lambda6-thiophen-3-yl)-N-[(pyridin- |
| | 4-yl)methyl]-2,3-dihydro-1H-indene-2-carboxamide |
| I-513 | 2-(4-cyanophenyl)-N-(1,1-dioxo-2,3-dihydro-1lambda6-thiophen- |
| | 3-yl)-N-[(pyridin-4-yl)methyl]acetamide |
| I-514 | N-(1,1-dioxo-2,3-dihydro-1lambda6-thiophen-3-yl)-N-[(pyridin- |
| | 4-yl)methyl]-2-(quinolin-6-yl)acetamide |
| I-515 | N-(1,1-dioxo-2,3-dihydro-1lambda6-thiophen-3-yl)-5-oxo-1- |
| | phenyl-N-[(pyridin-4-yl)methyl]pyrrolidine-3-carboxamide |
| I-516 | 2-(1,2-benzoxazol-3-yl)-N-(1,1-dioxo-2,3-dihydro-1lambda6- |
| | thiophen-3-yl)-N-[(pyridin-4-yl)methyl]acetamide |
| I-517 | (1S,2S)-2-(4-bromophenyl)-N-[(3R*)-1,1-dioxo-2,3-dihydro- |
| | 1lambda6-thiophen-3-yl]-N-[(pyridin-4-yl)methyl]cyclopropane- |
| | 1-carboxamide |
| I-518 | (1S,2S)-2-(4-bromophenyl)-N-[(3R*)-1,1-dioxo-2,3-dihydro- |
| | 1lambda6-thiophen-3-yl]-N-[(pyridin-4-yl)methyl]cyclopropane- |
| | 1-carboxamide |
| I-519 | N-(1,1-dioxo-2,3-dihydro-1lambda6-thiophen-3-yl)-N-[(pyridin- |
| | 4-yl)methyl]-3-[4-(1H-pyrrol-1-yl)phenyl]propanamide |
| I-520 | rel-N-[(3R)-1,1-dioxo-2,3-dihydro-1lambda6-thiophen-3-yl]-2- |
| | [2-fluoro-4-(1H-pyrrol-1-yl)phenyl]-N-[(pyridin-4- |
| | yl)methyl]acetamide |
| I-521 | N-[(2-chlorophenyl)methyl]-N-(1,1-dioxo-2,3-dihydro-1lambda6- |
| | thiophen-3-yl)-2-[4-(1H-pyrrol-1-yl)phenyl]acetamide |
| I-522 | N-[(3,5-difluorophenyl)methyl]-N-(1,1-dioxo-2,3-dihydro- |
| | 1lambda6-thiophen-3-yl)-2-[4-(1H-pyrrol-1-yl)phenyl]acetamide |
| I-523 | N-(1,1-dioxo-2,3-dihydro-1lambda6-thiophen-3-yl)-4-phenoxy- |
| | N-[(pyridin-4-yl)methyl]benzamide |
| I-524 | N-(1,1-dioxo-2,3-dihydro-1lambda6-thiophen-3-yl)-N-[(2- |
| | methoxyphenyl)methyl]-2-[4-(1H-pyrrol-1-yl)phenyl]acetamide |
| I-525 | N-(1,1-dioxo-2,3-dihydro-1lambda6-thiophen-3-yl)-N-(2- |
| | hydroxy-1-phenylethyl)-2-[4-(1H-pyrrol-1-yl)phenyl]acetamide |
| I-526 | 2,2-bis(4-chlorophenyl)-N-(1,1-dioxo-2,3-dihydro-1lambda6- |
| | thiophen-3-yl)-N-[(pyridin-4-yl)methyl]acetamide |
| I-527 | N-(1,1-dioxo-2,3-dihydro-1lambda6-thiophen-3-yl)-2-(4- |
| | methanesulfonylphenyl)-N-[(pyridin-4-yl)methyl]acetamide |
| I-528 | N-(1,1-dioxo-2,3-dihydro-1lambda6-thiophen-3-yl)-N-[(pyridin- |
| | 4-yl)methyl]-2-[3-(1H-pyrrol-1-yl)phenyl]acetamide |
| I-529 | (2E)-N-(1,1-dioxo-2,3-dihydro-1lambda6-thiophen-3-yl)-N- |
| | [(pyridin-4-yl)methyl]-3-[4-(trifluoromethyl)phenyl]prop-2- |
| | enamide |
| I-530 | (2E)-N-(1,1-dioxo-2,3-dihydro-1lambda6-thiophen-3-yl)-3- |
| | phenyl-N-[(pyridin-4-yl)methyl]prop-2-enamide |
| I-531 | N-(1,1-dioxo-2,3-dihydro-1lambda6-thiophen-3-yl)-N-[(pyridin- |
| | 4-yl)methyl]-1,2,3,4-tetrahydronaphthalene-2-carboxamide |
| I-532 | N-(1,1-dioxo-2,3-dihydro-1lambda6-thiophen-3-yl)-N-[(pyridin- |
| | 4-yl)methyl]-2-[4-(4H-1,2,4-triazol-4-yl)phenyl]acetamide |
| I-533 | N-cyclobutyl-N-(1,1-dioxo-2,3-dihydro-1lambda6-thiophen-3- |
| | yl)-2-[4-(1H-pyrrol-1-yl)phenyl]acetamide |
| I-534 | N-[(2-bromophenyl)methyl]-N-(1,1-dioxo-2,3-dihydro- |
| | 1lambda6-thiophen-3-yl)-2-[4-(1H-pyrrol-1-yl)phenyl]acetamide |
| I-535 | tert-butyl N-{[benzyl(1,1-dioxo-2,3-dihydro-1lambda6-thiophen- |
| | 3-yl)carbamoyl][4-(1H-pyrrol-1-yl)phenyl]methyl}carbamate |
| I-536 | N-(1,1-dioxo-2,3-dihydro-1lambda6-thiophen-3-yl)-N-[(pyridin- |
| | 4-yl)methyl]-2-[3-(trifluoromethyl)phenyl]acetamide |
| I-537 | N-benzyl-N-(1,1-dioxo-2,3-dihydro-1lambda6-thiophen-3-yl)-2- |
| | {4-[2-(hydroxymethyl)-1H-pyrrol-1-yl]phenyl}acetamide |
| I-538 | 1-benzyl-N-(1,1-dioxo-2,3-dihydro-1lambda6-thiophen-3-yl)-N- |
| | [(pyridin-4-yl)methyl]-1H-pyrazole-4-carboxamide |
| I-539 | N-(1,1-dioxo-2,3-dihydro-1lambda6-thiophen-3-yl)-1-phenyl-N- |
| | [(pyridin-4-yl)methyl]piperidine-3-carboxamide |
| I-540 | N-({bicyclo[1.1.1]pentan-1-yl}methyl)-N-(1,1-dioxo-2,3- |
| | dihydro-1lambda6-thiophen-3-yl)-2-[4-(1H-pyrrol-1- |
| | yl)phenyl]acetamide |
| I-541 | N-(1,1-dioxo-2,3-dihydro-1lambda6-thiophen-3-yl)-2-(6- |
| | methoxy-1H-indol-3-yl)-N-[(pyridin-4-yl)methyl]acetamide |
| I-542 | N-(1,1-dioxo-2,3-dihydro-1lambda6-thiophen-3-yl)-2-(2-oxo-2,3- |
| | dihydro-1H-indol-6-yl)-N-[(pyridin-4-yl)methyl]acetamide |
| I-543 | N-(1,1-dioxo-2,3-dihydro-1lambda6-thiophen-3-yl)-2-[2-(4- |
| | fluorophenyl)-1,3-oxazol-4-yl]-N-[(pyridin-4- |
| | yl)methyl]acetamide |
| I-544 | 2-(1-benzyl-1H-pyrazol-4-yl)-N-(1,1-dioxo-2,3-dihydro- |
| | 1lambda6-thiophen-3-yl)-N-[(pyridin-4-yl)methyl]acetamide |
| I-545 | N-({bicyclo[2.2.2]octan-1-yl}methyl)-N-(1,1-dioxo-2,3-dihydro- |
| | 1lambda6-thiophen-3-yl)-2-[4-(1H-pyrrol-1-yl)phenyl]acetamide |
| I-546 | 2-(6-cyano-1H-indol-3-yl)-N-(1,1-dioxo-2,3-dihydro-1lambda6- |
| | thiophen-3-yl)-N-[(pyridin-4-yl)methyl]acetamide |
| I-547 | N-(1,1-dioxo-2,3-dihydro-1lambda6-thiophen-3-yl)-2-(2-phenyl- |
| | 1,3-thiazol-4-yl)-N-[(pyridin-4-yl)methyl]acetamide |
| I-548 | N-(1,1-dioxo-2,3-dihydro-1lambda6-thiophen-3-yl)-2-{4′-fluoro- |
| | [1,1′-biphenyl]-4-yl}-2-(4-methanesulfonylpiperazin-1-yl)-N- |
| | [(pyridin-4-yl)methyl]acetamide |
| I-549 | tert-butyl N-{[1-(4-{[benzyl(1,1-dioxo-2,3-dihydro-1lambda6- |
| | thiophen-3-yl)carbamoyl]methyl}phenyl)-1H-pyrrol-2- |
| | yl]methyl}carbamate |
| I-550 | N-[(4S)-3,4-dihydro-2H-1-benzopyran-4-yl]-N-(1,1-dioxo-2,3- |
| | dihydro-1lambda6-thiophen-3-yl)-2-[4-(1H-pyrrol-1- |
| | yl)phenyl]acetamide |
| I-551 | N-(1,1-dioxo-2,3-dihydro-1lambda6-thiophen-3-yl)-N′,N′- |
| | dimethyl-N-[(pyridin-4-yl)methyl]-2-[4-(1H-pyrrol-1- |
| | yl)phenyl]butanediamide |
| I-552 | N-(1,1-dioxo-2,3-dihydro-1lambda6-thiophen-3-yl)-2-(1- |
| | phenylpiperidin-4-yl)-N-[(pyridin-4-yl)methyl]acetamide |
| I-553 | N-(1,1-dioxo-2,3-dihydro-1lambda6-thiophen-3-yl)-N-{2-[4-(1H- |
| | pyrrol-1-yl)phenyl]ethyl}pyridine-4-carboxamide |
| I-554 | N-(1,1-dioxo-2,3-dihydro-1lambda6-thiophen-3-yl)-2-(4- |
| | methanesulfonylpiperazin-1-yl)-N-[(pyridin-4-yl)methyl]-2-[4- |
| | (1H-pyrrol-1-yl)phenyl]acetamide |
| I-555 | N-(1,1-dioxo-2,3-dihydro-1lambda6-thiophen-3-yl)-2-{4′- |
| | methoxy-[1,1′-biphenyl]-4-yl}-N-[(pyridin-4- |
| | yl)methyl]acetamide |
| I-556 | N-(1,1-dioxo-2,3-dihydro-1lambda6-thiophen-3-yl)-2-{3′- |
| | methoxy-[1,1′-biphenyl]-4-yl}-N-[(pyridin-4- |
| | yl)methyl]acetamide |
| I-557 | N-(1,1-dioxo-2,3-dihydro-1lambda6-thiophen-3-yl)-2-(4- |
| | phenylpiperazin-1-yl)-N-[(pyridin-4-yl)methyl]acetamide |
| I-558 | N-(1,1-dioxo-2,3-dihydro-1lambda6-thiophen-3-yl)-2-[4-(1,3,4- |
| | oxadiazol-2-yl)phenyl]-N-[(pyridin-4-yl)methyl]acetamide |
| I-559 | N-(1,1-dioxo-2,3-dihydro-1lambda6-thiophen-3-yl)-3-(1H-indol- |
| | 3-yl)-2-(2-methylpropanamido)-N-[(pyridin-4- |
| | yl)methyl]propanamide |
| I-560 | N-(1,1-dioxo-2,3-dihydro-1lambda6-thiophen-3-yl)-3- |
| | {5H,6H,7H,8H,9H-imidazo[1,2-a]azepin-3-yl}-N-[(pyridin-4- |
| | yl)methyl]propanamide |
| I-561 | rel-(2R)-N-(1,1-dioxo-2,3-dihydro-1lambda6-thiophen-3-yl)-2- |
| | [(4-fluorophenyl)formamido]-3-(1H-indol-3-yl)-N-[(pyridin-4- |
| | yl)methyl]propanamide |
| I-562 | N-(1,1-dioxo-2,3-dihydro-1lambda6-thiophen-3-yl)-2-{4′-fluoro- |
| | [1,1′-biphenyl]-4-yl}-N′-methyl-N-[(pyridin-4- |
| | yl)methyl]butanediamide |
| I-563 | N-(1,1-dioxo-2,3-dihydro-1lambda6-thiophen-3-yl)-3-acetamido- |
| | 2-{4′-fluoro-[1,1′-biphenyl]-4-yl}-N-[(pyridin-4- |
| | yl)methyl]propanamide |
| I-564 | N-(1,1-dioxo-2,3-dihydro-1lambda6-thiophen-3-yl)-2-[4- |
| | (morpholin-4-yl)phenyl]-N-[(pyridin-4-yl)methyl]acetamide |
| I-565 | 3-(6-bromo-1-methyl-1H-indol-3-yl)-N-(1,1-dioxo-2,3-dihydro- |
| | 1lambda6-thiophen-3-yl)-N-[(pyridin-4-yl)methyl]propanamide |
| I-566 | N-benzyl-N-(5-methyl-1,1-dioxo-2,3-dihydro-1lambda6- |
| | thiophen-3-yl)-2-[4-(1H-pyrrol-1-yl)phenyl]acetamide |
| I-567 | N-benzyl-2-(4-{2-[(dimethylamino)methyl]-1H-pyrrol-1- |
| | yl}phenyl)-N-(1,1-dioxo-2,3-dihydro-1lambda6-thiophen-3- |
| | yl)acetamide |
| I-568 | N-benzyl-N-(1,1-dioxo-2,3-dihydro-1lambda6-thiophen-3-yl)-2- |
| | {4-[2-(methoxymethyl)-1H-pyrrol-1-yl]phenyl}acetamide |
| I-569 | tert-butyl 3-[(1,1-dioxo-2,3-dihydro-1lambda6-thiophen-3- |
| | yl)[(pyridin-4-yl)methyl]carbamoyl]-6-(4-fluorophenyl)-2,3- |
| | dihydro-1H-indole-1-carboxylate |
| I-570 | N-(1,1-dioxo-2,3-dihydro-1lambda6-thiophen-3-yl)-3-phenyl-N- |
| | [(pyridin-4-yl)methyl]cyclobutane-1-carboxamide |
| I-571 | N-(1,1-dioxo-2,3-dihydro-1lambda6-thiophen-3-yl)-2-(4- |
| | phenylpiperidin-1-yl)-N-[(pyridin-4-yl)methyl]acetamide |
| I-572 | N-(1,1-dioxo-2,3-dihydro-1lambda6-thiophen-3-yl)-N-[(pyridin- |
| | 4-yl)methyl]-2-[4-(trifluoromethyl)piperidin-1-yl]acetamide |
| I-573 | N-(1,1-dioxo-2,3-dihydro-1lambda6-thiophen-3-yl)-2-[2-fluoro- |
| | 4-(2-methyl-1,3-thiazol-4-yl)phenyl]-N-[(2-methoxypyridin-4- |
| | yl)methyl]acetamide |
| I-574 | N-(1,1-dioxo-2,3-dihydro-1lambda6-thiophen-3-yl)-2-{4′-fluoro- |
| | [1,1′-biphenyl]-4-yl}-N-[(2-methoxypyridin-4-yl)methyl]-N′- |
| | methylbutanediamide |
| I-575 | (2R*)-N-[(3R**)-1,1-dioxo-2,3-dihydro-1lambda6-thiophen-3- |
| | yl]-2-{4′-fluoro-[1,1′-biphenyl]-4-yl}-2-methoxy-N-[(pyridin-4- |
| | yl)methyl]acetamide |
| I-577 | (2R**)-N-[(3R*)-1,1-dioxo-2,3-dihydro-1lambda6-thiophen-3- |
| | yl]-N-[(2-methoxypyridin-4-yl)methyl]-N′,N′-dimethyl-2-{3,3′,4′- |
| | trifluoro-[1,1′-biphenyl]-4-yl}propanediamide |
| I-578 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2- |
| | methoxy-2-{3,3′,4′-trifluoro-[1,1′-biphenyl]-4-yl}acetamide |
| I-579 | 3-benzyl-N-(1,1-dioxo-2,3-dihydro-1lambda6-thiophen-3-yl)-N- |
| | [(pyridin-4-yl)methyl]-4,5-dihydro-1,2-oxazole-5-carboxamide |
| I-580 | 2-(4-cyclopropyl-1,3-thiazol-2-yl)-N-(1,1-dioxo-2,3-dihydro- |
| | 1lambda6-thiophen-3-yl)-N-[(pyridin-4-yl)methyl]acetamide |
| I-581 | N-(1,1-dioxo-2,3-dihydro-1lambda6-thiophen-3-yl)-2,2-difluoro- |
| | 2-(4-phenyl-1H-1,2,3-triazol-1-yl)-N-[(pyridin-4- |
| | yl)methyl]acetamide |
| I-582 | 2-(1-cyclopropyl-1H-pyrazol-4-yl)-N-(1,1-dioxo-2,3-dihydro- |
| | 1lambda6-thiophen-3-yl)-N-[(pyridin-4-yl)methyl]acetamide |
| I-583 | (1R,5S,6R)-N-(1,1-dioxo-2,3-dihydro-1lambda6-thiophen-3-yl)- |
| | N-[(pyridin-4-yl)methyl]bicyclo[3.1.0]hexane-6-carboxamide |
| I-584 | N-(1,1-dioxo-2,3-dihydro-1lambda6-thiophen-3-yl)-2-(3- |
| | phenylcyclobutyl)-N-[(pyridin-4-yl)methyl]acetamide |
| I-585 | N-(1,1-dioxo-2,3-dihydro-1lambda6-thiophen-3-yl)-3-(2-oxo-2,3- |
| | dihydro-1H-indol-3-yl)-N-[(pyridin-4-yl)methyl]propanamide |
| I-586 | N-(1,1-dioxo-2,3-dihydro-1lambda6-thiophen-3-yl)-2-fluoro-N- |
| | [(pyridin-4-yl)methyl]-3-[3- |
| | (trifluoromethyl)phenyl]bicyclo[1.1.1]pentane-1-carboxamide |
| I-587 | N-(1,1-dioxo-2,3-dihydro-1lambda6-thiophen-3-yl)-N-[(pyridin- |
| | 4-yl)methyl]-2-[4-(trimethylsilyl)phenyl]acetamide |
| I-588 | 2-(4-cyclopropoxyphenyl)-N-(1,1-dioxo-2,3-dihydro-1lambda6- |
| | thiophen-3-yl)-N-[(2-methoxypyridin-4-yl)methyl]acetamide |
| I-589 | N-(1,1-dioxo-2,3-dihydro-1lambda6-thiophen-3-yl)-N-[(pyridin- |
| | 4-yl)methyl]-2-[5-(trifluoromethyl)-2H-indazol-2-yl]acetamide |
| I-590 | N-(1,1-dioxo-2,3-dihydro-1lambda6-thiophen-3-yl)-N-[(pyridin- |
| | 4-yl)methyl]-2-[5-(trifluoromethyl)-1H-indazol-1-yl]acetamide |
| I-591 | N-[(3R)-1,1-dioxo-2,3-dihydro-1lambda6-thiophen-3-yl]-2-[2- |
| | fluoro-4-(3-fluorocyclobutyl)phenyl]-N-[(2-methoxypyridin-4- |
| | yl)methyl]acetamide |
| I-592 | 2-[6-(difluoromethyl)-2H-indazol-2-yl]-N-(1,1-dioxo-2,3- |
| | dihydro-1lambda6-thiophen-3-yl)-N-[(pyridin-4- |
| | yl)methyl]acetamide |
| I-593 | 2-[6-(difluoromethyl)-1H-indazol-1-yl]-N-(1,1-dioxo-2,3- |
| | dihydro-1lambda6-thiophen-3-yl)-N-[(pyridin-4- |
| | yl)methyl]acetamide |
| I-594 | N′-(1,1-dioxo-2,3-dihydro-1lambda6-thiophen-3-yl)-N′-{[2-(2- |
| | hydroxypropan-2-yl)pyridin-4-yl]methyl}-N,N-dimethyl-2-(4- |
| | phenyl-1H-pyrazol-1-yl)propanediamide |
| I-595 | N-[(3R)-1,1-dioxo-2,3-dihydro-1lambda6-thiophen-3-yl]-N-[(2- |
| | methoxypyridin-4-yl)methyl]-2-(5-methyl-4-phenyl-1H-pyrazol- |
| | 1-yl)acetamide |
| I-596 | N-[(3R)-1,1-dioxo-2,3-dihydro-1lambda6-thiophen-3-yl]-N-[(2- |
| | methoxypyridin-4-yl)methyl]-2-(3-methyl-4-phenyl-1H-pyrazol- |
| | 1-yl)acetamide |
| I-597 | N-[(3R)-1,1-dioxo-2,3-dihydro-1lambda6-thiophen-3-yl]-2-[4-(4- |
| | fluorophenyl)-1H-imidazol-1-yl]-N-[(2-methoxypyridin-4- |
| | yl)methyl]acetamide |
| I-598 | 2-[4-(3,4-difluorophenyl)-1H-pyrazol-1-yl]-N-[(3R)-1,1-dioxo- |
| | 2,3-dihydro-1lambda6-thiophen-3-yl]-N-[(2-methoxypyridin-4- |
| | yl)methyl]acetamide |
| I-599 | N-[(4-cyclopropyl-1,3-thiazol-2-yl)methyl]-2-[4-(3,4- |
| | difluorophenyl)-1H-pyrazol-1-yl]-N-(1,1-dioxo-2,3-dihydro- |
| | 1lambda6-thiophen-3-yl)acetamide |
| I-600 | N-(1,1-dioxo-2,3-dihydro-1lambda6-thiophen-3-yl)-N-[(6- |
| | methoxypyrimidin-4-yl)methyl]-2-[4-(pentafluoro-lambda6- |
| | sulfanyl)phenyl]acetamide |
| I-601 | rel-N-[(4-cyclopropyl-1,3-thiazol-2-yl)methyl]-N-[(3R)-1,1- |
| | dioxo-2,3-dihydro-1lambda6-thiophen-3-yl]-2-[4-(4- |
| | fluorophenyl)-1H-pyrazol-1-yl]acetamide |
| I-602 | rel-N-[(4-cyclopropyl-1,3-thiazol-2-yl)methyl]-N-[(3R)-1,1- |
| | dioxo-2,3-dihydro-1lambda6-thiophen-3-yl]-2-[4-(4- |
| | fluorophenyl)-1H-pyrazol-1-yl]acetamide |
| I-603 | N-[(3R)-1,1-dioxo-2,3-dihydro-1lambda6-thiophen-3-yl]-2-(2- |
| | hydroxyethoxy)-N-[(2-methoxypyridin-4-yl)methyl]-2-{3,3′,4′- |
| | trifluoro-[1,1′-biphenyl]-4-yl}acetamide |
| I-604 | rel-N-[(4-cyclopropyl-1,3-thiazol-2-yl)methyl]-2-[4-(3,4- |
| | difluorophenyl)-1H-pyrazol-1-yl]-N-[(3R)-1,1-dioxo-2,3- |
| | dihydro-1lambda6-thiophen-3-yl]acetamide |
| I-605 | N-{[2-(difluoromethoxy)pyridin-4-yl]methyl}-N-(1,1-dioxo-2,3- |
| | dihydro-1lambda6-thiophen-3-yl)-2-[4-(pentafluoro-lambda6- |
| | sulfanyl)phenyl]acetamide |
| I-606 | rel-N-[(3R)-1,1-dioxo-2,3-dihydro-1lambda6-thiophen-3-yl]-2- |
| | [4-(4-fluorophenyl)-2-oxo-1,2-dihydropyridin-1-yl]-N-{[2-(2- |
| | hydroxypropan-2-yl)pyridin-4-yl]methyl}acetamide |
| I-607 | rel-N-[(3R)-1,1-dioxo-2,3-dihydro-1lambda6-thiophen-3-yl]-N- |
| | {[2-(2-hydroxypropan-2-yl)pyridin-4-yl]methyl}-5-(2-methyl- |
| | 1H-pyrrol-1-yl)-2,3-dihydro-1-benzofuran-2-carboxamide |
| I-608 | N-(cyclopropylmethyl)-N-(1,1-dioxo-2,3-dihydro-1lambda6- |
| | thiophen-3-yl)-2-[4-(pentafluoro-lambda6- |
| | sulfanyl)phenyl]acetamide |
| I-609 | N-(1,1-dioxo-2,3-dihydro-1lambda6-thiophen-3-yl)-2-(1-methyl- |
| | 3-phenyl-1H-pyrazol-5-yl)-N-[(pyridin-4-yl)methyl]acetamide |
| I-610 | N-(1,1-dioxo-2,3-dihydro-1lambda6-thiophen-3-yl)-2-(2-methyl- |
| | 1,3-benzoxazol-5-yl)-N-[(pyridin-4-yl)methyl]acetamide |
| I-611 | (1s,3s)-N-(1,1-dioxo-2,3-dihydro-1lambda6-thiophen-3-yl)-3- |
| | phenyl-N-[(pyridin-4-yl)methyl]cyclobutane-1-carboxamide |
| I-612 | N-[(4,5-dimethyl-1,3-thiazol-2-yl)methyl]-N-(1,1-dioxo-2,3- |
| | dihydro-1lambda6-thiophen-3-yl)-2-[4-(pentafluoro-lambda6- |
| | sulfanyl)phenyl]acetamide |
| I-613 | 2-(3,4-dihydro-2H-1-benzopyran-3-yl)-N-(1,1-dioxo-2,3-dihydro- |
| | 1lambda6-thiophen-3-yl)-N-[(pyridin-4-yl)methyl]acetamide |
| I-614 | N-(1,1-dioxo-2,3-dihydro-1lambda6-thiophen-3-yl)-3-(4- |
| | fluorophenyl)-N-[(pyridin-4-yl)methyl]cyclobutane-1- |
| | carboxamide |
| I-615 | N-(1,1-dioxo-2,3-dihydro-1lambda6-thiophen-3-yl)-3-(4- |
| | fluorophenyl)-N-[(pyridin-4-yl)methyl]-1,2-thiazole-5- |
| | carboxamide |
| I-616 | 2-[3-(difluoromethyl)-4,5,6,7-tetrahydro-1H-indazol-1-yl]-N- |
| | (1,1-dioxo-2,3-dihydro-1lambda6-thiophen-3-yl)-N-[(pyridin-4- |
| | yl)methyl]acetamide |
| I-617 | (1R,5S,6R)-N-(1,1-dioxo-2,3-dihydro-1lambda6-thiophen-3-yl)- |
| | 3,3-difluoro-N-[(pyridin-4-yl)methyl]bicyclo[3.1.0]hexane-6- |
| | carboxamide |
| I-618 | N-(1,1-dioxo-2,3-dihydro-1lambda6-thiophen-3-yl)-N-[(pyridin- |
| | 4-yl)methyl]-3-(2,2,2-trifluoroethyl)-3-azabicyclo[3.1.0]hexane- |
| | 6-carboxamide |
| I-619 | N-(1,1-dioxo-2,3-dihydro-1lambda6-thiophen-3-yl)-N-[(pyridin- |
| | 4-yl)methyl]bicyclo[2.1.1]hexane-5-carboxamide |
| I-620 | N-(1,1-dioxo-2,3-dihydro-1lambda6-thiophen-3-yl)-2-(2H- |
| | indazol-2-yl)-N-[(pyridin-4-yl)methyl]propanamide |
| I-621 | N-(1,1-dioxo-2,3-dihydro-1lambda6-thiophen-3-yl)-3-(pyridin-4- |
| | yl)-N-[(pyridin-4-yl)methyl]-4,5-dihydro-1,2-oxazole-5- |
| | carboxamide |
| I-622 | N-(1,1-dioxo-2,3-dihydro-1lambda6-thiophen-3-yl)-N-[(pyridin- |
| | 4-yl)methyl]-2-(trifluoromethyl)-5H,6H,7H,8H-imidazo[1,2- |
| | alpyridine-7-carboxamide |
| I-623 | N-(1,1-dioxo-2,3-dihydro-1lambda6-thiophen-3-yl)-N-{[4-(2- |
| | hydroxypropan-2-yl)phenyl]methyl}-2-[4-(pentafluoro-lambda6- |
| | sulfanyl)phenyl]acetamide |
| I-624 | N-[(3-cyclopropyl-1,2-oxazol-5-yl)methyl]-N-(1,1-dioxo-2,3- |
| | dihydro-1lambda6-thiophen-3-yl)-2-[4-(pentafluoro-lambda6- |
| | sulfanyl)phenyl]acetamide |
| I-625 | N-{[4-(1,1-difluoroethyl)-1,3-thiazol-2-yl]methyl}-N-(1,1-dioxo- |
| | 2,3-dihydro-1lambda6-thiophen-3-yl)-2-[4-(pentafluoro-lambda6- |
| | sulfanyl)phenyl]acetamide |
| I-626 | N-(1,1-dioxo-2,3-dihydro-1lambda6-thiophen-3-yl)-2-[4- |
| | (pentafluoro-lambda6-sulfanyl)phenyl]-N-{[4-(2H-1,2,3,4- |
| | tetrazol-5-yl)-1,3-thiazol-2-yl]methyl}acetamide |
| I-627 | N-(1,1-dioxo-2,3-dihydro-1lambda6-thiophen-3-yl)-N-[(pyridin- |
| | 4-yl)methyl]-2-[5-(trifluoromethyl)pyrazin-2-yl]acetamide |
| I-628 | N-(1,1-dioxo-2,3-dihydro-1lambda6-thiophen-3-yl)-N-[(4-ethyl- |
| | 1,3-thiazol-2-yl)methyl]-2-[4-(pentafluoro-lambda6- |
| | sulfanyl)phenyl]acetamide |
| I-629 | N-[2-(4-cyclopropyl-1,3-thiazol-2-yl)ethyl]-N-(1,1-dioxo-2,3- |
| | dihydro-1lambda6-thiophen-3-yl)-2-[4-(pentafluoro-lambda6- |
| | sulfanyl)phenyl]acetamide |
| I-630 | 2-(3,4-dihydro-1H-2-benzopyran-6-yl)-N-(1,1-dioxo-2,3-dihydro- |
| | 1lambda6-thiophen-3-yl)-N-[(pyridin-4-yl)methyl]acetamide |
| I-631 | 2-(4-bromo-3,5-dimethoxyphenyl)-N-(1,1-dioxo-2,3-dihydro- |
| | 1lambda6-thiophen-3-yl)-N-[(pyridin-4-yl)methyl]acetamide |
| I-632 | rel-2-(difluoromethoxy)-N-[(3R)-1,1-dioxo-2,3-dihydro- |
| | 1lambda6-thiophen-3-yl]-N-{[2-(2-hydroxypropan-2-yl)pyridin- |
| | 4-yl]methyl}-2-{3,3′,4′-trifluoro-[1,1′-biphenyl]-4-yl}acetamide |
| I-633 | N-(1,1-dioxo-2,3-dihydro-1lambda6-thiophen-3-yl)-N-{[2-(3- |
| | hydroxyoxetan-3-yl)pyridin-4-yl]methyl}-2-[4-(pentafluoro- |
| | lambda6-sulfanyl)phenyl]acetamide |
| I-634 | N-{[6-(dimethylamino)-2-methoxypyrimidin-4-yl]methyl}-N- |
| | (1,1-dioxo-2,3-dihydro-1lambda6-thiophen-3-yl)-2-[4- |
| | (pentafluoro-lambda6-sulfanyl)phenyl]acetamide |
| I-635 | N-{[2-(dimethylamino)-6-methoxypyrimidin-4-yl]methyl}-N- |
| | (1,1-dioxo-2,3-dihydro-1lambda6-thiophen-3-yl)-2-[4- |
| | (pentafluoro-lambda6-sulfanyl)phenyl]acetamide |
| I-636 | rel-{[(3R)-1,1-dioxo-2,3-dihydro-1lambda6-thiophen-3-yl]({[2- |
| | (2-hydroxypropan-2-yl)pyridin-4-yl]methyl})carbamoyl}({3,3′,4′- |
| | trifluoro-[1,1′-biphenyl]-4-yl})methyl N,N-dimethylcarbamate |
| I-637 | N-(1,1-dioxo-2,3-dihydro-1lambda6-thiophen-3-yl)-N-{[4-(2- |
| | hydroxypropan-2-yl)-1,3-thiazol-2-yl]methyl}-2-[4-(pentafluoro- |
| | lambda6-sulfanyl)phenyl]acetamide |
| I-638 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-{4-[4- |
| | (trifluoromethyl)phenyl]-1H-pyrazol-1-yl}acetamide |
| I-639 | rel-2-{4-[4-(difluoromethyl)phenyl]-1H-pyrazol-1-yl}-N-[(3R)- |
| | 1,1-dioxo-2,3-dihydro-1lambda6-thiophen-3-yl]-N-{[2-(2- |
| | hydroxypropan-2-yl)pyridin-4-yl]methyl}acetamide |
| I-640 | N-benzyl-2-{4′-fluoro-[1,1′-biphenyl]-4-yl}-N-[5- |
| | (hydroxymethyl)-1,1-dioxo-2,3-dihydro-1lambda6-thiophen-3- |
| | yl]acetamide |
| I-641 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-(prop-2-en-1- |
| | yloxy)-2-{3,3′,4′-trifluoro-[1,1′-biphenyl]-4-yl}acetamide |
| I-642 | 2-(3,4-dihydro-1H-2-benzopyran-7-yl)-N-(1,1-dioxo-2,3-dihydro- |
| | 1lambda6-thiophen-3-yl)-N-[(pyridin-4-yl)methyl]acetamide |
| I-643 | N-(1,1-dioxo-2,3-dihydro-1lambda6-thiophen-3-yl)-N-[(pyridin- |
| | 4-yl)methyl]-2-(5,6,7,8-tetrahydronaphthalen-2-yl)acetamide |
| I-644 | N-[(5-cyclopropyl-1,2,4-oxadiazol-3-yl)methyl]-N-(1,1-dioxo- |
| | 2,3-dihydro-1lambda6-thiophen-3-yl)-2-[4-(pentafluoro-lambda6- |
| | sulfanyl)phenyl]acetamide |
| I-645 | N-[(5-cyclopropyl-1,3,4-thiadiazol-2-yl)methyl]-N-(1,1-dioxo- |
| | 2,3-dihydro-1lambda6-thiophen-3-yl)-2-[4-(pentafluoro-lambda6- |
| | sulfanyl)phenyl]acetamide |
| I-646 | N-(1,1-dioxo-2,3-dihydro-1lambda6-thiophen-3-yl)-2-[4- |
| | (pentafluoro-lambda6-sulfanyl)phenyl]-N-[(pyridazin-4- |
| | yl)methyl]acetamide |
| I-647 | N-(1,1-dioxo-2,3-dihydro-1lambda6-thiophen-3-yl)-4-phenyl-N- |
| | [(pyridin-4-yl)methyl]oxetane-2-carboxamide |
| I-648 | N-(1,1-dioxo-2,3-dihydro-1lambda6-thiophen-3-yl)-N-[(1,2- |
| | oxazol-4-yl)methyl]-2-[4-(pentafluoro-lambda6- |
| | sulfanyl)phenyl]acetamide |
| I-649 | N-(1,1-dioxo-2,3-dihydro-1lambda6-thiophen-3-yl)-2,2-difluoro- |
| | 2-(4-methoxyphenyl)-N-[(pyridin-4-yl)methyl]acetamide |
| I-650 | 4-chloro-N-(1,1-dioxo-2,3-dihydro-1lambda6-thiophen-3-yl)-N- |
| | [(pyridin-4-yl)methyl]bicyclo[4.2.0]octa-1,3,5-triene-7- |
| | carboxamide |
| I-651 | N-(1,1-dioxo-2,3-dihydro-1lambda6-thiophen-3-yl)-N-[(pyridin- |
| | 4-yl)methyl]-4-(trifluoromethyl)oxolane-2-carboxamide |
| I-652 | rac-(2R,5S)-N-(1,1-dioxo-2,3-dihydro-1lambda6-thiophen-3-yl)- |
| | 5-phenyl-N-[(pyridin-4-yl)methyl]oxolane-2-carboxamide |
| I-653 | 6-chloro-N-(1,1-dioxo-2,3-dihydro-1lambda6-thiophen-3-yl)-N- |
| | [(pyridin-4-yl)methyl]-3,4-dihydro-2H-1-benzopyran-2- |
| | carboxamide |
| I-654 | N-(1,1-dioxo-2,3-dihydro-1lambda6-thiophen-3-yl)-N-[(pyridin- |
| | 4-yl)methyl]-6-oxaspiro[3.5]nonane-7-carboxamide |
| I-655 | rac-(1R,3R)-N-(1,1-dioxo-2,3-dihydro-1lambda6-thiophen-3-yl)- |
| | 3-phenyl-N-[(pyridin-4-yl)methyl]cyclopentane-1-carboxamide |
| I-656 | N-(1,1-dioxo-2,3-dihydro-1lambda6-thiophen-3-yl)-2-(2-methyl- |
| | 2H-indazol-5-yl)-N-[(pyridin-4-yl)methyl]acetamide |
| I-657 | N-(1,1-dioxo-2,3-dihydro-1lambda6-thiophen-3-yl)-2-[4- |
| | (pentafluoro-lambda6-sulfanyl)phenyl]-N-[(1,3-thiazol-4- |
| | yl)methyl]acetamide |
| I-658 | 6-bromo-N-(1,1-dioxo-2,3-dihydro-1lambda6-thiophen-3-yl)-N- |
| | [(pyridin-4-yl)methyl]-2,3-dihydro-1H-indene-1-carboxamide |
| I-659 | N-(1,1-dioxo-2,3-dihydro-1lambda6-thiophen-3-yl)-2-[4- |
| | (pentafluoro-lambda6-sulfanyl)phenyl]-N-{[4-(propan-2-yl)-1,3- |
| | thiazol-2-yl]methyl}acetamide |
| I-660 | N-[(3R)-1,1-dioxo-2,3-dihydro-1lambda6-thiophen-3-yl]-2-[4-(4- |
| | fluorophenyl)-2-oxo-1,2-dihydropyridin-1-yl]-N-[(2- |
| | methoxypyridin-4-yl)methyl]acetamide |
| I-661 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[4-(1,1,2,2,2- |
| | pentafluoroethoxy)phenyl]acetamide |
| I-662 | N-{[2-(difluoromethoxy)pyridin-4-yl]methyl}-N-(1,1-dioxo-2,3- |
| | dihydro-1lambda6-thiophen-3-yl)-2-[4-(4-fluorophenyl)-1H- |
| | pyrazol-1-yl]acetamide |
| I-663 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-{4-[1- |
| | (trifluoromethyl)cyclopropyl]phenyl}acetamide |
| I-664 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2- |
| | methoxy-2-[4-(pentafluoro-lambda6-sulfanyl)phenyl]acetamide |
| I-665 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2- |
| | methoxy-2-[4-(pentafluoro-lambda6-sulfanyl)phenyl]acetamide |
| I-666 | N-(1,1-dioxo-2,3-dihydro-1lambda6-thiophen-3-yl)-2-{4′-fluoro- |
| | [1,1′-biphenyl]-4-yl}-3-(4-methanesulfonylpiperazin-1-yl)-3-oxo- |
| | N-[(pyridin-4-yl)methyl]propanamide |
| I-667 | 3-(4-acetylpiperazin-1-yl)-N-(1,1-dioxo-2,3-dihydro-1lambda6- |
| | thiophen-3-yl)-2-{4′-fluoro-[1,1′-biphenyl]-4-yl}-3-oxo-N- |
| | [(pyridin-4-yl)methyl]propanamide |
| I-668 | N-(1,1-dioxo-2,3-dihydro-1lambda6-thiophen-3-yl)-2-[4-(5- |
| | fluoropyridin-2-yl)-3-hydroxyphenyl]-N-[(2-methoxypyridin-4- |
| | yl)methyl]acetamide |
| I-669 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2-(1H- |
| | pyrazol-1-yl)-2-{3,3′,4′-trifluoro-[1,1′-biphenyl]-4-yl}acetamide |
| I-670 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2-(1H- |
| | pyrazol-1-yl)-2-{3,3′,4′-trifluoro-[1,1′-biphenyl]-4-yl}acetamide |
| I-671 | (2R*)-N-[(3R)-1,1-dioxo-2,3-dihydro-1lambda6-thiophen-3-yl]- |
| | N-[(2-methoxypyridin-4-yl)methyl]-2-(1H-pyrazol-1-yl)-2- |
| | {3,3′,4′-trifluoro-[1,1′-biphenyl]-4-yl}acetamide |
| I-672 | (2R*)-N-[(3R)-1,1-dioxo-2,3-dihydro-1lambda6-thiophen-3-yl]- |
| | N-[(2-methoxypyridin-4-yl)methyl]-2-(1H-pyrazol-1-yl)-2- |
| | {3,3′,4′-trifluoro-[1,1′-biphenyl]-4-yl}acetamide |
| I-673 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[4-(1,1,2,2,2- |
| | pentafluoroethyl)phenyl]acetamide |
| I-674 | rel-N-[(3R)-1,1-dioxo-2,3-dihydro-1lambda6-thiophen-3-yl]-N- |
| | {[2-(2-hydroxypropan-2-yl)pyridin-4-yl]methyl}-3-[4- |
| | (pentafluoro-lambda6-sulfanyl)phenyl]propanamide |
| I-675 | rel-N-[(3R)-1,1-dioxo-2,3-dihydro-1lambda6-thiophen-3-yl]-N- |
| | {[2-(2-hydroxypropan-2-yl)pyridin-4-yl]methyl}-3-methoxy-2- |
| | [4-(pentafluoro-lambda6-sulfanyl)phenyl]propanamide |
| I-676 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[5- |
| | (trifluoromethyl)-2H-indazol-2-yl]acetamide |
| I-677 | 2-cyclopropyl-N-(1,1-dioxo-2,3-dihydro-1lambda6-thiophen-3- |
| | yl)-2-{4′-fluoro-[1,1′-biphenyl]-4-yl}-N-[(pyridin-4- |
| | yl)methyl]acetamide |
| I-678 | 6-bromo-N-(1,1-dioxo-2,3-dihydro-1lambda6-thiophen-3-yl)-N- |
| | [(pyridin-4-yl)methyl]-3,4-dihydro-2H-1-benzopyran-2- |
| | carboxamide |
| I-679 | N-(1,1-dioxo-2,3-dihydro-1lambda6-thiophen-3-yl)-2-[4- |
| | (pentafluoro-lambda6-sulfanyl)phenyl]-N-[2-(1,3-thiazol-2- |
| | yl)ethyl]acetamide |
| I-680 | 2-[3-(cyclohex-1-en-1-yl)-2-oxo-2,3-dihydro-1H-1,3- |
| | benzodiazol-1-yl]-N-(1,1-dioxo-2,3-dihydro-1lambda6-thiophen- |
| | 3-yl)-N-[(pyridin-4-yl)methyl]acetamide |
| I-681 | rel-2-cyclopropoxy-N-[(3R)-1,1-dioxo-2,3-dihydro-1lambda6- |
| | thiophen-3-yl]-N-{[2-(2-hydroxypropan-2-yl)pyridin-4- |
| | yl]methyl}-2-{3,3′,4′-trifluoro-[1,1′-biphenyl]-4-yl}acetamide |
| I-682 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-(1H-pyrazol- |
| | 1-yl)-2-{3,3′,4′-trifluoro-[1,1′-biphenyl]-4-yl}acetamide |
| I-683 | N-[(3R)-1,1-dioxo-2,3-dihydro-1lambda6-thiophen-3-yl]-N-[(2- |
| | methoxypyridin-4-yl)methyl]-2-(1H-pyrazol-1-yl)-2-{3,3′,4′- |
| | trifluoro-[1,1′-biphenyl]-4-yl}acetamide |
| I-684 | N-[(2,6-dimethoxypyridin-4-yl)methyl]-N-(1,1-dioxo-2,3- |
| | dihydro-1lambda6-thiophen-3-yl)-2-[4-(pentafluoro-lambda6- |
| | sulfanyl)phenyl]acetamide |
| I-685 | (2R*)-2-[4-(difluoromethoxy)-1H-pyrazol-1-yl]-N-[(3R)-1,1- |
| | dioxo-2,3-dihydro-1lambda6-thiophen-3-yl]-N-[(2- |
| | methoxypyridin-4-yl)methyl]-2-{3,3′,4′-trifluoro-[1,1′-biphenyl]- |
| | 4-yl}acetamide |
| I-686 | (2R*)-2-(cyanomethoxy)-N-[(3R)-1,1-dioxo-2,3-dihydro- |
| | 1lambda6-thiophen-3-yl]-N-[(2-methoxypyridin-4-yl)methyl]-2- |
| | {3,3′,4′-trifluoro-[1,1′-biphenyl]-4-yl}acetamide |
| I-687 | N-[(3R)-1,1-dioxo-2,3-dihydro-1lambda6-thiophen-3-yl]-2-[(1- |
| | methanesulfonylpiperidin-4-yl)oxy]-N-[(2-methoxypyridin-4- |
| | yl)methyl]-2-{3,3′,4′-trifluoro-[1,1′-biphenyl]-4-yl}acetamide |
| I-688 | N′-[(3R)-1,1-dioxo-2,3-dihydro-1lambda6-thiophen-3-yl]-N′-[(2- |
| | methoxypyridin-4-yl)methyl]-N-methyl-2-{3,3′,4′-trifluoro-[1,1′- |
| | biphenyl]-4-yl}propanediamide |
| I-689 | (2R**)-N′-[(3R*)-1,1-dioxo-2,3-dihydro-1lambda6-thiophen-3- |
| | yl]-N′-{[2-(2-hydroxypropan-2-yl)pyridin-4-yl]methyl}-N- |
| | methyl-2-{3,3′,4′-trifluoro-[1,1′-biphenyl]-4-yl}propanediamide |
| I-690 | (2R**)-N′-[(3R*)-1,1-dioxo-2,3-dihydro-1lambda6-thiophen-3- |
| | yl]-N′-{[2-(2-hydroxypropan-2-yl)pyridin-4-yl]methyl}-N- |
| | methyl-2-{3,3′,4′-trifluoro-[1,1′-biphenyl]-4-yl}propanediamide |
| I-691 | 2-(cyanomethoxy)-N-[(3R)-1,1-dioxo-2,3-dihydro-1lambda6- |
| | thiophen-3-yl]-N-[(2-methoxypyridin-4-yl)methyl]-2-{3,3′,4′- |
| | trifluoro-[1,1′-biphenyl]-4-yl}acetamide |
| I-692 | (2R*)-2-(cyanomethoxy)-N-[(3R)-1,1-dioxo-2,3-dihydro- |
| | 1lambda6-thiophen-3-yl]-N-[(2-methoxypyridin-4-yl)methyl]-2- |
| | {3,3′,4′-trifluoro-[1,1′-biphenyl]-4-y]}acetamide |
| I-693 | (2R*)-N′-[(3R)-1,1-dioxo-2,3-dihydro-1lambda6-thiophen-3-yl]- |
| | N′-[(2-methoxypyridin-4-yl)methyl]-N-methyl-2-{3,3′,4′- |
| | trifluoro-[1,1′-biphenyl]-4-yl}propanediamide |
| I-694 | (2R*)-N′-[(3R)-1,1-dioxo-2,3-dihydro-1lambda6-thiophen-3-yl]- |
| | N′-[(2-methoxypyridin-4-yl)methyl]-N-methyl-2-{3,3′,4′- |
| | trifluoro-[1,1′-biphenyl]-4-yl}propanediamide |
| I-695 | 2-[4-(difluoromethoxy)-1H-pyrazol-1-yl]-N-[(3R)-1,1-dioxo-2,3- |
| | dihydro-1lambda6-thiophen-3-yl]-N-[(2-methoxypyridin-4- |
| | yl)methyl]-2-{3,3′,4′-trifluoro-[1,1′-biphenyl]-4-yl}acetamide |
| I-696 | (2R*)-2-[4-(difluoromethoxy)-1H-pyrazol-1-yl]-N-[(3R)-1,1- |
| | dioxo-2,3-dihydro-1lambda6-thiophen-3-yl]-N-[(2- |
| | methoxypyridin-4-yl)methyl]-2-{3,3′,4′-trifluoro-[1,1′-biphenyl]- |
| | 4-yl}acetamide |
| I-697 | 2-[3-(difluoromethoxy)-4-methoxyphenyl]-N-(1,1-dioxo-2,3- |
| | dihydro-1lambda6-thiophen-3-yl)-N-[(pyridin-4- |
| | yl)methyl]acetamide |
| I-698 | N-{[2-(difluoromethoxy)pyridin-4-yl]methyl}-2-[5-(3,4- |
| | difluorophenyl)-3-fluoropyridin-2-yl]-N-(1,1-dioxo-2,3-dihydro- |
| | 1lambda6-thiophen-3-yl)acetamide |
| I-699 | N-[(3R)-1,1-dioxo-2,3-dihydro-1lambda6-thiophen-3-yl]-N-[(2- |
| | methoxypyridin-4-yl)methyl]-2-[5-(trifluoromethyl)-2H-indazol- |
| | 2-yl]acetamide |
| I-700 | N-{[4-(2-aminopropan-2-yl)phenyl]methyl}-N-(1,1-dioxo-2,3- |
| | dihydro-1lambda6-thiophen-3-yl)-2-[4-(pentafluoro-lambda6- |
| | sulfanyl)phenyl]acetamide |
| I-701 | (2R*)-2-[4-(difluoromethyl)-1H-pyrazol-1-yl]-N-[(3R)-1,1- |
| | dioxo-2,3-dihydro-1lambda6-thiophen-3-yl]-N-[(2- |
| | methoxypyridin-4-yl)methyl]-2-{3,3′,4′-trifluoro-[1,1′-biphenyl]- |
| | 4-yl}acetamide |
| I-702 | (2R*)-N-[(3R)-1,1-dioxo-2,3-dihydro-1lambda6-thiophen-3-yl]- |
| | 2-(2-methanesulfonylethoxy)-N-[(2-methoxypyridin-4- |
| | yl)methyl]-2-{3,3′,4′-trifluoro-[1,1′-biphenyl]-4-yl}acetamide |
| I-703 | (2R*)-2-(4-cyano-1H-pyrazol-1-yl)-N-[(3R)-1,1-dioxo-2,3- |
| | dihydro-1lambda6-thiophen-3-yl]-N-[(2-methoxypyridin-4- |
| | yl)methyl]-2-{3,3′,4′-trifluoro-[1,1′-biphenyl]-4-yl}acetamide |
| I-704 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[4-(2,3,4- |
| | trifluorophenyl)-1H-pyrazol-1-yl]acetamide |
| I-705 | 2-[4-(difluoromethyl)-1H-pyrazol-1-yl]-N-[(3R)-1,1-dioxo-2,3- |
| | dihydro-1lambda6-thiophen-3-yl]-N-[(2-methoxypyridin-4- |
| | yl)methyl]-2-{3,3′,4′-trifluoro-[1,1′-biphenyl]-4-yl}acetamide |
| I-706 | 2-(4-cyano-1H-pyrazol-1-yl)-N-[(3R)-1,1-dioxo-2,3-dihydro- |
| | 1lambda6-thiophen-3-yl]-N-[(2-methoxypyridin-4-yl)methyl]-2- |
| | {3,3′,4′-trifluoro-[1,1′-biphenyl]-4-yl}acetamide |
| I-707 | N-[(3R)-1,1-dioxo-2,3-dihydro-1lambda6-thiophen-3-yl]-2-(2- |
| | methanesulfonylethoxy)-N-[(2-methoxypyridin-4-yl)methyl]-2- |
| | {3,3′,4′-trifluoro-[1,1′-biphenyl]-4-yl}acetamide |
| I-708 | rel-N-[(3R)-1,1-dioxo-2,3-dihydro-1lambda6-thiophen-3-yl]-N- |
| | {[4-(2-hydroxypropan-2-yl)-1,3-thiazol-2-yl]methyl}-2-[4- |
| | (pentafluoro-lambda6-sulfanyl)phenyl]acetamide |
| I-709 | rel-N-[(3R)-1,1-dioxo-2,3-dihydro-1lambda6-thiophen-3-yl]-N- |
| | {[4-(2-hydroxypropan-2-yl)-1,3-thiazol-2-yl]methyl}-2-[4- |
| | (pentafluoro-lambda6-sulfanyl)phenyl]acetamide |
| I-710 | (2R*)-N-[(3R)-1,1-dioxo-2,3-dihydro-1lambda6-thiophen-3-yl]- |
| | 2-(2-methanesulfonylethoxy)-N-[(2-methoxypyridin-4- |
| | yl)methyl]-2-{3,3′,4′-trifluoro-[1,1′-biphenyl]-4-yl}acetamide |
| I-711 | (2R*)-2-[4-(difluoromethyl)-1H-pyrazol-1-yl]-N-[(3R)-1,1- |
| | dioxo-2,3-dihydro-1lambda6-thiophen-3-yl]-N-[(2- |
| | methoxypyridin-4-yl)methyl]-2-{3,3′,4′-trifluoro-[1,1′-biphenyl]- |
| | 4-yl}acetamide |
| I-712 | (2R*)-2-(4-cyano-1H-pyrazol-1-yl)-N-[(3R)-1,1-dioxo-2,3- |
| | dihydro-1lambda6-thiophen-3-yl]-N-[(2-methoxypyridin-4- |
| | yl)methyl]-2-{3,3′,4′-trifluoro-[1,1′-biphenyl]-4-yl}acetamide |
| I-713 | 2-[4-(3,4-difluorophenyl)-3-methyl-1H-pyrazol-1-yl]-N-[(3R)- |
| | 1,1-dioxo-2,3-dihydro-1lambda6-thiophen-3-yl]-N-[(2- |
| | methoxypyridin-4-yl)methyl]acetamide |
| I-714 | rac-(2R,5S)-N-(1,1-dioxo-2,3-dihydro-1lambda6-thiophen-3-yl)- |
| | N-[(pyridin-4-yl)methyl]-5-(trifluoromethyl)oxolane-2- |
| | carboxamide |
| I-715 | rac-(1R,4S,5S)-N-(1,1-dioxo-2,3-dihydro-1lambda6-thiophen-3- |
| | yl)-4-(3-fluorophenyl)-N-[(pyridin-4-yl)methyl]-2- |
| | oxabicyclo[2.1.1]hexane-5-carboxamide |
| I-716 | rac-(2R,5S)-N-(1,1-dioxo-2,3-dihydro-1lambda6-thiophen-3-yl)- |
| | 5-[(1H-pyrazol-1-yl)methyl]-N-[(pyridin-4-yl)methyl]oxolane-2- |
| | carboxamide |
| I-717 | N-(1,1-dioxo-2,3-dihydro-1lambda6-thiophen-3-yl)-6-methoxy- |
| | N-[(pyridin-4-yl)methyl]-1,2,3,4-tetrahydronaphthalene-1- |
| | carboxamide |
| I-718 | N-{[4-(2,2-difluorocyclopropyl)-1,3-thiazol-2-yl]methyl}-2-[4- |
| | (3,4-difluorophenyl)-1H-pyrazol-1-yl]-N-(1,1-dioxo-2,3-dihydro- |
| | 1lambda6-thiophen-3-yl)acetamide |
| I-719 | N-(1,1-dioxo-2,3-dihydro-1lambda6-thiophen-3-yl)-N-{[2-(2- |
| | methoxyethoxy)pyridin-4-yl]methyl}-2-[4-(pentafluoro-lambda6- |
| | sulfanyl)phenyl]acetamide |
| I-720 | N-[(3R)-1,1-dioxo-2,3-dihydro-1lambda6-thiophen-3-yl]-N-[(2- |
| | methoxypyridin-4-yl)methyl]-2-[5-(trifluoromethoxy)-2H- |
| | indazol-2-yl]acetamide |
| I-721 | N-[(3R)-1,1-dioxo-2,3-dihydro-1lambda6-thiophen-3-yl]-N-[(2- |
| | methoxypyridin-4-yl)methyl]-2-[5-(trifluoromethoxy)-1H- |
| | indazol-1-yl]acetamide |
| I-722 | N-[(3R)-1,1-dioxo-2,3-dihydro-1lambda6-thiophen-3-yl]-N-[(2- |
| | methoxypyridin-4-yl)methyl]-2-[3-methyl-5-(trifluoromethyl)- |
| | 1H-indazol-1-yl]acetamide |
| I-723 | 2-[4-(3,4-difluorophenyl)-1H-pyrazol-1-yl]-N-(1,1-dioxo-2,3- |
| | dihydro-1lambda6-thiophen-3-yl)-N-({4-[(1S,2R)-2- |
| | fluorocyclopropyl]-1,3-thiazol-2-yl}methyl)acetamide |
| I-724 | 2-[4-(3,4-difluorophenyl)-1H-pyrazol-1-yl]-N-(1,1-dioxo-2,3- |
| | dihydro-1lambda6-thiophen-3-yl)-N-({4-[(1R,2R)-2- |
| | fluorocyclopropyl]-1,3-thiazol-2-yl}methyl)acetamide |
| I-725 | N-(1,1-dioxo-2,3-dihydro-1lambda6-thiophen-3-yl)-2-[4- |
| | (pentafluoro-lambda6-sulfanyl)phenyl]-N-{[2- |
| | (trifluoromethoxy)pyridin-4-yl]methyl}acetamide |
| I-726 | 2-[4-(3,4-difluorophenyl)-1H-pyrazol-1-yl]-N-(1,1-dioxo-2,3- |
| | dihydro-1lambda6-thiophen-3-yl)-N-{[4-(1-fluorocyclopropyl)- |
| | 1,3-thiazol-2-yl]methyl}acetamide |
| I-727 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[5- |
| | (pentafluoro-lambda6-sulfanyl)-1H-indazol-1-yl]acetamide |
| I-728 | N-benzyl-N-(5-cyano-1,1-dioxo-2,3-dihydro-1lambda6-thiophen- |
| | 3-yl)-2-{4′-fluoro-[1,1′-biphenyl]-4-yl}acetamide |
| I-729 | N-{[4-(difluoromethyl)-1,3-thiazol-2-yl]methyl}-N-(1,1-dioxo- |
| | 2,3-dihydro-1lambda6-thiophen-3-yl)-2-[4-(pentafluoro-lambda6- |
| | sulfanyl)phenyl]acetamide |
| I-730 | rel-N-[(3R)-1,1-dioxo-2,3-dihydro-1lambda6-thiophen-3-yl]-N- |
| | {[2-(2-hydroxypropan-2-yl)pyridin-4-yl]methyl}-5-(pentafluoro- |
| | lambda6-sulfanyl)-2,3-dihydro-1-benzofuran-2-carboxamide |
| I-731 | N-[(3R)-1,1-dioxo-2,3-dihydro-1lambda6-thiophen-3-yl]-N-[(2- |
| | methoxypyridin-4-yl)methyl]-2-(2H-1,2,3,4-tetrazol-2-yl)-2- |
| | {3,3′,4′-trifluoro-[1,1′-biphenyl]-4-yl}acetamide |
| I-732 | N-[(3R)-1,1-dioxo-2,3-dihydro-1lambda6-thiophen-3-yl]-N-[(2- |
| | methoxypyridin-4-yl)methyl]-2-(1H-1,2,3-triazol-1-yl)-2-{3,3′,4′- |
| | trifluoro-[1,1′-biphenyl]-4-yl}acetamide |
| I-733 | N-[(3R)-1,1-dioxo-2,3-dihydro-1lambda6-thiophen-3-yl]-N-[(2- |
| | methoxypyridin-4-yl)methyl]-2-(2H-1,2,3-triazol-2-yl)-2-{3,3′,4′- |
| | trifluoro-[1,1′-biphenyl]-4-yl}acetamide |
| I-734 | N-[(3R)-1,1-dioxo-2,3-dihydro-1lambda6-thiophen-3-yl]-N-[(2- |
| | methoxypyridin-4-yl)methyl]-2-(1H-1,2,3,4-tetrazol-1-yl)-2- |
| | {3,3′,4′-trifluoro-[1,1′-biphenyl]-4-yl}acetamide |
| I-735 | (2R*)-2-(2-cyanoethoxy)-N-[(3R)-1,1-dioxo-2,3-dihydro- |
| | 1lambda6-thiophen-3-yl]-N-[(2-methoxypyridin-4-yl)methyl]-2- |
| | {3,3′,4′-trifluoro-[1,1′-biphenyl]-4-yl}acetamide |
| I-736 | (2R*)-2-(2-cyanoethoxy)-N-[(3R)-1,1-dioxo-2,3-dihydro- |
| | 1lambda6-thiophen-3-yl]-N-[(2-methoxypyridin-4-yl)methyl]-2- |
| | {3,3′,4′-trifluoro-[1,1′-biphenyl]-4-yl}acetamide |
| I-737 | (2R*)-N-[(3R)-1,1-dioxo-2,3-dihydro-1lambda6-thiophen-3-yl]- |
| | N-[(2-methoxypyridin-4-yl)methyl]-2-(3-methyl-1H-pyrazol-1- |
| | yl)-2-{3,3′,4′-trifluoro-[1,1′-biphenyl]-4-yl}acetamide |
| I-738 | (2R*)-N-[(3R)-1,1-dioxo-2,3-dihydro-1lambda6-thiophen-3-yl]- |
| | N-[(2-methoxypyridin-4-yl)methyl]-2-(5-methyl-1H-pyrazol-1- |
| | yl)-2-{3,3′,4′-trifluoro-[1,1′-biphenyl]-4-yl}acetamide |
| I-739 | 2-[4-(3,4-difluorophenyl)-5-methyl-1H-pyrazol-1-yl]-N-[(3R)- |
| | 1,1-dioxo-2,3-dihydro-1lambda6-thiophen-3-yl]-N-[(2- |
| | methoxypyridin-4-yl)methyl]acetamide |
| I-740 | (2R*)-N-[(3R)-1,1-dioxo-2,3-dihydro-1lambda6-thiophen-3-yl]- |
| | N-[(2-methoxypyridin-4-yl)methyl]-2-(oxetan-3-yloxy)-2- |
| | {3,3′,4′-trifluoro-[1,1′-biphenyl]-4-yl}acetamide |
| I-741 | (2R*)-N-[(3R)-1,1-dioxo-2,3-dihydro-1lambda6-thiophen-3-yl]- |
| | N-[(2-methoxypyridin-4-yl)methyl]-2-(oxetan-3-yloxy)-2- |
| | {3,3′,4′-trifluoro-[1,1′-biphenyl]-4-yl}acetamide |
| I-742 | (2R*)-N-[(3R)-1,1-dioxo-2,3-dihydro-1lambda6-thiophen-3-yl]- |
| | N-[(2-methoxypyridin-4-yl)methyl]-2-[(1-methyl-1H-pyrazol-4- |
| | yl)methoxy]-2-{3,3′,4′-trifluoro-[1,1′-biphenyl]-4-yl}acetamide |
| I-743 | (2R*)-N-[(3R)-1,1-dioxo-2,3-dihydro-1lambda6-thiophen-3-yl]- |
| | N-[(2-methoxypyridin-4-yl)methyl]-2-[(1-methyl-1H-pyrazol-4- |
| | yl)methoxy]-2-{3,3′,4′-trifluoro-[1,1′-biphenyl]-4-yl}acetamide |
| I-744 | 2-(2-cyanoethoxy)-N-[(3R)-1,1-dioxo-2,3-dihydro-1lambda6- |
| | thiophen-3-yl]-N-[(2-methoxypyridin-4-yl)methyl]-2-{3,3′,4′- |
| | trifluoro-[1,1′-biphenyl]-4-yl}acetamide |
| I-745 | N-[(3R)-1,1-dioxo-2,3-dihydro-1lambda6-thiophen-3-yl]-N-[(2- |
| | methoxypyridin-4-yl)methyl]-2-[(1-methyl-1H-pyrazol-4- |
| | yl)methoxy]-2-{3,3′,4′-trifluoro-[1,1′-biphenyl]-4-yl}acetamide |
| I-746 | (2R*)-N-[(3R**)-1,1-dioxo-2,3-dihydro-1lambda6-thiophen-3- |
| | yl]-N-{[4-(2-hydroxypropan-2-yl)-1,3-thiazol-2-yl]methyl}-2- |
| | methoxy-2-{3,3′,4′-trifluoro-[1,1′-biphenyl]-4-yl}acetamide |
| I-747 | (2R*)-N-[(3R**)-1,1-dioxo-2,3-dihydro-1lambda6-thiophen-3- |
| | yl]-N-{[4-(2-hydroxypropan-2-yl)-1,3-thiazol-2-yl]methyl}-2- |
| | methoxy-2-{3,3′,4′-trifluoro-[1,1′-biphenyl]-4-yl}acetamide |
| I-748 | N-[(3R)-1,1-dioxo-2,3-dihydro-1lambda6-thiophen-3-yl]-2-(4- |
| | methoxy-1H-pyrazol-1-yl)-N-[(2-methoxypyridin-4-yl)methyl]-2- |
| | {3,3′,4′-trifluoro-[1,1′-biphenyl]-4-yl}acetamide |
| I-749 | (2R*)-N-[(3R)-1,1-dioxo-2,3-dihydro-1lambda6-thiophen-3-yl]- |
| | N-[(2-methoxypyridin-4-yl)methyl]-2-(5-methyl-1H-pyrazol-1- |
| | yl)-2-{3,3′,4′-trifluoro-[1,1′-biphenyl]-4-yl}acetamide |
| I-750 | (2R*)-N-[(3R)-1,1-dioxo-2,3-dihydro-1lambda6-thiophen-3-yl]- |
| | N-[(2-methoxypyridin-4-yl)methyl]-2-(3-methyl-1H-pyrazol-1- |
| | yl)-2-{3,3′,4′-trifluoro-[1,1′-biphenyl]-4-yl}acetamide |
| I-751 | N-[(3R)-1,1-dioxo-2,3-dihydro-1lambda6-thiophen-3-yl]-N-[(2- |
| | methoxypyridin-4-yl)methyl]-2-{1H-pyrazolo[3,4-c]pyridin-1- |
| | yl}-2-{3,3′,4′-trifluoro-[1,1′-biphenyl]-4-yl}acetamide |
| I-752 | N-[(3R)-1,1-dioxo-2,3-dihydro-1lambda6-thiophen-3-yl]-N-[(2- |
| | methoxypyridin-4-yl)methyl]-2-(oxetan-3-yloxy)-2-{3,3′,4′- |
| | trifluoro-[1,1′-biphenyl]-4-yl}acetamide |
| I-753 | N-(1,1-dioxo-2,3-dihydro-1lambda6-thiophen-3-yl)-2-[4-(4- |
| | fluorophenyl)-1H-pyrazol-1-yl]-N-{[2-(trifluoromethoxy)pyridin- |
| | 4-yl]methyl}acetamide |
| I-754 | N-[(4-cyclopropyl-5-methyl-1,3-thiazol-2-yl)methyl]-N-(1,1- |
| | dioxo-2,3-dihydro-1lambda6-thiophen-3-yl)-2-{3,3′,4′-trifluoro- |
| | [1,1′-biphenyl]-4-yl}acetamide |
| I-755 | rac-(1R,4R,5R)-1-(3-bromophenyl)-N-(1,1-dioxo-2,3-dihydro- |
| | 1lambda6-thiophen-3-yl)-N-[(pyridin-4- |
| | yl)methyl]bicyclo[2.1.1]hexane-5-carboxamide |
| I-756 | N-(1,1-dioxo-2,3-dihydro-1lambda6-thiophen-3-yl)-6-methoxy- |
| | N-[(pyridin-4-yl)methyl]-3,4-dihydro-2H-1-benzopyran-3- |
| | carboxamide |
| I-757 | N-(1,1-dioxo-2,3-dihydro-1lambda6-thiophen-3-yl)-2-[4- |
| | (pentafluoro-lambda6-sulfanyl)phenyl]-N-[(2H-1,2,3,4-tetrazol-5- |
| | yl)methyl]acetamide |
| I-758 | N-[(3R)-1,1-dioxo-2,3-dihydro-1lambda6-thiophen-3-yl]-2-[4-(2- |
| | hydroxypropan-2-yl)-1H-pyrazol-1-yl]-N-[(2-methoxypyridin-4- |
| | yl)methyl]-2-{3,3′,4′-trifluoro-[1,1′-biphenyl]-4-yl}acetamide |
| I-759 | 2-[4-(3,4-difluorophenyl)-1H-pyrazol-1-yl]-N-(1,1-dioxo-2,3- |
| | dihydro-1lambda6-thiophen-3-yl)-N-{[2-(morpholin-4- |
| | yl)pyridin-4-yl]methyl}acetamide |
| I-760 | N-{[2-(azetidin-1-yl)pyridin-4-yl]methyl}-2-[4-(3,4- |
| | difluorophenyl)-1H-pyrazol-1-yl]-N-(1,1-dioxo-2,3-dihydro- |
| | 1lambda6-thiophen-3-yl)acetamide |
| I-761 | 2-[4-(3,4-difluorophenyl)-1H-pyrazol-1-yl]-N-(1,1-dioxo-2,3- |
| | dihydro-1lambda6-thiophen-3-yl)-N-({2-[2-(morpholin-4- |
| | yl)ethoxy]pyridin-4-yl}methyl)acetamide |
| I-762 | 2-[4-(3,4-difluorophenyl)-1H-pyrazol-1-yl]-N-(1,1-dioxo-2,3- |
| | dihydro-1lambda6-thiophen-3-yl)-N-[(6-methoxypyridin-3- |
| | yl)methyl]acetamide |
| I-763 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[4-(2,4,5- |
| | trifluorophenyl)-1H-pyrazol-1-yl]acetamide |
| I-764 | N-[(3R)-1,1-dioxo-2,3-dihydro-1lambda6-thiophen-3-yl]-N-[(2- |
| | methoxypyridin-4-yl)methyl]-2-[4-(3,4,5-trifluorophenyl)-1H- |
| | pyrazol-1-yl]acetamide |
| I-765 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-(2- |
| | methoxyethoxy)-2-[4-(pentafluoro-lambda6- |
| | sulfanyl)phenyl]acetamide |
| I-766 | N-[(3R)-1,1-dioxo-2,3-dihydro-1lambda6-thiophen-3-yl]-N-[(2- |
| | methoxypyridin-4-yl)methyl]-2-[3-methyl-5-(trifluoromethyl)- |
| | 2H-indazol-2-yl]acetamide |
| I-767 | rel-2-[4-(3,4-difluorophenyl)-6-oxo-1,6-dihydropyridazin-1-yl]- |
| | N-[(3R)-1,1-dioxo-2,3-dihydro-1lambda6-thiophen-3-yl]-N-{[2- |
| | (2-hydroxypropan-2-yl)pyridin-4-yl]methyl}acetamide |
| I-768 | N-(1,1-dioxo-2,3-dihydro-1lambda6-thiophen-3-yl)-N-{[2-(2- |
| | hydroxy-2-methylpropoxy)pyridin-4-yl]methyl}-2-[4- |
| | (pentafluoro-lambda6-sulfanyl)phenyl]acetamide |
| I-769 | rel-2-[4-(3,4-difluorophenyl)-2-oxo-1,2-dihydropyridin-1-yl]-N- |
| | [(3R)-1,1-dioxo-2,3-dihydro-1lambda6-thiophen-3-yl]-N-{[2-(2- |
| | hydroxypropan-2-yl)pyridin-4-yl]methyl}propanamide |
| I-770 | N-[(3R)-1,1-dioxo-2,3-dihydro-1lambda6-thiophen-3-yl]-N-[(2- |
| | methoxypyridin-4-yl)methyl]-2-[5-(1,1,2,2,2-pentafluoroethyl)- |
| | 1H-indazol-1-yl]acetamide |
| I-771 | N-[(3R)-1,1-dioxo-2,3-dihydro-1lambda6-thiophen-3-yl]-N-[(2- |
| | methoxypyridin-4-yl)methyl]-2-[5-(1,1,2,2,2-pentafluoroethyl)- |
| | 2H-indazol-2-yl]acetamide |
| I-772 | N-(1,1-dioxo-2,3-dihydro-1lambda6-thiophen-3-yl)-N-{[2- |
| | (pyridin-4-yloxy)pyridin-4-yl]methyl}-2-[4-(3,4,5- |
| | trifluorophenyl)-1H-pyrazol-1-yl]acetamide |
| I-773 | rel-2-[5-chloro-4-(3,4,5-trifluorophenyl)-1H-pyrazol-1-yl]-N- |
| | [(3R)-1,1-dioxo-2,3-dihydro-1lambda6-thiophen-3-yl]-N-{[2-(2- |
| | hydroxypropan-2-yl)pyridin-4-yl]methyl}acetamide |
| I-774 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2-{4-[4- |
| | (trifluoromethyl)phenyl]-1H-pyrazol-1-yl}propanamide |
| I-775 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2-{4-[4- |
| | (trifluoromethyl)phenyl]-1H-pyrazol-1-yl}propanamide |
| I-776 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2-[4- |
| | (3,4,5-trifluorophenyl)-1H-pyrazol-1-yl]propanamide |
| I-777 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2-[4- |
| | (3,4,5-trifluorophenyl)-1H-pyrazol-1-yl]propanamide |
| I-778 | 4-{[N-(1,1-dioxo-2,3-dihydro-1lambda6-thiophen-3-yl)-2- |
| | {3,3′,4′-trifluoro-[1,1′-biphenyl]-4-yl}acetamido]methyl}-N- |
| | methyl-N-[(pyridin-4-yl)methyl]benzamide |
| I-779 | 4-{[N-(1,1-dioxo-2,3-dihydro-1lambda6-thiophen-3-yl)-2- |
| | {3,3′,4′-trifluoro-[1,1′-biphenyl]-4-yl}acetamido]methyl}-N,N- |
| | dimethylbenzamide |
| I-780 | N-(1,1-dioxo-2,3-dihydro-1lambda6-thiophen-3-yl)-N-{[4- |
| | (morpholine-4-carbonyl)phenyl]methyl}-2-{3,3′,4′-trifluoro-[1,1′- |
| | biphenyl]-4-yl}acetamide |
| I-781 | rel-2-[3-(3,4-difluorophenyl)-1H-pyrazol-5-yl]-N-[(3R)-1,1- |
| | dioxo-2,3-dihydro-1lambda6-thiophen-3-yl]-N-{[2-(2- |
| | hydroxypropan-2-yl)pyridin-4-yl]methyl}acetamide |
| I-782 | N-(1,1-dioxo-2,3-dihydro-1lambda6-thiophen-3-yl)-N-({4- |
| | [(pyridin-4-yl)methyl]phenyl}methyl)-2-[4-(3,4,5- |
| | trifluorophenyl)-1H-pyrazol-1-yl]acetamide |
| I-783 | N-(1,1-dioxo-2,3-dihydro-1lambda6-thiophen-3-yl)-N-({4- |
| | [(pyridin-4-yl)methoxy]phenyl}methyl)-2-[4-(3,4,5- |
| | trifluorophenyl)-1H-pyrazol-1-yl]acetamide |
| I-784 | N-[(3R)-1,1-dioxo-2,3-dihydro-1lambda6-thiophen-3-yl]-N-[(2- |
| | methoxypyridin-4-yl)methyl]-2-{4-[(1RS,2RS)-2- |
| | (trifluoromethyl)cyclopropyl]phenyl}acetamide |
| I-785 | 5-chloro-N-[(3R)-1,1-dioxo-2,3-dihydro-1lambda6-thiophen-3- |
| | yl]-N-[(2-methoxypyridin-4-yl)methyl]-2,3,4,9-tetrahydro-1H- |
| | carbazole-2-carboxamide |
| I-786 | 7-chloro-N-[(3R)-1,1-dioxo-2,3-dihydro-1lambda6-thiophen-3- |
| | yl]-N-[(2-methoxypyridin-4-yl)methyl]-2,3,4,9-tetrahydro-1H- |
| | carbazole-3-carboxamide |
| I-787 | 2-[4-(3,4-difluorophenyl)-1H-pyrazol-1-yl]-N-(1,1-dioxo-2,3- |
| | dihydro-1lambda6-thiophen-3-yl)-N-{[2-(piperazin-1-yl)pyridin- |
| | 4-yl]methyl}acetamide |
| I-788 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-{4-[4- |
| | (trifluoromethyl)phenyl]-1H-pyrazol-1-yl}propanamide |
| I-789 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[4-(3,4,5- |
| | trifluorophenyl)-1H-pyrazol-1-yl]propanamide |
| I-790 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-{3,3′,4′- |
| | trifluoro-[1,1′-biphenyl]-4-yl}-2-(3,3,3- |
| | trifluoropropoxy)acetamide |
| I-791 | 4-{[N-(1,1-dioxo-2,3-dihydro-1lambda6-thiophen-3-yl)-2- |
| | {3,3′,4′-trifluoro-[1,1′-biphenyl]-4-yl}acetamido]methyl}-N- |
| | methyl-N-[2-(pyridin-4-yl)ethyl]benzamide |
| I-792 | rel-2-[(3,3-difluorocyclobutyl)methoxy]-N-[(3R)-1,1-dioxo-2,3- |
| | dihydro-1lambda6-thiophen-3-yl]-N-{[2-(2-hydroxypropan-2- |
| | yl)pyridin-4-yl]methyl}-2-{3,3′,4′-trifluoro-[1,1′-biphenyl]-4- |
| | yl}acetamide |
| I-793 | N-[(3R)-1,1-dioxo-2,3-dihydro-1lambda6-thiophen-3-yl]-2- |
| | {[(1E)-2-methanesulfonylethenyl]oxy}-N-[(2-methoxypyridin-4- |
| | yl)methyl]-2-{3,3′,4′-trifluoro-[1,1′-biphenyl]-4-yl}acetamide |
| I-794 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-[5-(3,4,5- |
| | trifluorophenyl)-1,3-thiazol-2-yl]acetamide |
| I-795 | N-{[4-(2-aminopropan-2-yl)phenyl]methyl}-2-[4-(3,4- |
| | difluorophenyl)-1H-pyrazol-1-yl]-N-(1,1-dioxo-2,3-dihydro- |
| | 1lambda6-thiophen-3-yl)acetamide |
| I-796 | N-(1,1-dioxo-2,3-dihydro-1lambda6-thiophen-3-yl)-N-{[4- |
| | (piperidine-1-carbonyl)phenyl]methyl}-2-{3,3′,4′-trifluoro-[1,1′- |
| | biphenyl]-4-yl}acetamide |
| I-797 | N-(1,1-dioxo-2,3-dihydro-1lambda6-thiophen-3-yl)-N-{[4-(2- |
| | hydroxypropan-2-yl)-1,3-thiazol-2-yl]methyl}-2-[4-(3,4,5- |
| | trifluorophenyl)-1H-pyrazol-1-yl]acetamide |
| I-798 | rel-N-[(3R)-1,1-dioxo-2,3-dihydro-1lambda6-thiophen-3-yl]-N- |
| | {[4-(2-hydroxypropan-2-yl)-1,3-thiazol-2-yl]methyl}-2-[4-(3,4,5- |
| | trifluorophenyl)-1H-pyrazol-1-yl]acetamide |
| I-799 | rel-N-[(3R)-1,1-dioxo-2,3-dihydro-1lambda6-thiophen-3-yl]-N- |
| | {[4-(2-hydroxypropan-2-yl)-1,3-thiazol-2-yl]methyl}-2-[4-(3,4,5- |
| | trifluorophenyl)-1H-pyrazol-1-yl]acetamide |
| I-800 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-(1H-1,2,3- |
| | triazol-1-yl)-2-{3,3′,4′-trifluoro-[1,1′-biphenyl]-4-yl}acetamide |
| I-801 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2-(1H- |
| | 1,2,3-triazol-1-yl)-2-{3,3′,4′-trifluoro-[1,1′-biphenyl]-4- |
| | yl}acetamide |
| I-802 | (2R*)-N-[(3R**)-1,1-dioxo-2,3-dihydro-1lambda6-thiophen-3- |
| | yl]-N-{[2-(2-hydroxypropan-2-yl)pyridin-4-yl]methyl}-2-(1H- |
| | 1,2,3-triazol-1-yl)-2-{3,3′,4′-trifluoro-[1,1′-biphenyl]-4- |
| | yl}acetamide |
| I-803 | (2R*)-N-[(3R)-1,1-dioxo-2,3-dihydro-1lambda6-thiophen-3-yl]- |
| | 2-{[(1E)-2-methanesulfonylethenyl]oxy}-N-[(2-methoxypyridin- |
| | 4-yl)methyl]-2-{3,3′,4′-trifluoro-[1,1′-biphenyl]-4-yl}acetamide |
| I-804 | (2R*)-N-[(3R)-1,1-dioxo-2,3-dihydro-1lambda6-thiophen-3-yl]- |
| | 2-{[(1E)-2-methanesulfonylethenyl]oxy}-N-[(2-methoxypyridin- |
| | 4-yl)methyl]-2-{3,3′,4′-trifluoro-[1,1′-biphenyl]-4-yl}acetamide |
| I-805 | rel-N-[(3R)-1,1-dioxo-2,3-dihydro-1lambda6-thiophen-3-yl]-N- |
| | {[2-(2-hydroxypropan-2-yl)pyridin-4-yl]methyl}-2-{3,3′,4′- |
| | trifluoro-[1,1′-biphenyl]-4-yl}-2-(2,2,2-trifluoroethoxy)acetamide |
| I-806 | rel-N-[(3R)-1,1-dioxo-2,3-dihydro-1lambda6-thiophen-3-yl]-N- |
| | {[2-(2-hydroxypropan-2-yl)pyridin-4-yl]methyl}-3-(3,4,5- |
| | trifluorophenyl)-4H,5H,6H,7H-pyrazolo[1,5-a]pyridine-7- |
| | carboxamide |
| I-807 | N-[(3R)-1,1-dioxo-2,3-dihydro-1lambda6-thiophen-3-yl]-N-[(2- |
| | methoxypyridin-4-yl)methyl]-2-[(oxetan-3-yl)methoxy]-2- |
| | {3,3′,4′-trifluoro-[1,1′-biphenyl]-4-yl}acetamide |
| I-808 | N-[(3R)-1,1-dioxo-2,3-dihydro-1lambda6-thiophen-3-yl]-2-[(1- |
| | methanesulfonylpyrrolidin-3-yl)methoxy]-N-[(2-methoxypyridin- |
| | 4-yl)methyl]-2-{3,3′,4′-trifluoro-[1,1′-biphenyl]-4-yl}acetamide |
| I-809 | N-[(3R)-1,1-dioxo-2,3-dihydro-1lambda6-thiophen-3-yl]-2-[(1- |
| | methanesulfonylpiperidin-3-yl)methoxy]-N-[(2-methoxypyridin- |
| | 4-yl)methyl]-2-{3,3′,4′-trifluoro-[1,1′-biphenyl]-4-yl}acetamide |
| I-810 | N-[(3R)-1,1-dioxo-2,3-dihydro-1lambda6-thiophen-3-yl]-2-[(1- |
| | methanesulfonylazetidin-3-yl)methoxy]-N-[(2-methoxypyridin-4- |
| | yl)methyl]-2-{3,3′,4′-trifluoro-[1,1′-biphenyl]-4-yl}acetamide |
| I-811 | N-[(3R)-1,1-dioxo-2,3-dihydro-1lambda6-thiophen-3-yl]-N-[(2- |
| | methoxypyridin-4-yl)methyl]-2-[(oxan-4-yl)methoxy]-2-{3,3′,4′- |
| | trifluoro-[1,1′-biphenyl]-4-yl}acetamide |
| I-812 | rel-2-(3,3-difluoropropoxy)-N-[(3R)-1,1-dioxo-2,3-dihydro- |
| | 1lambda6-thiophen-3-yl]-N-{[2-(2-hydroxypropan-2-yl)pyridin- |
| | 4-yl]methyl}-2-{3,3′,4′-trifluoro-[1,1′-biphenyl]-4-yl}acetamide |
| I-813 | rel-2-(2,2-difluoroethoxy)-N-[(3R)-1,1-dioxo-2,3-dihydro- |
| | 1lambda6-thiophen-3-yl]-N-{[2-(2-hydroxypropan-2-yl)pyridin- |
| | 4-yl]methyl}-2-{3,3′,4′-trifluoro-[1,1′-biphenyl]-4-yl}acetamide |
| I-814 | rel-2-(benzyloxy)-N-[(3R)-1,1-dioxo-2,3-dihydro-1lambda6- |
| | thiophen-3-yl]-N-{[2-(2-hydroxypropan-2-yl)pyridin-4- |
| | yl]methyl}-2-{3,3′,4′-trifluoro-[1,1′-biphenyl]-4-yl}acetamide |
| I-815 | rel-2-(cyclopropylmethoxy)-N-[(3R)-1,1-dioxo-2,3-dihydro- |
| | 1lambda6-thiophen-3-yl]-N-{[2-(2-hydroxypropan-2-yl)pyridin- |
| | 4-yl]methyl}-2-{3,3′,4′-trifluoro-[1,1′-biphenyl]-4-yl}acetamide |
| I-816 | 2-[1-(2,4-difluorophenyl)-3,5-dimethyl-1H-pyrazol-4-yl]-N-(1,1- |
| | dioxo-2,3-dihydro-1lambda6-thiophen-3-yl)-N-[(pyridin-4- |
| | yl)methyl]acetamide |
| I-817 | N-(1,1-dioxo-2,3-dihydro-1lambda6-thiophen-3-yl)-N-[(6-oxo- |
| | 1,6-dihydropyridazin-3-yl)methyl]-2-[4-(pentafluoro-lambda6- |
| | sulfanyl)phenyl]acetamide |
| I-818 | N-(1,1-dioxo-2,3-dihydro-1lambda6-thiophen-3-yl)-3-(4- |
| | fluorophenyl)-N-[(pyridin-4-yl)methyl]-1H-pyrazole-5- |
| | carboxamide |
| I-819 | N-(1,1-dioxo-2,3-dihydro-1lambda6-thiophen-3-yl)-2-(2-oxo-4- |
| | phenyl-1,3-diazinan-1-yl)-N-[(pyridin-4-yl)methyl]acetamide |
| I-820 | N-(1,1-dioxo-2,3-dihydro-1lambda6-thiophen-3-yl)-N-[1-(2- |
| | methoxypyridin-4-yl)ethyl]-2-[4-(3,4,5-trifluorophenyl)-1H- |
| | pyrazol-1-yl]acetamide |
| I-821 | rel-2-[5-amino-3-(3,4,5-trifluorophenyl)-1H-pyrazol-1-yl]-N- |
| | [(3R)-1,1-dioxo-2,3-dihydro-1lambda6-thiophen-3-yl]-N-{[2-(2- |
| | hydroxypropan-2-yl)pyridin-4-yl]methyl}acetamide |
| I-822 | N-[(3,3-difluorocyclobutyl)methyl]-N′-[(3R)-1,1-dioxo-2,3- |
| | dihydro-1lambda6-thiophen-3-yl]-N′-[(2-methoxypyridin-4- |
| | yl)methyl]-N-methyl-2-{3,3′,4′-trifluoro-[1,1′-biphenyl]-4- |
| | yl}propanediamide |
| I-823 | [1-(2-{[(1,1-dioxo-2,3-dihydro-1lambda6-thiophen-3- |
| | yl)amino]methyl}-1,3-thiazol-4-yl)cyclopropyl]methyl 2-[4- |
| | (3,4,5-trifluorophenyl)-1H-pyrazol-1-yl]acetate |
| I-824 | N-[(3-cyclopropyl-1,2-thiazol-5-yl)methyl]-N-(1,1-dioxo-2,3- |
| | dihydro-1lambda6-thiophen-3-yl)-2-[4-(3,4,5-trifluorophenyl)- |
| | 1H-pyrazol-1-yl]acetamide |
| I-825 | 2-[5-(3,4-difluorophenyl)-1,2-thiazol-3-yl]-N-[(3R)-1,1-dioxo- |
| | 2,3-dihydro-1lambda6-thiophen-3-yl]-N-{[2-(2-hydroxypropan- |
| | 2-yl)pyridin-4-yl]methyl}acetamide |
| I-826 | N-{[2-(1-cyclopropyl-1-hydroxyethyl)pyridin-4-yl]methyl}-N- |
| | (1,1-dioxo-2,3-dihydro-1lambda6-thiophen-3-yl)-2-[4-(3,4,5- |
| | trifluorophenyl)-1H-pyrazol-1-yl]acetamide |
| I-827 | N-{[2-(1,1-difluoro-2-hydroxypropan-2-yl)pyridin-4-yl]methyl}- |
| | N-(1,1-dioxo-2,3-dihydro-1lambda6-thiophen-3-yl)-2-[4-(3,4,5- |
| | trifluorophenyl)-1H-pyrazol-1-yl]acetamide |
| I-828 | N-[(3R**)-1,1-dioxo-2,3-dihydro-1lambda6-thiophen-3-yl]-N- |
| | [(1R*)-1-(2-methoxypyridin-4-yl)ethyl]-2-[4-(3,4,5- |
| | trifluorophenyl)-1H-pyrazol-1-yl]acetamide |
| I-829 | N-[(3R)-1,1-dioxo-2,3-dihydro-1lambda6-thiophen-3-yl]-N- |
| | [(1S)-1-(2-methoxypyridin-4-yl)ethyl]-2-[4-(3,4,5- |
| | trifluorophenyl)-1H-pyrazol-1-yl]acetamide |
| I-830 | N-[(3R**)-1,1-dioxo-2,3-dihydro-1lambda6-thiophen-3-yl]-N- |
| | [(1R*)-1-(2-methoxypyridin-4-yl)ethyl]-2-[4-(3,4,5- |
| | trifluorophenyl)-1H-pyrazol-1-yl]acetamide |
| I-831 | N-[(3R**)-1,1-dioxo-2,3-dihydro-1lambda6-thiophen-3-yl]-N- |
| | [(1R*)-1-(2-methoxypyridin-4-yl)ethyl]-2-[4-(3,4,5- |
| | trifluorophenyl)-1H-pyrazol-1-yl]acetamide |
| I-832 | N-[(3R)-1,1-dioxo-2,3-dihydro-1lambda6-thiophen-3-yl]-2-[3- |
| | fluoro-4-(3,4,5-trifluorophenyl)-1H-pyrazol-1-yl]-N-{[2-(2- |
| | hydroxypropan-2-yl)pyridin-4-yl]methyl}acetamide |
| I-833 | 2-[(1,1-dioxo-1lambda6-thietan-3-yl)oxy]-N-[(3R)-1,1-dioxo-2,3- |
| | dihydro-1lambda6-thiophen-3-yl]-N-[(2-methoxypyridin-4- |
| | yl)methyl]-2-{3,3′,4′-trifluoro-[1,1′-biphenyl]-4-yl}acetamide |
| I-834 | (2R*)-2-[(1,1-dioxo-1lambda6-thietan-3-yl)oxy]-N-[(3R)-1,1- |
| | dioxo-2,3-dihydro-1lambda6-thiophen-3-yl]-N-[(2- |
| | methoxypyridin-4-yl)methyl]-2-{3,3′,4′-trifluoro-[1,1′-biphenyl]- |
| | 4-yl}acetamide |
| I-835 | (2R*)-2-[(1,1-dioxo-1lambda6-thietan-3-yl)oxy]-N-[(3R)-1,1- |
| | dioxo-2,3-dihydro-1lambda6-thiophen-3-yl]-N-[(2- |
| | methoxypyridin-4-yl)methyl]-2-{3,3′,4′-trifluoro-[1,1′-biphenyl]- |
| | 4-yl}acetamide |
| II-1 | 1-{5-[(3aS,4S,6aR)-2-oxo-hexahydro-1H-thieno[3,4-d]imidazol- |
| | 4-yl]pentanamido}-N-(6-{[4-({1-[4-({N-[(3R*)-1,1-dioxo-2,3- |
| | dihydro-1lambda6-thiophen-3-yl]-2-[2-fluoro-4-(2-methyl-1H- |
| | pyrrol-1-yl)phenyl]acetamido}methyl)phenyl]-N- |
| | methylformamido}methyl)-1H-1,2,3-triazol-1-yl]methyl}pyridin- |
| | 3-yl)-3,6,9,12-tetraoxapentadecan-15-amide |
| II-5 | 1-{5-[(3aS,4S,6aR)-2-oxo-hexahydro-1H-thieno[3,4-d]imidazol- |
| | 4-yl]pentanamido}-N-{6-[(4-{[1-(4-{[N-(1,1-dioxo-2,3-dihydro- |
| | 1lambda6-thiophen-3-yl)-2-[2-fluoro-4-(2-methyl-1H-pyrrol-1- |
| | yl)phenyl]acetamido]methyl}phenyl)-N- |
| | methylformamido]methyl}-1H-1,2,3-triazol-1-yl)methyl]pyridin- |
| | 3-yl}-3,6,9,12-tetraoxapentadecan-15-amide |
| II-2 | 1-{5-[(3aS,4S,6aR)-2-oxo-hexahydro-1H-thieno[3,4-d]imidazol- |
| | 4-yl]pentanamido}-N-{6-[(4-{4-[4-(4-{[N-(1,1-dioxo-2,3- |
| | dihydro-1lambda6-thiophen-3-yl)-2-[2-fluoro-4-(2-methyl-1H- |
| | pyrrol-1-yl)phenyl]acetamido]methyl}benzoyl)piperazin-1-yl]-4- |
| | oxobutyl}-1H-1,2,3-triazol-1-yl)methyl]pyridin-3-yl}-3,6,9,12- |
| | tetraoxapentadecan-15-amide |
| II-3 | 1-{5-[(3aS,4S,6aR)-2-oxo-hexahydro-1H-thieno[3,4-d]imidazol- |
| | 4-yl]pentanamido}-N-{6-[(4-{4-[4-(4-{[N-(1,1-dioxo-2,3- |
| | dihydro-1lambda6-thiophen-3-yl)-2-[2-fluoro-4-(1H-pyrrol-1- |
| | yl)phenyl]acetamido]methyl}benzoyl)piperazin-1-yl]-4- |
| | oxobutyl}-1H-1,2,3-triazol-1-yl)methyl]pyridin-3-yl}-3,6,9,12- |
| | tetraoxapentadecan-15-amide |
| II-4 | 5-[(3aS,4S,6aR)-2-oxo-hexahydro-1H-thieno[3,4-d]imidazol-4- |
| | yl]-N-(21-{4-[(4-{[N-(1,1-dioxo-2,3-dihydro-1lambda6-thiophen- |
| | 3-yl)-2-[2-fluoro-4-(1H-pyrrol-1- |
| | yl)phenyl]acetamido]methyl}phenyl)methyl]piperazin-1-yl}-21- |
| | oxo-3,6,9,12,15,18-hexaoxahenicosan-1-yl)pentanamide |

In another aspect, the disclosure provides a biotinylated compound selected from

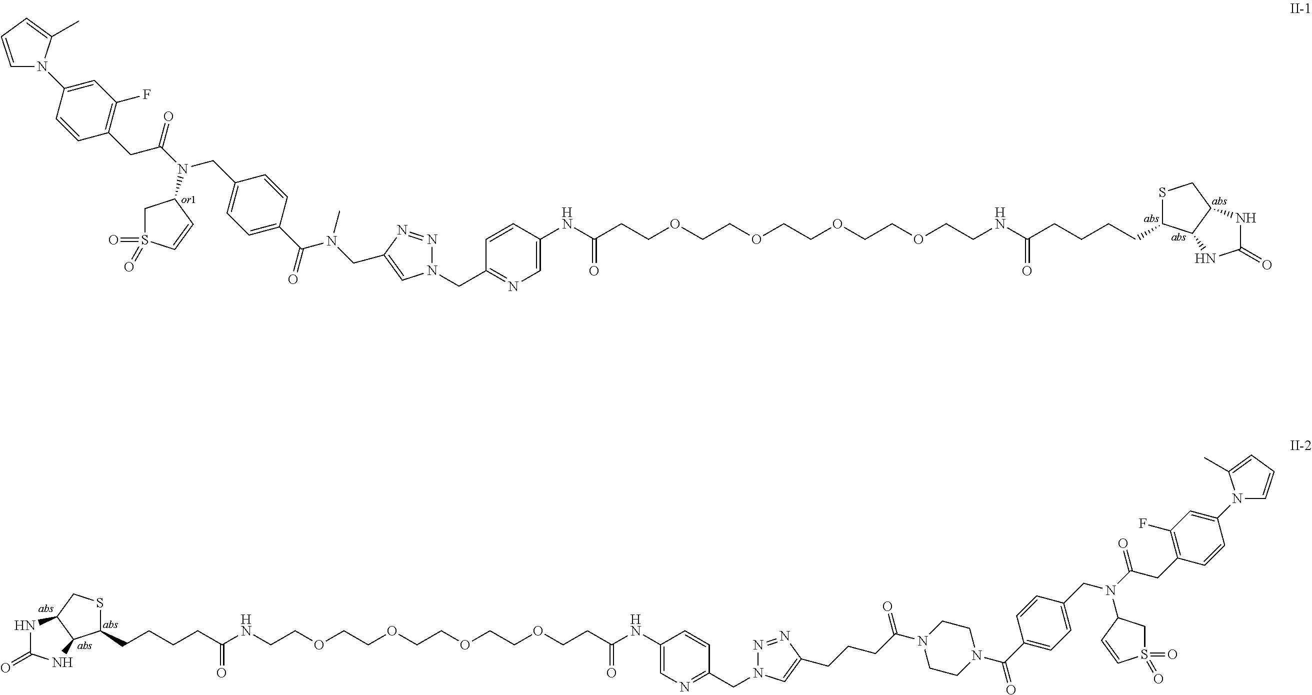 [SMILES: Cc1cccn1-c1ccc(CC(=O)N(Cc2ccc(C(=O)N(C)Cc3cn(Cc4ccc(NC(=O)CCOCCOCCOCCOCCNC(=O)CCCC[C@@H]5SC[C@@H]6NC(=O)N[C@@H]65)cn4)nn3)cc2)[C@@H]2C=CS(=O)(=O)C2)c(F)c1 ; Cc1cccn1-c1ccc(CC(=O)N(Cc2ccc(C(=O)N3CCN(C(=O)CCCc4cn(Cc5ccc(NC(=O)CCOCCOCCOCCOCCNC(=O)CCCC[C@@H]6SC[C@@H]7NC(=O)N[C@@H]76)cn5)nn4)CC3)cc2)C2C=CS(=O)(=O)C2)c(F)c1]

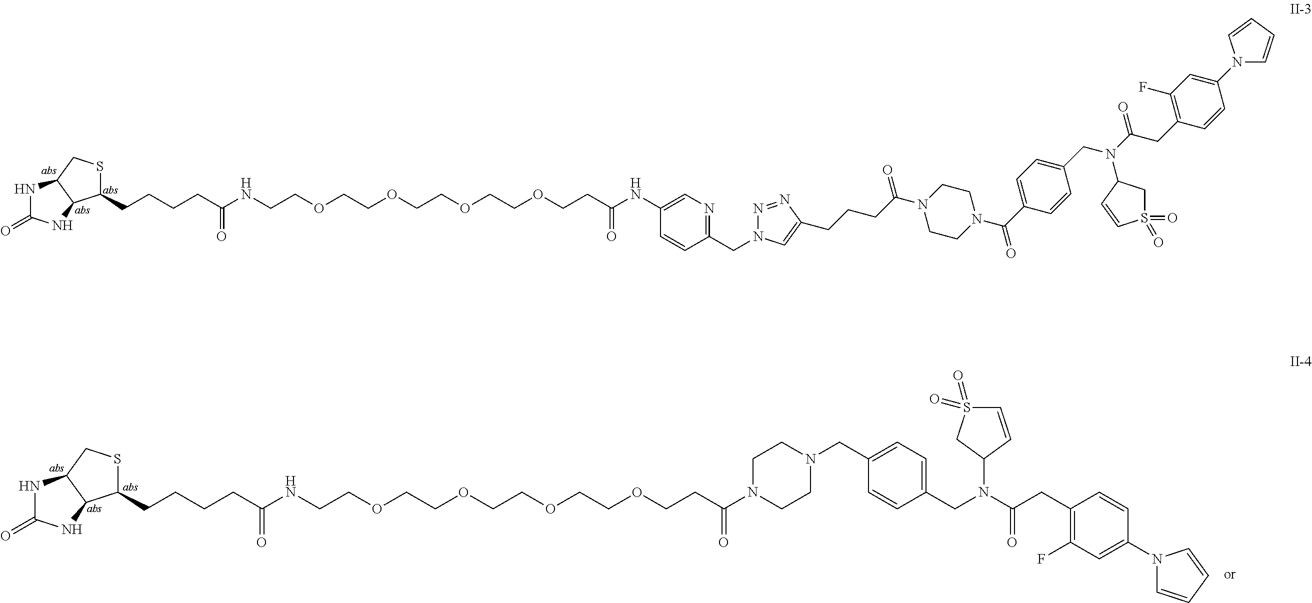 [SMILES: O=C(CCCC[C@@H]1SC[C@@H]2NC(=O)N[C@@H]21)NCCOCCOCCOCCOCCC(=O)Nc1ccc(Cn2cc(CCCC(=O)N3CCN(C(=O)c4ccc(CN(C(=O)Cc5ccc(-n6cccc6)cc5F)C5C=CS(=O)(=O)C5)cc4)CC3)nn2)nc1 ; O=C(CCCC[C@@H]1SC[C@@H]2NC(=O)N[C@@H]21)NCCOCCOCCOCCOCCC(=O)N1CCN(Cc2ccc(CN(C(=O)Cc3ccc(-n4cccc4)cc3F)C3C=CS(=O)(=O)C3)cc2)CC1]

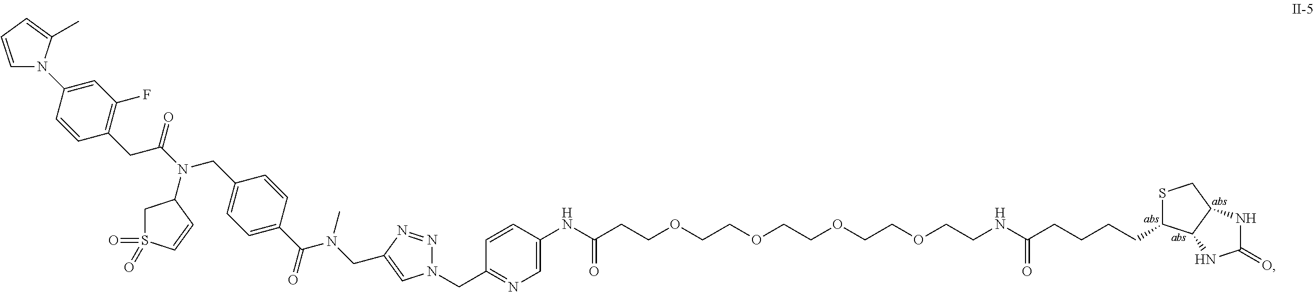 [SMILES: Cc1cccn1-c1ccc(CC(=O)N(Cc2ccc(C(=O)N(C)Cc3cn(Cc4ccc(NC(=O)CCOCCOCCOCCOCCNC(=O)CCCC[C@@H]5SC[C@@H]6NC(=O)N[C@@H]65)cn4)nn3)cc2)C2C=CS(=O)(=O)C2)c(F)c1]

or a pharmaceutically acceptable salt thereof. It is to be understood that all compositions, methods of treatment, methods of administration of compounds, uses as medicaments, described infra which reference “compounds of the invention” or compounds of Formula I, also may include the biotinylated compounds shown supra.

### 4. Pharmaceutical Compositions, Methods of Treatment and Uses of Compounds

In another aspect, the present invention provides a pharmaceutical composition comprising a compound of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration, in particular oral administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
g) diluents, e.g., lactose, dextrose, sucrose, mannitol, sorbitol, cellulose and/or glycine;
h) lubricants, e.g., silica, talcum, stearic acid, its magnesium or calcium salt and/or polyethyleneglycol; for tablets also
i) binders, e.g., magnesium aluminum silicate, starch paste, gelatin, tragacanth, methylcellulose, sodium carboxymethylcellulose and/or polyvinylpyrrolidone; if desired
j) disintegrants, e.g., starches, agar, alginic acid or its sodium salt, or effervescent mixtures; and
k) absorbents, colorants, flavors and sweeteners.

Typical approaches to solubilize compounds for parenteral administration are the optimization of the pH or the use of co-solvents (e.g. PEG300, PEG400, propylene glycol, or ethanol). If these approaches are, for any reason, not feasible, the use of surfactants may be considered (e.g. Tween® 80 or Cremophor EL®). Cyclodextrins are established as safe solubilizing agents. Compounds with a high solubility in natural oils may be solubilized in parenteral fat emulsions.

There is also provided a pharmaceutical composition comprising a compound of Formula I as described herein, or a pharmaceutically acceptable salt thereof, and one or more pharmaceutically acceptable carriers.

### USES

The compounds of Formula I of the present invention in free form or in pharmaceutically acceptable salt form, exhibit valuable pharmacological properties, e.g. WRN inhibiting properties, e.g. as indicated in vitro tests as provided in the next sections, and are therefore indicated for therapy, or for use as research chemicals, e.g. as a chemical probe, and as tool compounds.

Also provided is a compound of Formula I, in particular II, IIIa, IIIb, IVa, IVb, Va, or Vb, as described herein. Said compound can be used as a research chemical, a compound herein comprising an added biotin moeity, for example a tool compound or chemical probe, in particular for research on WRN. In another embodiment there is provided the use of a compound of Formula I, in particular II, IIIa, IIIb, IVa, IVb, Va, or Vb, as described herein, as a research chemical, for example tool compound or chemical probe, in particular for research on WRN.

There is also provided a compound of Formula I as described herein, in particular II, IIIa, IIIb, IVa, IVb, Va, or Vb, or a pharmaceutically acceptable salt thereof, for use in the treatment of cancer. Cancers that may be treated by WRN inhibition include cancers that are characterized as microsatellite instability-high (MSI-H) or mismatch repair deficient (dMMR). In particular, a compound of Formula I as described herein, or a pharmaceutically acceptable salt thereof, may be useful in the treatment of a cancer that is characterized as microsatellite instability-high (MSI-H) or mismatch repair deficient (dMMR).

There is also provided a compound of Formula I as described herein, in particular II, IIIa, IIIb, IVa, IVb, Va, or Vb, or a pharmaceutically acceptable salt thereof, for use as a medicament. In particular, said use is:
for the treatment of a disease that is treated by WRN inhibition,
for the treatment of cancer,
for the treatment of cancer that is characterized as microsatellite instability-high (MSI-H) or mismatch repair deficient (dMMR),
for the treatment of cancer that is characterized as microsatellite instability-high (MSI-H) or mismatch repair deficient (dMMR), such as colorectal, gastric, prostate, endometrial, adrenocortical, uterine, cervical, esophageal, breast, kidney and ovarian cancer,
for the treatment of cancer that is characterized as microsatellite instability-high (MSI-H) or mismatch repair deficient (dMMR) is selected from colorectal, gastric, prostate and endometrial cancer, or
for the treatment of cancer wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided a method of:
modulating WRN activity in a subject, wherein the method comprises administering to the subject a therapeutically effective amount of the compound of Formula I as described herein, in particular II, IIIa, IIIb, IVa, IVb, Va, or Vb, or a pharmaceutically acceptable salt thereof,
inhibiting WRN in a subject, wherein the method comprises administering to the subject a therapeutically effective amount of the compound of Formula I as described herein, in particular II, IIIa, IIIb, IVa, IVb, Va, or Vb, or a pharmaceutically acceptable salt thereof,
treating a disorder or disease which can be treated by WRN inhibition in a subject, comprising administering to the subject a therapeutically effective amount of the compound of Formula I as described herein, in particular II, IIIa, IIIb, IVa, IVb, Va, or Vb, or a pharmaceutically acceptable salt thereof,
treating cancer in a subject, comprising administering to the subject a therapeutically effective amount of the compound of Formula I as described herein, in particular II, IIIa, IIIb, IVa, IVb, Va, or Vb, or a pharmaceutically acceptable salt thereof,
treating cancer in a subject, comprising administering a compound of Formula I as described herein, in particular II, IIIa, IIIb, IVa, IVb, Va, or Vb, wherein the cancer is characterized as microsatellite instability-high (MSI-H) or mismatch repair deficient (dMMR). In particular, the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer. More particularly, the cancer characterized as microsatellite instability-high (MSI-H) or mismatch repair deficient (dMMR) is selected from colorectal, gastric, prostate and endometrial cancer. Examples include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided the use of a compound of Formula I as described herein, in particular II, IIIa, IIIb, IVa, IVb, Va, or Vb, or a pharmaceutically acceptable salt thereof:
in therapy,
in the manufacture of a medicament,
in the manufacture of a medicament for the treatment of cancer. In particular, said cancer is characterized as microsatellite instability-high (MSI-H) or mismatch repair deficient (dMMR),
in the manufacture of a medicament for treatment of a disease which may be treated by WRN inhibition,
   wherein in particular, the cancer is characterized by microsatellite instability-high (MSI-H) or mismatch repair deficient (dMMR), for example colorectal, gastric, prostate, endometrial, adrenocortical, uterine, cervical, esophageal, breast, kidney and ovarian cancer, in particular, colorectal, gastric, prostate or endometrial cancer, or uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

In some embodiments, the subject has or is identified as having a microsatellite instable (MSI-H) cancer, e.g., in reference to a control, e.g., a normal, subject. In one embodiment, the subject has MSI-H advanced solid tumors, a colorectal cancer (CRC), endometrial, uterine, stomach or other MSI-H cancer. In some embodiments, the subject has a colorectal (CRC), endometrial or stomach cancer, which cancer has or is identified as having a microsatellite instability (MSI-H), e.g., in reference to a control, e.g., a normal, subject. Such identification techniques are known in the art.

### Forms

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any formula given herein is intended to represent unlabeled forms as well as isotopically labeled forms of the compounds, in addition to the deuteration specifically claimed in Formula I. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen.

Further, incorporation of certain isotopes, particularly deuterium (i.e., 2H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as 3H, 11C, 13C, 14C, 15N, 18F 31P, 32P, 35S, 36Cl, 123I, 124I, 125I respectively. Accordingly it should be understood that the invention includes compounds that incorporate one or more of any of the aforementioned isotopes, including for example, radioactive isotopes, such as 3H and 14C, or those into which non-radioactive isotopes, such as 2H and 13C are present. Such isotopically labelled compounds are useful in metabolic studies (with 14C), reaction kinetic studies (with, for example 2H or 3H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

A “compound of the present invention” or a “compound of Formula I” includes a zwitterion thereof, a non-zwitterion thereof (non-charged form), or a pharmaceutically acceptable salt of said zwitterionic or non-zwitterionic form thereof.

“zwitterion” or “zwitterionic form” means a compound containing both positive and negatively charged functional groups.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to colorectal, gastric, endometrial, prostate, adrenocortical, uterine, cervical, esophageal, breast, kidney, ovarian cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“WRN inhibitor” or “WRN helicase inhibitor” as used herein means a compound that inhibits Werner Syndrome RecQ DNA helicase (WRN). The term “WRN” as used herein refers to the protein of Werner Syndrome RecQ DNA helicase. The term “WRN” includes mutants, fragments, variants, isoforms, and homologs of full-length wild-type WRN. In one embodiment, the protein is encoded by the WRN gene (Entrez gene ID 7486; Ensembl ID ENSG00000165392). Exemplary WRN sequences are available at the Uniprot database under accession number Q14191.

“Disease or condition mediated by WRN” includes a disease or condition, such as cancer, which is treated by WRN inhibition. In particular this can include cancers characterized as microsatellite instability-high (MSI-H) or mismatch repair deficient (dMMR).

“Microsatellite unstable cancer,” “microsatellite instability-high cancer,” “microsatellite high cancer” and “MSI-high cancer,” “MSIhi” and “MSI-H” when used herein, are used interchangeably, and describe cancers that have a high number of alterations in the length of simple repetitive genomic sequences within microsatellites.

The determination of MSI-H or dMMR tumor status for patients can be performed using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; and Kawakami et al. Curr Treat Options Oncol. 2015; 16(7): 30).

Microsatellite instability can be found in colorectal cancer, gastric cancer and endometrial cancer in particular, but also in adrenocortical, uterine, cervical, esophageal, breast, kidney, prostate and ovarian cancers. Examples of microsatellite high cancers include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

A cancer that has “defective mismatch repair” (dMMR) or “dMMR character” includes cancer types associated with documented MLH1, PMS2, MSH2, MSH3, MSH6, MLH3, and PMS1 mutations or epigenetic silencing, microsatellite fragile sites, or other gene inactivation mechanisms, including but not limited to cancers of the lung, breast, kidney, large intestine, ovary, prostate, upper aerodigestive tract, stomach, endometrium, liver, pancreas, haematopoietic and lymphoid tissue, skin, thyroid, pleura, autonomic ganglia, central nervous system, soft tissue, pediatric rhabdoid sarcomas, melanomas and other cancers. A cell or cancer with “defective” mismatch repair has a significantly reduced (e.g., at least about 25%, 30%, 40%, 50%, 60%, 70%, 80% or 90% decrease) amount of mismatch repair. In some cases, a cell or cancer which is defective in mismatch repair will perform no mismatch repair.

As used herein, the term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The terms “synthetic lethality,” and “synthetically lethal” are used to refer to reduced cell viability and/or a reduced rate of cell proliferation caused by a combination of mutations or approaches to cause loss of function (e.g., RNA interference or protein function inhibition) in two or more genes but not by the loss of function of only one of these genes.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In some embodiments, the methods of the invention comprise administration of a therapeutically effective amount of a compound herein.

In one embodiment, the term “a therapeutically effective amount” refers to the amount of the compound of the present invention that, when administered to a subject, is effective to (1) at least partially alleviate, prevent and/or ameliorate a condition, or a disorder or a disease (i) mediated by WRN, or (ii) associated with WRN activity, or (iii) characterized by activity (normal or abnormal) of WRN; or (2) reduce or inhibit the activity of WRN.

In another embodiment, the term “a therapeutically effective amount” refers to the amount of the compound of the present invention that, when administered to a cell, or a tissue, or a non-cellular biological material, or a medium, is effective to at least partially reducing or inhibiting the activity of WRN, or reducing WRN protein levels.

As used herein, the term “subject” refers to primates (e.g., humans, male or female), dogs, rabbits, guinea pigs, pigs, rats and mice. In certain embodiments, the subject is a primate, a rat or a mouse. In yet other embodiments, the subject is a human.

As used herein, the term “inhibit,” “inhibition” or “inhibiting” refers to the reduction or suppression of a given condition, symptom, or disorder, or disease, or a significant decrease in the baseline activity of a biological activity or process.

As used herein, the term “treat,” “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent,” “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

“May join” means joins or does not join.

“May be replaced by deuterium” means is replaced by deuterium, or is not replaced by deuterium.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### Isomeric Forms

Any asymmetric atom (e.g., carbon or the like) of the compound(s) of the present invention can be present in racemic or enantiomerically enriched, for example the (R)-, (S)- or (R, S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

Compounds of the invention, i.e. compounds of Formula I that contain groups capable of acting as donors and/or acceptors for hydrogen bonds may be capable of forming co-crystals with suitable co-crystal formers. These co-crystals may be prepared from compounds of Formula I by known co-crystal forming procedures. Such procedures include grinding, heating, co-subliming, co-melting, or contacting in solution compounds of Formula I with the co-crystal former under crystallization conditions and isolating co-crystals thereby formed. Suitable co-crystal formers include those described in WO 2004/078163. Hence the invention further provides co-crystals comprising a compound of Formula I.

Furthermore, the compounds of the present invention, including their salts, can also be obtained in the form of their hydrates, or include other solvents used for their crystallization.

The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

### Dosage Forms

The pharmaceutical composition or combination of the present invention may, for example, be in unit dosage of about 1-1000 mg of active ingredient(s) for a subject of about 50-70 kg.

### Combinations

“Combination” refers to either a fixed combination in one dosage unit form, or a combined administration where a compound of Formula I, or a pharmaceutically acceptable salt thereof, and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage.

The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The combinations described herein can include a compound of Formula I and one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the treatment.

There is also provided a combination comprising a compound of Formula I as described herein, in particular II, IIIa, IIIb, IVa, IVb, Va, or Vb, or a pharmaceutically acceptable salt thereof, as described herein, and one or more additional therapeutically active agents. The additional therapeutic agent is, for example, a chemical compound, peptide, antibody, antibody fragment or nucleic acid, which is therapeutically active or enhances the therapeutic activity when administered to a patient in combination with a compound of the present disclosure. In particular, an additional therapeutically active agent is:
an anti-cancer agent,
a chemotherapy,
a chemotherapy selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate,
liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEXO), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), in particular fluorouracil (5-FU) and irinotecan (Camptosar®).
a PD-1 inhibitor,
an anti-PD-1 antibody molecule,
a PD-1 inhibitor selected from spartalizumab (Novartis), nivolumab (Bristol-Myers Squibb), pembrolizumab (Merck & Co), pidilizumab (CureTech), MED10680 (Medimmune), cemiplimab (REGN2810, Regeneron), dostarlimab (TSR-042, Tesaro), PF-06801591 (Pfizer), tislelizumab (BGB-A317, Beigene), BGB-108 (Beigene), INCSHR1210 (Incyte), balstilimab (AGEN2035, Agenus), sintilimab (InnoVent), toripalimab (Shanghai Junshi Bioscience), camrelizumab (Jiangsu Hengrui Medicine Co.), AMP-224 (Amplimmune), penpulimab (Akeso Biopharma Inc), zimberelimab (Arcus Biosciences Inc), and prolgolimab (Biocad Ltd),
spartalizumab, or
tislelizumab (BGB-A317, Beigene).

In a further embodiment, the additional therapeutically active agent is the chemotherapy irinotecan (Camptosar®).

In another embodiment, the additional therapeutically active agent is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. In another embodiment, the additional therapeutically active agent is an anti-PD-1 antibody molecule.

In a further embodiment, the PD-1 inhibitor is an anti-PD-1 antibody molecule as described in US 2015/0210769, published on Jul. 30, 2015, entitled “Antibody Molecules to PD-1 and Uses Thereof.”

In another embodiment, there is provided a combination of a compound of Formula I or a pharmaceutically acceptable salt thereof, and a chemotherapy, and a PD-1 inhibitor. In particular, the chemotherapy and PD-1 inhibitor are selected from those described above.

The above-mentioned compounds, which can be used in combination with a compound of the present invention, can be prepared and administered as described in the art.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by WRN. Products provided as a combined preparation include a composition comprising the compound of Formula I and the other therapeutic agent(s) together in the same pharmaceutical composition, or the compound of the present invention and the other therapeutic agent(s) in separate form, e.g. in the form of a kit.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of the present invention for treating a disease or condition mediated by WRN, wherein the medicament is prepared for administration with another therapeutic agent. The invention also provides the use of another therapeutic agent for treating a disease or condition mediated by WRN, wherein the medicament is administered with a compound of the present invention.

The invention also provides a compound of the present invention for use in treating a disease or condition mediated by WRN, wherein the compound of the present invention is prepared for administration with another therapeutic agent. The invention also provides another therapeutic agent for use in treating a disease or condition mediated by WRN, wherein the other therapeutic agent is prepared for administration with a compound of the present invention. The invention also provides a compound of the present invention for use in treating a disease or condition mediated by WRN, wherein the compound of the present invention is administered with another therapeutic agent. The invention also provides another therapeutic agent for use in a method of treating a disease or condition mediated by WRN, wherein the other therapeutic agent is administered with a compound of the present invention.

The invention also provides the use of a compound of the present invention for treating a disease or condition mediated by WRN, wherein the patient has previously (e.g. within 24 hours) been treated with another therapeutic agent. The invention also provides the use of another therapeutic agent for treating a disease or condition mediated by WRN, wherein the patient has previously (e.g. within 24 hours) been treated with compound of the present invention.

### 5. General Synthetic Methods of Producing Compounds of the Disclosure

Compounds of the present disclosure, in general, may be prepared by amide coupling methods known to those of skill in the art. Coupling of a carboxylic acid A and cyclic sulfone B produce compounds according to the disclosure as shown in Scheme 1 below.

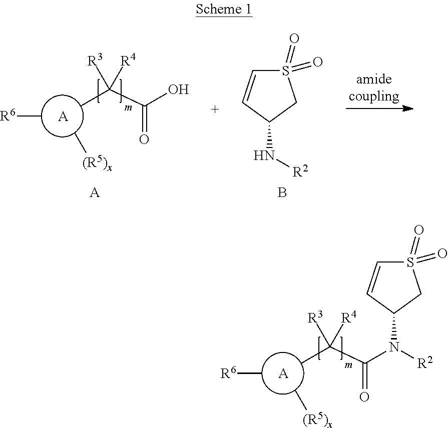

Cyclic sulfone B may be prepared as shown below in Scheme 2. 2,5-dihydrothiophene 1,1-dioxide C may undergo bromination with NBS to produce sulfone D. If stereoisomerically pure material is desired, an acetylation/enzymatic resolution of D may be used to produce chirally pure material E. E may be treated with a base to eliminate bromide and establish the unsaturation of F, with concurrent hydrolysis of the acetyl moiety of E, may produce alcohol F. Coupling of alcohol F with carbonochloridate G may produce intermediate H. Finally, nucleophilic addition of an amine I to H produces amide coupling substrate B.

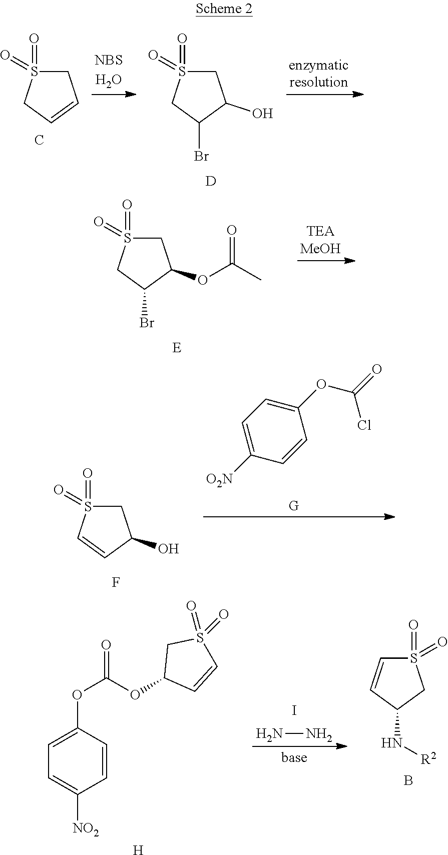

Carboxylic acid coupling partner A of Scheme 1 may be prepared according to simple methods known in the art. For example, see syntheses of intermediates 4, 15, 20, 22, and 25 infra. Those having ordinary skill in the art will be able to adapt such synthetic procedures to afford variably substituted carboxylic acids A for synthesis of the compounds of the disclosure.

### Examples

As depicted in the Examples below, in certain exemplary embodiments, compounds are prepared according to the procedures provided herein. It will be appreciated that, although the methods depict the synthesis of certain compounds of the present disclosure, the methods, and other methods known to one of ordinary skill in the art, can be applied to all compounds and subclasses and species of each of these compounds, as described herein.

### List of Abbreviations

TEA: triethylamine
H₂O: water
ACN: acetonitrile
THF: tetrahydrofuran
FA: formic acid
AcOH: acetic acid
Na₂SO₄: sodium sulfate
LiHMDS: lithium hexamethyldisilazide
K₂CO₃: potassium carbonate
EtOAc: ethyl acetate
NH₄HCO₃: ammonium bicarbonate
NaHCO₃: sodium bicarbonate
HCl: hydrochloric acid
NH₄Cl: ammonium chloride
DCM: dichloromethane
NaOH: sodium hydroxide
DEA: diethylamine
DMF: N,N-dimethyl formamide
EtOH: ethanol
HATU: 1-[Bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide
MeOH: methanol
CHCl₃: chloroform
CDCl₃: Deuterated chloroform
Zn: zinc
Br₂: bromine
DIEA: N,N-diisopropylethylamine
T₃P: Propanephosphonic acid anhydride
Zn(CN)₂: zinc cyanide
POCl₃: phosphoryl chloride
TBSCl: t-butyldimethylchlorosilane
HF-TEA: triethylamine trihydrofluoride
H₂: hydrogen
N₂: nitrogen
Boc₂O: di-tert-butyl pyrocarbonate
DMSO: dimethylsulfoxide
DMSO-d₆: Deuterated dimethyl sulfoxide
NH₃·H₂O: ammonia water
NH₃: ammonia
TCFH: Chloro-N,N,N′,N′-tetramethylformamidinium-hexafluorophosphate
NMI: 1-methyl-1H-imidazole
TFA: trifluoroacetic acid
Ac₂O: acetic anhydride
Pd(OAc)₂: palladium(II) acetate
t-BuOH: tert-Butyl alcohol
Na₂CO₃: sodium carbonate
TMSCN: trimethylsilyl cyanide
Dabco: 1,4-diazabicyclo[2.2.2]octane
SOCl₂: thionyl chloride
dppf: 1,1′-Ferrocenediyl-bis(diphenylphosphine)
Pd(dppf)Cl₂: 1,1′-bis(diphenylphosphino)ferrocene]dichloropalladium(II), complex
Pd(dppf)₂Cl₂—CH₂Cl₂: 1,1′-Bis(diphenylphosphino)ferrocene-palladium(II)dichloride dichloromethane complex
Pd(DtBPF)Cl₂: 1,1′-Bis-(di-tert-butylphosphino-)ferrocene-palladium(II)dichloride
MnO₂: manganese(IV) oxide
DAST: diethylaminosulfur trifluoride
MeMgBr: methylmagnesium bromide
NBS: N-bromosuccinimide
BPO: benzoyl peroxide
LiOH: lithium hydroxide
EDCI: N-(3-dimethylaminopropyl)-N′-ethylcarbodiimide hydrochloride
MeSNa: sodium methanethiolate
NaH: sodium hydride
DPPA: Diphenylphosphoryl azide
NaBH(OAc)₃: Sodium triacetoxyborohydride
3H₂O*NaOAc: Sodium acetate trihydrate
NaOAc: Sodium acetate
BOPCl: Bis(2-oxooxazolidin-3-yl)phosphinic chloride
TMSCHN₂: Trimethylsilyldiazomethane
TMSI: Trimethylsilyl iodide
Rh₂(OAc)₄: Rhodium(II) diacetate dimer
DBU: 1,8-Diazabicyclo[5.4.0]undec-7-ene
LCMS: Liquid chromatography-mass spectrometry
HPLC: High-performance liquid chromatography
CH₃I: Methyl iodide
TBD: 1H,2H,3H,4H,6H,7H,8H-[1,3]diazino[1,2-a]pyrimidine
Pd(amphos)Cl₂: Bis-(di-tert.-butyl-(4-dimethylaminophenyl)-phosphino)-dichloro-palladium(II)
KF: Potassium fluoride
DMAP: N,N-Dimethylpyridin-4-amine
Pd/C: Palladium on carbon
HMTA: 1,3,5,7-Tetraazaadamantane
PE: Petroleum ether
Prep-TLC: Preparative Thin Layer Chromatography
RuPhos: 2-Dicyclohexylphosphino-2′,6′-diisopropoxybiphenyl
RuPhos-Pd-G3: (2-Dicyclohexylphosphino-2′,6′-diisopropoxy-1,1′-biphenyl)[2-(2′-amino-1,1′-biphenyl)]palladium(II)methansulfonat
Xantphos: 4,5-Bis-(diphenylphosphino)-9,9-dimethylxanthen
aq.: aqueous
M: molar concentration
N: normality
Ar: Argon
Ag₂O: Silver(I) oxide
(NH₄)₂S₂O₈: ammonium peroxydisulfate
AgNO₃: silver nitrate
2-MeTHF: 2-methyltetrahydrofuran
TLC: Thin Layer Chromatography
[(t-Bu)₃PH]BF₄: Tri-tert.-butylphosphonium-tetrafluoroborat
br: broad

### Example 1: Synthesis of Compounds of the Disclosure

### Synthesis of N-(1,1-dioxido-2,3-dihydrothiophen-3-yl)-N-(pyridin-4-ylmethyl)-3-(1H-pyrrol-1-yl)bicyclo[4.2.0]octa-1(6),2,4-triene-7-carboxamide I-233

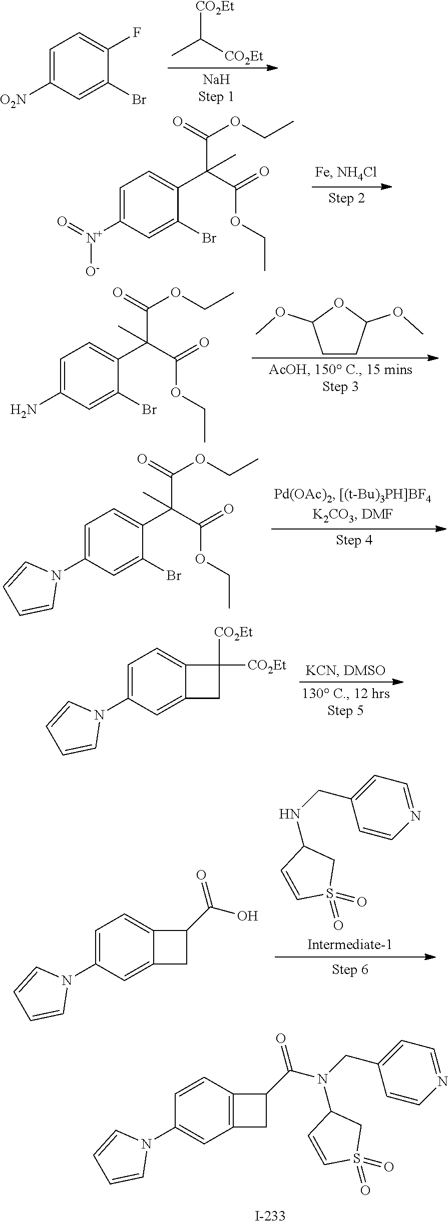

Step 1. Synthesis of diethyl 2-(2-bromo-4-nitrophenyl)-2-methylmalonate: To a mixture of diethyl 2-methylmalonate (1.00 g, 5.74 mmol, 980 μL, 1 eq) in THE (20 mL) was added NaH (252 mg, 6.31 mmol, 60% dispersion in mineral oil, 1.1 eq) at 0° C. The resulting mixture was warmed to room temperature and stirred for 0.5 hr. Then, the reaction was cooled to 0° C., 2-bromo-1-fluoro-4-nitrobenzene (1.26 g, 5.74 mmol, 1 eq) was added at 0° C. After addition, the reaction mixture was allowed to warm to room temperature and stirred for 2 hrs. LCMS showed desired mass was detected. To the mixture was added saturated aqueous NH₄Cl solution (50 mL) and extracted with EtOAc (50 mL). The organic layer was dried over Na₂SO₄ and filtered. The filtrate was concentrated under reduced pressure. The residue was purified by flash silica gel chromatography (Eluent of EtOAc/Petroleum ether) to afford the title compound. LCMS: 374.0[M+H]+.

Step 2. Synthesis of diethyl 2-(4-amino-2-bromophenyl)-2-methylmalonate: To a mixture of diethyl 2-(2-bromo-4-nitrophenyl)-2-methylmalonate (300 mg, 802 μmol, 1 eq) and NH₄Cl (172 mg, 3.21 mmol, 4 eq) in THE (5 mL), EtOH (5 mL) and H₂O (2.5 mL) was added iron powder (179 mg, 3.21 mmol, 4 eq) portion-wise at room temperature. After addition, the resulting mixture was heated to 60° C. and stirred at 60° C. for 4 hrs. LCMS showed desired mass was detected. The mixture was filtered and the filtrate was concentrated under reduced pressure. The residue was dissolved into DCM (50 mL) and filtered. The filtrate was concentrated under reduced pressure. The crude product was used for the next step without further purification. LCMS: 345.0[M+H]+.

Step 3. Synthesis of diethyl 2-(2-bromo-4-(1H-pyrrol-1-yl)phenyl)-2-methylmalonate: To a solution of diethyl 2-(4-amino-2-bromophenyl)-2-methylmalonate (100 mg, 291 μmol, 1 eq) in AcOH (2 mL) was added 2,5-dimethoxytetrahydrofuran (42 mg, 320 mol, 1.1 eq) at room temperature. The mixture was then irradiated under microwave at 150° C. for 15 mins. LCMS showed desired mass was detected. The mixture was concentrated under reduced pressure. The residue was purified by flash silica gel chromatography (Eluent of EtOAc/Petroleum ether) to afford the title compound. LCMS: 396.0[M+H]+.

Step 4. Synthesis of diethyl 3-(1H-pyrrol-1-yl)bicyclo[4.2.0]octa-1,3,5-triene-7,7-dicarboxylate: A mixture of diethyl 2-(2-bromo-4-(1H-pyrrol-1-yl)phenyl)-2-methylmalonate (30 mg, 76.09 μmol, 1 eq), diacetoxypalladium (2 mg, 8 mol, 0.1 eq), [(t-Bu)₃PH]BF₄ (4 mg, 15 gmol, 0.2 eq) and K₂CO₃ (32 mg, 228 μmol, 3 eq) in DMF (1 mL) was stirred at 140° C. for 2 hrs under N₂ atmosphere. LCMS showed desired mass was detected. The mixture was cooled to room temperature and then concentrated under reduced pressure. The residue was purified by flash silica gel chromatography (Eluent of EtOAc/Petroleum ether) to afford the title compound. LCMS: 314.1[M+H]+.

Step 5. Synthesis of 3-(1H-pyrrol-1-yl)bicyclo[4.2.0]octa-1,3,5-triene-7-carboxylic acid: To a mixture of diethyl 3-(1H-pyrrol-1-yl)bicyclo[4.2.0]octa-1,3,5-triene-7,7-dicarboxylate (40 mg, 128 gmol, 1 eq) in DMSO (1 mL) was added KCN (10 mg, 153 μmol, 1.20 eq) at room temperature. Then, the resulting solution was then heated to 130° C. and stirred at 130° C. for 12 hrs. LCMS showed desired mass was detected. After cooling to room temperature, the reaction was quenched with 1N HCl (3 mL) and stirred for 1 hr at room temperature. To the resulting solution was added EtOAc (10 mL) and separated. The organic layer was dried over Na₂SO₄ and filtered. The filtrate was concentrated under reduced pressure to afford the title compound. LCMS: 214.1[M+H]+.

Step 6. Synthesis of N-(1,1-dioxido-2,3-dihydrothiophen-3-yl)-N-(pyridin-4-ylmethyl)-3-(1H-pyrrol-1-yl)bicyclo[4.2.0]octa-1(6),2,4-triene-7-carboxamide: To a mixture of 3-(1H-pyrrol-1-yl)bicyclo[4.2.0]octa-1,3,5-triene-7-carboxylic acid (30 mg, 141 μmol, 1 eq) and 3-((pyridin-4-ylmethyl)amino)-2,3-dihydrothiophene 1,1-dioxide (38 mg, 169 mol, 1.2 eq) in pyridine (1 mL) was added EDCI (54 mg, 281 μmol, 2 eq) at room temperature and then it was stirred at room temperature for 2 hrs. LCMS showed desired mass was detected. The reaction mixture was concentrated under reduced pressure. The residue was purified by prep-HPLC (water (NH₄HCO₃)-ACN) to afford the title compound. ¹H NMR (400 MHz, CDCl₃) δ=8.82-8.36 (m, 2H), 7.28-6.99 (m, 7H), 6.88-6.43 (m, 2H), 6.35 (br s, 2H), 6.09-5.56 (m, 1H), 5.07-4.52 (m, 2H), 4.46-4.23 (m, 1H), 3.81-3.56 (m, 2H), 3.49 (br s, 2H). LCMS: 420.2[M+H]+.

### Synthesis of 2-(4-(1H-pyrrol-1-yl)phenyl)-N-benzyl-N-(5-bromo-1,1-dioxido-2,3-dihydrothiophen-3-yl)acetamide I-269

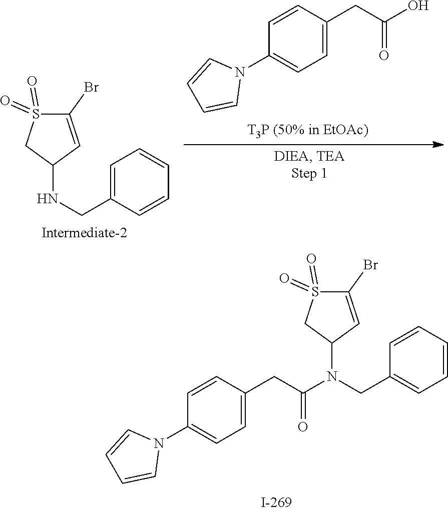 [SMILES: O=S1(=O)CC(NCc2ccccc2)C=C1Br ; O=C(O)Cc1ccc(-n2cccc2)cc1 ; O=C(Cc1ccc(-n2cccc2)cc1)N(Cc1ccccc1)C1C=C(Br)S(=O)(=O)C1]

Step 1. Synthesis of 2-(4-(1H-pyrrol-1-yl)phenyl)-N-benzyl-N-(5-bromo-1,1-dioxido-2,3-dihydrothiophen-3-yl)acetamide: To a stirred mixture of 3-(benzylamino)-5-bromo-2,3-dihydrothiophene 1,1-dioxide (Intermediate-2) (20 mg, 66 μmol, 1.00 eq) and 2-(4-(1H-pyrrol-1-yl)phenyl)acetic acid (13 mg, 66 μmol, 1.00 eq) in THF (3 mL) were added T₃P (126 mg, 198 μmol, 3.00 eq, 50% in EtOAc) and DIEA (34 mg, 264 mol, 4.00 eq) at 0° C. After addition, the reaction mixture was allowed to warm to room temperature and stirred for 2 h. The mixture was concentrated. The residue was purified by flash silica gel chromatography (Eluent of MeOH/DCM) and was further purified by reverse phase HPLC (C18 column, water (0.1% NH₄HCO₃+0.1% NH₃·H₂O)-ACN) to afford the title compound. LCMS: 485.0 [M+H]⁺.

¹H NMR (400 MHz, CDCl₃) δ ppm 7.52-7.41 (m, 6H), 7.37-7.35 (s, 1H), 7.23-7.17 (m, 2H), 7.00 (s, 2H), 6.41-6.38 (m, 3H), 5.81-5.78 (m, 1H), 4.69 (q, 2H), 3.75 (s, 2H), 3.71-3.60 (m, 1H), 3.28-3.25 (m, 1H).

### Synthesis of 4-((N-(1,1-dioxido-2,3-dihydrothiophen-3-yl)-2-(2-fluoro-4-(2-methyl-1H-pyrrol-1-yl)phenyl)acetamido)methyl)-N-methyl-N-((1-((5-(17-oxo-21-((3aS,4S,6aR)-2-oxohexahydro-1H-thieno[3,4-d]imidazol-4-yl)-4,7,10,13-tetraoxa-16-azahenicosanamido)pyridin-2-yl)methyl)-1H-1,2,3-triazol-4-yl)methyl)benzamide 11-1 (single isomer biotinylated compound series)

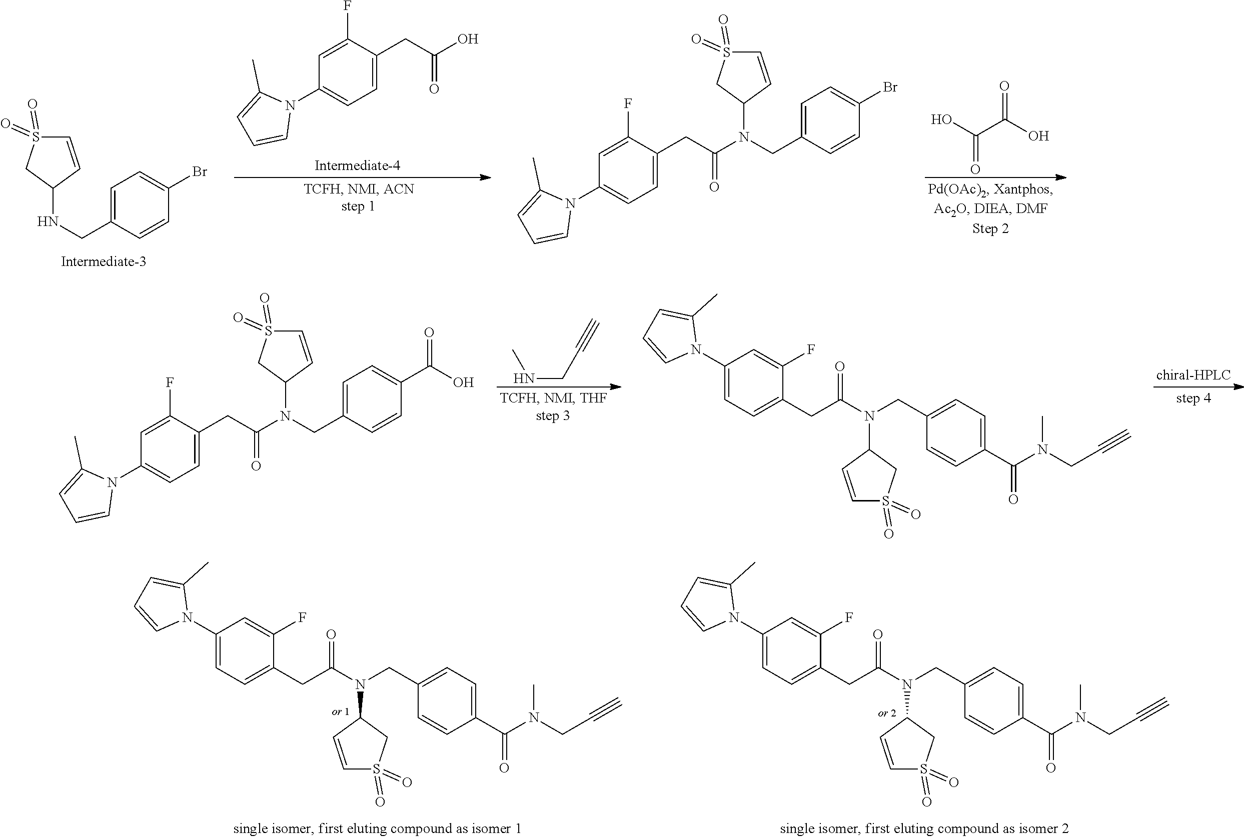

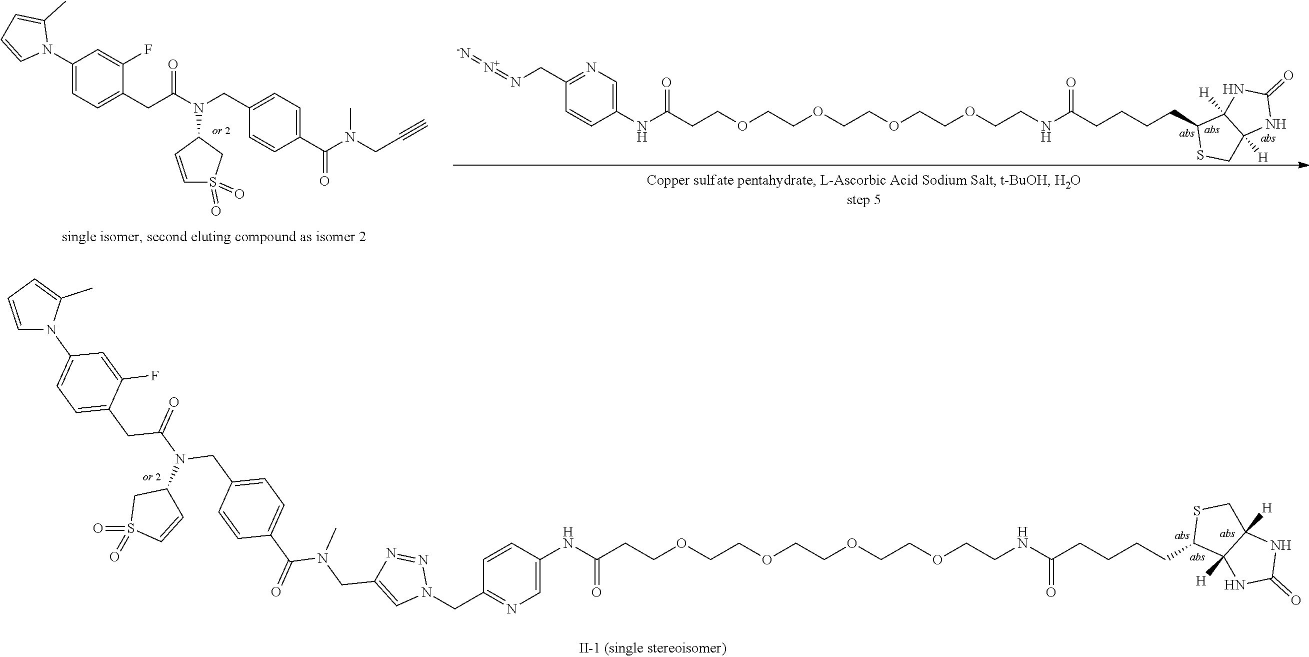

Step 1. Synthesis of N-(4-bromobenzyl)-N-(1,1-dioxido-2,3-dihydrothiophen-3-yl)-2-(2-fluoro-4-(2-methyl-1H-pyrrol-1-yl)phenyl)acetamide: To a solution of 3-((4-bromobenzyl)amino)-2,3-dihydrothiophene 1,1-dioxide (Intermediate-3) (260 mg, 864 μmol, 1.00 eq) and 2-(2-fluoro-4-(2-methyl-1H-pyrrol-1-yl)phenyl)acetic acid (Intermediate-4) (221 mg, 950 μmol, 1.10 eq) in ACN (8.00 mL) were added TCFH (485 mg, 1.72 mmol, 2.00 eq) and NMI (284 mg, 3.46 mmol, 4.00 eq) at 0° C. under N₂ atmosphere. The resulting mixture was stirred for 1 h at room temperature. The resulting mixture was diluted with H₂O (50 mL) and extracted with EtOAc (3×50 mL). The combined organic layers were washed with brine (2×30 mL), dried over anhydrous Na₂SO₄ and filtered. The filtrate was concentrated under reduced pressure. The residue was purified by flash silica gel chromatography (Eluent of EtOAc/PE) to afford the title compound. LCMS: 517.0 [M+H]⁺.

Step 2. Synthesis of 4-((N-(1,1-dioxido-2,3-dihydrothiophen-3-yl)-2-(2-fluoro-4-(2-methyl-1H-pyrrol-1-yl)phenyl)acetamido)methyl)benzoic acid: To a solution of N-(4-bromobenzyl)-N-(1,1-dioxido-2,3-dihydrothiophen-3-yl)-2-(2-fluoro-4-(2-methyl-1H-pyrrol-1-yl)phenyl)acetamide (340 mg, 659 μmol, 1.0 eq) and oxalic acid (89 mg, 988 μmol, 1.50 eq) in DMF (6.00 mL) were added Ac₂O (101 mg, 988 μmol, 1.50 eq), DIEA (127 mg, 988 μmol, 1.50 eq), Xantphos (114 mg, 198 μmol, 0.30 eq) and Pd(OAc)₂ (15 mg, 66 mol, 0.10 eq) at room temperature under N₂ atmosphere. The resulting mixture was stirred for 1 h at 80° C. The mixture was cooled to room temperature and concentrated under reduced pressure. The residue was purified by flash silica gel chromatography (Eluent of EtOAc/PE) to afford the title compound. LCMS: 483.0 [M+H]⁺.

Step 3. Synthesis of 4-((N-(1,1-dioxido-2,3-dihydrothiophen-3-yl)-2-(2-fluoro-4-(2-methyl-1H-pyrrol-1-yl)phenyl)acetamido)methyl)-N-methyl-N-(prop-2-yn-1-yl)benzamide: To a solution of 4-((N-(1,1-dioxido-2,3-dihydrothiophen-3-yl)-2-(2-fluoro-4-(2-methyl-1H-pyrrol-1-yl)phenyl)acetamido)methyl)benzoic acid (220 mg, 455 mol, 1.00 eq) and N-methylprop-2-yn-1-amine (47 mg, 683 μmol, 1.50 eq) in THF (6.00 mL) were added TCFH (256 mg, 911 μmol, 2.00 eq) and NMI (150 mg, 1.82 mmol, 4.00 eq) at 0° C. under N₂ atmosphere. The resulting mixture was stirred for 1 h at room temperature. The resulting mixture was diluted with H₂O (30 mL) and extracted with EtOAc (3×30 mL). The combined organic layers were washed with brine (30 mL), dried over anhydrous Na₂SO₄ and filtered. The filtrate was concentrated under reduced pressure. The residue was purified by reverse phase Prep-HPLC (C18 column, (water (10 mmol/L NH₄HCO₃)-ACN) to afford the title compound. LCMS: 534.0 [M+H]⁺.

Step 4. Separation of 4-((N-(1,1-dioxido-2,3-dihydrothiophen-3-yl)-2-(2-fluoro-4-(2-methyl-1H-pyrrol-1-yl)phenyl)acetamido)methyl)-N-methyl-N-(prop-2-yn-1-yl)benzamide (single isomer, second eluting compound as isomer 2): The 4-((N-(1,1-dioxido-2,3-dihydrothiophen-3-yl)-2-(2-fluoro-4-(2-methyl-1H-pyrrol-1-yl)phenyl)acetamido)methyl)-N-methyl-N-(prop-2-yn-1-yl)benzamide was separated by Chiral-HPLC with the following condition: Column: CHIRALPAK-IF 20×250 mm, 5 μm; Mobile Phase A: EtOH:DCM=3:4, Mobile Phase B: Hex (0.5% 2M NH₃-MeOH); Flow rate: 14 mL/min; Gradient: 65% B to 65% B in 36 min; Wave Length: 220/254 nm. 4-((N-(1,1-dioxido-2,3-dihydrothiophen-3-yl)-2-(2-fluoro-4-(2-methyl-1H-pyrrol-1-yl)phenyl)acetamido)methyl)-N-methyl-N-(prop-2-yn-1-yl)benzamide, single isomer, first eluting compound as isomer 1, retention time: 24.49 minutes. LCMS: 534.0 [M+H]⁺. 4-((N-(1,1-dioxido-2,3-dihydrothiophen-3-yl)-2-(2-fluoro-4-(2-methyl-1H-pyrrol-1-yl)phenyl)acetamido)methyl)-N-methyl-N-(prop-2-yn-1-yl)benzamide, single isomer, second eluting compound as isomer 2, retention time: 29.48 minutes. LCMS: 534.0 [M+H]⁺.

Step 5. Synthesis of 4-((N-(1,1-dioxido-2,3-dihydrothiophen-3-yl)-2-(2-fluoro-4-(2-methyl-1H-pyrrol-1-yl)phenyl)acetamido)methyl)-N-methyl-N-((1-((5-(17-oxo-21-((3aS,4S,6aR)-2-oxohexahydro-1H-thieno[3,4-d]imidazol-4-yl)-4,7,10,13-tetraoxa-16-azahenicosanamido)pyridin-2-yl)methyl)-1H-1,2,3-triazol-4-yl)methyl)benzamide (single isomer): A solution of 4-((N-(1,1-dioxido-2,3-dihydrothiophen-3-yl)-2-(2-fluoro-4-(2-methyl-1H-pyrrol-1-yl)phenyl)acetamido)methyl)-N-methyl-N-(prop-2-yn-1-yl)benzamide (single isomer, second eluting compound as isomer 2) (15 mg, 28 μmol, 1.00 eq) and N-(6-(azidomethyl)pyridin-3-yl)-1-(5-((3aS,4S,6aR)-2-oxohexahydro-1H-thieno[3,4-d]imidazol-4-yl)pentanamido)-3,6,9,12-tetraoxapentadecan-15-amide (30 mg, 48 μmol, 1.70 eq) in t-BuOH (1.5 mL) was stirred for 3 min at room temperature under N₂ atmosphere. To the above mixture was added copper sulfate pentahydrate (7 mg, 28 μmol, 1.00 eq) and L-Ascorbic Acid sodium salt (22 mg, 113 mol, 4.04 eq) in H₂O (1.50 mL) at room temperature under N₂ atmosphere. The resulting mixture was stirred for 1 h at room temperature. The reaction mixture was concentrated under reduced pressure. The residue was purified by reverse phase HPLC (C18 column, water (10 mmol/L NH₄HCO₃)-ACN) to afford the title compound. LCMS: 1156.8 [M+H]⁺, ¹H NMR (400 MHz, CDCl₃) δ ppm 10.23 (s, 1H), 8.70 (d, 1H), 8.14 (s, 1H), 8.06 (d, 1H), 7.82 (t, 1H), 7.62-7.07 (m, 9H), 6.91-6.71 (m, 2H), 6.39 (d, 2H), 6.12-6.07 (m, 1H), 5.98-5.48 (m, 3H), 4.85-4.63 (m, 2H), 4.52-4.46 (m, 1H), 4.31-4.01 (m, 4H), 3.94-3.60 (m, 4H), 3.55-3.42 (m, 12H), 3.38-3.07 (m, 7H), 2.92 (s, 3H), 2.82-2.80 (m, 1H), 2.63-2.54 (m, 3H), 2.21 (d, 3H), 2.06 (t, 2H), 1.70-1.55 (m, 1H), 1.52-1.43 (m, 3H), 1.38-1.27 (m, 2H).

### Synthesis of (4-((2-(4-(1H-pyrrol-1-yl)phenyl)-N-(1,1-dioxido-2,3-dihydrothiophen-3-yl)acetamido)methyl)phenyl)boronic acid I-321

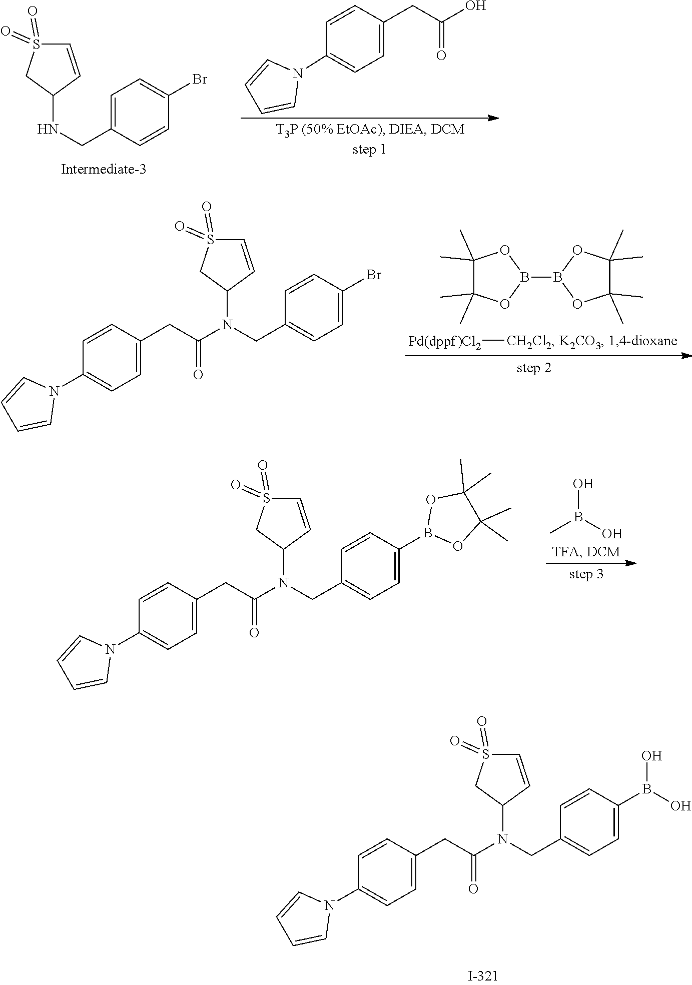

Step 1. Synthesis of 2-(4-(1H-pyrrol-1-yl)phenyl)-N-(4-bromobenzyl)-N-(1,1-dioxido-2,3-dihydrothiophen-3-yl)acetamide: To a solution of 3-((4-bromobenzyl)amino)-2,3-dihydrothiophene 1,1-dioxide (Intermediate-3) (230 mg, 764 μmol, 1.00 eq) and 2-(4-(1H-pyrrol-1-yl)phenyl)acetic acid (154 mg, 764 gmol, 1.00 eq) in DCM (10.0 mL) was added DIEA (296 mg, 2.29 mmol, 3.00 eq) and T₃P (731 mg, 1.15 mmol, 1.50 eq, 50% in EtOAc) at 0° C. The mixture was stirred for 2 h at room temperature. Then to the reaction mixture was added water (10.0 mL) and DCM (10.0 mL). The organic layer was separated, dried over anhydrous Na₂SO₄ and filtered. The filtrate was concentrated under reduced pressure. The residue was purified by Prep-TLC (Eluent of EtOAc/PE) to afford the title compound. LCMS: 485.0 [M+H]⁺.

Step 2. Synthesis of 2-(4-(1H-pyrrol-1-yl)phenyl)-N-(1,1-dioxido-2,3-dihydrothiophen-3-yl)-N-(4-(4,4,5,5-tetramethyl-1,3,2-dioxaborolan-2-yl)benzyl)acetamide: To a mixture of 2-(4-(1H-pyrrol-1-yl)phenyl)-N-(4-bromobenzyl)-N-(1,1-dioxido-2,3-dihydrothiophen-3-yl)acetamide (100 mg, 207 μmol, 1.00 eq) and 4,4,4′,4′,5,5,5′,5′-octamethyl-2,2′-bi(1,3,2-dioxaborolane) (78 mg, 310 μmol, 1.50 eq) in 1,4-dioxane (8.00 mL) was added K₂CO₃ (57 mg, 413 μmol, 2.00 eq) and Pd(dppf)₂Cl₂—CH₂Cl₂ (34 mg, 41 gmol, 0.20 eq) at room temperature under N₂ atmosphere. The resulting mixture was then stirred for 1.5 h at 100° C. The resulting mixture was cooled to room temperature and concentrated under reduced pressure. The residue was purified by Prep-TLC (Eluent of EtOAc/PE) to afford the title compound. LCMS: 533.0 [M+H]⁺.

Step 3. Synthesis of (4-((2-(4-(1H-pyrrol-1-yl)phenyl)-N-(1,1-dioxido-2,3-dihydrothiophen-3-yl)acetamido)methyl)phenyl)boronic acid: A solution of 2-(4-(1H-pyrrol-1-yl)phenyl)-N-(1,1-dioxido-2,3-dihydrothiophen-3-yl)-N-(4-(4,4,5,5-tetramethyl-1,3,2-dioxaborolan-2-yl)benzyl)acetamide (100 mg, 188 mol, 1.00 eq) and methylboronic acid (22 mg, 376 μmol, 2.00 eq) in DCM (10 mL) and TFA (1.00 mL) was stirred for 3 h at room temperature under N₂ atmosphere. The resulting mixture was concentrated under reduced pressure. The residue was purified by reverse phase HPLC (C18 column, water (0.1% FA)-ACN) to afford the title compound. LCMS: 451.0 [M+H]⁺. ¹H NMR (400 MHz, DMSO-d₆) δ ppm 8.07-8.00 (m, 2H), 7.82-7.69 (m, 2H), 7.56-7.46 (m, 2H), 7.37-7.32 (m, 3H), 7.27-7.18 (m, 3H), 7.12-7.08 (m, 1H), 6.78-6.53 (m, 1H), 6.25 (d, 2H), 5.79-5.48 (m, 1H), 4.76-3.95 (m, 3H), 3.74-3.68 (m, 2H), 3.33-3.08 (m, 1H).

### Synthesis of 2-(4-(1H-pyrrol-1-yl)phenyl)-N-benzyl-N-(5-methyl-1,1-dioxido-2,3-dihydrothiophen-3-yl)acetamide I-566

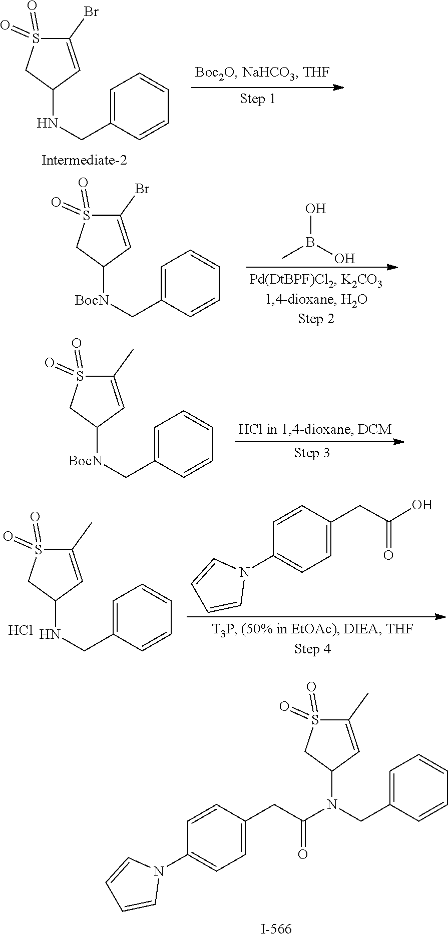

Step 1. Synthesis of tert-butyl benzyl(5-bromo-1,1-dioxido-2,3-dihydrothiophen-3-yl)carbamate: To a stirred mixture of 3-(benzylamino)-5-bromo-2,3-dihydrothiophene 1,1-dioxide (Intermediate-2) (569 mg, 1.88 mmol, 1.00 eq) in THE (15 mL) were added NaHCO₃ (953 mg, 11.3 mmol, 6.00 eq) and Boc₂O (2.47 g, 11.3 mmol, 6.00 eq) at room temperature. The resulting mixture was heated to 60° C. and stirred at 60° C. for 4 h. The mixture was cooled to room temperature. The resulting mixture was diluted with H₂O (30 mL). The resulting mixture was extracted with EtOAc (3×30 mL). The combined organic layers were washed with brine (2×30 mL), dried over anhydrous Na₂SO₄ and filtered. The filtrate was concentrated under reduced pressure. The residue was purified by flash silica gel chromatography (Eluent of EtOAc/PE) to afford title compound. LCMS: 402.0 [M+H]⁺.

Step 2. Synthesis of tert-butyl benzyl(5-bromo-1,1-dioxido-2,3-dihydrothiophen-3-yl)carbamate: To a solution of tert-butyl benzyl(5-bromo-1,1-dioxido-2,3-dihydrothiophen-3-yl)carbamate (60 mg, 150 μmol, 1.00 eq) and methylboronic acid (268 mg, 4.49 mmol, 30.0 eq) in 1,4-dioxane (4.00 mL) and H₂O (1.00 mL) were added K₂CO₃ (61 mg, 449 μmol, 3.00 eq) and Pd(DtBPF)Cl₂ (10 mg, 15 μmol, 0.10 eq) at room temperature under N₂ atmosphere. The resulting mixture was then stirred at 100° C. for 2 h under N₂ atmosphere. The mixture was cooled to room temperature. The mixture was concentrated under vacuum. The residue was purified by flash silica gel chromatography (Eluent of EtOAc/PE) to afford the title compound. LCMS: 338.1 [M+H]⁺.

Step 3. Synthesis of 3-(benzylamino)-5-methyl-2,3-dihydrothiophene 1,1-dioxide hydrochloride: To a stirred mixture of tert-butyl benzyl(5-bromo-1,1-dioxido-2,3-dihydrothiophen-3-yl)carbamate (43 mg, 128 μmol, 1.00 eq) in DCM (3.00 mL) was added 4 M HCl solution in 1,4-dioxane (1.00 mL, 4.00 mmol, 31.3 eq) at 0° C. The resulting mixture was allowed to warm to room temperature and stirred for 2 h. The resulting mixture was concentrated under reduced pressure. The crude product was used in the next step directly without further purification. LCMS: 238.1 [M+H]⁺.

Step 4. Synthesis of 2-(4-(1H-pyrrol-1-yl)phenyl)-N-benzyl-N-(5-methyl-1,1-dioxido-2,3-dihydrothiophen-3-yl)acetamide: To a solution of 3-(benzylamino)-5-methyl-2,3-dihydrothiophene 1,1-dioxide hydrochloride (55 mg, 201 mol, 1.00 eq) and 2-(4-(1H-pyrrol-1-yl)phenyl)acetic acid (20 mg, 100 mol, 0.50 eq) in THE (5.00 mL) were added DIEA (156 mg, 1.21 mmol, 6.00 eq) and T₃P (1.03 g, 1.61 mmol, 8.00 eq, 50% in EtOAc) at 0° C. and then stirred at 0° C. for 0.5 h. The resulting mixture was concentrated under reduced pressure. The residue was purified by flash silica gel chromatography (Eluent of EtOAc/PE) and further purified by reverse phase HPLC (C18 column, water (10 mmol/L NH₄HCO₃)-ACN) to afford the title compound. LCMS: 421.2 [M+H]⁺. ¹H NMR (400 MHz, CDCl₃) δ ppm 7.52-7.39 (m, 2H), 7.35-7.26 (m, 3H), 7.23-7.18 (m, 4H), 7.07-7.00 (m, 2H), 6.35 (s, 2H), 5.92-5.88 (m, 2H), 4.55 (q, 2H), 3.72 (s, 2H), 3.64-3.59 (m, 1H), 3.12-3.06 (m, 1H), 1.95 (s, 3H).

### Synthesis of N-(1,1-dioxido-2,3-dihydrothiophen-3-yl)-2-(2-fluoro-4-(2-methyl-1H-pyrrol-1-yl)phenyl)-N-((2-(1-hydroxycyclobutyl)pyridin-4-yl)methyl)acetamide I-65

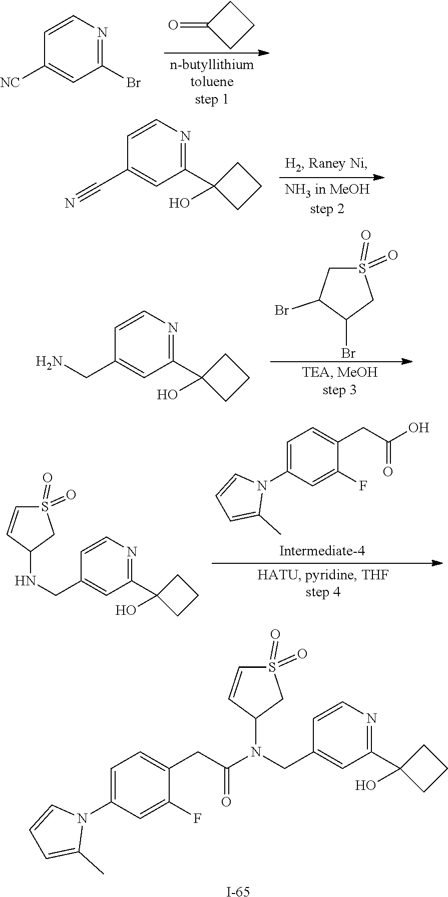

Step 1. Synthesis of 2-(1-hydroxycyclobutyl)isonicotinonitrile: To a solution of 2-bromopyridine-4-carbonitrile (1.00 g, 5.49 mmol, 1.00 eq) in toluene (20.0 mL) was added n-butyllithium (2.8 mL, 7.03 mmol, 1.28 eq, 2.5 M in hexanes) dropwise at −78° C. under N₂ atmosphere. The resulting mixture was stirred for 30 min at −78° C. To the above mixture was added cyclobutanone (769 mg, 10.9 mmol, 2.00 eq) dropwise at −78° C. The resulting mixture was stirred for additional 2 h at −78° C. The reaction was quenched with saturated NH₄Cl solution at 0° C. and diluted with water (200 mL). The mixture was extracted with DCM (3×100 mL). The organic layer was dried over anhydrous Na₂SO₄ and filtered. The filtrate was concentrated under reduced pressure. The residue was purified by Prep-TLC (Eluent of EtOAc/PE) to afford the title compound. LCMS: 175.1 [M+H]⁺.

Step 2. Synthesis of 1-[4-(aminomethyl)pyridin-2-yl]cyclobutan-1-ol: To a solution of 2-(1-hydroxycyclobutyl)pyridine-4-carbonitrile (700 mg, 4.02 mmol, 1.00 eq) in 7 M NH₃ solution in MeOH (100 mL) was added Raney Ni (150 mg, 1.75 mmol, 0.40 eq) at room temperature under N₂ atmosphere. The resulting mixture was degassed with H₂ for three times and stirred for 2 h at room temperature. The resulting mixture was filtered, and the filter cake was washed with MeOH. The filtrate was concentrated under reduced pressure. The crude product was used in the next step directly without further purification. LCMS: 179.0 [M+H]⁺.

Step 3. Synthesis of 3-(((2-(1-hydroxycyclobutyl)pyridin-4-yl)methyl)amino)-2,3-dihydrothiophene 1,1-dioxide: To a stirred mixture of 3,4-dibromotetrahydrothiophene 1,1-dioxide (2.23 g, 8.04 mmol, 2.00 eq) in MeOH (20.0 mL) was added TEA (2.03 g, 20.1 mmol, 5.00 eq) at 0° C. under N₂ atmosphere. The resulting mixture was stirred for 40 min at 0° C. To the above mixture was added 1-[4-(aminomethyl)pyridine-2-yl]cyclobutan-1-ol (716 mg, 4.02 mmol, 1.00 eq) at 0° C. The resulting mixture was stirred for overnight at room temperature. The resulting mixture was concentrated under reduced pressure. The residue was purified by Prep-TLC (Eluent of EtOAc) to afford the title compound. LCMS: 295.2 [M+H]⁺.

Step 4. Synthesis of N-(1,1-dioxido-2,3-dihydrothiophen-3-yl)-2-(2-fluoro-4-(2-methyl-1H-pyrrol-1-yl)phenyl)-N-((2-(1-hydroxycyclobutyl)pyridin-4-yl)methyl)acetamide: To a stirred mixture of 3-(((2-(1-hydroxycyclobutyl)pyridin-4-yl)methyl)amino)-2,3-dihydrothiophene 1,1-dioxide (87 mg, 296 μmol, 1.00 eq) and [2-fluoro-4-(2-methylpyrrol-1-yl)phenyl]acetic acid (Intermediate-4) (83 mg, 355 mol, 1.20 eq) in THF (10.0 mL) were added pyridine (94 mg, 1.18 mmol, 4.00 eq) and HATU (169 mg, 444 μmol, 1.50 eq) at 0° C. under N₂ atmosphere. The resulting mixture was stirred for 2 h at 0° C. and concentrated. The residue was purified by reverse phase HPLC (C18 column, water (10 mmol/L NH₄HCO₃)-ACN) to afford the title compound. LCMS: 510.2 [M+H]⁺. ¹H NMR (400 MHz, DMSO-d₆) δ ppm 8.56-8.45 (m, 1H), 7.51-7.04 (m, 6H), 6.89-6.77 (m, 2H), 6.10-6.09 (m, 1H), 5.98 (s, 1H), 5.90-5.53 (m, 2H), 4.89-4.64 (m, 1.5H), 4.21-3.85 (m, 1.5H), 3.80-3.64 (m, 2H), 3.35-3.28 (m, 1H), 2.54-2.50 (m, 2H), 2.26-2.21 (m, 5H), 1.93-1.76 (m, 2H).

### Synthesis of N-(1,1-dioxido-2,3-dihydrothiophen-3-yl)-2-(6-(4-fluoro-2-hydroxyphenyl)pyridin-3-yl)-N-((2-methoxypyridin-4-yl)methyl)acetamide I-194

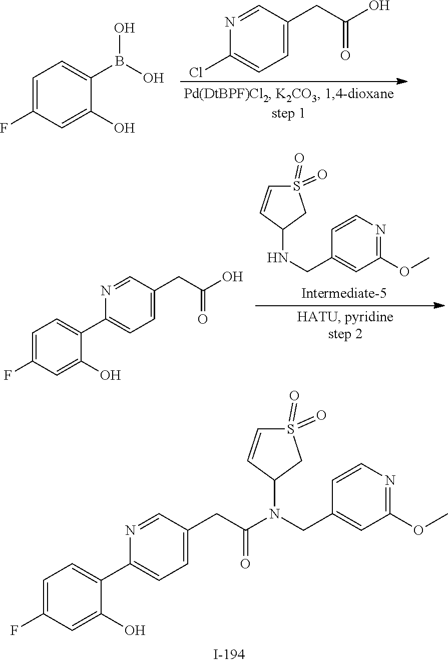 [SMILES: OB(O)c1ccc(F)cc1O ; O=C(O)Cc1ccc(Cl)nc1 ; COc1cc(CNC2C=CS(=O)(=O)C2)ccn1 ; O=C(O)Cc1ccc(-c2ccc(F)cc2O)nc1 ; COc1cc(CN(C(=O)Cc2ccc(-c3ccc(F)cc3O)nc2)C2C=CS(=O)(=O)C2)ccn1]

Step 1. Synthesis of 2-(6-(4-fluoro-2-hydroxyphenyl)pyridin-3-yl)acetic acid: To a solution of 4-fluoro-2-hydroxyphenylboronic acid (301 mg, 1.93 mmol, 1.00 eq) and 2-(6-chloropyridin-3-yl)acetic acid (330 mg, 1.93 mmol, 1.00 eq) in 1,4-dioxane (10.0 mL) were added K₂CO₃ (799 mg, 5.79 mmol, 3.00 eq) and Pd(DtBPF)Cl₂ (125 mg, 193 μmol, 0.10 eq) at room temperature under N₂ atmosphere. The resulting mixture was stirred for 1 h at room temperature under N₂ atmosphere. The mixture was diluted with water (50 mL) and extracted with EtOAc (3×50 mL). The combined organic layers were washed with brine (2×50 mL). The organic layer was dried over anhydrous Na₂SO₄ and filtered. The filtrate was concentrated under reduced pressure. The residue was purified by Prep-TLC (Eluent of EtOAc/PE) to afford the title compound. LCMS: 248.1 [M+H]⁺.

Step 2. Synthesis of N-(1,1-dioxido-2,3-dihydrothiophen-3-yl)-2-(6-(4-fluoro-2-hydroxyphenyl)pyridin-3-yl)-N-((2-methoxypyridin-4-yl)methyl)acetamide: To a solution of 2-(6-(4-fluoro-2-hydroxyphenyl)pyridin-3-yl)acetic acid (40 mg, 162 mol, 1.00 eq) and 3-(((2-methoxypyridin-4-yl)methyl)amino)-2,3-dihydrothiophene 1,1-dioxide (Intermediate-5) (41 mg, 162 μmol, 1.00 eq) in pyridine (2.00 mL) was added HATU (123 mg, 324 μmol, 2.00 eq) at room temperature. The resulting mixture was stirred for 1 h at room temperature and concentrated under reduced pressure. The residue was purified by reverse phase HPLC (C18 column, water (10 mmol/L NH₄HCO₃)-ACN) to afford the title compound. LCMS: 484.3 [M+H]⁺. ¹H NMR (400 MHz, CDCl₃) δ ppm 14.42 (br s, 1H), 8.26-8.23 (m, 2H), 7.83-7.73 (m, 3H), 6.78-6.73 (m, 3H), 6.68-6.52 (m, 3H), 6.05-6.04 (m, 1H), 4.60 (q, 2H), 3.97 (s, 3H), 3.72-3.64 (m, 3H), 3.12-3.08 (m, 1H).

### Synthesis of N1-(-1,1-dioxido-2,3-dihydrothiophen-3-yl)-2-(4′-fluoro-[1,1′-biphenyl]-4-yl)-N1-((2-(2-hydroxypropan-2-yl)pyridin-4-yl)methyl)-N3,N3-dimethylmalonamide—alternative stereoisomers of I-8, third eluting compound as isomer 3)

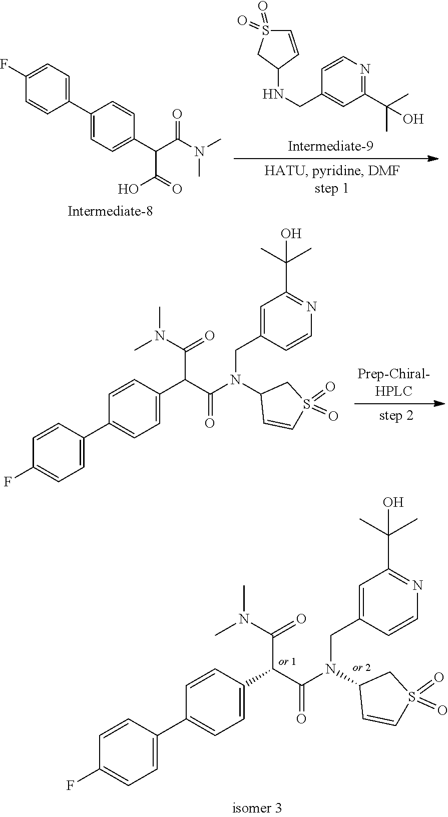 [SMILES: CN(C)C(=O)C(C(=O)O)c1ccc(-c2ccc(F)cc2)cc1 ; CC(C)(O)c1cc(CNC2C=CS(=O)(=O)C2)ccn1 ; CN(C)C(=O)C(C(=O)N(Cc1ccnc(C(C)(C)O)c1)C1C=CS(=O)(=O)C1)c1ccc(-c2ccc(F)cc2)cc1 ; CN(C)C(=O)[C@H](C(=O)N(Cc1ccnc(C(C)(C)O)c1)[C@H]1C=CS(=O)(=O)C1)c1ccc(-c2ccc(F)cc2)cc1]

Step 1. Synthesis of N₁-(1,1-dioxido-2,3-dihydrothiophen-3-yl)-2-(4′-fluoro-[1,1′-biphenyl]-4-yl)-N₁-((2-(2-hydroxypropan-2-yl)pyridin-4-yl)methyl)-N₃,N₃-dimethylmalonamide: To a stirred mixture of 3-(dimethylamino)-2-(4′-fluoro-[1,1′-biphenyl]-4-yl)-3-oxopropanoic acid (Intermediate-8) (150 mg, 498 gmol, 1.00 eq) and 3-(((2-(2-hydroxypropan-2-yl)pyridin-4-yl)methyl)amino)-2,3-dihydrothiophene 1,1-dioxide (Intermediate-9) (352 mg, 1.25 mmol, 2.50 eq) in DMF (8.00 mL) were added pyridine (787 mg, 9.97 mmol, 20.0 eq) and HATU (379 mg, 1.00 mmol, 2.00 eq) at 0° C. After addition, the reaction mixture was allowed to warm to room temperature and stirred for 0.5 h. The residue was purified directly by C18 reverse phase HPLC (water (10 mmol/L NH₄HCO₃)-ACN) to afford the title compound. LCMS: 566.2 [M+H]⁺.

Step 2. Separation of N₁-(1,1-dioxido-2,3-dihydrothiophen-3-yl)-2-(4′-fluoro-[1,1′-biphenyl]-4-yl)-N1-((2-(2-hydroxypropan-2-yl)pyridin-4-yl)methyl)-N₃,N₃-dimethylmalonamide (single isomer, third eluting compound as isomer 3). The N₁-(1,1-dioxido-2,3-dihydrothiophen-3-yl)-2-(4′-fluoro-[1,1′-biphenyl]-4-yl)-N₁-((2-(2-hydroxypropan-2-yl)pyridin-4-yl)methyl)-N₃,N₃-dimethylmalonamide (180 mg) was resolved by Chiral-Prep-HPLC (Column: CHIRAL-Amylose-SC 20×250 mm, 5 μm; Mobile Phase A: EtOH:DCM=1:2, Mobile Phase B: Hex (0.5% 2M NH₃-MeOH); Flow rate: 20 mL/min; Gradient: 60% B to 60% B in 28 min; Wave Length: 220/254 nm. N₁-(1,1-dioxido-2,3-dihydrothiophen-3-yl)-2-(4′-fluoro-[1,1′-biphenyl]-4-yl)-N₁-((2-(2-hydroxypropan-2-yl)pyridin-4-yl)methyl)-N₃,N₃-dimethylmalonamide, single isomer, first eluting compound as isomer 1, retention time: 15.75 minutes. LCMS: 566.2 [M+H]⁺. N₁-(1,1-dioxido-2,3-dihydrothiophen-3-yl)-2-(4′-fluoro-[1,1′-biphenyl]-4-yl)-N₁-((2-(2-hydroxypropan-2-yl)165yridine-4-yl)methyl)-N₃,N₃-dimethylmalonamide, single isomer, second eluting compound as isomer 2, retention time: 18.92 minutes. LCMS: 566.2 [M+H]⁺.

N₁-(1,1-dioxido-2,3-dihydrothiophen-3-yl)-2-(4′-fluoro-[1,1′-biphenyl]-4-yl)-N₁-((2-(2-hydroxypropan-2-yl)pyridin-4-yl)methyl)-N₃,N₃-dimethylmalonamide, single isomer, third eluting compound as isomer 3, retention time: 25.15 minutes. LCMS: 566.2 [M+H]⁺.

The third eluting fractions were combined and concentrated under reduced pressure to afford the product. The product was further purified by reverse Phase HPLC (C18 column, water (0.1% FA)-ACN) to afford the title compound. LCMS: 566.2 [M+H]⁺. ¹H NMR (400 MHz, CD₃OD) δ ppm 8.46-8.37 (m, 1H), 7.67-7.55 (m, 5H), 7.43-7.19 (m, 2H), 7.18-7.14 (m, 3H), 6.86-6.38 (m, 2H), 5.76-5.21 (m, 2H), 4.86-4.37 (m, 2H), 3.64-3.46 (m, 1H), 3.40-3.17 (m, 1H), 3.06-2.19 (m, 6H), 1.55-1.52 (m, 6H).

### Synthesis of 2-(5-(3,4-difluorophenyl)-3-fluoropyridin-2-yl)-N-(1,1-dioxido-2,3-dihydrothiophen-3-yl)-N-((2-(2-hydroxypropan-2-yl)pyridin-4-yl)methyl)acetamide (single isomer, first eluting compound as isomer 1) I-50

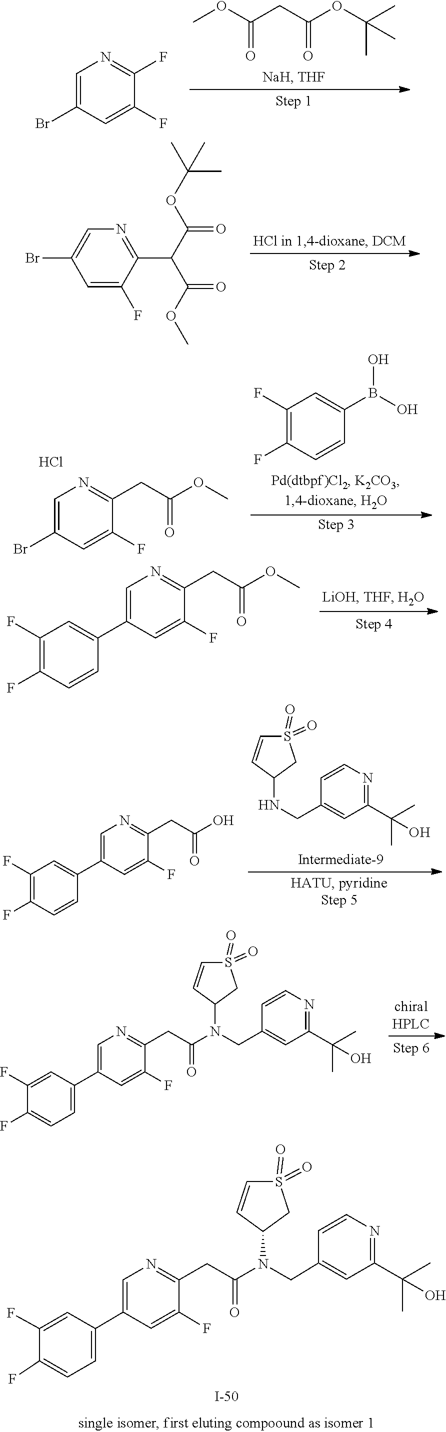

Step 1. Synthesis of 1-(tert-butyl) 3-methyl 2-(5-bromo-3-fluoropyridin-2-yl)malonate: To a solution of 1-tert-butyl 3-methyl propanedioate (900 mg, 5.17 mmol, 1.00 eq) in THF (15.0 mL) was added NaH (414 mg, 10.3 mmol, 2.00 eq, 60% dispersion in mineral oil) in portions over 30 min at 0° C., followed by the addition of 5-bromo-2,3-difluoropyridine (1.00 g, 5.16 mmol, 1.00 eq) at 0° C. The resulting mixture was stirred for overnight at room temperature. This is quenched by the addition of ice/H₂O (30 mL) at 0° C. The resulting mixture was extracted: EtOAc (5×30 mL). The combined organic layers were washed with brine (1×50 mL), dry over Na₂SO₄. After filtration, the filtrate was concentrated under reduced pressure to afford the title compound, which was used in the next step without further purification. LCMS: 320.0 [M+H]⁺.

Step 2. Synthesis of methyl 2-(5-bromo-3-fluoropyridin-2-yl)acetate hydrochloride: To a solution of 1-ethyl 3-methyl 2-(5-bromo-3-fluoropyridin-2-yl)propanedioate (700 mg, 2.19 mmol, 1.00 eq) in DCM (6.00 mL) were added a solution of 4 M HCl solution in 1,4-dioxane (6 mL, 24.0 mmol, 11.0 eq) at 0° C. under N₂ atmosphere. The resulting mixture was stirred for 1 h at 60° C. under N₂ atmosphere. After cooling to room temperature, the resulting mixture was concentrated under vacuum. The residue was used for next step directly without further purification. LCMS: 248.0 [M+H]⁺.

Step 3. Synthesis of methyl 2-(5-(3,4-difluorophenyl)-3-fluoropyridin-2-yl)acetate: To a solution of methyl 2-(5-bromo-3-fluoropyridin-2-yl)acetate hydrochloride (201 mg, 710 μmol, 1.00 eq) and 3,4-difluorophenylboronic acid (112 mg, 710 μmol, 1.00 eq) in 1,4-dioxane (10.0 mL) and H₂O (1.00 mL) was added K₂CO₃ (294 mg, 2.13 mmol, 3.00 eq) and Pd(DtBPF)Cl₂ (46 mg, 71 mol, 0.10 eq) at room temperature under N₂ atmosphere. The mixture was stirred for 1 h at room temperature and concentrated under vacuum. The residue was purified by flash silica gel chromatography (Eluent of EtOAc/PE) to afford the title compound. LCMS: 282.1 [M+H]⁺.

Step 4. Synthesis of 2-(5-(3,4-difluorophenyl)-3-fluoropyridin-2-yl)acetic acid: To a solution of methyl 2-[5-(3,4-difluorophenyl)-3-fluoropyridin-2-yl]acetate (650 mg, 2.31 mmol, 1.00 eq) in THE (10.0 mL) and H₂O (2.00 mL) was added LiOH (166 mg, 6.93 mmol, 3.00 eq) at room temperature. The resulting mixture was stirred for 1 h at room temperature. The mixture was diluted with H₂O and acidified to pH 2~3 with 1 N HCl (aq.). The resulting mixture was extracted with EtOAc (3×50 mL). The combined organic layers were washed with brine (3×50 mL), dried over anhydrous Na₂SO₄. After filtration, the filtrate was concentrated under reduced pressure to afford the title compound. LCMS: 268.1 [M+H]⁺.

Step 5. Synthesis of 2-(5-(3,4-difluorophenyl)-3-fluoropyridin-2-yl)-N-(1,1-dioxido-2,3-dihydrothiophen-3-yl)-N-((2-(2-hydroxypropan-2-yl)pyridin-4-yl)methyl)acetamide: To a solution of [5-(3,4-difluorophenyl)-3-fluoropyridin-2-yl]acetic acid (340 mg, 1.27 mmol, 1.00 eq) and 3-(((2-(2-hydroxypropan-2-yl)pyridin-4-yl)methyl)amino)-2,3-dihydrothiophene 1,1-dioxide (Intermediate-9) (359 mg, 1.27 mmol, 1.00 eq) in pyridine (5.00 mL) was added HATU (967 mg, 2.54 mmol, 2.00 eq) at room temperature. The resulting mixture was stirred for 1 h at room temperature and concentrated under vacuum. The residue was purified by reverse phase Prep-HPLC (C1 8 column, water (0.1% FA)-ACN) to afford the title compound. LCMS: 532.1 [M+H]⁺.

Step 6. Separation of 2-(5-(3,4-difluorophenyl)-3-fluoropyridin-2-yl)-N-(1,1-dioxido-2,3-dihydrothiophen-3-yl)-N-((2-(2-hydroxypropan-2-yl)pyridin-4-yl)methyl)acetamide (single isomer, first eluting compound as isomer 1): The 2-(5-(3,4-difluorophenyl)-3-fluoropyridin-2-yl)-N-(1,1-dioxido-2,3-dihydrothiophen-3-yl)-N-((2-(2-hydroxypropan-2-yl)pyridin-4-yl)methyl)acetamide was separated by Prep chiral-HPLC with the following conditions (Column: CHIRAL ART Cellulose-SC, 20×250 mm, Sum; Mobile Phase A: EtOH-HPLC, Mobile Phase B: MTBE-HPLC; Flow rate: 20 mL/min; Gradient: 80% B to 80% B in 18 min; Wave Length: 220/254 nm). 2-(5-(3,4-difluorophenyl)-3-fluoropyridin-2-yl)-N-(1,1-dioxido-2,3-dihydrothiophen-3-yl)-N-((2-(2-hydroxypropan-2-yl)pyridin-4-yl)methyl)acetamide, single isomer, first eluting compound as isomer 1, retention time: 9.21 minutes. LCMS: 532.1 [M+H]⁺. ¹H NMR (400 MHz, DMSO-d₆) δ ppm 8.74 (d, 1H), 8.44 (d, 1H), 8.14-7.91 (m, 2H), 7.72-7.49 (m, 3H), 7.27-7.06 (m, 2H), 6.82-6.79 (m, 1H), 5.91-5.49 (m, 1H), 5.23 (d, 1H), 4.92-4.10 (m, 3H), 4.01-3.69 (m, 2H), 3.40-3.29 (m, 1H), 1.51-1.36 (m, 6H). 2-(5-(3,4-difluorophenyl)-3-fluoropyridin-2-yl)-N-(1,1-dioxido-2,3-dihydrothiophen-3-yl)-N-((2-(2-hydroxypropan-2-yl)pyridin-4-yl)methyl)acetamide, single isomer, second eluting compound as isomer 2. LCMS: 532.1 [M+H]⁺.

### Synthesis of (R)—N-(1,1-dioxido-2,3-dihydrothiophen-3-yl)-N-((2-(2-hydroxypropan-2-yl)pyridin-4-yl)methyl)-2-(4-(pentafluoro-λ⁶-sulfaneyl)phenyl)acetamide I-16

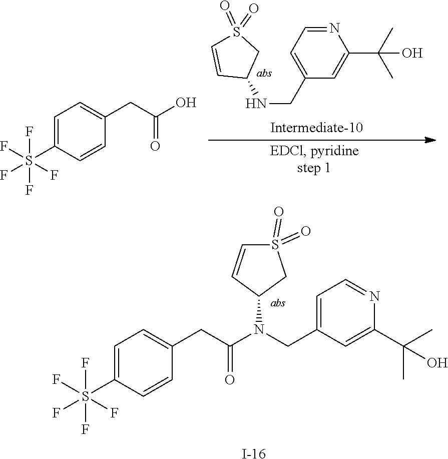

Step 1. Synthesis of (R)—N-(1,1-dioxido-2,3-dihydrothiophen-3-yl)-N-((2-(2-hydroxypropan-2-yl)pyridin-4-yl)methyl)-2-(4-(pentafluoro-λ⁶-sulfaneyl)phenyl)acetamide: To a solution of 2-(4-(pentafluoro-λ⁶-sulfaneyl)phenyl)acetic acid (200 mg, 763 gmol, 1.00 eq) and (R)-3-(((2-(2-hydroxypropan-2-yl)pyridin-4-yl)methyl)amino)-2,3-dihydrothiophene 1,1-dioxide (Intermediate-10) (258 mg, 916 gmol, 1.20 eq) in pyridine (15.0 mL) was added EDCI (292 mg, 1.53 mmol, 2.00 eq) at room temperature under N₂ atmosphere. The resulting mixture was stirred for 1 h at room temperature and concentrated under reduced pressure. The residue was purified by reverse Phase HPLC (C18 column, water (10 mmol/L NH₄HCO₃)-ACN) to afford the title compound. LCMS: 527.1 [M+H]⁺. ¹H NMR (400 MHz, DMSO-d₆) δ ppm 8.45-8.37 (m, 1H), 7.88-7.79 (m, 2H), 7.57-7.38 (m, 3H), 7.25-7.01 (m, 2H), 6.81-6.78 (m, 1H), 5.89-5.45 (m, 1H), 5.24 (d, 1H), 4.84-4.24 (m, 2H), 4.13-4.11 (m, 1H), 3.88-3.80 (m, 1H), 3.73-3.63 (m, 1H), 3.33-3.30 (m, 1H), 1.45-1.41 (m, 6H).

### Synthesis of N-(1,1-dioxido-2,3-dihydrothiophen-3-yl)-2-(2-fluoro-4-(2-methyl-1H-pyrrol-1-yl)phenyl)-N-((7-hydroxy-6,7-dihydro-5H-cyclopenta[b]pyridin-4-yl)methyl)acetamide I-103

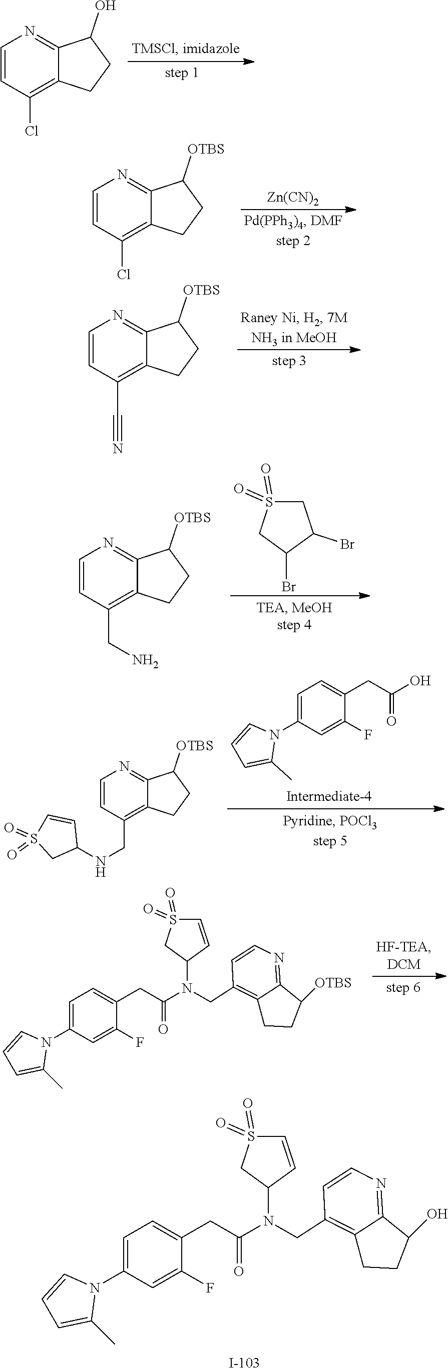

Step 1. Synthesis of 7-((tert-butyldimethylsilyl)oxy)-4-chloro-6,7-dihydro-5H-cyclopenta[b]pyridine: To a solution of 4-chloro-6,7-dihydro-5H-cyclopenta[b]pyridin-7-ol (200 mg, 1.18 mmol, 1.00 eq) and imidazole (322 mg, 4.73 mmol, 4.00 eq) in DCM (5.00 mL) was added TBSCl (713 mg, 4.73 mmol, 4.00 eq) at 0° C. After addition, the resulting mixture was heated to 60° C. and stirred at 60° C. for 4 h. The mixture was cooled to room temperature. The resulting mixture was diluted with H₂O (30 mL), extracted with DCM (3×30 mL). The combined organic layers were washed with brine (2×30 mL), dried over anhydrous Na₂SO₄ and filtered. The filtrate was concentrated under reduced pressure. The residue was purified by flash silica gel chromatography (Eluent of EtOAc/PE) to afford the title compound. LCMS: 284.1 [M+H]⁺.

Step 2. Synthesis of 7-((tert-butyldimethylsilyl)oxy)-6,7-dihydro-5H-cyclopenta[b]pyridine-4-carbonitrile: To a solution of 7-((tert-butyldimethylsilyl)oxy)-4-chloro-6,7-dihydro-5H-cyclopenta[b]pyridine (200 mg, 0.71 mmol, 1.00 eq) and Zn(CN)₂ (246 mg, 2.12 mmol, 3.00 eq) in DMF (6.00 mL) was added Pd(PPh₃)₄ (326 mg, 0.28 mmol, 0.40 eq) under N₂ atmosphere. The resulting mixture was then stirred at 100° C. for 1 h under N₂ atmosphere. The mixture was cooled to room temperature. The resulting mixture was diluted with H₂O (30 mL), extracted with EtOAc (3×30 mL). The combined organic layers were washed with brine (2×30 mL), dried over anhydrous Na₂SO₄ and filtered. The filtrate was concentrated under reduced pressure. The residue was purified by reverse phase HPLC (water (10 mmol/L NH₄HCO₃)-ACN) to afford the title compound. LCMS: 274.2 [M+H]⁺.

Step 3. (7-((tert-butyldimethylsilyl)oxy)-6,7-dihydro-5H-cyclopenta[b]pyridin-4-yl)methanamine: To a solution of 7-((tert-butyldimethylsilyl)oxy)-6,7-dihydro-5H-cyclopenta[b]pyridine-4-carbonitrile (120 mg, 44 μmol, 1.00 eq) in 7M NH₃ in MeOH (6.00 mL) was added Raney Ni (225 mg, 2.63 mmol, 6.00 eq) under N₂ atmosphere. The resulting mixture was degassed with H₂ for three times, then stirred at room temperature for 2 h under H₂ atmosphere. The mixture was filtered, and the filtrate was concentrated under reduced pressure. The residue was dissolved in DCM (20 mL) and filtered. The filtrate was concentrated under reduced pressure. The crude product was used for next step without further purification. LCMS: 279.2 [M+H]⁺.

Step 4. Synthesis of 3-(((7-((tert-butyldimethylsilyl)oxy)-6,7-dihydro-5H-cyclopenta[b]pyridin-4-yl)methyl)amino)-2,3-dihydrothiophene 1,1-dioxide: To a solution of 3,4-dibromotetrahydrothiophene 1,1-dioxide (150 mg, 0.54 mmol, 1.50 eq) in MeOH (3.00 mL) was added TEA (182 mg, 1.80 mmol, 5.00 eq) at 0° C. and then stirred at 0° C. for 0.5 h. To the above mixture was added (7-((tert-butyldimethylsilyl)oxy)-6,7-dihydro-5H-cyclopenta[b]pyridin-4-yl)methanamine (100 mg, 360 μmol, 1.00 eq) at 0° C. After addition, the reaction mixture was allowed to warm to room temperature and stirred for 16 h. The mixture was concentrated and the residue was purified by reverse phase HPLC (water (10 mmol/L NH₄HCO₃)-ACN) to afford the title compound. LCMS: 395.2 [M+H]⁺.

Step 5. Synthesis of N-((7-((tert-butyldimethylsilyl)oxy)-6,7-dihydro-5H-cyclopenta[b]pyridin-4-yl)methyl)-N-(1,1-dioxido-2,3-dihydrothiophen-3-yl)-2-(2-fluoro-4-(2-methyl-1H-pyrrol-1-yl)phenyl)acetamide: To a solution of 3-(((7-((tert-butyldimethylsilyl)oxy)-6,7-dihydro-5H-cyclopenta[b]pyridin-4-yl)methyl)amino)-2,3-dihydrothiophene 1,1-dioxide (30 mg, 76 μmol, 1.00 eq) and 2-(2-fluoro-4-(2-methyl-1H-pyrrol-1-yl)phenyl)acetic acid (Intermediate-4) (21 mg, 91 μmol, 1.20 eq) in pyridine (4.00 mL) was added a solution of POCl₃ (117 mg, 761 μmol, 10.0 eq) in pyridine (1.00 mL) dropwise at 0° C. After addition, the reaction mixture was allowed to warm to room temperature and stirred for 0.5 h. The mixture was concentrated. The residue was purified by reverse phase HPLC (water (10 mmol/L NH₄HCO₃)-ACN) to afford the title compound. LCMS: 610.2 [M+H]⁺.

Step 6. Synthesis of N-(1,1-dioxido-2,3-dihydrothiophen-3-yl)-2-(2-fluoro-4-(2-methyl-1H-pyrrol-1-yl)phenyl)-N-((7-hydroxy-6,7-dihydro-5H-cyclopenta[b]pyridin-4-yl)methyl)acetamide: To a solution of N-((7-((tert-butyldimethylsilyl)oxy)-6,7-dihydro-5H-cyclopenta[b]pyridin-4-yl)methyl)-N-(1,1-dioxido-2,3-dihydrothiophen-3-yl)-2-(2-fluoro-4-(2-methyl-1H-pyrrol-1-yl)phenyl)acetamide (20 mg, 33 μmol, 1.00 eq) in DCM (2.00 mL) was added HF-TEA (106 mg, 657 mol, 20.0 eq) at room temperature. After addition, the resulting mixture was heated to 60° C. and stirred at 60° C. for 1 h. The mixture was cooled to room temperature and concentrated under reduced pressure. The residue was purified by reverse phase HPLC (C18 column, water (0.1% NH₄HCO₃)-ACN) to afford the title compound. LCMS: 496.4 [M+H]⁺. ¹H NMR (400 MHz, DMSO-d₆) δ ppm 8.44-8.33 (m, 1H), 7.25-6.86 (m, 7H), 6.10-6.08 (m, 2H), 5.98-5.57 (m, 1H), 5.41-5.30 (m, 1H), 4.98-4.78 (m, 1H), 4.75-4.53 (m, 1H), 4.26-4.00 (m, 1H), 3.91-3.60 (m, 3H), 2.94-2.73 (m, 1H), 2.72-2.60 (m, 2H), 2.35-2.32 (m, 1H), 2.20-2.10 (m, 3H), 1.87-1.83 (m, 1H).

### Synthesis of N-(1,1-dioxido-2,3-dihydrothiophen-3-yl)-2-(1H-pyrazol-1-yl)-N-(pyridin-4-ylmethyl)-2-(3,3′,4′-trifluoro-[1,1′-biphenyl]-4-yl)acetamide I-48

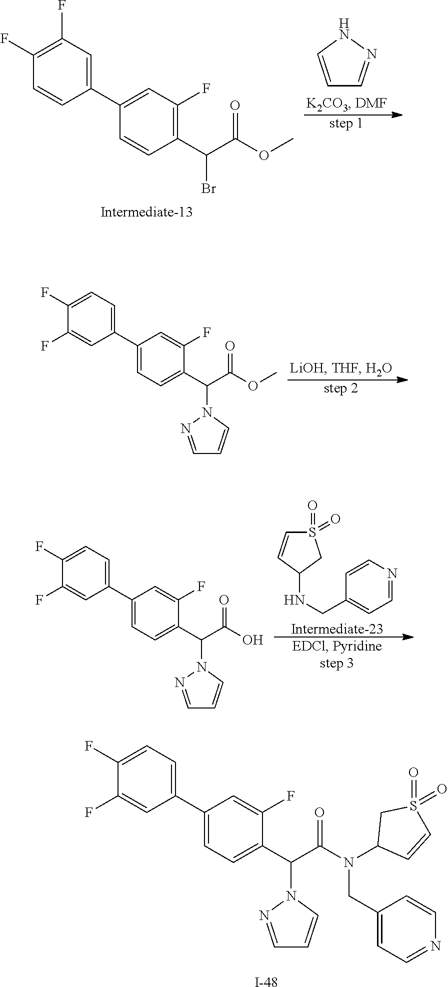

Step 1. Synthesis of methyl 2-(1H-pyrazol-1-yl)-2-(3,3′,4′-trifluoro-[1,1′-biphenyl]-4-yl)acetate: A mixture of 1H-pyrazole (193 mg, 2.84 mmol, 3.00 eq) and K₂CO₃ (393 mg, 2.84 mmol, 3.00 eq) in DMF (8.00 mL) was stirred for 30 min at room temperature. To the above mixture was added a solution of methyl 2-bromo-2-(3,3′,4′-trifluoro-[1,1′-biphenyl]-4-yl)acetate (Intermediate-13) (340 mg, 950 μmol, 1.00 eq) in DMF (2.00 mL) dropwise at 0° C. under N₂ atmosphere. The resulting mixture was stirred for additional 2 h at room temperature. The resulting mixture was diluted with water (50 mL) and extracted with EtOAc (5×50 mL). The organic layer was dried over anhydrous Na₂SO₄ and filtered. The filtrate was concentrated under reduced pressure. The residue was purified by Prep-TLC (Eluent of EtOAc/PE) to afford the title compound. LCMS: 347.1 [M+H]⁺.

Step 2. Synthesis of 2-(1H-pyrazol-1-yl)-2-(3,3′,4′-trifluoro-[1,1′-biphenyl]-4-yl)acetic acid: To a solution of methyl 2-(1H-pyrazol-1-yl)-2-(3,3′,4′-trifluoro-[1,1′-biphenyl]-4-yl)acetate (140 mg, 405 μmol, 1.00 eq) in THF (5.00 mL) and H₂O (1.00 mL) was added LiOH (29 mg, 1.21 mmol, 3.00 eq) at 0° C. The resulting mixture was stirred for 1 h at room temperature. The mixture was diluted with water, acidified to pH 2 with 1 N HCl (aq.) and extracted with EtOAc (3×40 mL). The organic layer was dried over anhydrous Na₂SO₄ and filtered. After filtration, the filtrate was concentrated under reduced pressure to afford the title compound. LCMS: 333.1 [M+H]⁺.

Step 3. Synthesis of N-(1,1-dioxido-2,3-dihydrothiophen-3-yl)-2-(1H-pyrazol-1-yl)-N-(pyridin-4-ylmethyl)-2-(3,3′,4′-trifluoro-[1,1′-biphenyl]-4-yl)acetamide: To a solution of 2-(1H-pyrazol-1-yl)-2-(3,3′,4′-trifluoro-[1,1′-biphenyl]-4-yl)acetic acid (100 mg, 300 mol, 1.00 eq) and 3-((pyridin-4-ylmethyl)amino)-2,3-dihydrothiophene 1,1-dioxide (Intermediate-23) (67 mg, 300 mol, 1.00 eq) in pyridine (3.00 mL) was added EDCI (288 mg, 1.50 mmol, 5.00 eq) at room temperature. The resulting mixture was stirred for 1 h at 60° C. under nitrogen atmosphere. The resulting mixture was cooled to room temperature and concentrated under reduced pressure. The residue was purified by Prep-TLC (Eluent of EtOAc/PE) and further purified by reverse Phase HPLC (C18 column, water (10 mmol/L NH₄HCO₃)-ACN) to afford the title compound. LCMS: 539.1 [M+H]⁺. ¹H NMR (400 MHz, DMSO-d₆) δ ppm 8.51-8.54 (m, 2H), 7.97-7.43 (m, 8H), 7.32-7.09 (m, 3H), 7.07-6.62 (m, 2H), 6.57-6.31 (m, 1H), 5.94-5.21 (m, 1H), 4.72-4.58 (m, 2H), 3.73-2.35 (m, 2H).

### Synthesis of N-(1,1-dioxido-2,3-dihydrothiophen-3-yl)-N-((2-(2-hydroxypropan-2-yl)pyridin-4-yl)methyl)-2-(methylthio)-2-(3,3′,4′-trifluoro-[1,1′-biphenyl]-4-yl)acetamide I-36

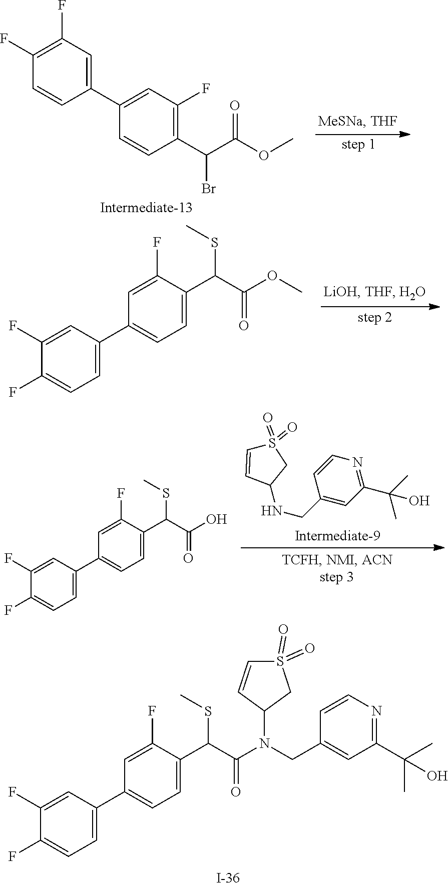

Step 1. Synthesis of methyl 2-(methylsulfanyl)-2-{3,3′,4′-trifluoro-[1,1′-biphenyl]-4-yl}acetate: To a stirred mixture of methyl 2-bromo-2-(3,3′,4′-trifluoro-[1,1′-biphenyl]-4-yl)acetate (Intermediate-13) (320 mg, 891 gmol, 1.00 eq) in THF (5.00 mL) was added MeSNa (312 mg, 4.46 mmol, 5.00 eq) at room temperature under N₂ atmosphere. The mixture was stirred for 1 h at room temperature and diluted with H₂O (50 mL). The resulting mixture was extracted with EtOAc (3×50 mL). The combined organic layers were washed with brine (1×30 mL), dried over anhydrous Na₂SO₄ and filtered. The filtrate was concentrated under reduced pressure. The residue was purified by Prep-TLC (Eluent of EtOAc/PE) to afford the title compound. LCMS: 327.1[M+H]⁺.

Step 2. Synthesis of 2-(methylthio)-2-(3,3′,4′-trifluoro-[1,1′-biphenyl]-4-yl)acetic acid

To a solution of methyl 2-(methylsulfanyl)-2-{3,3′,4′-trifluoro-[1,1′-biphenyl]-4-yl}acetate (147 mg, 450 gmol, 1.00 eq) in THF (5.00 mL) and H₂O (1.00 mL) was added LiOH (32 mg, 1.35 mmol, 3.00 eq) at room temperature. The mixture was stirred for 1 h at room temperature. The mixture was diluted with H₂O (30 mL) and acidified to pH 2~3 with 1 N HCl (aq.). The resulting mixture was extracted with EtOAc (3×30 mL). The combined organic layers were washed with water (1×150 mL), dried over anhydrous Na₂SO₄ and filtered. The filtrate was concentrated under reduced pressure. The crude product was used for next step without further purification. LCMS: 313.0[M+H]⁺.

Step 3. Synthesis of N-(1,1-dioxido-2,3-dihydrothiophen-3-yl)-N-((2-(2-hydroxypropan-2-yl)pyridin-4-yl)methyl)-2-(methylthio)-2-(3,3′,4′-trifluoro-[1,1′-biphenyl]-4-yl)acetamide: To a stirred mixture of (methylsulfanyl)({3,3′,4′-trifluoro-[1,1′-biphenyl]-4-yl})acetic acid (130 mg, 417 μmol, 1.00 eq) and 3-(((2-(2-hydroxypropan-2-yl)pyridin-4-yl)methyl)amino)-2,3-dihydrothiophene 1,1-dioxide (Intermediate-9) (176 mg, 625 μmol, 1.50 eq) in ACN (5.00 mL) were added TCFH (584 mg, 2.08 mmol, 5.00 eq) and NMI (342 mg, 4.17 mmol, 10.0 eq) at 0° C. under N₂ atmosphere. The mixture was stirred for 1 h at room temperature and concentrated. The residue was purified by reverse phase HPLC (C18 column, water (0.1% FA)-ACN) to afford the title compound. LCMS: 577.1 [M+H]⁺. ¹H NMR (400 MHz, CDCl₃) δ ppm 8.40-8.32 (m, 1H), 7.81-7.69 (m, 1H), 7.67-7.50 (m, 5H), 7.45-7.41 (m, 1H), 7.17-7.14 (m, 1H), 6.96-6.97 (m, 1H), 6.83-6.82 (m, 1H), 6.33-5.40 (m, 1H), 5.33-4.98 (m, 2H), 4.82-3.80 (m, 2H), 3.71-3.50 (m, 1H), 3.40-3.08 (m, 1H), 2.17-1.92 (m, 3H), 1.44-1.33 (m, 6H).

### Synthesis of 2-(3,4′-difluoro-[1,1′-biphenyl]-4-yl)-N-(1,1-dioxido-2,3-dihydrothiophen-3-yl)-N-((4-(2-hydroxypropan-2-yl)thiazol-2-yl)methyl)acetamide I-79

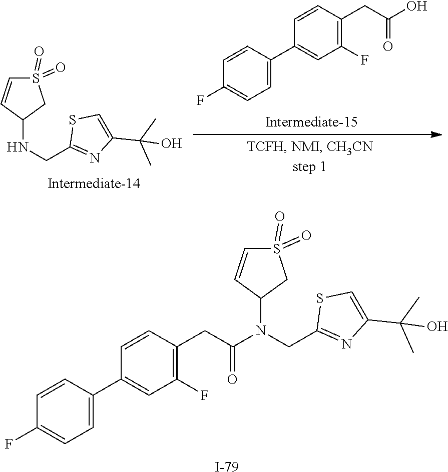

Step 1. Synthesis of 2-(3,4′-difluoro-[1,1′-biphenyl]-4-yl)-N-(1,1-dioxido-2,3-dihydrothiophen-3-yl)-N-((4-(2-hydroxypropan-2-yl)thiazol-2-yl)methyl)acetamide: To a solution of 3-(((4-(2-hydroxypropan-2-yl)thiazol-2-yl)methyl)amino)-2,3-dihydrothiophene 1,1-dioxide (Intermediate-14) (20 mg, 70 gmol, 1.00 eq) and 2-(3,4′-difluoro-[1,1′-biphenyl]-4-yl)acetic acid (Intermediate-15) (17 mg, 70.0 gmol, 1.00 eq) in ACN (2.00 mL) were added NMI (57 mg, 690 gmol, 10.0 eq) and TCFH (97 mg, 350 gmol, 5.00 eq) at 0° C. The mixture was stirred for 1 h at room temperature and concentrated under reduced pressure. The residue was purified by reverse phase HPLC (C18 column, water (0.1% FA)-ACN) to afford the title compound. LCMS: 519.1 [M+H]⁺. ¹H NMR (400 MHz, DMSO-d₆) δ ppm 7.75-7.71 (m, 2H), 7.50-7.12 (m, 7H), 6.94-6.80 (m, 1H), 5.86-5.53 (m, 1H), 5.02 (q, 1H), 4.53 (q, 2H), 4.10-3.88 (m, 2H), 3.65-3.39 (m, 2H), 1.45 (d, 6H).

### Synthesis of (R)—N-(1,1-dioxido-2,3-dihydrothiophen-3-yl)-2,2-difluoro-N-((2-(2-hydroxypropan-2-yl)pyridin-4-yl)methyl)-2-(3,3′,4′-trifluoro-[1,1′-biphenyl]-4-yl)acetamide I-38

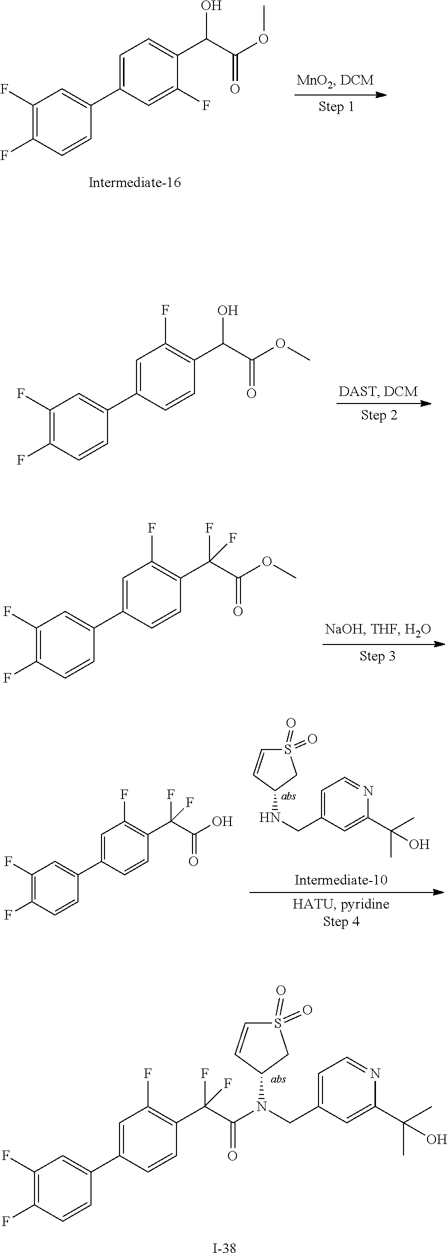

Step 1. Synthesis of methyl 2-oxo-2-(3,3′,4′-trifluoro-[1,1′-biphenyl]-4-yl)acetate: To a solution of methyl 2-hydroxy-2-(3,3′,4′-trifluoro-[1,1′-biphenyl]-4-yl)acetate (Intermediate-16) (300 mg, 1.01 mmol, 1.00 eq) in DCM (15.0 mL) were added MnO₂ (882 mg, 10.1 mmol, 10.0 eq) at room temperature. The mixture was stirred for 1 h at room temperature. The mixture was filtered, and the filtrate was concentrated under reduced pressure. The crude product was used for the next step directly. LCMS: 295.1 [M+H]⁺.

Step 2. Synthesis of methyl 2,2-difluoro-2-(3,3′,4′-trifluoro-[1,1′-biphenyl]-4-yl)acetate: To a solution of methyl 2-oxo-2-{3,3′,4′-trifluoro-[1,1′-biphenyl]-4-yl}acetate (240 mg, 816 μm ol, 1.00 eq) in DCM (8.00 mL) was added DAST (263 mg, 1.63 mmol, 2.00 eq) at room temp. under N₂ atmosphere. The mixture was stirred for 1 h at room temperature. The resulting mixture was concentrated under reduced pressure. The residue was purified by Prep-TLC (Eluent of EtOAc/PE) to afford the title compound.

Step 3. Synthesis of 2,2-difluoro-2-(3,3′,4′-trifluoro-[1,1′-biphenyl]-4-yl)acetic acid: To a solution of methyl 2,2-difluoro-2-(3,3′,4′-trifluoro-[1,1′-biphenyl]-4-yl)acetate (50 mg, 0.16 mmol, 1.00 eq) in H₂O (5.00 mL) and THF (1.00 mL) was added NaOH (8 mg, 0.20 mmol, 1.30 eq) at room temperature. The resulting mixture was stirred for 1 h at room temperature. The mixture was diluted with H₂O and acidified to pH 2~3 with 1 N HCl (aq.) at 0° C., then the mixture was extracted with EtOAc (3×30 mL). The combined organic layers were washed with brine (1×30 mL), dried over anhydrous Na₂SO₄. After filtration, the filtrate was concentrated under reduced pressure. The crude product was used for next step without further purification. LCMS: 301.0 [M−H]⁻.

Step 4. Synthesis of (R)—N-(1,1-dioxido-2,3-dihydrothiophen-3-yl)-2,2-difluoro-N-((2-(2-hydroxypropan-2-yl)pyridin-4-yl)methyl)-2-(3,3′,4′-trifluoro-[1,1′-biphenyl]-4-yl)acetamide: To a solution of 2,2-difluoro-2-(3,3′,4′-trifluoro-[1,1′-biphenyl]-4-yl)acetic acid (40 mg, 130 j mol, 1.00 eq) and (R)-3-(((2-(2-hydroxypropan-2-yl)pyridin-4-yl)methyl)amino)-2,3-dihydrothiophene 1,1-dioxide (Intermediate-10) (41 mg, 140 gmol, 1.10 eq) in pyridine (1.50 mL) was added HATU (101 mg, 260 gmol, 2.00 eq) at room temperature. The resulting mixture was stir red for 1 h at room temperature. The resulting mixture was concentrated under reduced pressure. The residue was purified by reverse Phase HPLC (C18 column, water (10 mmol/L NH₄HCO₃)-ACN) to afford the title compound. LCMS: 567.1 [M+H]⁺. ¹H NMR (400 MHz, CDCl₃) S ppm 8.58-8.51 (m, 1H), 7.72-7.66 (m, 1H), 7.54-7.27 (m, 6H), 7.13 (d, 1H), 6.91 (dd, 1H), 6.69 (d, 1H), 5.98-5.55 (m, 1H), 5.07-4.84 (m, 1H), 4.76-4.45 (m, 2H), 3.70-3.59 (m, 1H) 3.33-3.29 (m, 1H), 1.58 (d, 6H).

### Synthesis of (R)-2-(4-(3,4-difluorophenyl)-1H-pyrazol-1-yl)-N-(1,1-dioxido-2,3-dihydrothiophen-3-yl)-N-((2-(2-hydroxypropan-2-yl)pyridin-4-yl)methyl)acetamide I-6

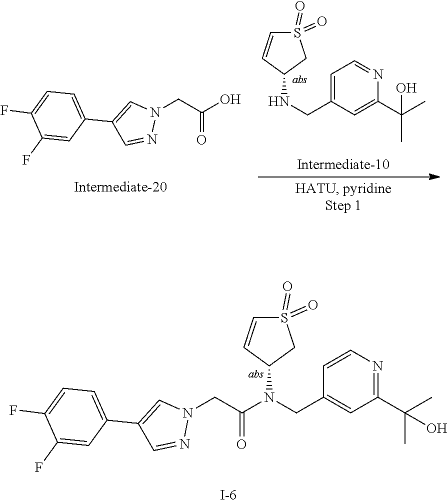

Step 1. Synthesis of (R)-2-(4-(3,4-difluorophenyl)-1H-pyrazol-1-yl)-N-(1,1-dioxido-2,3-dihydrothiophen-3-yl)-N-((2-(2-hydroxypropan-2-yl)pyridin-4-yl)methyl)acetamide: To a solution of [4-(3,4-difluorophenyl)pyrazol-1-yl]acetic acid (Intermediate-20) (60 mg, 252 μmol, 1.00 eq) and (R)-3-(((2-(2-hydroxypropan-2-yl)pyridin-4-yl)methyl)amino)-2,3-dihydrothiophene 1,1-dioxide (Intermediate-10) (71 mg, 252 μmol, 1.00 eq) in pyridine (3.00 mL) was added HATU (192 mg, 504 μmol, 2.00 eq) at room temperature under N₂ atmosphere. The resulting mixture was stirred for 1 h at room temperature. The resulting mixture was concentrated under reduced pressure, and the residue was purified by reverse phase HPLC (C18 column, water (10 mmol/L NH₄HCO₃)-ACN) to afford the title compound. LCMS: 503.1 [M+H]⁺. ¹H NMR (400 MHz, DMSO-d₆) δ ppm 8.46 (dd, 1H), 8.15 (d, 1H), 7.96 (d, 1H), 7.69 (q, 1H), 7.62-7.36 (m, 3H), 7.15-7.04 (m, 2H), 6.80-6.74 (m, 1H), 5.59-5.40 (m, 2H), 5.26 (d, 1H), 5.12 (q, 1H), 4.74 (q, 1H), 4.37 (q, 1H), 3.92-3.68 (m, 1H), 3.35-3.31 (m, 1H), 1.44 (d, 6H).

### Synthesis of N-((4-cyclopropylthiazol-2-yl)methyl)-2-(4-(3,4-difluorophenyl)-1H-pyrazol-1-yl)-N-(1,1-dioxido-2,3-dihydrothiophen-3-yl)acetamide I-5

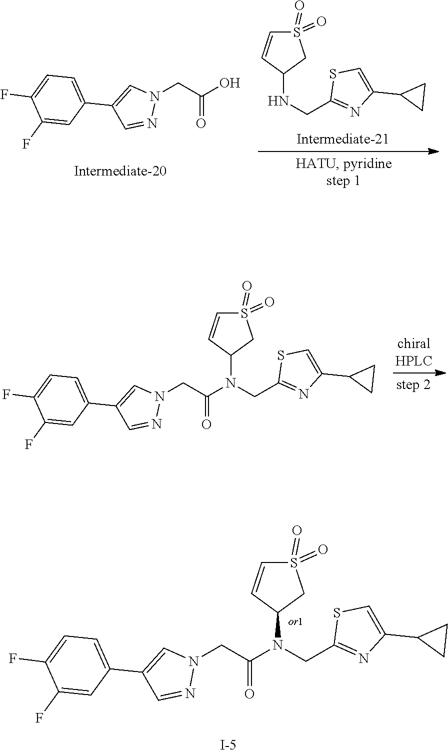

Step 1. Synthesis of N-((4-cyclopropylthiazol-2-yl)methyl)-2-(4-(3,4-difluorophenyl)-1H-pyrazol-1-yl)-N-(1,1-dioxido-2,3-dihydrothiophen-3-yl)acetamide: To a solution of [4-(3,4-difluorophenyl)pyrazol-1-yl]acetic acid (Intermediate-20) (200 mg, 840 μmol, 1.00 eq) and 3-(((4-cyclopropylthiazol-2-yl)methyl)amino)-2,3-dihydrothiophene 1,1-dioxide (Intermediate-21) (227 mg, 840 gmol, 1.00 eq) in pyridine (8.00 mL) was added HATU (639 mg, 1.68 mmol, 2.00 eq) at room temperature under N₂ atmosphere. The resulting mixture was stirred for 1 h at room temperature and concentrated under reduced pressure. The residue was purified by reverse Phase HPLC (C18 column, water (0.1% FA)-ACN) to afford the title compound. LCMS: 491.1 [M+H]⁺.

Step 2. Separation of N-((4-cyclopropylthiazol-2-yl)methyl)-2-(4-(3,4-difluorophenyl)-1H-pyrazol-1-yl)-N-(1,1-dioxido-2,3-dihydrothiophen-3-yl)acetamide (single isomer, first eluting compound as isomer 1). The N-((4-cyclopropylthiazol-2-yl)methyl)-2-(4-(3,4-difluorophenyl)-1H-pyrazol-1-yl)-N-(1,1-dioxido-2,3-dihydrothiophen-3-yl)acetamide was separated by Chiral-HPLC with the following conditions: Column: CHIRALPAK IF, 20×250 mm, 5 μm; Mobile Phase A: EtOH-HPLC, Mobile Phase B: Hex:DCM=1:1-HPLC; Flow rate: 13 mL/min; Gradient: 50% B to 50% B in 18 min, Wave Length: 220/254 nm. N-((4-cyclopropylthiazol-2-yl)methyl)-2-(4-(3,4-difluorophenyl)-1H-pyrazol-1-yl)-N-(1,1-dioxido-2,3-dihydrothiophen-3-yl)acetamide, single isomer, first eluting compound as isomer 1, retention time: 11.98 minutes. LCMS: 491.1 [M+H]⁺. N-((4-cyclopropylthiazol-2-yl)methyl)-2-(4-(3,4-difluorophenyl)-1H-pyrazol-1-yl)-N-(1,1-dioxido-2,3-dihydrothiophen-3-yl)acetamide, single isomer, second eluting compound as isomer 2, retention time: 15.88 minutes. LCMS: 491.1 [M+H]⁺. The first eluting fractions were collected and concentrated under vacuum, the residue was re-dissolved in CH₃CN and H₂O, and then was lyophilized to afford the title compound. 1H NMR (400 MHz, DMSO-d₆) δ ppm 8.11 (d, 1H), 7.95 (d, 1H), 7.72-7.70 (m, 1H), 7.44-7.33 (m, 3H), 7.19-7.17 (m, 1H), 6.89-6.73 (m, 1H), 5.76-5.34 (m, 2H), 5.22 (q, 1H), 4.95 (q, 1H), 4.46 (q, 1H), 3.91-3.33 (m, 2H), 2.15-2.01 (m, 1H), 0.97-0.78 (m, 4H).

### N-(1,1-dioxido-2,3-dihydrothiophen-3-yl)-N-((2-methoxypyridin-4-yl)methyl)-5-(2-methyl-1H-pyrrol-1-yl)-2,3-dihydrobenzofuran-2-carboxamide I-109

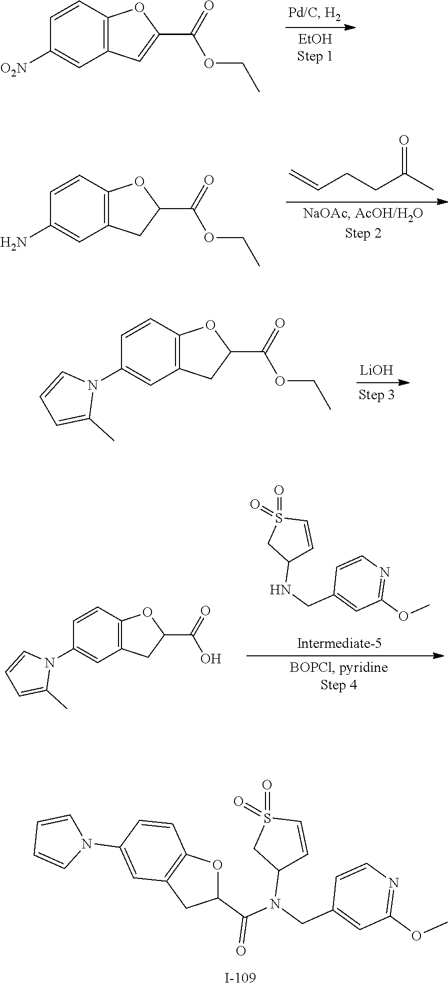

Step 1. Synthesis of ethyl 5-amino-2,3-dihydrobenzofuran-2-carboxylate: To a mixture of ethyl 5-nitrobenzofuran-2-carboxylate (5.00 g, 21.3 mmol, 1.00 eq) and AcOH (1.28 g, 21.3 mmol, 1.00 eq) in EtOH (50.0 mL) was added wet Pd/C (2.00 g, 5%). The mixture was degassed three times with H₂ and stirred at 45° C. for 15 h under H₂ atmosphere (15 psi). The reaction mixture was filtered, and the filtrate was concentrated under vacuum. The residue was purified by flash silica gel chromatography (Eluent of EtOAc/PE) to afford the title compound. LCMS: 208.0 [M+H]⁺.

Step 2. Synthesis of ethyl 5-(2-methyl-1H-pyrrol-1-yl)-2,3-dihydrobenzofuran-2-carboxylate: To a mixture of ethyl 5-amino-2,3-dihydrobenzofuran-2-carboxylate (400 mg, 1.93 mmol, 1.00 eq) and 4-oxopentanal (289 mg, 2.90 mmol, 1.50 eq) in AcOH (1.00 mL) and H₂O (4.00 mL) was added anhydrous NaOAc (158 mg, 1.93 mmol, 1.00 eq) at room temperature. The mixture was stirred at 70° C. for 1 h. To the reaction mixture was added H₂O (5 mL) and EtOAc (5 mL). The organic phase was separated, dried over anhydrous Na₂SO₄, filtered and concentrated under reduced pressure to give a residue. The residue was purified by flash silica gel chromatography (Eluent of EtOAc/PE) to afford the title compound. LCMS: 272.0 [M+H]⁺.

Step 3. Synthesis of 5-(2-methyl-1H-pyrrol-1-yl)-2,3-dihydrobenzofuran-2-carboxylic acid: To a mixture of ethyl 5-(2-methyl-1H-pyrrol-1-yl)-2,3-dihydrobenzofuran-2-carboxylate (87 mg, 320 μmol, 1.00 eq) in MeOH (1.00 mL), THF (1.00 mL) and H₂O (1.00 mL) was added LiOH (40 mg, 0.96 mmol, 3.00 eq). The mixture was stirred at room temperature for 2 h. EtOAc (5 mL) and water (5 mL) were added to the reaction mixture, the organic phase was separated. The aqueous phase was adjusted to pH=4 by 1 N HCl (aq.). The resulting mixture was extracted with EtOAc (3×10 mL). The combined organic layers were washed with brine (1×50 ml), dried over anhydrous Na₂SO₄ and filtered. The filtrate was concentrated under reduced pressure to afford the crude title compound, which was used in the next step without further purification. LCMS: 244.0 [M+H]⁺.

Step 4. Synthesis of N-(1,1-dioxido-2,3-dihydrothiophen-3-yl)-N-((2-methoxypyridin-4-yl)methyl)-5-(2-methyl-1H-pyrrol-1-yl)-2,3-dihydrobenzofuran-2-carboxamide: To a mixture of 5-(2-methyl-1H-pyrrol-1-yl)-2,3-dihydrobenzofuran-2-carboxylic acid (60 mg, 246 μmol, 1.00 eq) and pyridine (117 mg, 1.48 mmol, 6.00 eq) in DCM (3.00 mL) was added 3-(((2-methoxypyridin-4-yl)methyl)amino)-2,3-dihydrothiophene 1,1-dioxide (Intermediate-5) (62 mg, 246 μmol, 1.00 eq) at 0° C. under N₂ atmosphere. The mixture was stirred at 0° C. for 10 min. BOPCl (125 mg, 493 μmol, 2.00 eq) was added at 0° C. and then the reaction was stirred at room temperature for 16 h. The reaction mixture was poured into water (10 mL) and then extracted with EtOAc (3×10 mL). The combined organic phases were washed with brine (3×10 mL), dried over anhydrous Na₂SO₄, filtered and concentrated in vacuum. The residue was purified by reverse phase HPLC (C18 column, water (10 mmol/L NH₄HCO₃)-ACN) to afford the title compound. ¹H NMR (400 MHz, CDCl₃) δ ppm 8.43-7.99 (m, 1H), 7.23-6.99 (m, 2H), 6.91-6.60 (m, 5H), 6.57-6.44 (m, 1H), 6.18 (br d, 1H), 6.02 (br s, 1H), 5.88-5.73 (m, 1H), 5.71-5.26 (m, 1H), 5.13-4.36 (m, 2H), 4.25-4.03 (m, 1H), 4.02-3.93 (m, 3H), 3.71-3.56 (m, 1H), 3.51-3.14 (m, 2H), 2.22-2.13 (m, 3H). LCMS: 480.0 [M+H]⁺.

### Synthesis of N-(1,1-dioxido-2,3-dihydrothiophen-3-yl)-N-((2-(2-hydroxypropan-2-yl)pyridin-4-yl)methyl)-2-methoxy-2-(4-(pentafluoro-λ⁶-sulfaneyl)phenyl)acetamide I-81

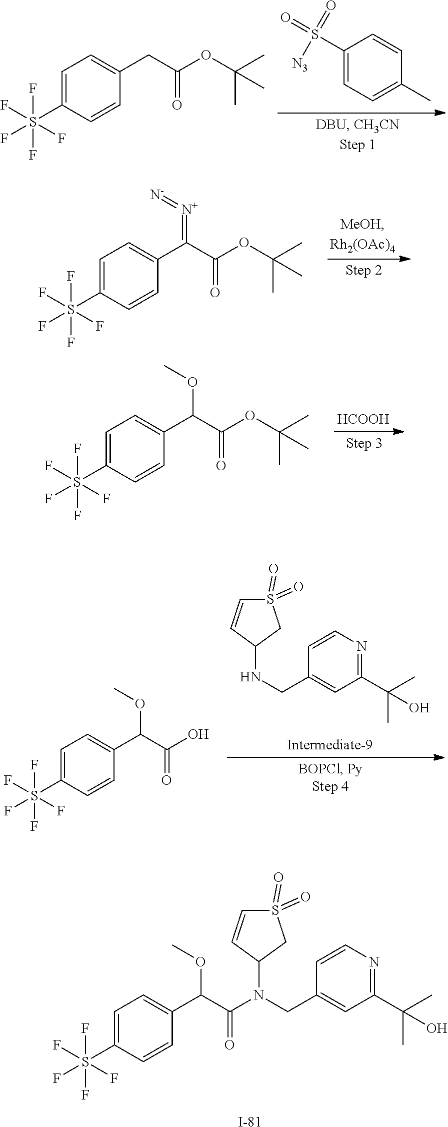

Step 1. Synthesis of tert-butyl 2-diazo-2-(4-(pentafluoro-λ⁶-sulfaneyl)phenyl)acetate: To a solution of tert-butyl 2-(4-(pentafluoro-λ⁶-sulfaneyl)phenyl)acetate (1.00 g, 3.14 mmol, 1.00 eq) and 4-methylbenzenesulfonyl azide (743 mg, 3.77 mmol, 1.20 eq) in ACN (10.0 mL) was added DBU (717 mg, 4.71 mmol, 1.50 eq) at 0° C., then the mixture was stirred at room temperature for 16 h. The reaction mixture was quenched with saturated NH₄Cl (aq.) solution (50 mL), extracted with EtOAc (2×50 mL). The combined organic layers were washed with brine (1×50 mL), dried over anhydrous Na₂SO₄, filtered and the filtrate was concentrated under reduced pressure. The residue was purified by flash silica gel chromatography (Eluent of EtOAc/PE) to afford the title compound. ¹H NMR (400 MHz, CDCl₃) δ ppm 7.73 (d, 2H), 7.57 (d, 2H), 1.57 (s, 9H).

Step-2. Synthesis of tert-butyl 2-methoxy-2-(4-(pentafluoro-λ⁶-sulfaneyl)phenyl)acetate: A mixture of Rh₂(OAc)₄ (68 mg, 0.16 mmol, 0.10 eq) in DCM (5.00 mL) and MeOH (1.00 mL) was cooled to 0° C., then a solution of tert-butyl 2-diazo-2-(4-(pentafluoro-λ⁶-sulfaneyl)phenyl)acetate (537 mg, 1.56 mmol, 1.00 eq) in DCM (2.00 mL) was added dropwise at 0° C. After addition, the resulting mixture was warmed to 10° C. and stirred at 10° C. for 30 min. The reaction was quenched with H₂O (30 mL), extracted with EtOAc (2×30 mL). The combined organic layers were washed with H₂O (30 mL), dried over anhydrous Na₂SO₄, filtered and the filtrate was concentrated under reduced pressure to afford the title compound, which was used in the next step without further purification. ¹H NMR (400 MHz, CDCl₃) δ ppm 7.76 (d, 2H), 7.55 (d, 2H), 4.69 (s, 1H), 3.44 (s, 3H), 1.42 (s, 9H).

Step-3. Synthesis of 2-methoxy-2-(4-(pentafluoro-λ⁶-sulfaneyl)phenyl)acetic acid: A solution of tert-butyl 2-methoxy-2-(4-(pentafluoro-λ⁶-sulfaneyl)phenyl)acetate (688 mg, 1.98 mmol, 1.00 eq) in FA (5.00 mL) was stirred at room temperature for 2 h. The reaction was concentrated under vacuum, then the residue was dissolved in saturated NaHCO₃ solution (50 mL). The resulting mixture was washed with EtOAc (2×30 mL). The aqueous phase was adjusted to pH around 2 with 1 N HCl (aq.), then extracted with EtOAc (2×30 mL). The combined organic layers were dried over anhydrous Na₂SO₄, filtered and the filtrate was concentrated under reduced pressure to afford the title compound, which was used in the next step without further purification. ¹H NMR (400 MHz, CDCl₃) δ ppm 7.79 (d, 2H), 7.58 (d, 2H), 4.86 (s, 1H), 3.48 (s, 3H)

Step 4. Synthesis of N-(1,1-dioxido-2,3-dihydrothiophen-3-yl)-N-((2-(2-hydroxypropan-2-yl)pyridin-4-yl)methyl)-2-methoxy-2-(4-(pentafluoro-λ⁶-sulfaneyl)phenyl)acetamide: To a solution of 2-methoxy-2-(4-(pentafluoro-λ⁶-sulfaneyl)phenyl)acetic acid (100 mg, 342 μmol, 1.00 eq) and 3-(((2-(2-hydroxypropan-2-yl)pyridin-4-yl)methyl)amino)-2,3-dihydrothiophene 1,1-dioxide (Intermediate-9) (96 mg, 342 μmol, 1.00 eq) in DCM (5.00 mL) were added pyridine (216 mg, 2.74 mmol, 8.00 eq), 4 Å molecular sieve (5.00 g) and BOPCl (261 mg, 1.03 mmol, 3.00 eq) at room temperature. The mixture was stirred at room temperature for 1 h. The reaction was diluted with DCM (10 mL) and then filtered, the filtrate was washed with DCM (20 mL), the organic phase was concentrated under reduced pressure. The residue was purified by reverse phase HPLC (water (10 mmol/L NH₄HCO₃)-ACN) to afford the title compound. ¹H NMR (400 MHz, CDCl₃) δ ppm 8.52-8.42 (m, 1H), 7.90-7.71 (m, 2H), 7.66-7.43 (m, 2H), 7.26-7.16 (m, 1H), 7.08-6.81 (m, 1H), 6.70-6.53 (m, 1H), 6.41-6.27 (m, 1H), 5.92-5.58 (m, 1H), 5.38-4.97 (m, 1H), 4.86-4.71 (m, 1H), 4.59-4.21 (m, 1H), 3.77-3.34 (m, 4H), 3.18-2.62 (m, 1H), 1.58-1.49 (m, 6H). LCMS: 557.1 [M+H]⁺.

### Synthesis of N-(1,1-dioxido-2,3-dihydrothiophen-3-yl)-2-(4-(4-fluorophenyl)-2-oxopyridin-1(2H)-yl)-N-(pyridin-4-ylmethyl)acetamide I-147

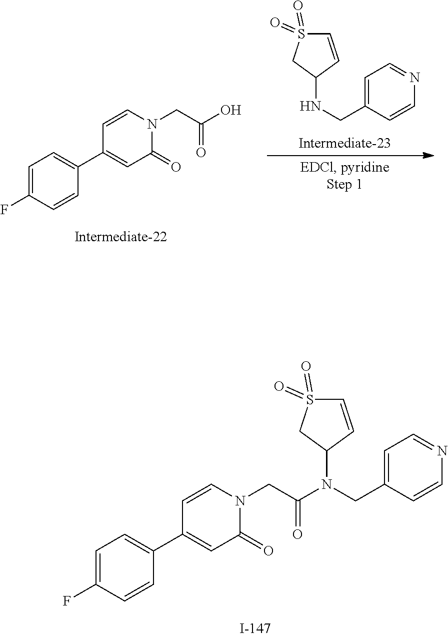 [SMILES: O=C(O)Cn1ccc(-c2ccc(F)cc2)cc1=O ; O=S1(=O)C=CC(NCc2ccncc2)C1 ; O=C(Cn1ccc(-c2ccc(F)cc2)cc1=O)N(Cc1ccncc1)C1C=CS(=O)(=O)C1]

Step 1. Synthesis of N-(1,1-dioxido-2,3-dihydrothiophen-3-yl)-2-(4-(4-fluorophenyl)-2-oxopyridin-1(2H)-yl)-N-(pyridin-4-ylmethyl)acetamide. To a mixture of 2-(4-(4-fluorophenyl)-2-oxopyridin-1(2H)-yl)acetic acid (Intermediate-22) (150 mg, 606 μmol, 1.00 eq) and 3-((pyridin-4-ylmethyl)amino)-2,3-dihydrothiophene 1,1-dioxide (Intermediate-23) (136 mg, 606 mol, 1.00 eq) in pyridine (2.00 mL) was added EDCI (232 mg, 1.21 mmol, 2.00 eq) at room temperature. The mixture was stirred at room temperature for 1 h. The reaction mixture was concentrated under vacuum. The residue was diluted by saturated NH₄Cl (aq.) solution (10 mL) and extracted with EtOAc (3×20 mL). The combined organic layers were washed with brine (1×30 mL), dried over anhydrous Na₂SO₄ and filtered. The filtrate was concentrated under reduced pressure. The residue was purified by reverse phase HPLC (water (10 mmol/L NH₄HCO₃)-ACN) to afford the title compound. ¹H NMR (400 MHz, CDCl₃) δ ppm 8.71 (br d, 2H), 7.57 (br dd, 2H), 7.52-7.30 (m, 3H), 7.17 (t, 2H), 6.85-6.67 (m, 2H), 6.49 (br d, 2H), 5.97 (br d, 1H), 5.01-4.31 (m, 4H), 3.87-3.54 (m, 1H), 3.19 (br dd, 1H). LCMS: 454.1 [M+H]⁺.

### Synthesis of N-(1,1-dioxido-2,3-dihydrothiophen-3-yl)-6-(4-fluorophenyl)-N-((2-methoxypyridin-4-yl)methyl)indoline-3-carboxamide I-53

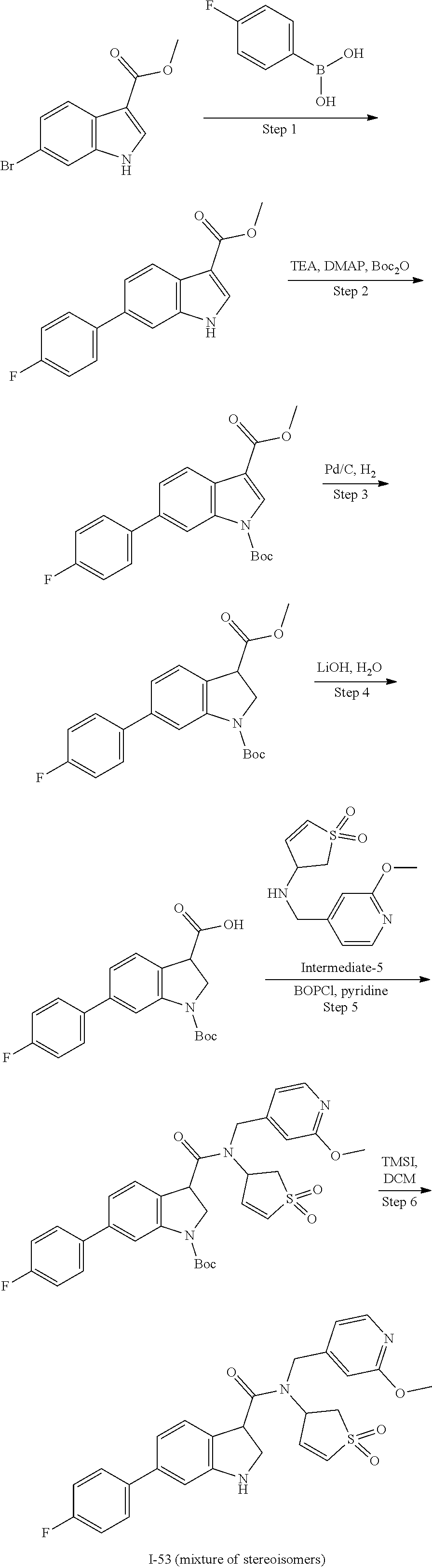

Step 1. Synthesis of methyl 6-(4-fluorophenyl)-1H-indole-3-carboxylate: To a mixture of methyl 6-bromo-1H-indole-3-carboxylate (5.00 g, 19.7 mmol, 1.00 eq) and (4-fluorophenyl)boronic acid (5.51 g, 39.4 mmol, 2.00 eq) in 1,4-dioxane (50.0 mL) and H₂O (5.00 mL) were added KF (3.43 g, 59.0 mmol, 3.00 eq) and Pd(amphos)Cl₂ (1.39 g, 1.97 mmol, 0.10 eq) at room temperature. The mixture was stirred at 40° C. for 15 h under N₂ atmosphere. The reaction mixture was cooled to room temperature and poured into ice-water (50 mL), extracted with EtOAc (3×50 mL), the combined organic layers were dried over anhydrous Na₂SO₄, filtered and concentrated under vacuum. The residue was triturated with PE/EtOAc (5:1) at room temperature for 30 min. The resulting suspension was filtered, the filter cake was collected by filtration and dried in vacuum to afford the title compound, which was used in the next step without further purification. LCMS: 270.0 [M+H]⁺.

Step 2: Synthesis of 1-(tert-butyl) 3-methyl 6-(4-fluorophenyl)-1H-indole-1,3-dicarboxylate: To a mixture of methyl 6-(4-fluorophenyl)-1H-indole-3-carboxylate (5.30 g, 19.7 mmol, 1.00 eq) and (Boc)₂O (4.73 g, 21.6 mmol, 1.10 eq) in DCM (50.0 mL) was added TEA (2.39 g, 23.6 mmol, 1.20 eq) and DMAP (120 mg, 984 μmol, 0.05 eq) at room temperature. The mixture was stirred at room temperature for 1 h. The reaction mixture was poured into ice-water (100 mL) and extracted with EtOAc (3×80 mL). The combined organic layers were washed with brine (1×30 mL), dried over anhydrous Na₂SO₄, filtered and the filtrate was concentrated in vacuum. The residue was purified by flash silica gel chromatography (Eluent of EtOAc/PE) to afford the title compound. LCMS: 370.1 [M+H]⁺.

Step 3: Synthesis of 1-(tert-butyl) 3-methyl 6-(4-fluorophenyl)indoline-1,3-dicarboxylate: To a mixture of 1-(tert-butyl) 3-methyl 6-(4-fluorophenyl)-1H-indole-1,3-dicarboxylate (6.50 g, 17.6 mmol, 1.00 eq) in EtOAc (30.0 mL) and MeOH (40.0 mL) was added dry Pd/C (2.00 g, 5%). The mixture was degassed and purged with H₂ gas (15 psi). Then it was stirred at 30° C. for 15 h. The mixture was filtered, and the filtrate was concentrated under vacuum. The residue was purified by flash silica gel chromatography (Eluent of EtOAc/PE) to afford the title compound. LCMS: 372.1 [M+H]⁺.

Step 4: Synthesis of 1-(tert-butoxycarbonyl)-6-(4-fluorophenyl)indoline-3-carboxylic acid: To a mixture of 1-(tert-butyl) 3-methyl 6-(4-fluorophenyl)indoline-1,3-dicarboxylate (1.40 g, 3.77 mmol, 1.00 eq) in THE (10.0 mL), MeOH (5.00 mL) and H₂O (5.00 mL) was added LiOH*H₂O (316 mg, 7.54 mmol, 2.00 eq) at room temperature. The reaction mixture was stirred at room temperature for 1 h. The reaction mixture was acidified with 1 N HCl (aq.) solution to pH 5 and then extracted with EtOAc (3×10 mL). The combined organic layers were washed with brine (1×20 mL), dried over anhydrous Na₂SO₄, filtered and the filtrate was concentrated in vacuum to afford the title compound, which was used in the next step without further purification. LCMS: 358.1 [M+H]⁺.

Step 5. Synthesis of tert-butyl 3-((1,1-dioxido-2,3-dihydrothiophen-3-yl)((2-methoxypyridin-4-yl)methyl)carbamoyl)-6-(4-fluorophenyl)indoline-1-carboxylate: To a mixture of 1-(tert-butoxycarbonyl)-6-(4-fluorophenyl)indoline-3-carboxylic acid (130 mg, 364 gmol, 1.00 eq) and 3-(((2-methoxypyridin-4-yl)methyl)amino)-2,3-dihydrothiophene 1,1-dioxide (Intermediate-5) (97 mg, 382 gmol, 1.00 eq) in DCM (3.00 mL) was added pyridine (144 mg, 1.82 mmol, 5.00 eq) and BOPCl (185 mg, 728 μmol, 2.00 eq) at 0° C. Then the mixture was stirred at room temperature for 15 h. The mixture was poured into ice-water (10 mL) and extracted with DCM (3×10 mL). The combined organic layers were washed with brine (1×10 mL), dried over anhydrous Na₂SO₄, filtered and the filtrate was concentrated in vacuum. The residue was purified by flash silica gel chromatography (Eluent of EtOAc/PE) to afford the title compound. LCMS: 594.2 [M+H]⁺.

Step 6: Synthesis of N-(1,1-dioxido-2,3-dihydrothiophen-3-yl)-6-(4-fluorophenyl)-N-((2-methoxypyridin-4-yl)methyl)indoline-3-carboxamide: To a mixture of tert-butyl 3-((1,1-dioxido-2,3-dihydrothiophen-3-yl)((2-methoxypyridin-4-yl)methyl)carbamoyl)-6-(4-fluorophenyl)indoline-1-carboxylate (80 mg, 135 gmol, 1.0 eq) in DCM (1.00 mL) was added TMSI (135 mg, 674 gmol, 5.00 eq) at 0° C. under N₂ atmosphere. The mixture was then stirred at room temperature for 1 h. The reaction mixture was diluted with ice-water (10 mL) and extracted with DCM (3×10 mL). The combined organic layers were washed with brine (1×10 mL), dried over anhydrous Na₂SO₄, filtered and the filtrate was concentrated in vacuum. The residue was purified by reverse Phase HPLC (C18 column, water (10 mmol/L NH₄HCO₃)-ACN) to afford the title compound. ¹H NMR (400 MHz, CDCl₃) δ ppm 8.22-8.02 (m, 1H), 7.67-7.42 (m, 3H), 7.21-6.48 (m, 8H), 6.18-5.84 (m, 1H), 5.07-4.46 (m, 2H), 4.35-4.04 (m, 1H), 4.03-3.49 (m, 6H), 3.41-3.07 (m, 1H). LCMS: 494.1 [M+H]⁺.

### Synthesis of N-(1,1-dioxido-2,3-dihydrothiophen-3-yl)-2-(2-fluoro-4-(pentafluoro-λ⁶-sulfaneyl)phenyl)-N-((2-methoxypyridin-4-yl)methyl)acetamide I-22

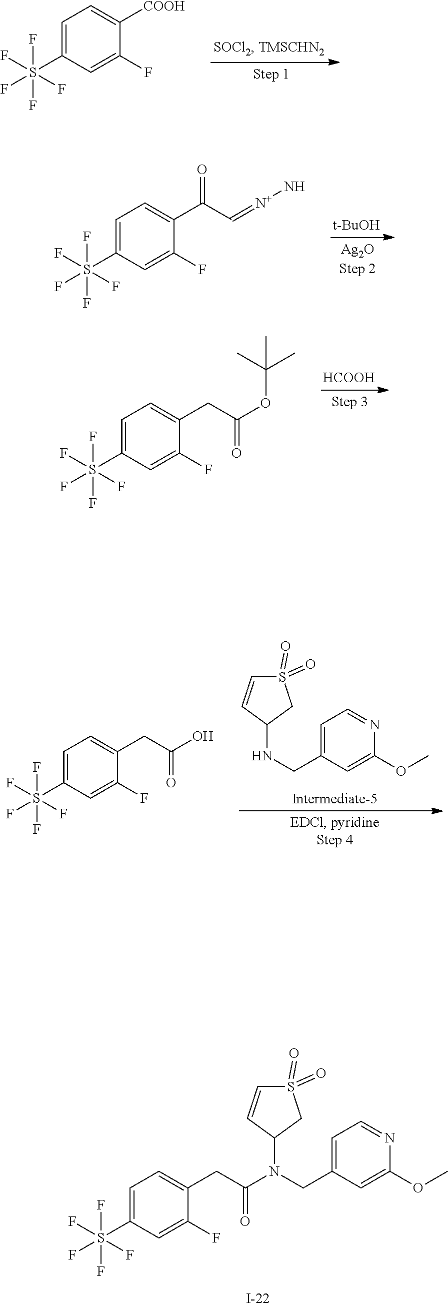

Step 1. Synthesis of 1-(2-(2-fluoro-4-(pentafluoro-λ⁶-sulfaneyl)phenyl)-2-oxoethylidene)diazen-1-ium: To a solution of 2-fluoro-4-(pentafluoro-λ⁶-sulfaneyl)benzoic acid (300 mg, 1.13 mmol, 1.00 eq) in toluene (12.0 mL) was added SOCl₂ (6.70 g, 56.4 mmol, 4.09 mL, 50.0 eq) at room temperature under nitrogen atmosphere. Then the mixture was stirred at 85° C. for 2 h. The reaction mixture was concentrated under vacuum. The residue was re-dissolved in toluene (5.00 mL) and cooled to 0° C. TMSCHN₂ in hexane (1.13 mL, 2.26 mmol, 2.00 eq, 2M) was added to the mixture. After addition, the mixture was stirred at 0° C. for 1 h. The reaction mixture was quenched carefully with 0.5 N HCl (aq.) solution (5 mL), extracted with EtOAc (2×10 mL). The combined organic layers were washed with saturated Na₂CO₃ (aq.) solution (2×10 mL), dried over anhydrous Na₂SO₄ and filtered, the filtrate was concentrated under reduced pressure to afford the title compound, which was used in the next step without further purification. ¹H NMR (400 MHz, CDCl₃) δ ppm 8.16-8.00 (m, 1H), 7.76-7.53 (m, 2H), 4.71 (d, 1H).

Step 2. Synthesis of tert-butyl 2-(2-fluoro-4-(pentafluoro-λ⁶-sulfaneyl)phenyl)acetate: To a solution of 1-(2-(2-fluoro-4-(pentafluoro-λ⁶-sulfaneyl)phenyl)-2-oxoethylidene)diazen-1-ium (50 mg, 172 μmol, 1.00 eq) in t-BuOH (2.00 mL) was added Ag₂O (39 mg, 172 μmol, 1.00 eq) at room temperature. The mixture was stirred at 65° C. for 2 h. The reaction mixture was concentrated under reduced pressure. The residue was diluted with DCM (10 mL) and filtered. The filtrate was concentrated under reduced pressure to afford the title compound, which was used in the next step without further purification. ¹H NMR (400 MHz, CDCl₃) δ ppm 7.56-7.47 (m, 2H), 7.39 (t, 1H), 3.64 (s, 2H), 1.47 (s, 9H).

Step 3. Synthesis of 2-(2-fluoro-4-(pentafluoro-λ⁶-sulfaneyl)phenyl)acetic acid: A solution of tert-butyl 2-(2-fluoro-4-(pentafluoro-λ⁶-sulfaneyl)phenyl)acetate (45 mg, 134 μmol, 1.00 eq) in FA (2.00 mL) was stirred at room temperature for 1 h. The reaction mixture was concentrated under vacuum. The residue was dissolved in saturated NaHCO₃ (aq.) solution (10 mL), washed with EtOAc (2×10 mL). The pH of the aqueous phase was adjusted to 2 with concentrated HCl (aq.) solution and extracted with EtOAc (2×10 mL). The combined organic layers were dried over anhydrous Na₂SO₄ and filtered, the filtrate was concentrated under reduced pressure to afford the title compound, which was used in the next step without further purification. ¹H NMR (400 MHz, CDCl₃) δ ppm 7.58-7.49 (m, 2H), 7.41 (t, 1H), 3.78 (s, 2H)

Step 4. Synthesis of N-(1,1-dioxido-2,3-dihydrothiophen-3-yl)-2-(2-fluoro-4-(pentafluoro-λ⁶-sulfaneyl)phenyl)-N-((2-methoxypyridin-4-yl)methyl)acetamide: To a mixture of 2-(2-fluoro-4-(pentafluoro-λ⁶-sulfaneyl)phenyl)acetic acid (20 mg, 71.0 μmol, 1.00 eq) and 3-(((2-methoxypyridin-4-yl)methyl)amino)-2,3-dihydrothiophene 1,1-dioxide (Intermediate-5) (18 mg, 71.0 μmol, 1.00 eq) in pyridine (2.00 mL) was added EDCI (27 mg, 143 μmol, 2.00 eq) at room temperature. The mixture was stirred at room temperature for 3 h. The reaction mixture was concentrated under reduced pressure. The residue was purified by reverse phase HPLC (water (10 mmol/L NH₄HCO₃)-ACN) to afford the title compound. ¹H NMR (400 MHz, CDCl₃) δ ppm 8.21 (d, 1H), 7.59-7.46 (m, 2H), 7.36 (br t, 1H), 6.78-6.72 (m, 2H), 6.59 (s, 1H), 6.50 (dd, 1H), 6.02-5.94 (m, 1H), 4.65 (d, 1H), 4.55 (d, 1H), 3.97 (s, 3H), 3.76-3.60 (m, 3H), 3.10 (dd, 1H). LCMS: 517.2 [M+H]⁺.

### N1-(1,1-dioxido-2,3-dihydrothiophen-3-yl)-N1-((2-(2-hydroxypropan-2-yl)pyridin-4-yl)methyl)-N3-methyl-2-(3,3′,4′-trifluoro-[1,1′-biphenyl]-4-yl)malonamide I-3 (isomeric mixture)

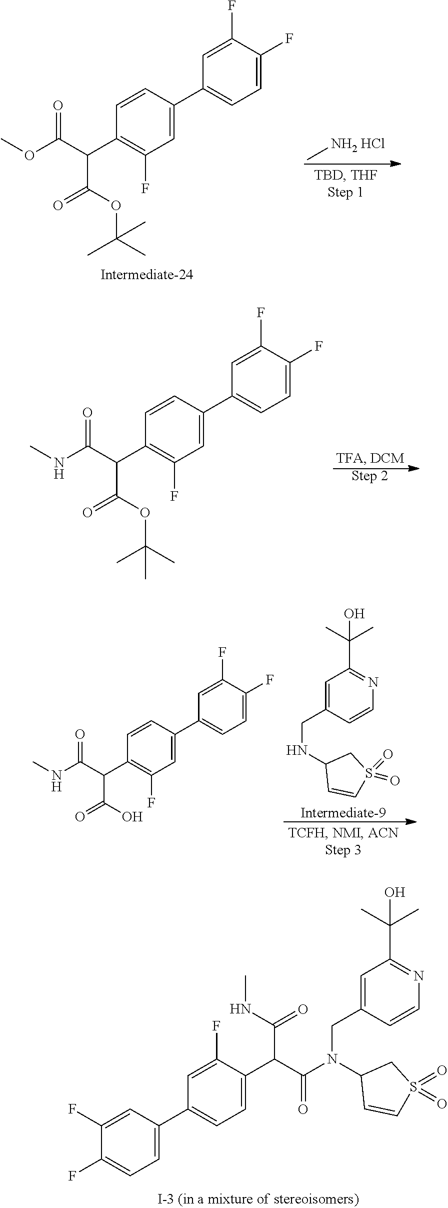

Step 1. Synthesis of tert-butyl 3-(methylamino)-3-oxo-2-(3,3′,4′-trifluoro-[1,1′-biphenyl]-4-yl)propanoate. To a stirred mixture of 1-(tert-butyl) 3-methyl 2-(3,3′,4′-trifluoro-[1,1′-biphenyl]-4-yl)malonate (Intermediate-24) (100 mg, 263 μmol, 1.00 eq) and a 2 M of methylamine solution in THF (5.00 mL, 10.0 mmol, 38.0 eq) was added 1H,2H,3H,4H,6H,7H,8H-[1,3]diazino[1,2-a]pyrimidine (73 mg, 526 μmol, 2.00 eq) at room temperature. The resulting mixture was stirred for 2 h at 60° C. The reaction was cooled to room temperature and concentrated under reduced pressure. The residue was purified by Prep-TLC (Eluent of EtOAc/PE) to afford the title compound. LCMS: 380.1 [M+H]⁺.

Step 2. Synthesis of 3-(methylamino)-3-oxo-2-(3,3′,4′-trifluoro-[1,1′-biphenyl]-4-yl)propanoic acid: To a stirred mixture of tert-butyl 3-(methylamino)-3-oxo-2-(3,3′,4′-trifluoro-[1,1′-biphenyl]-4-yl)propanoate (70 mg, 185 μmol, 1.00 eq) in DCM (14.0 mL) was added TFA (3.50 mL) at 0° C. The resulting mixture was stirred for 40 min at room temperature. The mixture was concentrated under reduced pressure to afford the title compound, which was used in the next step directly without further purification. LCMS: 324.0 [M+H]⁺.

Step 3. Synthesis of N1-(1,1-dioxido-2,3-dihydrothiophen-3-yl)-N1-((2-(2-hydroxypropan-2-yl)pyridin-4-yl)methyl)-N3-methyl-2-(3,3′,4′-trifluoro-[1,1′-biphenyl]-4-yl)malonamide: To a stirred mixture of tert-butyl 3-(methylamino)-3-oxo-2-(3,3′,4′-trifluoro-[1,1′-biphenyl]-4-yl)propanoate (70 mg, 217 mol, 1.00 eq), 3-(((2-(2-hydroxypropan-2-yl)pyridin-4-yl)methyl)amino)-2,3-dihydrothiophene 1,1-dioxide (Intermediate-9) (73 mg, 260 μmol, 1.20 eq) and NMI (178 mg, 2.17 mmol, 10.0 eq) in ACN (10.0 mL) was added TCFH (304 mg, 1.08 mmol, 5.00 eq) at room temperature under N₂ atmosphere. The resulting mixture was stirred for 1 h at room temperature. The resulting mixture was concentrated under reduced pressure. The residue was purified by reverse Phase HPLC (C18 column, water (10 mmol/L NH₄HCO₃)-ACN) to afford the title compound. LCMS: 588.1 [M+H]⁺. ¹H NMR (400 MHz, DMSO-d6) δ ppm 8.43 (m, 1H), 8.21-8.02 (m, 1H), 7.84 (m, 1H), 7.65-7.20 (m, 7H), 7.11 (m, 1H), 6.96-6.56 (m, 1H), 5.70-5.12 (m, 2H), 5.11-4.98 (m, 1H), 4.86-4.65 (m, 1H), 4.64-4.18 (m, 1H), 3.67 (m, 1H), 3.15 (m, 1H), 2.77-2.52 (m, 3H), 1.44 (q, 6H).

### Synthesis of N—((R)-1,1-dioxido-2,3-dihydrothiophen-3-yl)-N-((2-(2-hydroxypropan-2-yl)pyridin-4-yl)methyl)-2-methoxy-2-(3,3′,4′-trifluoro-[1,1′-biphenyl]-4-yl)acetamide 1-2

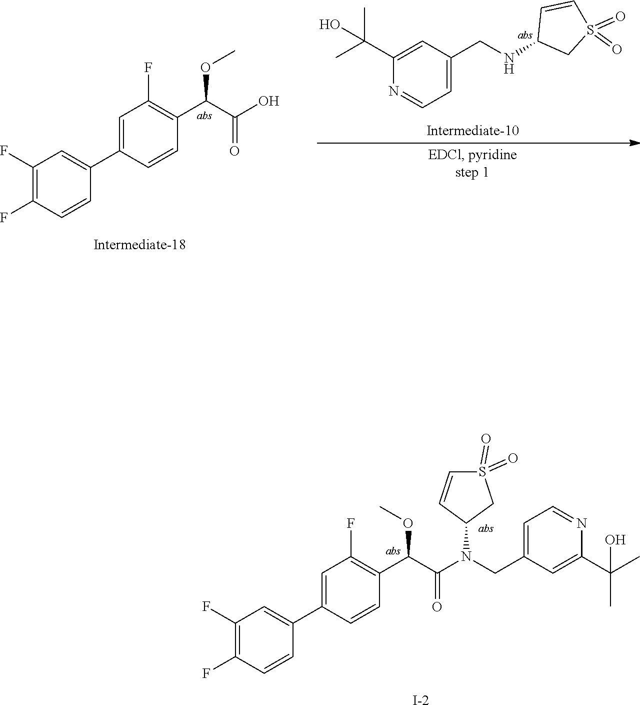

Step 1. Synthesis of N—((R)-1,1-dioxido-2,3-dihydrothiophen-3-yl)-N-((2-(2-hydroxypropan-2-yl)pyridin-4-yl)methyl)-2-methoxy-2-(3,3′,4′-trifluoro-[1,1′-biphenyl]-4-yl)acetamide, single isomer: To a stirred mixture of 2-methoxy-2-(3,3′,4′-trifluoro-[1,1′-biphenyl]-4-yl)acetic acid (single isomer, first eluting compound as isomer 1) (Intermediate-18) (100 mg, 340 gmol, 1.00 eq) and (R)-3-(((2-(2-hydroxypropan-2-yl)pyridin-4-yl)methyl)amino)-2,3-dihydrothiophene 1,1-dioxide (Intermediate-10) (96 mg, 340 μmol, 1.00 eq) in pyridine (5.00 mL) was added EDCI (86 mg, 680 gmol, 2.00 eq) at room temperature. The mixture was stirred for 1 h at room temperature. The resulting mixture was concentrated under vacuum. The residue was purified by reverse phase HPLC (C18 column, water (10 mmol/L NH₄HCO₃)-ACN) to afford the title compound. LCMS: 561.4 [M+H]⁺. ¹H NMR (400 MHz, DMSO-d₆) δ ppm 8.41-8.37 (m, 1H), 7.92-7.88 (m, 1H), 7.71-7.47 (m, 6H), 7.28-7.21 (m, 1H), 7.18-6.93 (m, 1H), 6.82-6.54 (m, 1H), 5.84-5.79 (m, 1H), 5.39-5.17 (m, 2H), 4.66-4.14 (m, 2H), 3.91-3.69 (m, 1H), 3.41-3.20 (m, 4H), 1.41 (s, 6H).

### Synthesis of (R)—N-(1,1-dioxido-2,3-dihydrothiophen-3-yl)-N-((2-(2-hydroxypropan-2-yl)pyridin-4-yl)methyl)-2-(5-(pentafluoro-λ⁶-sulfaneyl)-2H-indazol-2-yl)acetamide I-7

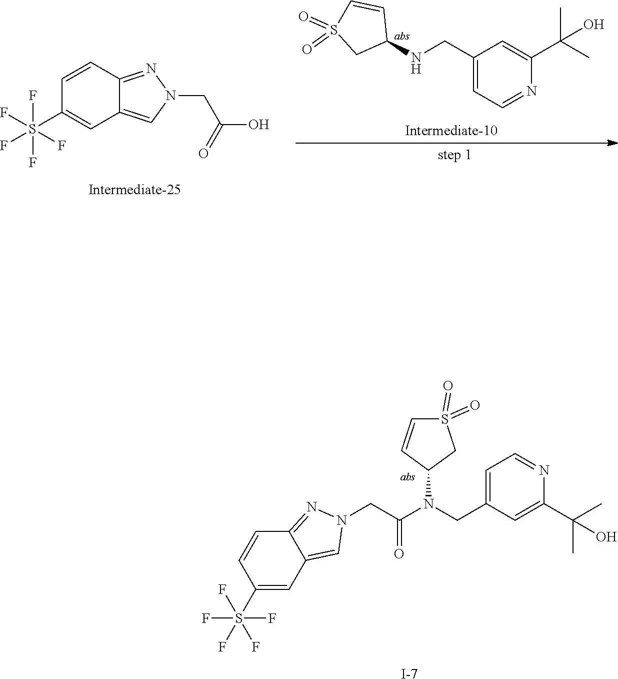

Step 1. Synthesis of (R)—N-(1,1-dioxido-2,3-dihydrothiophen-3-yl)-N-((2-(2-hydroxypropan-2-yl)pyridin-4-yl)methyl)-2-(5-(pentafluoro-λ⁶-sulfaneyl)-2H-indazol-2-yl)acetamide: To a solution of 2-(5-(pentafluoro-λ⁶-sulfaneyl)-2H-indazol-2-yl)acetic acid (Intermediate-25) (30 mg, 99.0 μmol, 1.00 eq) in pyridine (1.00 mL) was added EDCI (38 mg, 199 gmol, 2.00 eq) and (R)-3-(((2-(2-hydroxypropan-2-yl)pyridin-4-yl)methyl)amino)-2,3-dihydrothiophene 1,1-dioxide (Intermediate-10) (28 mg, 99.0 gmol, 1.00 eq) at room temperature. The resulting mixture was stirred at room temperature for 2 h. The reaction mixture was poured into H₂O (10 mL) and then extracted with EtOAc (3×10 mL). The combined organic layers were dried over anhydrous Na₂SO₄, filtered and concentrated under reduced pressure. The residue was purified by reverse phase HPLC (C18 column, water (10 mmol/L NH₄HCO₃)-ACN) to afford the title compound. ¹H NMR (400 MHz, CDCl₃) δ ppm 8.71-8.06 (m, 3H), 7.69 (br s, 3H), 7.19-6.92 (m, 1H), 6.84-6.63 (m, 1H), 6.53-6.25 (m, 1H) 6.15-5.82 (m, 1H), 5.21 (br s, 2H), 5.02-4.62 (m, 2H), 3.61 (br d, 2H), 1.64-1.54 (m, 6H). LCMS: 567.1[M+H]⁺.

### Synthesis of (R)-2-(4-(3,4-difluorophenyl)-2-oxopyridin-1(2H)-yl)-N-(1,1-dioxido-2,3-dihydrothiophen-3-yl)-N-((2-(2-hydroxypropan-2-yl)pyridin-4-yl)methyl)acetamide I-13

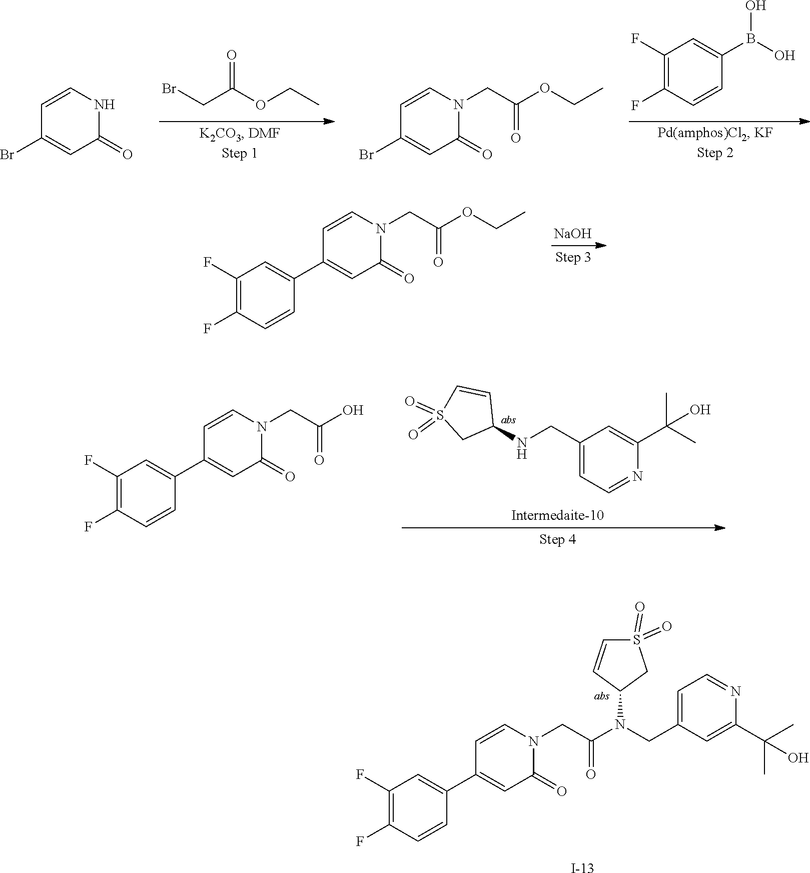

Step 1: Synthesis of ethyl 2-(4-bromo-2-oxopyridin-1(2H)-yl)acetate: To a mixture of 4-bromopyridin-2(1H)-one (2.00 g, 11.5 mmol, 1.00 eq) and ethyl 2-bromoacetate (2.11 g, 12.6 mmol, 1.40 mL, 1.10 eq) in DMF (20.0 mL) was added K₂CO₃ (4.77 g, 34.5 mmol, 3.00 eq) at 30° C. The mixture was stirred at 30° C. for 12 h. The reaction mixture was quenched by saturated NH₄Cl (aq.) solution (100 mL) and extracted with EtOAc (3×50 mL). The combined organic layers were washed with brine (1×50 mL), dried over anhydrous Na₂SO₄, filtered and concentrated. The residue was purified by flash silica gel chromatography (Eluent of EtOAc/PE) to afford the title compound. LCMS: 261.9 [M+H]⁺.

Step 2: Synthesis of ethyl 2-(4-(3,4-difluorophenyl)-2-oxopyridin-1(2H)-yl)acetate: To a mixture of ethyl 2-(4-bromo-2-oxopyridin-1(2H)-yl)acetate (500 mg, 1.92 mmol, 1.00 eq) and (3,4-difluorophenyl)boronic acid (304 mg, 1.92 mmol, 1.00 eq) in dioxane (10.0 mL) and H₂O (1.00 mL) was added Pd(amphos)Cl₂ (136 mg, 192 μmol, 0.10 eq) and KF (335 mg, 5.77 mmol, 3.00 eq) at room temperature under Ar atmosphere, then the reaction was stirred at 50° C. for 12 h. The reaction mixture was quenched by saturated NH₄Cl (aq.) solution (50 mL) and extracted with EtOAc (3×50 mL). The combined organic layers were washed with brine (1×30 mL), dried over anhydrous Na₂SO₄, filtered and concentrated. The residue was purified by flash silica gel chromatography (Eluent of EtOAc/PE) to afford the title compound. LCMS: 294.0 [M+H]⁺.

Step 3: Synthesis of 2-(4-(3,4-difluorophenyl)-2-oxopyridin-1(2H)-yl)acetic acid: To a mixture of ethyl 2-(4-(3,4-difluorophenyl)-2-oxopyridin-1(2H)-yl)acetate (500 mg, 1.70 mmol, 1.00 eq) in H₂O (6.00 mL), MeOH (3.00 mL) and THF (3.00 mL) was added 1 N NaOH (aq.) solution (6.00 mL, 6 mmol, 3.53 eq) at room temperature and the resulting mixture was stirred at room temperature for 1 h. After completion, 0.5 N HCl (aq.) solution (50 mL) was added, and the mixture was extracted with EtOAc (3×50 mL). The combined organic layers were washed with brine (1×30 mL), dried over anhydrous Na₂SO₄, filtered and concentrated to afford the title compound, which was used in the next step directly without further purification. LCMS: 266.0 [M+H]⁺.

Step 4: Synthesis of (R)-2-(4-(3,4-difluorophenyl)-2-oxopyridin-1(2H)-yl)-N-(1,1-dioxido-2,3-dihydrothiophen-3-yl)-N-((2-(2-hydroxypropan-2-yl)pyridin-4-yl)methyl)acetamide: To a mixture of 2-(4-(3,4-difluorophenyl)-2-oxopyridin-1(2H)-yl)acetic acid (50 mg, 188 μmol, 1.00 eq) and (R)-3-(((2-(2-hydroxypropan-2-yl)pyridin-4-yl)methyl)amino)-2,3-dihydrothiophene 1,1-dioxide (Intermediate-10) (53 mg, 188 mol, 1.00 eq) in pyridine (1.00 mL) was added EDCI (72 mg, 377 μmol, 2.00 eq) at room temperature and the mixture was stirred at room temperature for 1 h. The reaction mixture was quenched by saturated NH₄C1 (aq.) (20 mL) and extracted with EtOAc (3×20 mL). The combined organic layers were washed with brine (1×10 mL), dried over anhydrous Na₂SO₄, filtered and concentrated in vacuum. The residue was purified by reverse phase HPLC (C18 column, water (10 mmol/L NH₄HCO₃)-ACN) to afford the title compound. LCMS: 530.3 [M+H]⁺. ¹H NMR (400 MHz, CDCl₃) δ ppm 8.69-8.39 (m, 1H), 7.72-7.50 (m, 1H), 7.49-7.28 (m, 4H), 7.21 (br s, 1H), 6.83-6.68 (m, 2H), 6.68-6.38 (m, 2H), 6.06-5.86 (m, 1H), 5.06-4.32 (m, 5H), 3.86-3.56 (m, 1H), 3.39-3.08 (m, 1H), 1.61 (s, 6H).

The compounds of the disclosure are listed below in Table 2, along with the LCMS method used to isolate them (see the listed “II-#” series of compound below Table 2 also, II-1 to II-5) and their respective retention times and masses.

Compound structures shown throughout the present specification and in the following Table contain designations at certain stereocenters which indicate the following. “or1” or “or2” is intended to cover stereochemically pure compounds wherein the stereochemistry at the stereocenter marked with “or1” or “or2” is either the stereochemistry shown in the diagram or wherein the marked stereocenter has a configuration opposite to what is shown in the diagram. Stereocenters marked with “abs” intend to cover material wherein the marked stereocenter is of the stereochemistry shown in the diagram. Stereocenters marked with “& 1” indicate that the compound material has a mixture of R and S-configured stereoisomers with respect to the marked stereocenter (for example, if the compound only contains one stereocenter and it is marked with “&1,” the material is racemic).

Lengthy table referenced here
US20260297070A1-20261001-T00001
Please refer to the end of the specification for access instructions.

The following list of compounds II-1 to II-5 contains the following information under each structure: II-#; LCMS method-; mass observed; retention time (min).

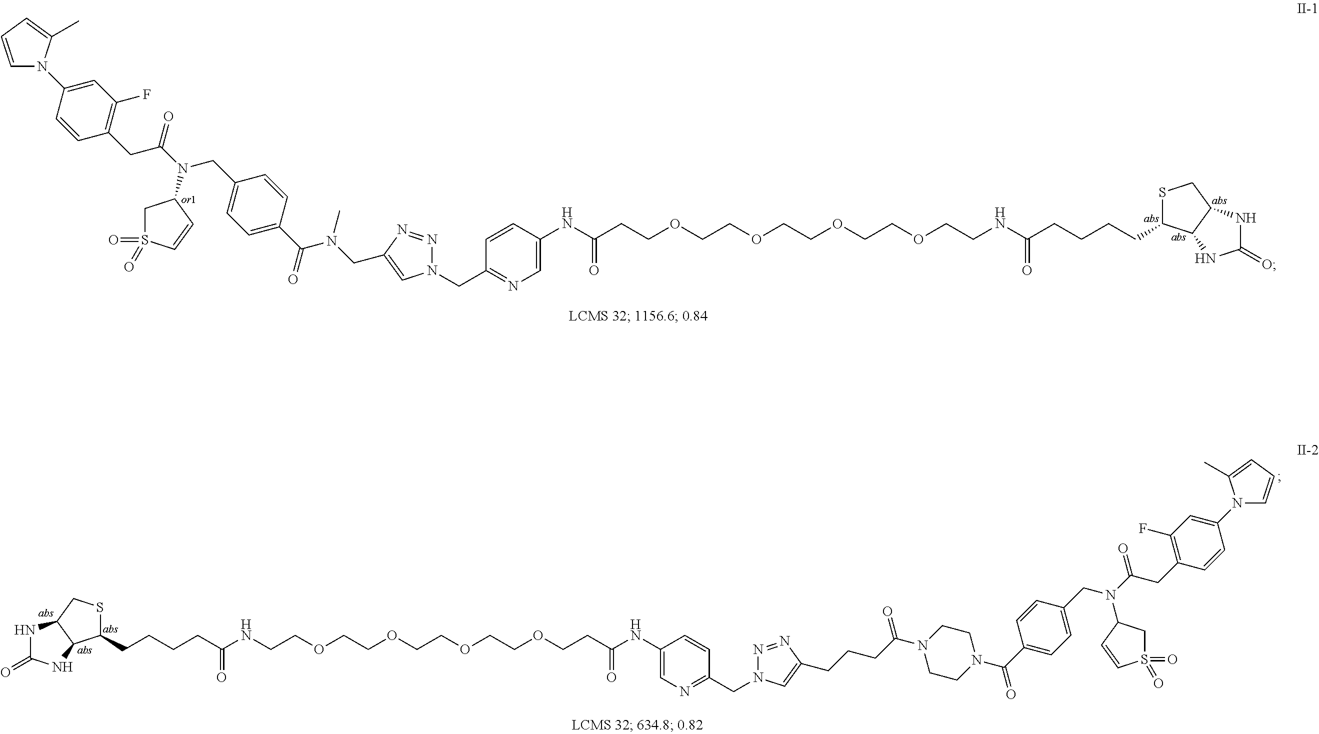 [SMILES: Cc1cccn1-c1ccc(CC(=O)N(Cc2ccc(C(=O)N(C)Cc3cn(Cc4ccc(NC(=O)CCOCCOCCOCCOCCNC(=O)CCCC[C@@H]5SC[C@@H]6NC(=O)N[C@@H]65)cn4)nn3)cc2)[C@@H]2C=CS(=O)(=O)C2)c(F)c1 ; Cc1cccn1-c1ccc(CC(=O)N(Cc2ccc(C(=O)N3CCN(C(=O)CCCc4cn(Cc5ccc(NC(=O)CCOCCOCCOCCOCCNC(=O)CCCC[C@@H]6SC[C@@H]7NC(=O)N[C@@H]76)cn5)nn4)CC3)cc2)C2C=CS(=O)(=O)C2)c(F)c1]

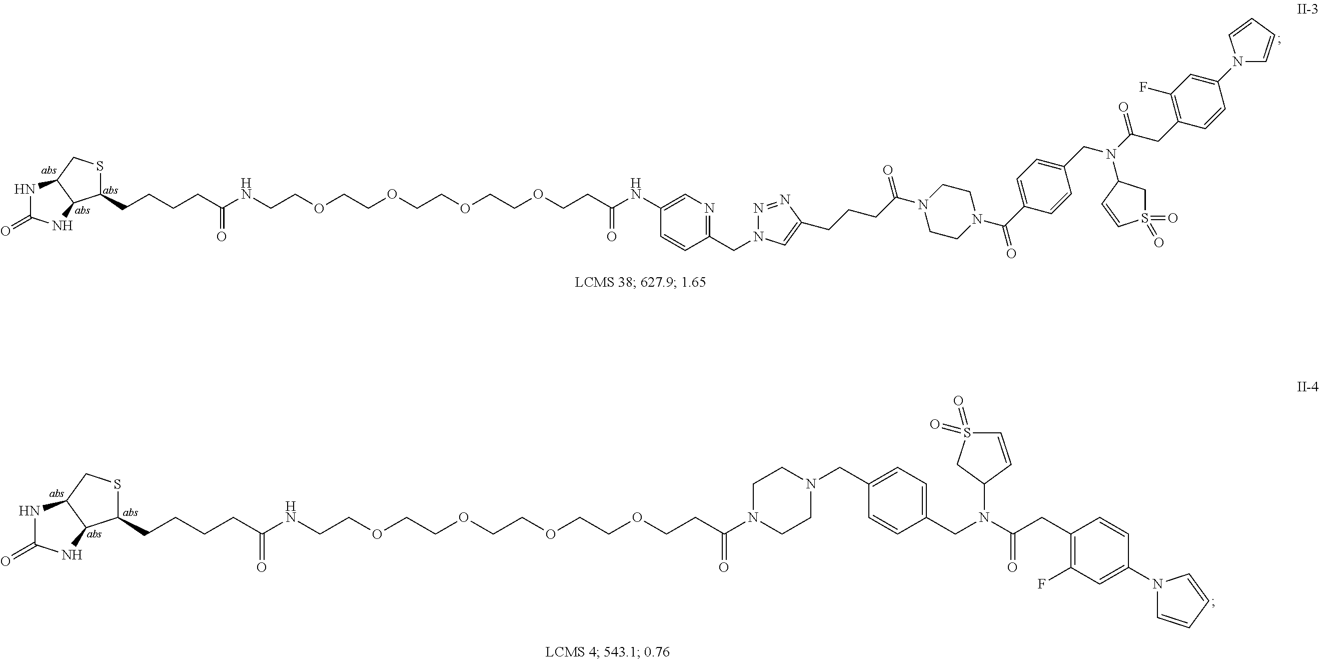 [SMILES: O=C(CCCC[C@@H]1SC[C@@H]2NC(=O)N[C@@H]21)NCCOCCOCCOCCOCCC(=O)Nc1ccc(Cn2cc(CCCC(=O)N3CCN(C(=O)c4ccc(CN(C(=O)Cc5ccc(-n6cccc6)cc5F)C5C=CS(=O)(=O)C5)cc4)CC3)nn2)nc1 ; O=C(CCCC[C@@H]1SC[C@@H]2NC(=O)N[C@@H]21)NCCOCCOCCOCCOCCC(=O)N1CCN(Cc2ccc(CN(C(=O)Cc3ccc(-n4cccc4)cc3F)C3C=CS(=O)(=O)C3)cc2)CC1]

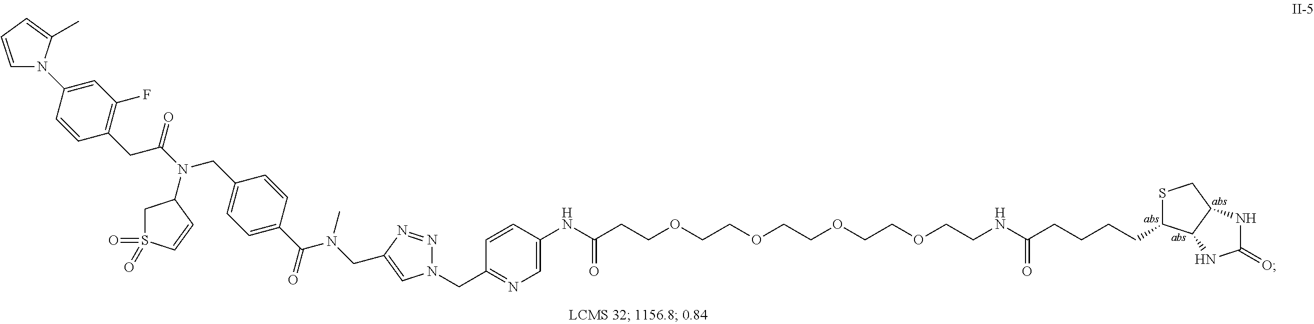 [SMILES: Cc1cccn1-c1ccc(CC(=O)N(Cc2ccc(C(=O)N(C)Cc3cn(Cc4ccc(NC(=O)CCOCCOCCOCCOCCNC(=O)CCCC[C@@H]5SC[C@@H]6NC(=O)N[C@@H]65)cn4)nn3)cc2)C2C=CS(=O)(=O)C2)c(F)c1]

### LCMS Method Conditions

### LCMS 1

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 5% to 60% B in 1.7 min, 60% to 100% B in 0.6 |
| | min, hold 100% |
| Flow Rate (L/min) | 1.2 |
| Column Temperature | 40° C. |
| Column Dimensions | 30 × 2.1 mm, 3.0 μm |

### LCMS 2

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 5% to 100% B in 1.2 min, hold 100% B in |
| | 0.6 min |
| Flow Rate (L/min) | 1.5 |
| Column Temperature | 40° C. |
| Column Dimensions | 30 × 3.0 mm, 2.0 μm |

### LCMS 3

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 5% to 50% B in 1.7 min, 50% to 95% B in 0.6 |
| | min, hold 95% B |
| Flow Rate (L/min) | 1.5 |
| Column Temperature | 40° C. |
| Column Dimensions | 30 × 3.0 mm, 2.0 μm |

### LCMS 4

| | |
|---|---|
| Instrument | Shimadzu LCMS-2020 |
| Stationary Phase | Shim-pack Scepter C18 |
| Mode | Binary Gradient |
| Mobile Phase A | Water/5 mM NH₄HCO₃ |
| Mobile Phase B | ACN |
| Gradient | 10% to 95% B in 1 min, hold 95% B in 0.4 min |
| Flow Rate (L/min) | 1.5 |
| Column Temperature | 40° C. |
| Column Dimensions | 33 × 3.0 mm, 3.0 μm |

### LCMS 5

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 20% to 60% B in 1.7 min, 60% to 95% B in 0.6 |
| | min, hold 95% B |
| Flow Rate (L/min) | 1.5 |
| Column Temperature | 40° C. |
| Column Dimensions | 30 × 3.0 mm, 2.0 μm |

### LCMS 6

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 5% to 60% B in 1.7 min, 60% to 100% B in 0.6 |
| | min, hold 100% |
| Flow Rate (L/min) | 1.5 |
| Column Temperature | 40° C. |
| Column Dimensions | 30 × 3.0 mm, 2.0 μm |

### LCMS 7

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 5% to 50% B in 1.7 min, 50% to 100% B in 0.6 |
| | min, hold 100% |
| Flow Rate (L/min) | 1.2 |
| Column Temperature | 40° C. |
| Column Dimensions | 30 × 2.1 mm, 3.0 μm |

### LCMS 8

| | |
|---|---|
| Instrument | Shimadzu LCMS-2020 |
| Stationary Phase | Shim-pack Scepter C18 |
| Mode | Binary Gradient |
| Mobile Phase A | water/5 mMNH₄HCO₃ |
| Mobile Phase B | ACN |
| Gradient | 20% to 95% B in 1.7 min, hold 95% B in 0.5 min |
| Flow Rate (L/min) | 1.5 |
| Column Temperature | 40° C. |
| Column Dimensions | 33 × 3.0 mm, 3.0 μm |

### LCMS 9

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 5% to 40% B in 1.7 min, 40% to 100% B in 0.6 |
| | min, hold 100% |
| Flow Rate (L/min) | 1.2 |
| Column Temperature | 40° C. |
| Column Dimensions | 30 × 2.1 mm, 3.0 μm |

### LCMS 10

| | |
|---|---|
| Instrument | Shimadzu LCMS-2020 |
| Stationary Phase | HALO C18 |
| Mode | Binary Gradient |
| Mobile Phase A | water/0.1% FA |
| Mobile Phase B | ACN/0.1% FA |
| Gradient | 10% to 60% B in 6.0 min, 60% to 100% B in 2.0 |
| | min, hold |
| Flow Rate (L/min) | 1.5 |
| Column Temperature | 40° C. |
| Column Dimensions | 100 × 2.7 mm |

### LCMS 11

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 5% to 70% B in 1.7 min, 70% to 95% B in 0.6 |
| | min, hold 95% B |
| Flow Rate (L/min) | 1.5 |
| Column Temperature | 40° C. |
| Column Dimensions | 30 × 3.0 mm, 2.0 μm |

### LCMS 12

| | |
|---|---|
| Instrument | Shimadzu LCMS-2020 |
| Stationary Phase | Shim-Pack-C18 |
| Mode | Binary Gradient |
| Mobile Phase A | Water/5 mM NH₄HCO₃ |
| Mobile Phase B | ACN |
| Gradient | 10% to 60% B in 2.2 min, 60% to 90% B in 0.5 |
| | min, hold 95% B |
| Flow Rate (L/min) | 1.5 |
| Column Temperature | 40° C. |
| Column Dimensions | 33 × 3.0 mm, 3.0 μm |

### LCMS 13

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 20% to 60% B in 1.7 min, 60% to 100% B in 0.6 |
| | min, hold |
| Flow Rate (L/min) | 1.2 |
| Column Temperature | 40° C. |
| Column Dimensions | 30 × 2.1 mm, 3.0 μm |

### LCMS 14

| | |
|---|---|
| Instrument | Shimadzu LCMS-2020 |
| Stationary Phase | Shim-pack Scepter C18 |
| Mode | Binary Gradient |
| Mobile Phase A | water/5 mM NH₄HCO₃ |
| Mobile Phase B | ACN |
| Gradient | 10% to 95% B in 1.2 min, hold 95% B in 0.6 min |
| Flow Rate (L/min) | 1.5 |
| Column Temperature | 40° C. |
| Column Dimensions | 33 × 3.0 mm, 3.0 μm |

### LCMS 15

| | |
|---|---|
| Instrument | Shimadzu LCMS-2020 |
| Stationary Phase | Shim-pack Scepter C18 |
| Mode | Binary Gradient |
| Mobile Phase A | water/5 mM NH4HCO3 |
| Mobile Phase B | ACN |
| Gradient | 10% to 40% B in 1.7 min, 40% to 95% |
| | B in 0.6 min, hold 95% B |
| Flow Rate (L/min) | 1.5 |
| Column Temperature | 40° C. |
| Column Dimensions | 33 × 3.0 mm, 3.0 μm |

### LCMS 16

| | |
|---|---|
| Instrument | Agilent HPLC-1290 |
| Stationary Phase | HALO C18 |
| Mode | Binary Gradient |
| Mobile Phase A | H₂O + 0.05% TFA |
| Mobile Phase B | ACN + 0.05% TFA |
| Gradient | 10% to 70% B in 6 min, 70% to 95% B |
| | in 2 min, hold 95% B in |
| Flow Rate (L/min) | 1.5 |
| Column Temperature | 40° C. |
| Column Dimensions | 100 × 4.6 mm, 3.0 μm |

### LCMS 17

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 5% to 50% B in 1.7 min, 50% to 100% |
| | B in 0.6 min, hold 100% |
| Flow Rate (L/min) | 1.2 |
| Column Temperature | 40° C. |
| Column Dimensions | 30 × 2.1 mm, 3.0 μm |

### LCMS 18

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 5% to 100% B in 1.2 min, hold 100% |
| | B in 0.6 min |
| Flow Rate (L/min) | 1.2 |
| Column Temperature | 40° C. |
| Column Dimensions | 30 × 2.1 mm, 3.0 μm |

### LCMS 19

| | |
|---|---|
| Instrument | Shimadzu LCMS-2021 |
| Stationary Phase | HALO C18 |
| Mode | Binary Gradient |
| Mobile Phase A | water/0.05% TFA |
| Mobile Phase B | ACN/0.05% TFA |
| Gradient | 30% to 70% B in 1.7 min, 70% to 100% |
| | B in 0.6 min, hold |
| Flow Rate (L/min) | 1.5 |
| Column Temperature | 40° C. |
| Column Dimensions | 30 × 3.0 mm, 2.0 μm |

### LCMS 20

| | |
|---|---|
| Instrument | Shimadzu LCMS-2020 |
| Stationary Phase | Shim-pack Scepter C18 |
| Mode | Binary Gradient |
| Mobile Phase A | water/5 mM NH₄HCO₃ |
| Mobile Phase B | ACN |
| Gradient | 40% to 60% B in 1.7 min, 60% to 95% |
| | B in 0.6 min, hold 95% B |
| Flow Rate (L/min) | 1.5 |
| Column Temperature | 40° C. |
| Column Dimensions | 33*3.0 mm, 3.0 μm |

### LCMS 21

| | |
|---|---|
| Instrument | Agilent HPLC-1290 |
| Stationary Phase | HALO C18 |
| Mode | Binary Gradient |
| Mobile Phase A | H₂O + 0.05% TFA |
| Mobile Phase B | ACN + 0.05% TFA |
| Gradient | 20% to 60% B in 6 min, 60% to 85% B |
| | in 2 min, hold 85% B in |
| Flow Rate (L/min) | 1.5 |
| Column Temperature | 40° C. |
| Column Dimensions | 100 × 4.6 mm, 2.7 μm |

### LCMS 22

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 20% to 60% B in 1.7 min, 60% to 100% |
| | B in 0.6 min, hold |
| Flow Rate (L/min) | 1.5 |
| Column Temperature | 40° C. |
| Column Dimensions | 30 × 3.0 mm, 2.0 μm |

### LCMS 23

| | |
|---|---|
| Instrument | Shimadzu LCMS-2020 |
| Stationary Phase | HALO C18 3.0 × 30 mm, 5.0 μm |
| Mode | Binary Gradient |
| Mobile Phase A | 0.0375% TFA in water (v/v) |
| Mobile Phase B | 0.01875% TFA in Acetonitrile (v/v) |
| Gradient | 5 to 95% B in 0.5 min, 95% B for 0.3 |
| | min, 95 to 5% B in 0.25 min |
| Flow Rate (L/min) | 1.5 |
| Column Temperature | 50° C. |
| Column Dimensions | 3.0 × 30 mm, 5.0 μm |

### LCMS 24

| | |
|---|---|
| Instrument | Shimadzu LCMS-2020 |
| Stationary Phase | HALO C18 3.0 × 30 mm, 5.0 μm |
| Mode | Binary Gradient |
| Mobile Phase A | 0.0375% TFA in water (v/v) |
| Mobile Phase B | 0.01875% TFA in Acetonitrile (v/v) |
| Gradient | 0 to 60% B in 0.5 min, 60% B for 0.3 |
| | min, 60 to 0% B in 0.25 min |
| Flow Rate (L/min) | 1.5 |
| Column Temperature | 50° C. |
| Column Dimensions | 3.0 × 30 mm, 5.0 μm |

### LCMS 25

| | |
|---|---|
| Instrument | Shimadzu LCMS-2020 |
| Stationary Phase | Kinetex EVO C18 2.1 × 30 mm, 5 μm |
| Mode | Binary Gradient |
| Mobile Phase A | 0.025% NH₃•H₂O in water (v/v) |
| Mobile Phase B | Acetonitrile |
| Gradient | 5 to 95% B in 0.8 min, 95% B for 0.42 |
| | min, 95 to 5% B in 0.01 |
| Flow Rate (L/min) | 1.5 |
| Column Temperature | 40° C. |
| Column Dimensions | 2.1 × 30 mm, 5 μm |

### LCMS 26

| | |
|---|---|
| Instrument | Shimadzu LCMS-2020 |
| Stationary Phase | Kinetex EVO C18 2.1 × 30 mm, 5 um |
| Mode | Binary Gradient |
| Mobile Phase A | 0.025% NH₃•H₂O in water (v/v) |
| Mobile Phase B | Acetonitrile |
| Gradient | 0 to 60% B in 0.8 min, 60% B for 0.42 |
| | min, 60 to 0% B in 0.01 |
| Flow Rate (L/min) | 1.5 |
| Column Temperature | 40° C. |
| Column Dimensions | 2.1 × 30 mm, 5 μm |

### LCMS 27

| | |
|---|---|
| Instrument | Agilent 1100/1200/1290/6100 |
| | Series/Alltech 2000ES/3300 |
| Stationary Phase | Agilent Poroshell 120 SB-C18 4.6 × 30 mm 2.7 μm |
| Mode | Binary Gradient |
| Mobile Phase A | water (0.1% formic acid) |
| Mobile Phase B | acetonitrile (0.1% formic acid) |
| Gradient | 0.01 min -1% B, 1.5 min - 100% B, 1.73 min - 100% B |
| Flow Rate (L/min) | 3 |
| Column Temperature | 60° C. |
| Column Dimensions | 4.6 × 30 mm 2.7 μm |

### LCMS 28

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 5% to 100% B in 1.2 min, hold 100% |
| | B in 0.6 min |
| Flow Rate (L/min) | 1.5 |
| Column Temperature | 40° C. |
| Column Dimensions | 30 × 3.0 mm, 2.0 μm |

### LCMS 29

| | |
|---|---|
| Instrument | Shimadzu LCMS-2020 |
| Stationary Phase | Shim-pack Scepter C18 |
| Mode | Binary Gradient |
| Mobile Phase A | water/5 mMNH₄HCO₃ |
| Mobile Phase B | ACN |
| Gradient | 30% to 70% B in 1.7 min, 70% to 95% B in 0.6 |
| | min, hold 95% B |
| Flow Rate (L/min) | 1.5 |
| Column Temperature | 40° C. |
| Column Dimensions | 33 × 3.0 mm, 3.0 μm |

### LCMS 30

| | |
|---|---|
| Instrument | Shimadzu LCMS-2020 |
| Stationary Phase | Shim-pack Scepter C18 |
| Mode | Binary Gradient |
| Mobile Phase A | water/5 mM NH₄HCO₃ |
| Mobile Phase B | ACN |
| Gradient | 10% to 95% B in 1.2 min, hold 95% B in 0.6 min |
| Flow Rate (L/min) | 1.5 |
| Column Temperature | 40° C. |
| Column Dimensions | 33 × 3.0 mm, 3.0 μm |

### LCMS 31

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 5% to 100% B in 1.2 min, hold 100% B in 0.6 |
| | min |
| Flow Rate (L/min) | 1.2 |
| Column Temperature | 40° C. |
| Column Dimensions | 30 × 2.1 mm, 3.0 μm |

### LCMS 32

| | |
|---|---|
| Instrument | Shimadzu LCMS-2020 |
| Stationary Phase | Shim-pack Scepter C18 |
| Mode | Binary Gradient |
| Mobile Phase A | water/5 mM NH₄HCO₃ |
| Mobile Phase B | ACN |
| Gradient | 10% to 95% B in 1.2 min, hold 95% B in 0.6 min |
| Flow Rate (L/min) | 1.5 |
| Column Temperature | 40° C. |
| Column Dimensions | 33 × 3.0 mm, 3.0 μm |

### LCMS 33

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 5% to 60% B in 1.7 min, 60% to 100% B in 0.6 |
| | min, hold 100% |
| Flow Rate (L/min) | 1.2 |
| Column Temperature | 40° C. |
| Column Dimensions | 30 × 2.1 mm, 3.0 μm |

### LCMS 34

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 40% to 70% B in 1.7 min, 70% to 100% B in 0.6 |
| | min, hold |
| Flow Rate (L/min) | 1.5 |
| Column Temperature | 40° C. |
| Column Dimensions | 30 × 3.0 mm, 2.0 μm |

### LCMS 35

| | |
|---|---|
| Instrument | Shimadzu LCMS-2020 |
| Stationary Phase | Shim-pack Scepter C18 |
| Mode | Binary Gradient |
| Mobile Phase A | water/5 mMNH₄HCO₃ |
| Mobile Phase B | ACN |
| Gradient | 40% to 95% B in 2.3 min, hold 95% B in 0.5 min |
| Flow Rate (L/min) | 1.5 |
| Column Temperature | 40° C. |
| Column Dimensions | 33 × 3.0 mm, 3.0 μm |

### LCMS 36

| | |
|---|---|
| Instrument | Shimadzu LCMS-2020 |
| Stationary Phase | HALO C18 |
| Mode | Binary Gradient |
| Mobile Phase A | water/0.05% TFA |
| Mobile Phase B | ACN/0.05% TFA |
| Gradient | 5% to 60% B in 1.7 min, 60% to 100% B in 0.6 |
| | min, hold 100% |
| Flow Rate (L/min) | 1.5 |
| Column Temperature | 40° C. |
| Column Dimensions | 30 × 3.0 mm, 2.0 μm |

### LCMS 37

| | |
|---|---|
| Instrument | Shimadzu LCMS-2020 |
| Stationary Phase | Shim-pack Scepter C18 |
| Mode | Binary Gradient |
| Mobile Phase A | water/5 mMNH₄HCO₃ |
| Mobile Phase B | ACN |
| Gradient | 10% to 60% B in 1.7 min, 60% to 95% B in 0.6 |
| | min, hold 95% B |
| Flow Rate (L/min) | 1.5 |
| Column Temperature | 40° C. |
| Column Dimensions | 33 × 3.0 mm, 3.0 μm |

### LCMS 38

| | |
|---|---|
| Instrument | Shimadzu LCMS-2020 |
| Stationary Phase | Shim-pack Scepter C18 |
| Mode | Binary Gradient |
| Mobile Phase A | water/5 mMNH₄HCO₃ |
| Mobile Phase B | ACN |
| Gradient | 10% to 50% B in 1.7 min, 50% to 95% B in 0.6 |
| | min, hold 95% B |
| Flow Rate (L/min) | 1.5 |
| Column Temperature | 40° C. |
| Column Dimensions | 33 × 3.0 mm, 3.0 μm |

### LCMS 39

| | |
|---|---|
| Instrument | Shimadzu LCMS-2020 |
| Stationary Phase | Luna Omega PS C18 |
| Mode | Binary Gradient |
| Mobile Phase A | water/0.1% FA |
| Mobile Phase B | ACN/0.1% FA |
| Gradient | 5% to 60% B in 1.7 min, 60% to 100% B in 0.6 |
| | min, hold 100% |
| Flow Rate (L/min) | 1.2 |
| Column Temperature | 40° C. |
| Column Dimensions | 30 × 2.1 mm, 3.0 μm |

### LCMS 40

| | |
|---|---|
| Instrument | Shimadzu LCMS-2020 |
| Stationary Phase | Shim-pack Scepter C18 |
| Mode | Binary Gradient |
| Mobile Phase A | water/5 mM NH₄HCO₃ |
| Mobile Phase B | ACN |
| Gradient | 30% to 70% B in 1.7 min, 70% to 95% B in 0.6 |
| | min, hold 95% B |
| Flow Rate (L/min) | 1.5 |
| Column Temperature | 40° C. |
| Column Dimensions | 33 × 3.0 mm, 3.0 μm |

### LCMS 41

| | |
|---|---|
| Instrument | Shimadzu LCMS-2020 |
| Stationary Phase | Shim-pack Scepter C18 |
| Mode | Binary Gradient |
| Mobile Phase A | Water/5 mM NH₄HCO₃ |
| Mobile Phase B | ACN |
| Gradient | 20% to 60% B in 1.7 min, 60% to 95% B in 0.6 |
| | min, hold 95% B |
| Flow Rate (L/min) | 1.5 |
| Column Temperature | 40° C. |
| Column Dimensions | 33 × 3.0 mm, 3.0 μm |

### LCMS 42

| | |
|---|---|
| Instrument | Shimadzu LCMS-2020 |
| Stationary Phase | Shim-pack Scepter C18 |
| Mode | Binary Gradient |
| Mobile Phase A | Water/5 mM NH₄HCO₄ |
| Mobile Phase B | ACN |
| Gradient | 30% to 60% B in 1.7 min, 60% to 95% B in 0.6 |
| | min, hold 95% B |
| Flow Rate (L/min) | 1.5 |
| Column Temperature | 40° C. |
| Column Dimensions | 33 × 3.0 mm, 3.0 μm |

### Intermediates

### Intermediate-1: 3-(benzylamino)-2,3-dihydrothiophene 1,1-dioxide

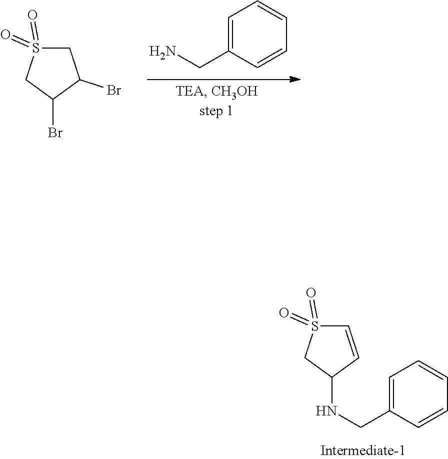 [SMILES: O=S1(=O)CC(Br)C(Br)C1 ; NCc1ccccc1 ; O=S1(=O)C=CC(NCc2ccccc2)C1]

Step 1. Synthesis of 3-(benzylamino)-2,3-dihydrothiophene 1,1-dioxide: A solution of 3,4-dibromotetrahydrothiophene 1,1-dioxide (4.00 g, 14.4 mmol, 1.00 eq) and TEA (4.37 g, 43.2 mmol, 3.00 eq) in MeOH (30.0 mL) was stirred for 40 min at 0° C. To the above mixture was added phenylmethanamine (2.31 g, 21.6 mmol, 1.50 eq) dropwise at 0° C. After addition, the reaction mixture was allowed to warm to room temperature and stirred for 2 h. The mixture was concentrated, and the residue was purified by flash silica gel chromatography (Eluent of DCM/MeOH) to afford the title compound. LCMS: 224.1[M+H]⁺.

### Intermediate-2: Synthesis of 3-(benzylamino)-5-bromo-2,3-dihydrothiophene 1,1-dioxide

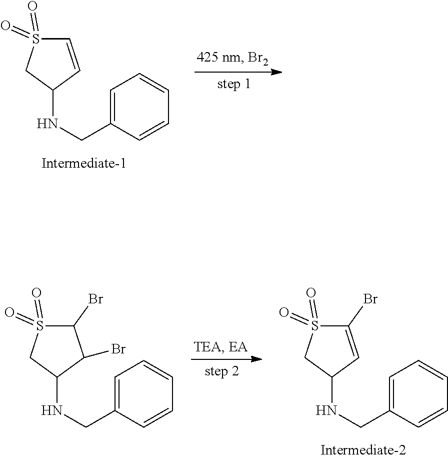 [SMILES: O=S1(=O)C=CC(NCc2ccccc2)C1 ; O=S1(=O)CC(NCc2ccccc2)C(Br)C1Br ; O=S1(=O)CC(NCc2ccccc2)C=C1Br]

Step 1. Synthesis of 4-(benzylamino)-2,3-dibromotetrahydrothiophene 1,1-dioxide: To a solution of 3-(benzylamino)-2,3-dihydrothiophene 1,1-dioxide (Intermediate-1) (1.20 g, 5.38 mmol, 1.00 eq) in CHCl₃ (20 mL) was added Br₂ (2.57 g, 16.1 mmol, 3.00 eq) dropwise at 0° C. After addition, the reaction mixture was allowed to warm to room temperature and stirred for 16 h under UV (425 nm). The precipitated solids were collected by filtration and washed with CHCl₃. The filter cake was dried to afford the title compound, which was used in the next step without further purification. LCMS: 381.9 [M+H]⁺.

Step 2. Synthesis of 3-(benzylamino)-5-bromo-2,3-dihydrothiophene 1,1-dioxide: To a solution of 4-(benzylamino)-2,3-dibromotetrahydrothiophene 1,1-dioxide (1.50 g, 3.92 mmol, 1.00 eq) in EtOAc (30.0 mL) was added TEA (435 mg, 4.31 mmol, 1.10 eq) dropwise at 0° C. and then stirred for 30 min at 0° C. The resulting mixture was filtered, and the filter cake was washed with EtOAc. The filtrate was concentrated under reduced pressure. The residue was purified by reverse Phase HPLC (C18 column, water (10 mmol/L NH₄HCO₃)-ACN) to afford the title compound. LCMS: 302.0 [M+H]⁺.

### Intermediate-3: 3-((4-bromobenzyl)amino)-2,3-dihydrothiophene 1,1-dioxide

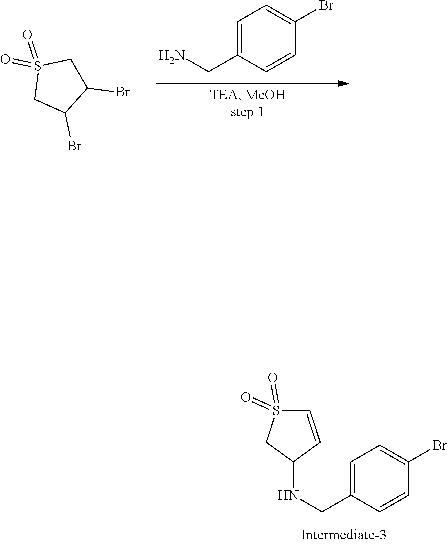 [SMILES: O=S1(=O)CC(Br)C(Br)C1 ; NCc1ccc(Br)cc1 ; O=S1(=O)C=CC(NCc2ccc(Br)cc2)C1]

Step 1. Synthesis of 3-((4-bromobenzyl)amino)-2,3-dihydrothiophene 1,1-dioxide: To a solution of 3,4-dibromotetrahydrothiophene 1,1-dioxide (3.00 g, 10.8 mmol, 1.00 eq) in MeOH (30 mL) was added TEA (5.46 g, 53.9 mmol, 5.00 eq) for 30 min at 0° C. under N₂ atmosphere followed by the addition of 4-bromo-benzylamine (1.00 g, 5.39 mmol, 0.50 eq) at 0° C. The resulting mixture was stirred for overnight at room temperature. The resulting mixture was concentrated under reduced pressure. The residue was purified by flash silica gel chromatography (Eluent of DCM/MeOH) to afford the title compound. LCMS: 302/304 [M+H]⁺.

### Intermediate-4: 2-(2-fluoro-4-(2-methyl-1H-pyrrol-1-yl)phenyl)acetic acid

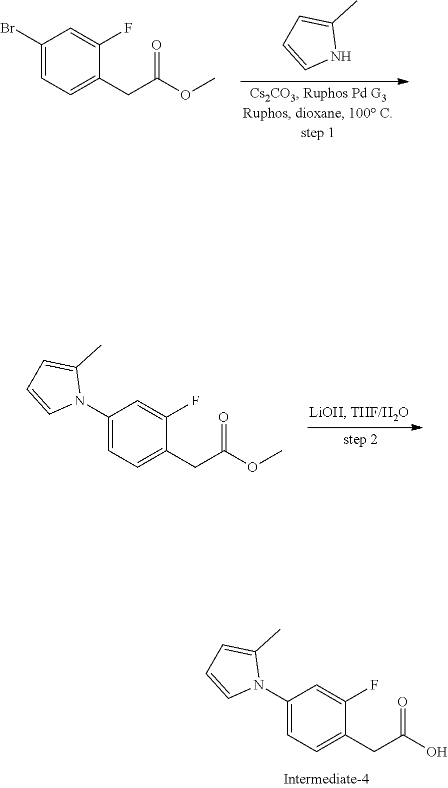

Step 1. Synthesis of methyl 2-(2-fluoro-4-(2-methyl-1H-pyrrol-1-yl)phenyl)acetate: To a solution of methyl 2-(4-bromo-2-fluorophenyl)acetate (300 mg, 1.21 mmol, 1.00 eq), 2-methylpyrrole (492 mg, 6.07 mmol, 5.00 eq) and Cs₂CO₃ (1.19 g, 3.64 mmol, 3.00 eq) in 1,4-dioxane (8.00 mL) were added RuPhos (113 mg, 242 μmol, 0.20 eq) and RuPhos-Pd-G3 (101 mg, 121 μmol, 0.10 eq) at room temperature under N₂ atmosphere. The mixture was stirred for 2 h at 100° C. under N₂ atmosphere. The mixture was cooled to room temperature and diluted with water (50 mL), extracted with EtOAc (3×50 mL). The combined organic layer was washed with brine (1×30 mL), dried over anhydrous Na₂SO₄ and filtered. The filtrate was concentrated under reduced pressure. The residue was purified by reverse phase HPLC (C18 column, water (0.1% FA)-ACN) to afford the title compound. LCMS: 248.1 [M+H]⁺.

Step 2. Synthesis of 2-(2-fluoro-4-(2-methyl-1H-pyrrol-1-yl)phenyl)acetic acid: To a solution of methyl 2-[2-fluoro-4-(2-methylpyrrol-1-yl)phenyl]acetate (152 mg, 438 μmol, 1.00 eq) in THE (8.00 mL) and H₂O (2.00 mL) was added LiOH (52 mg, 2.19 mmol, 5.00 eq) at room temperature. The mixture was stirred for 1 h at room temperature. The pH value of the solution was adjusted to 3 with 1 N HCl (aq.) solution. The resulting mixture was diluted with H₂O (50 mL) and then extracted with EtOAc (3×50 mL). The combined organic layers were dried over anhydrous Na₂SO₄ and filtered. The filtrate was concentrated under reduced pressure. The residue was purified by reverse phase HPLC (C18 column, water (0.1% FA)-ACN) to afford the title compound. LCMS: 234.1 [M+H]⁺.

### Intermediate-5: 3-(((2-methoxypyridin-4-yl)methyl)amino)-2,3-dihydrothiophene 1,1-dioxide

### Intermediate-6: (S)-3-(((2-methoxypyridin-4-yl)methyl)amino)-2,3-dihydrothiophene 1,1-dioxide (single isomer, first eluting compound as isomer 1)

### Intermediate-7: (R)-3-(((2-methoxypyridin-4-yl)methyl)amino)-2,3-dihydrothiophene 1,1-dioxide (single isomer, second eluting compound as isomer 2)

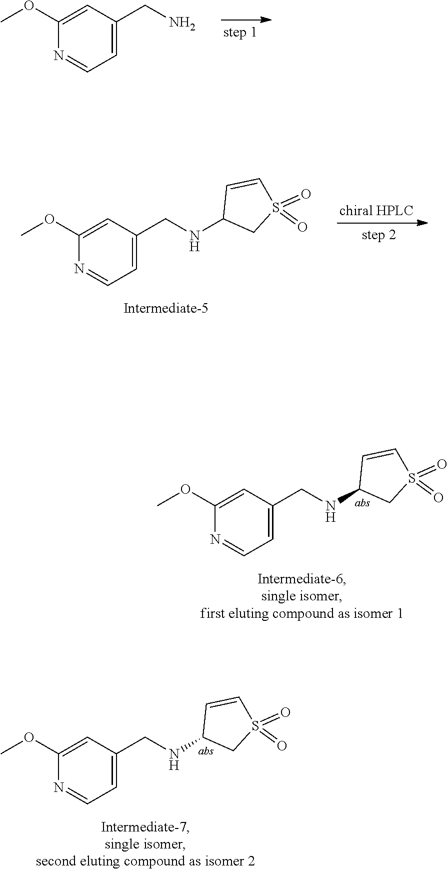

Step 1. Synthesis of 3-(((2-methoxypyridin-4-yl)methyl)amino)-2,3-dihydrothiophene 1,1-dioxide: To a solution of 3,4-dibromotetrahydrothiophene 1,1-dioxide (3.00 g, 10.8 mmol, 1.30 eq) in MeOH (45.0 mL) was added TEA (4.20 g, 41.5 mmol, 5.00 eq) and 1-(2-methoxypyridin-4-yl)methanamine (1.14 g, 8.30 mmol, 1.00 eq) at 0° C. under N₂ atmosphere. The resulting mixture was stirred for 2 h at room temperature under N₂ atmosphere and concentrated. The residue was purified by flash silica gel chromatography (eluent of MeOH/DCM) to afford the title compound. LCMS: 255 [M+H]⁺.

Step 2. Separation of (S)-3-(((2-methoxypyridin-4-yl)methyl)amino)-2,3-dihydrothiophene 1,1-dioxide (single isomer, first eluting compound as isomer 1) and (R)-3-(((2-methoxypyridin-4-yl)methyl)amino)-2,3-dihydrothiophene 1,1-dioxide (single isomer, second eluting compound as isomer 2): 3-(((2-methoxypyridin-4-yl)methyl)amino)-2,3-dihydrothiophene 1,1-dioxide (410 mg) (Intermediate-5) was separated by Prep-chiral-HPLC with the following conditions (Column: JW-CHIRALPAK IH, 20×250 mm; 5 μm; Mobile Phase A: EtOH:DCM=1:2-HPLC, Mobile Phase B: Hex-HPLC; Flow rate: 20 mL/min; Gradient: 10% to 10% B in 9 min, Wave Length: 220/254 nm).

Intermediate-6, (S)-3-(((2-methoxypyridin-4-yl)methyl)amino)-2,3-dihydrothiophene 1,1-dioxide, single isomer, first eluting compound as isomer 1, retention time: 4.00 minutes. LCMS: 255 [M+H]⁺.

Intermediate-7, (R)-3-(((2-methoxypyridin-4-yl)methyl)amino)-2,3-dihydrothiophene 1,1-dioxide, single isomer, second eluting compound as isomer 2, retention time: 5.81 minutes. LCMS: 255 [M+H]⁺.

### Intermediate-8: 3-(dimethylamino)-2-(4′-fluoro-[1,1′-biphenyl]-4-yl)-3-oxopropanoic acid

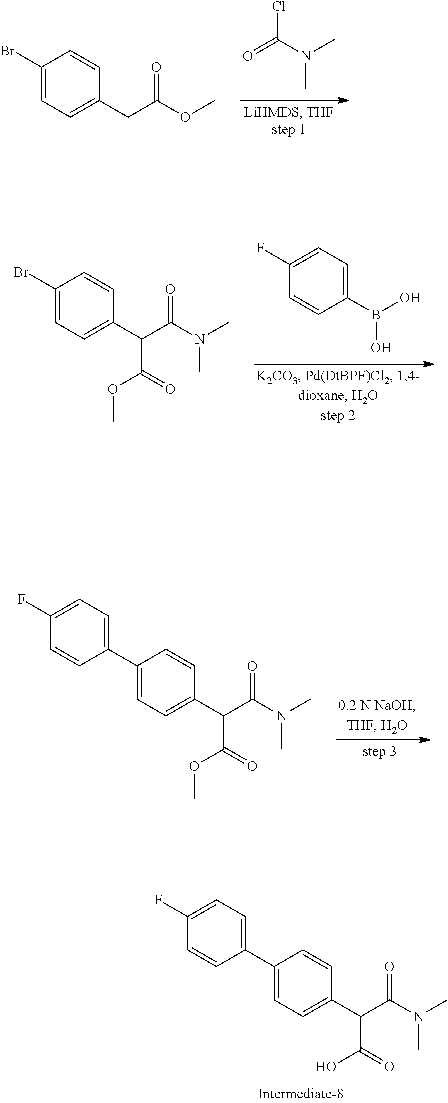

Step 1. Synthesis of methyl 2-(4-bromophenyl)-3-(dimethylamino)-3-oxopropanoate: To a solution of methyl 2-(4-bromophenyl)acetate (2.00 g, 8.73 mmol, 1.00 eq) and dimethylcarbamyl chloride (1.13 g, 10.5 mmol, 1.20 eq) in THF (30.0 mL) were added a solution of LiHMDS in THF (26.0 mL, 26.0 mmol, 3.00 eq, 1 M) dropwise at −78° C. under N₂ atmosphere. After addition, the reaction mixture was allowed to warm to 0° C. and stirred for 5 h under N₂ atmosphere. To the mixture was added saturated NH₄Cl (aq.) solution (50 mL) and extracted with EtOAc (200 mL). The organic layer was dried over anhydrous Na₂SO₄ and filtered. The filtrate was concentrated under reduced pressure to afford the title compound, which was used for next step without further purification. LCMS: 300.0 [M+H]⁺.

Step 2. Synthesis of methyl 3-(dimethylamino)-2-(4′-fluoro-[1,1′-biphenyl]-4-yl)-3-oxopropanoate: To a solution of methyl 2-(4-bromophenyl)-3-(dimethylamino)-3-oxopropanoate (900 mg, 3.00 mmol, 1.00 eq) and 4-fluorophenylboronic acid (503 mg, 3.60 mmol, 1.20 eq) in 1,4-dioxane (15.0 mL) and H₂O (3.00 mL) were added K₂CO₃ (1.24 g, 8.99 mmol, 3.00 eq) and Pd(DtBPF)Cl₂ (390 mg, 0.60 mmol, 0.20 eq) under N₂ atmosphere. The resulting mixture was stirred at 100° C. for 1 h under N₂ atmosphere. The mixture was cooled to room temperature and then concentrated under reduced pressure. The residue was purified by flash silica gel chromatography (eluent of EtOAc/PE) to afford the title compound. LCMS: 316.1 [M+H]⁺.

Step 3. Synthesis of 3-(dimethylamino)-2-(4′-fluoro-[1,1′-biphenyl]-4-yl)-3-oxopropanoic acid: To a solution of 3-(dimethylamino)-2-(4′-fluoro-[1,1′-biphenyl]-4-yl)-3-oxopropanoate (287 mg, 910 μmol, 1.00 eq) in THF (1.50 mL) and H₂O (15.0 mL) were added a solution of NaOH in H₂O (3.00 mL, 0.6 mmol, 0.66 eq, 0.2 N) dropwise at 0° C. After addition, the reaction mixture was allowed to warm to room temperature and stirred for 16 h. The reaction was acidified with 1 N HCl (aq.) solution and extracted with EtOAc (100 mL). The organic layer was dried over anhydrous Na₂SO₄ and filtered. The filtrate was concentrated under reduced pressure to afford the title compound, which was used for next step without further purification. LCMS: 302.1 [M+H]⁺.

### Intermediate-9: 3-(((2-(2-hydroxypropan-2-yl)pyridin-4-yl)methyl)amino)-2,3-dihydrothiophene 1,1-dioxide

### Intermediate-10: (R)-3-(((2-(2-hydroxypropan-2-yl)pyridin-4-yl)methyl)amino)-2,3-dihydrothiophene 1,1-dioxide (single isomer, first eluting compound as isomer 1)

### Intermediate-11: (S)-3-(((2-(2-hydroxypropan-2-yl)pyridin-4-yl)methyl)amino)-2,3-dihydrothiophene 1,1-dioxide (single isomer, second eluting compound as isomer 2)

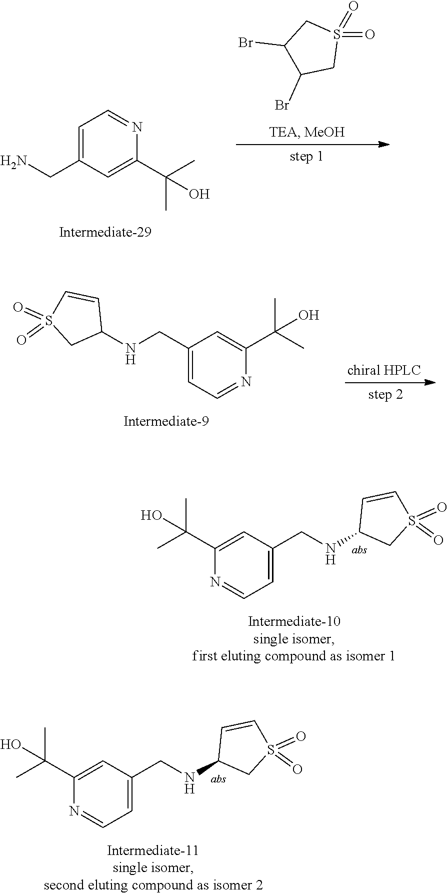 [SMILES: O=S1(=O)CC(Br)C(Br)C1 ; CC(C)(O)c1cc(CN)ccn1 ; CC(C)(O)c1cc(CNC2C=CS(=O)(=O)C2)ccn1 ; CC(C)(O)c1cc(CN[C@@H]2C=CS(=O)(=O)C2)ccn1 ; CC(C)(O)c1cc(CN[C@H]2C=CS(=O)(=O)C2)ccn1]

Step 1. Synthesis of 3-(((2-(2-hydroxypropan-2-yl)pyridin-4-yl)methyl)amino)-2,3-dihydrothiophene 1,1-dioxide: To a mixture of 2-[4-(aminomethyl)pyridin-2-yl]propan-2-ol (Intermediate-29) (10.0 g, 60.1 mmol, 1.00 eq) and TEA (30.4 g, 300 mmol, 5.00 eq) in MeOH (200 mL) was added 3,4-dibromotetrahydrothiophene 1,1-dioxide (16.7 g, 60.1 mmol, 1.00 eq) at 0° C. and the mixture was stirred at room temperature for 2 h. The resulting mixture was cooled to 0° C., and TEA (30.4 g, 300 mmol, 5.00 eq) and 3,4-dibromotetrahydrothiophene 1,1-dioxide (16.7 g, 60.1 mmol, 1.00 eq) were added and stirred for additional 16 h at room temperature. The resulting mixture was cooled to 0° C., and 3,4-dibromotetrahydrothiophene 1,1-dioxide (16.7 g, 60.1 mmol, 1.00 eq) was added and the mixture was stirred for 16 h at room temperature. The resulting mixture was concentrated under reduced pressure. The residue was treated with EtOAc (100 mL), filtered and the filtrate was concentrated under vacuum. This work up was repeated three times. The residue was purified by silica gel column chromatography (eluent of EtOAc/MeOH) to afford the title compound. LCMS: 283 [M+H]⁺.

Step 2. Separation of (R)-3-(((2-(2-hydroxypropan-2-yl)pyridin-4-yl)methyl)amino)-2,3-dihydrothiophene 1,1-dioxide (single isomer, first eluting compound as isomer 1) and (S)-3-(((2-(2-hydroxypropan-2-yl)pyridin-4-yl)methyl)amino)-2,3-dihydrothiophene 1,1-dioxide (single isomer, second eluting compound as isomer 2): 3-(((2-(2-hydroxypropan-2-yl)417yridine-4-yl)methyl)amino)-2,3-dihydrothiophene 1,1-dioxide (16.0 g) (Intermediate-9) was separated by chiral-HPLC with the following conditions: Column: JW-CHIRALPAK IG-3, 3.0×50 mm; 3 μm; Mobile Phase A: IPA-HPLC, Mobile Phase B: Hex (0.5% 2M NH₃-MeOH)-HPLC; Flow rate: 29 mL/min; Gradient: 50% to 50% B in 17 min; Wave Length: 220/254 nm.

Intermediate-10, (R)-3-(((2-(2-hydroxypropan-2-yl)pyridin-4-yl)methyl)amino)-2,3-dihydrothiophene 1,1-dioxide, single isomer, first eluting compound as isomer 1 retention time: 11.5 minutes. LCMS: 283 [M+H]⁺.

Intermediate-11, (S)-3-(((2-(2-hydroxypropan-2-yl)pyridin-4-yl)methyl)amino)-2,3-dihydrothiophene 1,1-dioxide, single isomer, second eluting compound as isomer 2, retention time: 14.2 minutes. LCMS: 283 [M+H]⁺.

### Intermediate-12: methyl 2-(3,3′,4′-trifluoro-[1,1′-biphenyl]-4-yl)acetate

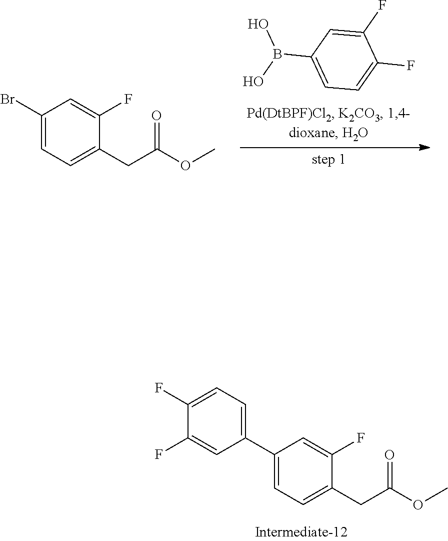 [SMILES: OB(O)c1ccc(F)c(F)c1 ; COC(=O)Cc1ccc(Br)cc1F ; COC(=O)Cc1ccc(-c2ccc(F)c(F)c2)cc1F]

Step 1. Synthesis of methyl 2-(3,3′,4′-trifluoro-[1,1′-biphenyl]-4-yl)acetate: To a mixture of methyl 2-(4-bromo-2-fluorophenyl)acetate (30.0 g, 121 mmol, 1.00 eq), 3,4-difluorophenylboronic acid (19.2 g, 121 mmol, 1.00 eq) and K₂CO₃ (50.4 g, 364 mmol, 3.00 eq) in 1,4-dioxane (420 mL) and H₂O (42.0 mL) was added Pd(DtBPF)Cl₂ (7.91 g, 12.1 mmol, 0.10 eq) at room temperature. The mixture was degassed three times with N2. After stirring at room temperature for 2 h, the resulting mixture was filtered, the filter cake was washed with DCM. The filtrate was concentrated under reduced pressure. The residue was purified by flash silica gel column chromatography (Eluent of EtOAc/PE) to afford the title compound. ¹H NMR (400 MHz, DMSO-d₆) δ ppm 7.88-7.83 (m, 1H), 7.65-7.37 (m, 5H), 3.79 (d, 2H), 3.65 (s, 3H).

### Intermediate-13: methyl 2-bromo-2-(3,3′,4′-trifluoro-[1,1′-biphenyl]-4-yl)acetate

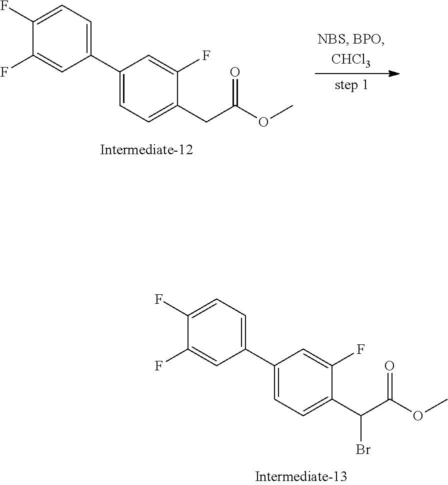 [SMILES: COC(=O)Cc1ccc(-c2ccc(F)c(F)c2)cc1F ; COC(=O)C(Br)c1ccc(-c2ccc(F)c(F)c2)cc1F]

Step 1. Synthesis of methyl 2-bromo-2-(3,3′,4′-trifluoro-[1,1′-biphenyl]-4-yl)acetate: To a mixture of methyl 2-(3,3′,4′-trifluoro-[1,1′-biphenyl]-4-yl)acetate (Intermediate-12) (1.00 g, 3.57 mmol, 1.00 eq) and NBS (0.95 g, 5.35 mmol, 1.50 eq) in CHCl₃ (5.00 mL) was added BPO (274 mg, 1.07 mmol, 0.30 eq) at room temperature. The resulting mixture was degassed three times with N₂. After stirring at 80° C. for 4 h, the resulting mixture was concentrated under vacuum. The residue was purified via reverse phase HPLC (C18 column, water (10 mmol/L NH₄HCO₃)-ACN) to afford the title compound. ¹H NMR (400 MHz, DMSO-d₆) δ ppm 7.92-7.87 (m, 1H), 7.70-7.56 (m, 5H), 6.23 (s, 1H), 3.76 (s, 3H).

### Intermediate-14: 3-(((4-(2-hydroxypropan-2-yl)thiazol-2-yl)methyl)amino)-2,3-dihydrothiophene 1,1-dioxide

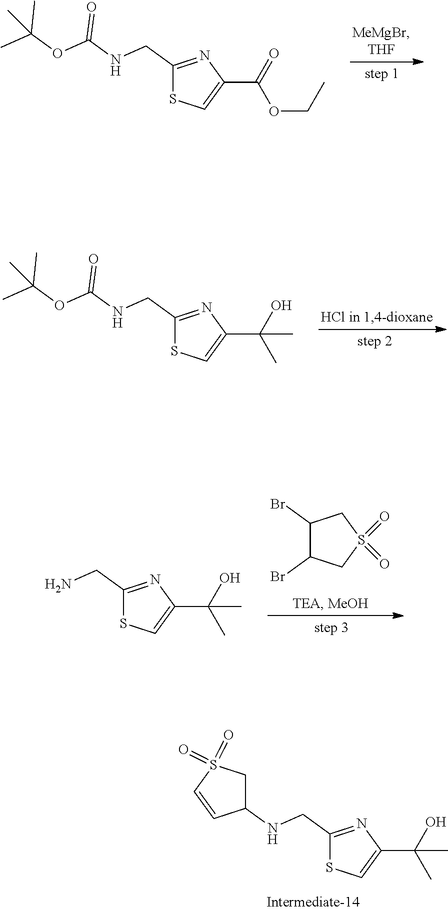

Step 1. Synthesis of tert-butyl ((4-(2-hydroxypropan-2-yl)thiazol-2-yl)methyl)carbamate: A solution of ethyl 2-{[(tert-butoxycarbonyl)amino]methyl}-1,3-thiazole-4-carboxylate (5.00 g, 17.5 mmol, 1.00 eq) in THF (70.0 mL) was degassed three times with N₂, and then was cooled to −78° C. To the solution was added a solution of MeMgBr in THF (65 mL, 65 mmol, 3.70 eq, 1 M) dropwise at −78° C. The resulting mixture was stirred for 1 h at room temperature. The reaction was quenched with sat. NH₄Cl (aq.) at 0° C. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by flash silica gel column chromatography (eluent of EtOAc/PE) to afford the title compound. LCMS: 273 [M+H]⁺.

Step 2. Synthesis of 2-(2-(aminomethyl)thiazol-4-yl)propan-2-ol hydrochloride: To a solution of tert-butyl ((4-(2-hydroxypropan-2-yl)thiazol-2-yl)methyl)carbamate (2.80 g, 10.3 mmol, 1.00 eq) in DCM (50.0 mL) was added a solution of 4 M HCl in 1,4-dioxane (28 mL, 112 mmol, 10.9 eq) at room temperature. After stirring for 1 h at room temperature, the resulting mixture was concentrated and to give the title compound, which was used in the next step directly without further purification. LCMS: 173 [M+H]⁺.

Step 3. Synthesis of 3-(((4-(2-hydroxypropan-2-yl)thiazol-2-yl)methyl)amino)-2,3-dihydrothiophene 1,1-dioxide: To a solution of 3,4-dibromotetrahydrothiophene 1,1-dioxide (3.43 g, 12.3 mmol, 1.20 eq) in MeOH (20.0 mL) was added TEA (5.20 g, 51.4 mmol, 5.00 eq) at 0° C. To the above mixture was added 2-[2-(aminomethyl)-1,3-thiazol-4-yl]propan-2-ol hydrochloride (2.10 g, 10.3 mmol, 1.00 eq) in portions over 5 min at 0° C. The resulting mixture was stirred for overnight at room temperature. The resulting mixture was filtered and concentrated under reduced pressure. The residue was purified by flash silica gel column chromatography (eluent of DCM/MeOH) to afford the title compound. LCMS: 289 [M+H]⁺.

### Intermediate-15: 2-(3,4′-difluoro-[1,1′-biphenyl]-4-yl)acetic acid

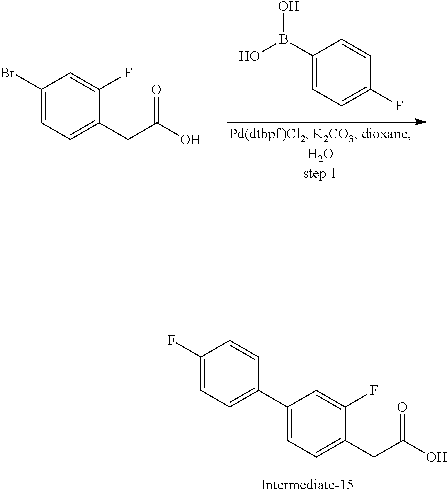 [SMILES: O=C(O)Cc1ccc(Br)cc1F ; OB(O)c1ccc(F)cc1 ; O=C(O)Cc1ccc(-c2ccc(F)cc2)cc1F]

Step 1. Synthesis of 2-(3,4′-difluoro-[1,1′-biphenyl]-4-yl)acetic acid: To a solution of 2-(4-bromo-2-fluorophenyl)acetic acid (1.00 g, 4.29 mmol, 1.00 eq) and (4-fluorophenyl)boronic acid (721 mg, 5.15 mmol, 1.20 eq) in 1,4-dioxane (10.0 mL) and H₂O (2.00 mL) were added K₂CO₃ (1.77 g, 12.9 mmol, 3.00 eq) and Pd(dtbpf)Cl₂ (279 mg, 429 μmol, 0.10 eq) at room temperature under N₂ atmosphere. The resulting mixture was stirred at 100° C. for 2 h under N₂ atmosphere. The mixture was cooled to room temperature and concentrated under vacuum. The residue was purified by reverse phase HPLC (C18 column, water (0.1% FA)-ACN) to afford the title compound. LCMS: 249.1 [M+H]⁺.

### Intermediate-16: methyl 2-hydroxy-2-(3,3′,4′-trifluoro-[1,1′-biphenyl]-4-yl)acetate

### Intermediate-17: 2-methoxy-2-(3,3′,4′-trifluoro-[1,1′-biphenyl]-4-yl)acetic acid

### Intermediate-18: 2-methoxy-2-(3,3′,4′-trifluoro-[1,1′-biphenyl]-4-yl)acetic acid (single isomer, first eluting compound as isomer 1)

### Intermediate-19: 2-methoxy-2-(3,3′,4′-trifluoro-[1,1′-biphenyl]-4-yl)acetic acid (single isomer, second eluting compound as isomer 2)

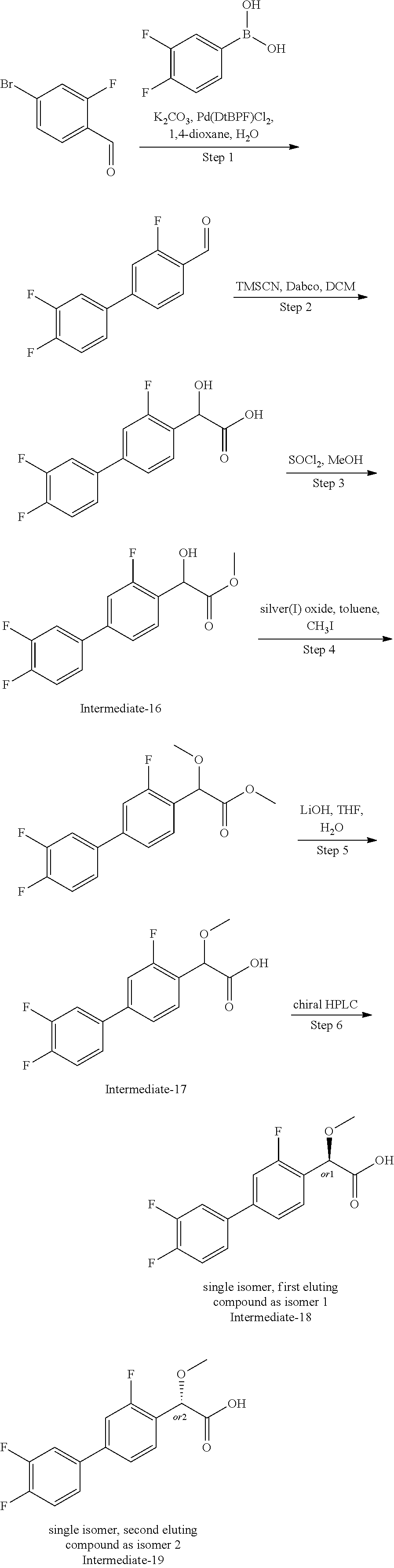

Step 1. Synthesis of 3,3′,4′-trifluoro-[1,1′-biphenyl]-4-carbaldehyde. To a mixture of 4-bromo-2-fluorobenzaldehyde (15.0 g, 73.9 mmol, 1.00 eq) and 3,4-difluorophenylboronic acid (11.7 g, 73.9 mmol, 1.00 eq) in 1,4-dioxane (150 mL) and H₂O (15.0 mL) were added Pd(DtBPF)Cl₂ (4.82 g, 7.39 mmol, 0.10 eq) and K₂CO₃ (30.6 g, 222 mmol, 3.00 eq) at room temperature. The mixture was degassed three times with N₂, and then was stirred for 1 h at room temperature. The mixture was diluted with water and extracted with EtOAc (5×150 mL). The combined organic layer was washed with brine (3×100 mL), dried over anhydrous Na₂SO₄, filtered and concentrated under reduced pressure. The residue was purified by flash silica gel column chromatography (eluent of EtOAc/PE) to afford the title compound. LCMS: 237 [M+H]⁺.

Step 2. Synthesis of 2-hydroxy-2-(3,3′,4′-trifluoro-[1,1′-biphenyl]-4-yl)acetic acid: To a mixture of 3,3′,4′-trifluoro-[1,1′-biphenyl]-4-carbaldehyde (14.0 g, 59.3 mmol, 1.00 eq) and TMSCN (5.88 g, 59.3 mmol, 1.00 eq) in DCM (140 mL) was added Dabco (0.24 g, 2.13 mmol, 0.04 eq) at 0° C. The mixture was degassed three times with N₂, and then was stirred for 30 min at 0° C. and 2 h at room temperature. The resulting mixture was poured into water, extracted with DCM (3×150 mL). The combined organic layers were washed with brine (3×100 mL), dried over anhydrous Na₂SO₄. After filtration, the filtrate was concentrated under reduced pressure. To the residue was added water (93 mL) and conc. HCl (279 mL). The resulting mixture was refluxed for 2 h, and then was cooled to room temperature. The resulting mixture was extracted with EtOAc (3×100 mL). The combined organic layers were washed with water (3×100 mL), dried over anhydrous Na₂SO₄. After filtration, the filtrate was concentrated under reduced pressure. The residue was purified by reverse phase HPLC (C18 column, water (0.1% FA)-ACN) to afford the title compound. LCMS: 281 [M−H]⁻.

Step 3. Synthesis of methyl 2-hydroxy-2-(3,3′,4′-trifluoro-[1,1′-biphenyl]-4-yl)acetate To a stirred mixture of 2-hydroxy-2-(3,3′,4′-trifluoro-[1,1′-biphenyl]-4-yl)acetic acid (10.8 g, 38.3 mmol, 1.00 eq) in MeOH (100 mL) was added SOCl₂ (6.83 g, 57.4 mmol, 1.50 eq) in 10 min at 0° C. The mixture was degassed three times with N₂, and then was stirred for 2 h at room temperature. The resulting mixture was concentrated under reduced pressure. The residue was diluted with water and then extracted with EtOAc (3×100 mL). The combined organic layers were washed with H₂O (3×100 mL), dried over anhydrous Na₂SO₄. After filtration, the filtrate was concentrated under reduced pressure to afford the title compound, which was used in the next step without further purification. LCMS: 295 [M−H]⁻.

Step 4. Synthesis of methyl 2-methoxy-2-(3,3′,4′-trifluoro-[1,1′-biphenyl]-4-yl)acetate: To a stirred mixture of methyl 2-hydroxy-2-(3,3′,4′-trifluoro-[1,1′-biphenyl]-4-yl)acetate (Intermediate-16) (9.00 g, 30.4 mmol, 1.00 eq) and CH₃I (13.0 g, 30.4 mmol, 3.00 eq) in toluene (90 mL) was added Ag₂O (10.6 g, 45.6 mmol, 1.50 eq) at 0° C. The mixture was degassed three times with N₂, and then was stirred for overnight at 100° C. The resulting mixture was cooled to room temperature, filtered, the filter cake was washed with EtOAc. The filtrate was concentrated under reduced pressure. The residue was purified by flash silica gel chromatography (eluent of EtOAc/PE) to afford the title compound. LCMS: 311 [M+H]⁺.

Step 5. Synthesis of 2-methoxy-2-(3,3′,4′-trifluoro-[1,1′-biphenyl]-4-yl)acetic acid: To a stirred mixture of methyl 2-methoxy-2-(3,3′,4′-trifluoro-[1,1′-biphenyl]-4-yl)acetate (4.00 g, 12.9 mmol, 1.00 eq) in THE (40.0 mL) and H₂O (8.00 mL) was added LiOH (0.93 g, 38.7 mmol, 3.00 eq) at room temperature. The mixture was stirred for 1 h at room temperature. The pH value of the solution was adjusted to 3 with 1 N HCl (aq.) solution. The resulting mixture was extracted with EtOAc (3×100 mL). The combined organic layers were washed with water (3×100 mL), dried over anhydrous Na₂SO₄. After filtration, the filtrate was concentrated under reduced pressure to give the title compound, which was used in the next step without further purification. LCMS: 295 [M−H]⁻.

Step 6. Separation of 2-methoxy-2-(3,3′,4′-trifluoro-[1,1′-biphenyl]-4-yl)acetic acid (single isomer, first eluting compound as isomer 1) and 2-methoxy-2-(3,3′,4′-trifluoro-[1,1′-biphenyl]-4-yl)acetic acid (single isomer, second eluting compound as isomer 2): 2-Methoxy-2-(3,3′,4′-trifluoro-[1,1′-biphenyl]-4-yl)acetic acid (3.90 g) (Intermediate-17) was separated by Prep-chiral-HPLC with the following conditions (Column: CHIRALPAK IG-3, 3.0×50 mm; 3 μm; Mobile Phase A: EtOH-HPLC, Mobile Phase B: Hex (0.1% TFA)-HPLC; Flow rate: 40 mL/min; Gradient: 90% to 90% B in 25 min; Wave Length: 220/254 nm; Sample Solvent: MeOH:EtOH=1:1-HPLC).

Intermediate-18, 2-Methoxy-2-(3,3′,4′-trifluoro-[1,1′-biphenyl]-4-yl)acetic acid, single isomer, first eluting compound as isomer 1, retention time: 16.32 minutes. LCMS: 295 [M−H]⁻.

Intermediate-19, 2-Methoxy-2-(3,3′,4′-trifluoro-[1,1′-biphenyl]-4-yl)acetic acid, single isomer, second eluting compound as isomer 2, retention time: 20.52 min. LCMS: 295 [M−H]⁻.

### Intermediate-20: [4-(3,4-difluorophenyl)pyrazol-1-yl]acetic acid

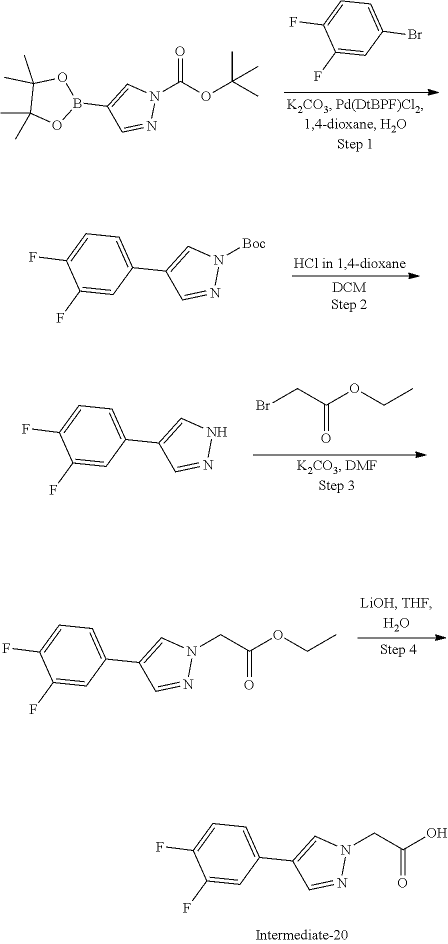

Step 1. Synthesis of tert-butyl 4-(3,4-difluorophenyl)pyrazole-1-carboxylate: To a solution of tert-butyl 4-(4,4,5,5-tetramethyl-1,3,2-dioxaborolan-2-yl)pyrazole-1-carboxylate (900 mg, 3.06 mmol, 1.00 eq) and 4-bromo-1,2-difluorobenzene (590 mg, 3.06 mmol, 1.00 eq) in 1,4-dioxane (10.0 mL) and H₂O (1.00 mL) were added K₂CO₃ (1.27 g, 9.18 mmol, 3.00 eq) and Pd(DtBPF)Cl₂ (200 mg, 310 μmol, 0.10 eq) at room temperature under N₂ atmosphere. The resulting mixture was stirred for 1 h at room temperature and diluted with H₂O (60 mL). The resulting mixture was extracted with EtOAc (3×60 mL). The combined organic layers were washed with brine (1×60 mL), dried over anhydrous Na₂SO₄ and filtered. The filtrate was concentrated under reduced pressure. The residue was purified by flash silica gel chromatography (Eluent of EtOAc/PE) to afford the title compound. LCMS: 181.0 [M-100+H]⁺.

Step 2. Synthesis of 4-(3,4-difluorophenyl)-1H-pyrazole: To a solution of tert-butyl 4-(3,4-difluorophenyl)pyrazole-1-carboxylate (500 mg, 1.78 mmol, 1.00 eq) in DCM (12.0 mL) was added 4 M HCl in 1,4-dioxane (6.00 mL) dropwise under N₂ atmosphere. The resulting mixture was stirred for 1 h at room temperature and concentrated under reduced pressure to afford the title compound, which was used for next step without further purification. LCMS: 181.0 [M+H]⁺.

Step 3. Synthesis of ethyl 2-[4-(3,4-difluorophenyl)pyrazol-1-yl]acetate: To a stirred mixture of 4-(3,4-difluorophenyl)-1H-pyrazole (150 mg, 0.83 mmol, 1.00 eq) and ethyl bromoacetate (278 mg, 1.67 mmol, 2.00 eq) in DMF (10.0 mL) was added K₂CO₃ (345 mg, 2.50 mmol, 3.00 eq) under N₂ atmosphere. The resulting mixture was stirred for 2 h at 60° C. under N₂ atmosphere. The resulting mixture was cooled to room temperature and diluted with H₂O (30 mL). The resulting mixture was extracted with EtOAc (3×30 mL). The combined organic layers were washed with brine (1×30 mL), dried over anhydrous Na₂SO₄ and filtered. The filtrate was concentrated under reduced pressure. The residue was purified by Prep-TLC (EtOAc/PE) to afford the title compound. LCMS: 267.1 [M+H]⁺.

Step 4. Synthesis of [4-(3,4-difluorophenyl)pyrazol-1-yl]acetic acid: To a solution of ethyl 2-[4-(3,4-difluorophenyl)pyrazol-1-yl]acetate (100 mg, 380 gmol, 1.00 eq) in THF (5.00 mL) and H₂O (1.00 mL) were added LiOH (27 mg, 1.19 mmol, 3.00 eq) at room temperature. The resulting mixture was stirred for 2 h at room temperature. The mixture was diluted with water and acidified to pH 2-3 with 1 N HCl (aq.) at 0° C., then was extracted with EtOAc (3×40 mL). The combined organic layers were washed with brine (1×30 mL), dried over anhydrous Na₂SO₄. After filtration, the filtrate was concentrated under reduced pressure to afford the title compound which was used for next step directly. LCMS: 239.1 [M+H]⁺.

### Intermediate-21: 3-(((4-cyclopropylthiazol-2-yl)methyl)amino)-2,3-dihydrothiophene 1,1-dioxide

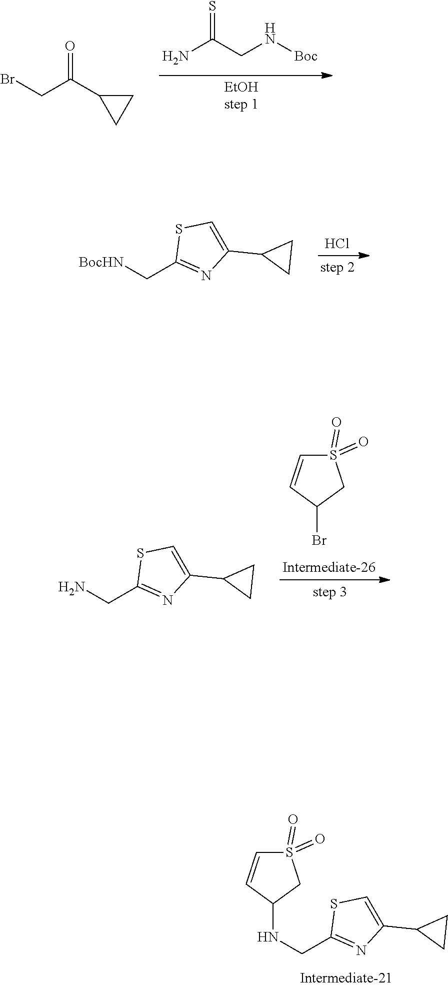

Step1: Synthesis of tert-butyl ((4-cyclopropylthiazol-2-yl)methyl)carbamate: To a mixture of tert-butyl (2-amino-2-thioxoethyl)carbamate (5.00 g, 26.28 mmol, 1.00 eq) in EtOH (50 mL) was added 2-bromo-1-cyclopropylethan-1-one (5.57 g, 34.2 mmol, 1.30 eq) and Na₂CO₃ (2.79 g, 26.3 mmol, 1.00 eq) at room temperature. Then the reaction was heated to 80° C. and stirred at 80° C. for 15 hours. The mixture was cooled to room temperature and poured into ice-water (20 mL). The resulting mixture was extracted with EtOAc (20 mL×3). The combined organic layers were washed with brine (20 mL), dried over anhydrous Na₂SO₄, filtered and the filtrate was concentrated in vacuum. The residue was purified by silica gel chromatography (eluent of EtOAc/PE) to afford the title compound. LCMS: 255.1 [M+1]⁺.

Step 2: Synthesis of 4-cyclopropylthiazol-2-yl)methanamine, hydrochloride acid: To a solution of tert-butyl ((4-cyclopropylthiazol-2-yl)methyl)carbamate (5.30 g, 20.8 mmol, 1.00 eq) in DCM (5 mL) was added 4 M HCl solution in 1,4-dioxane (15 mL) at room temperature. The mixture was stirred at room temperature for 2 hours. After completion, the mixture was concentrated in vacuum to afford the title compound, which was used in the next step without further purification. LCMS: 155.1 [M+1]⁺.

Step 3: Synthesis of 3-(((4-cyclopropylthiazol-2-yl)methyl)amino)-2,3-dihydrothiophene 1,1-dioxide: To a mixture of 3-bromo-2,3-dihydrothiophene 1,1-dioxide (Intermediate-26) (2.50 g, 12.7 mmol, 1.00 eq) and (4-cyclopropylthiazol-2-yl)methanamine (1.96 g, 12.7 mmol, 1.00 eq) in MeOH (30 mL) was added TEA (3.85 g, 38.1 mmol, 5.30 mL, 3.00 eq) at room temperature. Then the reaction mixture was heated to 60° C. stirred at 60° C. for 2 hours. The mixture was poured into ice-water (10 mL) and the resulting mixture was extracted with ethyl acetate (10 mL×3). The combined organic layers were washed with brine (10 mL), dried with anhydrous Na₂SO₄, filtered and the filtrate was concentrated in vacuum. The residue was purified by prep-HPLC (water (NH₄HCO₃)-ACN) to afford the title compound. LCMS: 271.1[M+1]⁺.

### Intermediate-22: 2-(4-(4-fluorophenyl)-2-oxopyridin-1(2H)-yl)acetic acid

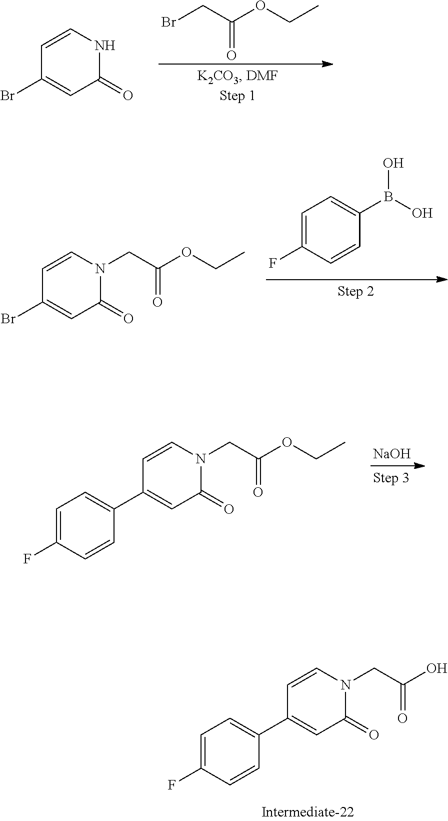

Step 1. Synthesis of ethyl 2-(4-bromo-2-oxopyridin-1(2H)-yl)acetate: To a mixture of 4-bromopyridin-2(1H)-one (1.00 g, 5.75 mmol, 1.00 eq) and ethyl 2-bromoacetate (1.06 g, 6.32 mmol, 699 μL, 1.10 eq) in DMF (10.0 mL) was added K₂CO₃ (2.38 g, 17.2 mmol, 3.00 eq) at room temperature and then warmed to 30° C. and stirred at 30° C. for 12 h. The reaction was quenched by saturated NH₄Cl (aq.) solution (100 mL) and extracted with EtOAc (3×50 mL). The combined organic layers were dried over anhydrous Na₂SO₄ and filtered. The filtrate was concentrated under reduced pressure. The residue was purified by flash silica gel chromatography (Eluent of EtOAc/PE) to afford the title compound. LCMS: 262.1[M+H]⁺.

Step 2. Synthesis of ethyl 2-(4-(4-fluorophenyl)-2-oxopyridin-1(2H)-yl)acetate: To a mixture of ethyl 2-(4-bromo-2-oxopyridin-1(2H)-yl)acetate (500 mg, 1.92 mmol, 1.00 eq) and (4-fluorophenyl)boronic acid (268 mg, 1.92 mmol, 1.00 eq) in 1,4-dioxane (10.0 mL) and H₂O (1.00 mL) was added Pd(amphos)Cl₂ (136 mg, 192 μmol, 0.10 eq) and KF (335 mg, 5.77 mmol, 3.00 eq) at room temperature under Ar atmosphere. The mixture was then stirred for 12 h at 50° C. under Ar atmosphere. The reaction mixture was washed by saturated NH₄Cl (aq.) solution (30 mL) and extracted with EtOAc (3×20 mL). The combined organic layers were washed with brine (1×30 mL), dried over anhydrous Na₂SO₄ and filtered. The filtrate was concentrated under reduced pressure. The residue was purified by flash silica gel chromatography (Eluent of EtOAc/PE) to afford the title compound. LCMS: 276.1[M+H]⁺.

Step 3. Synthesis of 2-(4-(4-fluorophenyl)-2-oxopyridin-1(2H)-yl)acetic acid: To a solution of ethyl 2-(4-(4-fluorophenyl)-2-oxopyridin-1(2H)-yl)acetate (200 mg, 726 μmol, 1.00 eq) in MeOH (1.00 mL), THF (1.00 mL) and H₂O (2.00 mL) was added 1 N NaOH (aq.) solution (1.90 mL, 1.90 mmol, 2.60 eq) at room temperature and the resulting mixture was stirred at room temperature for 1 h. The reaction mixture was acidified by 0.5 N HCl (aq.) solution and then extracted with EtOAc (3×20 mL). The combined organic layers were washed with brine (30 mL), dried over anhydrous Na₂SO₄ and filtered. The filtrate was concentrated under reduced pressure to afford the title compound, which was used in the next step without further purification. LCMS: 248.1 [M+H]⁺.

### Intermediate-23: 3-((pyridin-4-ylmethyl)amino)-2,3-dihydrothiophene 1,1-dioxide

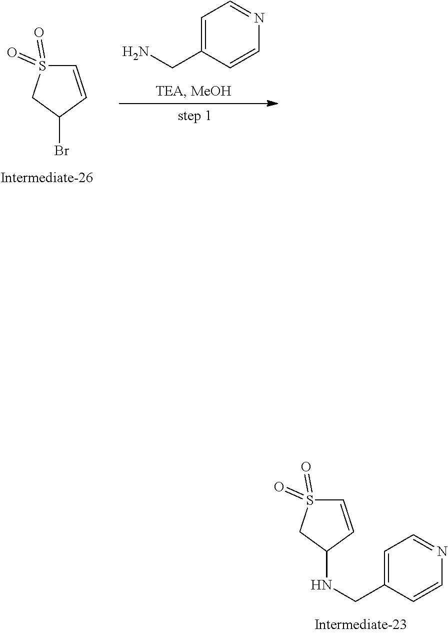 [SMILES: O=S1(=O)C=CC(Br)C1 ; NCc1ccncc1 ; O=S1(=O)C=CC(NCc2ccncc2)C1]

Step 1. Synthesis of 3-((pyridin-4-ylmethyl)amino)-2,3-dihydrothiophene 1,1-dioxide: To a solution of 3-bromo-2,3-dihydrothiophene 1,1-dioxide (Intermediate-26) (5.00 g, 25.4 mmol, 1.00 eq) in MeOH (60.0 mL) were added TEA (7.70 g, 76.1 mmol, 3.00 eq) and 4-pyridinemethaneamine (4.12 g, 38.1 mmol, 1.50 eq) at room temperature. The resulting mixture was stirred for 3 h at 60° C. The resulting mixture was cooled to room temperature and concentrated under vacuum. The residue was purified via silica gel column chromatography (Eluent of DCM/MeOH) to afford the title compound. LCMS: 225 [M+H]⁺.

### Intermediate-24: 1-(tert-butyl) 3-methyl 2-(3,3′,4′-trifluoro-[1,1′-biphenyl]-4-yl)malonate

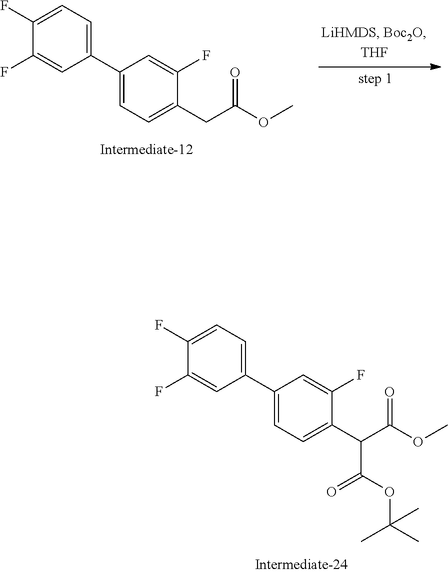 [SMILES: COC(=O)Cc1ccc(-c2ccc(F)c(F)c2)cc1F ; COC(=O)C(C(=O)OC(C)(C)C)c1ccc(-c2ccc(F)c(F)c2)cc1F]

Step 1. Synthesis of 1-(tert-butyl) 3-methyl 2-(3,3′,4′-trifluoro-[1,1′-biphenyl]-4-yl)malonate: To a stirred mixture of methyl 2-(3,3′,4′-trifluoro-[1,1′-biphenyl]-4-yl)acetate (Intermediate-12) (10.0 g, 35.7 mmol, 1.00 eq) in THF (60.0 mL) was added a solution of LiHMDS in THE (84.0 mL, 84.0 mmol, 2.35 eq, 1M) dropwise at −78° C. under N₂ atmosphere. After stirring for 30 min at −78° C. under N₂ atmosphere, to the above mixture was added a solution of (Boc)₂O (9.40 g, 43.1 mmol, 1.21 eq) in THF (40.0 mL) dropwise over 20 min at −78° C. The resulting mixture was stirred for additional 16 h at room temperature. The reaction was quenched with saturated NH₄Cl (aq.) solution (100 mL) at room temperature. The resulting mixture was extracted with EtOAc (3×100 mL). The combined organic layers were washed with brine, dried over anhydrous Na₂SO₄. After filtration, the filtrate was concentrated under reduced pressure to afford the title compound, which was used in the next step directly without further purification. LCMS: 379 [M−H]⁻.

### Intermediate-25: 2-(5-(pentafluoro-λ⁶-sulfaneyl)-2H-indazol-2-yl)acetic acid

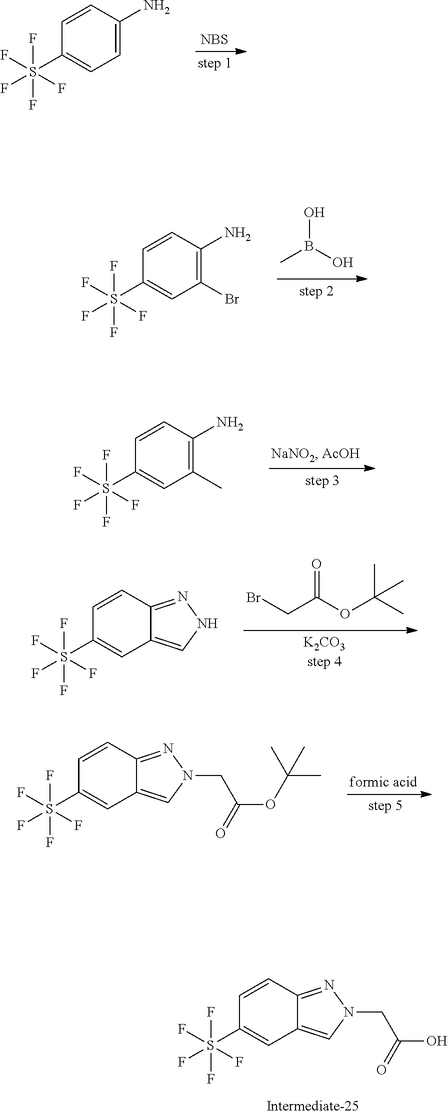

Step 1. Synthesis of 2-bromo-4-(pentafluoro-λ⁶-sulfaneyl)aniline: To a solution of 4-(pentafluoro-λ⁶-sulfaneyl)aniline (1.80 g, 8.21 mmol, 1.00 eq) in DMF (20.0 mL) was added NBS (1.75 g, 9.86 mmol, 1.20 eq) at room temperature and the mixture was stirred at room temperature for 2 h. The reaction mixture was poured into H₂O (20 mL) and extracted with EtOAc (20 mL×2). The combined organic layers were dried over anhydrous Na₂SO₄, filtered and concentrated under reduced pressure. The residue was purified by flash silica gel chromatography (eluent of EtOAc/PE) to afford the title compound. ¹H NMR (400 MHz, CDCl₃) δ ppm 7.82 (d, 1H), 7.50 (dd, 1H), 6.73 (d, 1H), 4.98-3.81 (m, 2H).

Step 2. Synthesis of 2-methyl-4-(pentafluoro-λ⁶-sulfaneyl)aniline: To a solution of 2-bromo-4-(pentafluoro-λ⁶-sulfaneyl)aniline (1.00 g, 3.35 mmol, 1.00 eq) in 1,4-dioxane (10.0 mL) was added Pd(dppf)Cl₂ (274 mg, 335 mol, 0.10 eq), Cs₂CO₃ (3.28 g, 10.1 mmol, 3.00 eq) and methylboronic acid (402 mg, 6.71 mmol, 2.00 eq) at room temperature. Then the mixture was heated to 105° C. and stirred at 105° C. for 16 h. The reaction mixture cooled to room temperature and then diluted with water (20 mL). The mixture was extracted with EtOAc 30 mL (3×10 mL), dried over anhydrous Na₂SO₄, filtered and concentrated under reduced pressure. The residue was purified by flash silica gel chromatography (eluent of EtOAc/PE) to afford the title compound. ¹H NMR (400 MHz, CDCl₃) δ ppm 7.50-7.36 (m, 2H), 6.61 (d, 1H), 3.95 (br s, 2H), 2.19 (s, 3H).

Step 3. Synthesis of 5-(pentafluoro-λ⁶-sulfaneyl)-2H-indazole: To a solution of 2-methyl-4-(pentafluoro-λ⁶-sulfaneyl)aniline (600 mg, 2.57 mmol, 1.00 eq) in AcOH (6.00 mL) was added a solution of NaNO₂ (178 mg, 2.57 mmol, 1.00 eq) in H₂O (2.00 mL) at 0° C. The mixture was stirred at 0° C. for 2 h. The reaction mixture was diluted with water (20 mL) and extracted with EtOAc (2×20 mL). The organic layers were dried over anhydrous Na₂SO₄, filtered and concentrated under reduced pressure. The residue was purified by flash silica gel chromatography (eluent of EtOAc/PE) to afford the title compound. ¹H NMR (400 MHz, CDCl₃) δ ppm 7.56 (d, 1H) 7.82 (dd, 1H) 8.23 (s, 1H) 8.26 (d, 1H).

Step 4. Synthesis of tert-butyl 2-(5-(pentafluoro-λ⁶-sulfaneyl)-2H-indazol-2-yl)acetate: To a solution of 5-(pentafluoro-λ⁶-sulfaneyl)-2H-indazole (150 mg, 614 gmol, 1.00 eq) in DMF (3.00 mL) was added K₂CO₃ (255 mg, 1.84 mmol, 3.00 eq) and tert-butyl 2-bromoacetate (144 mg, 737 μmol, 1.20 eq) at room temperature. Then the mixture was stirred at room temperature for 2 h. The reaction mixture was diluted with water (10 mL), extracted with EtOAc (3×10 mL). The combined organic layers were dried over anhydrous Na₂SO₄, filtered and concentrated under reduced pressure. The residue was purified by flash silica gel chromatography (eluent of EtOAc/PE) to afford the title compound. ¹H NMR (400 MHz, CDCl₃) δ ppm 8.25-8.14 (m, 2H), 7.78-7.71 (m, 1H), 7.67-7.61 (m, 1H), 5.15 (s, 2H), 1.50 (s, 9H). LCMS: 359.1 [M+H]⁺.

Step 5. Synthesis of 2-(5-(pentafluoro-_⁶-sulfaneyl)-2H-indazol-2-yl)acetic acid: A solution of tert-butyl 2-(5-(pentafluoro-λ⁶-sulfaneyl)-2H-indazol-2-yl)acetate (30 mg, 84 μmol, 1 eq) in formic acid (3 mL) was stirred at 50° C. for 2 h. The reaction mixture was concentrated under reduced pressure to afford the title compound, which was used in the next step directly without further purification. LCMS: 244.1 [M+H]⁺.

### Intermediate-26: 3-bromo-2,3-dihydrothiophene 1,1-dioxide

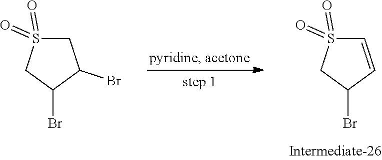 [SMILES: O=S1(=O)CC(Br)C(Br)C1 ; O=S1(=O)C=CC(Br)C1]

Step 1. Synthesis of 3-bromo-2,3-dihydrothiophene 1,1-dioxide: To a solution of 3,4-dibromotetrahydrothiophene 1,1-dioxide (20.0 g, 72.0 mmol, 1.00 eq) in acetone (100 mL) was added pyridine (8.54 g, 0.18 mol, 1.50 eq) dropwise at room temperature. After stirring overnight at room temperature, the resulting mixture was filtered. The filter cake was washed with acetone, and the filtrate was concentrated under reduced pressure to afford the title compound, which was used in the next step without further purification. ¹H NMR (400 MHz, CDCl₃) δ ppm 6.84 (d, 1H), 6.77 (d, 11H), 5.19 (m, 1H), 3.85 (dd, 1H), 3.52 (dd, 1H).

### Intermediate-27: (S)-3-hydroxy-2,3-dihydrothiophene 1,1-dioxide (single isomer)

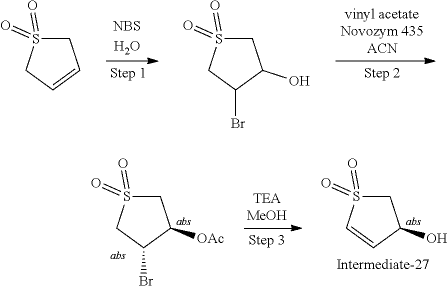 [SMILES: O=S1(=O)CC=CC1 ; O=S1(=O)CC(O)C(Br)C1 ; CC(=O)O[C@@H]1CS(=O)(=O)C[C@H]1Br ; O=S1(=O)C=C[C@H](O)C1]

Step 1. Synthesis of 3-bromo-4-hydroxytetrahydrothiophene 1,1-dioxide: NBS (1150 g, 6.44 mol, 2.00 eq) was added to a mixture of 2,5-dihydrothiophene 1,1-dioxide (380 g, 3.22 mol, 1.00 eq) in H₂O (7.60 L), the mixture was stirred at 70° C. for 3 h. The mixture was cooled to 10° C., during this period, crystallization of the product was observed, the filter cake was collected by filtration, then stirred in DCM/MeOH (10/1, 3 volumes, 30 min, room temperature) and filtered to afford the title compound. ¹H NMR (400 MHz, DMSO-d₆)₆ ppm 6.33 (1H), 4.62-4.52 (2H), 3.94-3.84 (1H), 3.64-3.55 (1H), 3.55-3.45 (1H), 3.16-3.07 (1H).

Step 2. Synthesis of (3R,4S)-4-bromo-1,1-dioxidotetrahydrothiophen-3-yl acetate (single isomer): To a solution of 3-bromo-4-hydroxytetrahydrothiophene 1,1-dioxide (350 g, 1630 mmol, 1.00 eq) in ACN (10.5 L) was added vinyl acetate (700 g, 8140 mmol, 4.99 eq) and Novozym 435 (350 g, 1:1 w:w) and the mixture was stirred for 3 days by overhead (100 rpm) at 30° C. The mixture was filtered, and the filtrate was concentrated and stirred with DCM (4 volumes) for 30 minutes at room temperature. The mixture was filtered, and the filtrate was collected and concentrated. The mixture was dissolved in EtOAc (6 volumes) and then heptane (30 V) was added at room temperature without stirring. After standing for 30 min the filter cake was collected by filtration to afford the title compound. ¹H NMR (300 MHz, DMSO-d₆) δ ppm 5.53 (1H), 4.82 (1H), 3.92 (1H), 3.85-3.73 (1H), 3.67 (1H), 3.38 (1H), 2.09 (3H).

Step 3. Synthesis of (S)-3-hydroxy-2,3-dihydrothiophene 1,1-dioxide (single isomer): TEA (197 g, 1950 mmol, 5.01 eq) was added to a solution of (3R,4S)-4-bromo-1,1-dioxidotetrahydrothiophen-3-yl acetate (100 g, 389 mmol, 1 eq) in MeOH (1000 mL) and the mixture was stirred at 30° C. for 16 h. The mixture was concentrated to 200 mL, and EtOAc (800 mL) was added. The mixture was filtered, and the filtrate was collected. The residue was purified by flash silica gel chromatography (eluent of EtOAc/PE) to afford the title compound. ¹H NMR (300 MHz, CDCl₃) δ ppm 6.78 (1H), 6.72 (1H), 5.15 (1H), 3.63 (1H), 3.20 (1H), 2.70 (1H).

### Intermediate-28: (S)-3-hydroxy-2,3-dihydrothiophene 1,1-dioxide (single isomer)

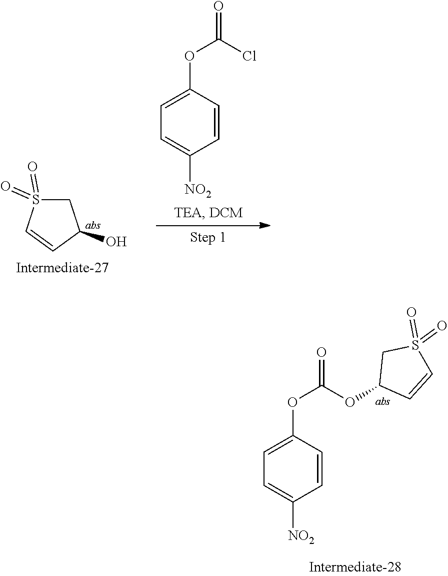 [SMILES: O=S1(=O)C=C[C@H](O)C1 ; O=C(Cl)Oc1ccc([N+](=O)[O-])cc1 ; O=C(Oc1ccc([N+](=O)[O-])cc1)O[C@H]1C=CS(=O)(=O)C1]

Step 1. Synthesis of (S)-1,1-dioxido-2,3-dihydrothiophen-3-yl (4-nitrophenyl) carbonate (single isomer): To a solution of (S)-3-hydroxy-2,3-dihydrothiophene 1,1-dioxide (Intermediate-27) (54.0 g, 402 mmol, 1.00 eq) in DCM (1000 mL) was added 4-nitrophenyl chloroformate (81.1 g, 402 mmol, 1.00 eq) at 0° C. Then TEA (61.0 g, 603 mmol, 1.50 eq) was added and the mixture was stirred at 0° C. for 30 min. 80% DCM was concentrated under vacuum, then the mixture was filtered, and the filter cake was collected and stirred in H₂O (3 volumes) at room temperature. The residue was purified by recrystallization by acetone (5 volumes) to afford the title compound. LCMS: 321.9 [M+Na]⁺.

Alternative procedure for Intermediate-10: (R)-3-(((2-(2-hydroxypropan-2-yl)pyridin-4-yl)methyl)amino)-2,3-dihydrothiophene 1,1-dioxide (single isomer)

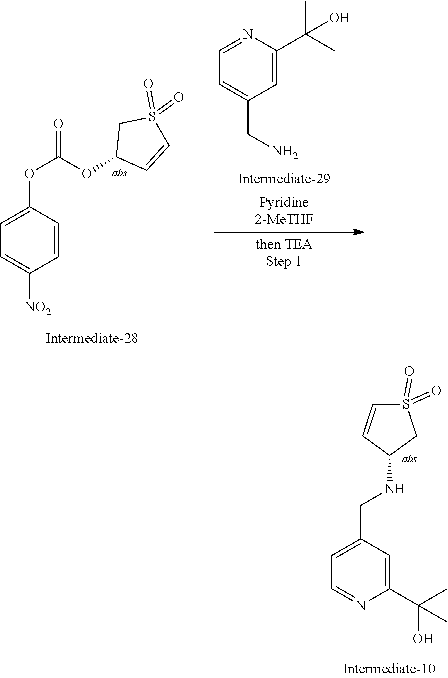

Step 1. Synthesis of (R)-3-(((2-(2-hydroxypropan-2-yl)433yridine-4-yl)methyl)amino)-2,3-dihydrothiophene 1,1-dioxide (single isomer): To a solution of (S)-1,1-dioxido-2,3-dihydrothiophen-3-yl (4-nitrophenyl) carbonate (Intermediate-28) (72.0 g, 241 mmol, 1.00 eq) and 2-(4-(aminomethyl)pyridin-2-yl)propan-2-ol (Intermediate-29) (40.0 g, 241 mmol, 1.00 eq) in 2-MeTHF (800 mL) was added pyridine (19.0 g, 241 mmol, 1.00 eq) at 0° C. The mixture was stirred at 25° C. for 3 h. Then TEA (48.7 g, 481 mmol, 2.00 eq) was added and the mixture was stirred at reflux for 16 h. The mixture was concentrated under vacuum and the residue was purified by flash silica gel chromatography (eluent of MeOH/DCM) to afford the title compound. LCMS: 283.0 [M+H]⁺. ¹H NMR (300 MHz, CDCl₃) δ ppm 8.47 (1H), 7.38 (1H), 7.25-7.17 (1H), 6.77 (1H), 6.70 (1H), 4.93 (1H), 4.28 (1H), 3.99-3.81 (m, 2H), 3.52 (1H), 3.16 (1H), 1.54 (6H).

### Intermediate-29: 2-[4-(aminomethyl)pyridin-2-yl]propan-2-ol

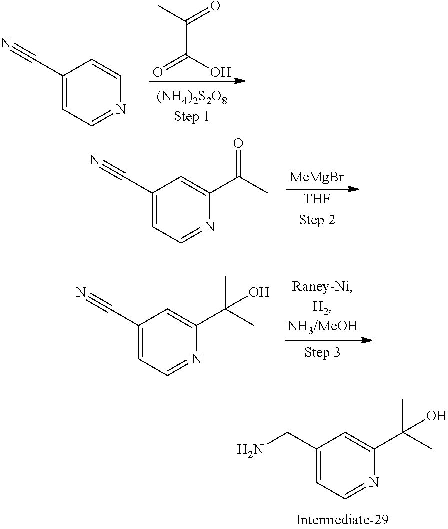

Step 1. Synthesis of 2-acetylisonicotinonitrile: To a solution of pyridine-4-carbonitrile (30.00 g, 288.2 mmol, 1.00 eq), AgNO₃ (3.92 g, 23.1 mmol, 0.08 eq) and 2-oxopropanoic acid (63.44 g, 720.4 mmol, 50.75 mL, 2.50 eq) in DCM (600 mL) and H₂O (120 mL) was added a solution of (NH₄)₂S₂O₈ (98.64 g, 432.2 mmol, 93.94 mL, 1.5 eq) and 98% sulfuric acid (56.52 g, 576.3 mmol, 30.72 mL, 2.00 eq) in H₂O (300 mL) dropwise at 0° C. for 1 h. The mixture was warmed to 25° C. A lot of gas was released, and the temperature rose to 40° C. The reaction was stirred for another 1 h. The mixture was adjusted to pH around 8 with 1 M NaOH (aq.) and then filtered through a celite. The filtrate was extracted with DCM (100 mL*2). Combined extracts were washed with H₂O (100 mL*2) and brine (100 mL), dried over anhydrous Na₂SO₄, filtered and concentrated to dryness. The residue was purified by flash silica gel chromatography (Eluent of EtOAc/PE) to afford the title compound. LCMS: 147.0 [M+H]⁺.

Step 2. 2-(2-hydroxypropan-2-yl)isonicotinonitrile: To a solution of 2-acetylisonicotinonitrile (30.0 g, 205 mmol, 1.00 eq) in THF (300 mL) was added MeMgBr in THE (75.3 mL, 226 mmol, 1.10 eq, 3 M) dropwise at −42~−38° C. during 1 h. The reaction mixture was stirred at −40° C. for another 4 h, then quenched by saturated NH₄Cl (300 mL) at −40° C. and extracted with EtOAc (200 mL*2). Combined extracts were washed with H₂O (200 mL), dried over anhydrous Na₂SO₄ and concentrated to dryness. The residue was purified by flash silica gel chromatography (Eluent of EtOAc/Petroleum ether) to afford the title compound. ¹H NMR (400 MHz, CDCl₃) δ ppm 8.71 (d, 1H), 7.71 (s, 1H), 7.43 (d, 1H), 4.06 (s, 1H), 1.58 (s, 6H).

Step 3. Synthesis of 2-[4-(aminomethyl)pyridin-2-yl]propan-2-ol: To a solution of 2-(2-hydroxypropan-2-yl)isonicotinonitrile (8.00 g, 49.3 mmol, 1.00 eq) in 7 M NH₃/MeOH (200 mL) was added Raney nickel (1.69 g, 19.7 mmol, 0.4 eq) under N₂ atmosphere. The reaction mixture was charged with H₂ three times and stirred at 25° C. for 2 h under H₂ atmosphere at 15 Psi. The mixture was filtered and the filtrate was concentrated in vacuum. The residue was purified by flash silica gel chromatography (eluent of MeOH/DCM) to afford the title compound. LCMS: 167.4 [M+H]⁺.

### Example 2: WRN (BV08) ADP-Glo Assay Protocol

Bovine skin gelatin (BSG), dimethyl sulfoxide (DMSO), Pluronic F-127 and Tris(2-carboxyethyl)phosphine hydrochloride solution (TCEP) were purchased from Sigma-Aldrich at the highest level of purity possible. Bicine buffer solution was purchased from Alfa Aesar and compound NSC-617145 was purchased from Tocris. DNA duplex was synthesized at BGI (Shenzhen, China) and was composed of strand 1 with the sequence 5′-GCACTGGCCGTCGTTTTACGGTCG-3′ (SEQ ID NO.: 1) and strand 2 with the sequence 5′-TCCAAGTAAAACGACGGCCAGTGC-3′ (SEQ ID NO.: 2). DNA strands were annealed by heating to 95° C. for 5 minutes followed by slow cooling to room temperature. Compounds in 100% DMSO (0.1 μl) were spotted into a 384-well white polystyrene Optiplate-384 (Perkin Elmer) assay plate using a LabCyte Echo 550 (Agilent). DMSO (0.1 μl) was added to columns 12, rows A-H and column 24, rows I-P for the maximum signal control. Compound NSC-617145 (0.1 μl) was added to columns 12, rows I-P and 24, rows A-H for the minimum signal control (100% inhibition). Compounds/DMSO were preincubated for 15 minutes at 25° C. with 5 uL 2×WRN (BV08), prepared as described below, in assay buffer containing 20 mM Bicine (pH=7.5), 1 mM MgCl₂, 10 mM KCl, 0.1% Pluronic F-127, 0.005% BSG, 1 mM TCEP. The reaction was initiated by the addition of 5 μl 2× substrate mixture in assay buffer and incubated for 60 minutes at 25° C. The final concentrations of the assay components were 0.15 nM WRN, 5 μM ATP, and 0.1 nM DNA duplex. The final DMSO concentration was 1% and the reference compound concentration (NSC-617145) used for the minimal signal control was 20 μM. The reaction was stopped by the addition of the ADP-Glo Kit components (Promega) as directed and the relative luminescence units (RLU) were read on an Envision 2104 (Perkin Elmer).

### % Inhibition Calculation:

$$\% INH = ( RLU MAX - RLU sample ) / ( RLU MAX - RLU MIN ) ) \times 100$$

Where RLU=relative luminescence units, sample=signal in sample well, and MIN and MAX are the respective minimum and maximum signal controls.

### Four-Parameter IC₅₀ Fit Equation:

$$Y = Bottom + ( Top - Bottom ) / ( 1 + ( IC 50 / X ) \hat{} Hill Slope )$$

Where top and bottom are normally allowed to float but may be fixed at 100 or 0 respectively in a 3-parameter fit. Y is the % inhibition and X is the compound concentration.

### WRN Protein Production

Molecular Biology and virus production. The DNA encoding human Werner helicase (Uniprot Q14191, amino acids 517-1235 with L1074F point mutation) was generated with codon-optimization for E. coli expression and subcloned into the pFastBac vector with a TEV cleavable 8×His tag (WRN-BV08). The baculovirus from the expression plasmid WRN-BV08 was generated from transfection and amplification following the manufacturer's instructions.

Gene sequence of WRN-BV08 [pFastBac1-WRN-(517-1235 L1074F)-TEV-8His]
(SEQ ID NO.: 3)
ATGAACGAGGGCGAAGAAGACGACGACAAGGACTTCCTGTGGCCTGCCCCTAACGA
AGAACAAGTGACATGCCTGAAGATGTACTTCGGACACAGTAGCTTCAAGCCTGTGC
AATGGAAGGTCATCCACTCCGTGCTGGAAGAAAGAAGGGACAACGTGGCTGTGATG
GCTACCGGATACGGTAAGTCCCTGTGCTTCCAGTACCCTCCCGTGTACGTGGGCAAG
ATCGGTCTGGTGATCTCCCCTCTGATCTCTCTGATGGAGGACCAGGTGCTGCAATTG
AAGATGTCCAACATCCCCGCTTGCTTCCTGGGTTCCGCTCAAAGTGAGAACGTGCTG
ACAGACATCAAGCTGGGCAAGTACCGCATCGTGTACGTGACCCCTGAGTACTGCTCC
GGTAACATGGGTCTGCTGCAACAGCTGGAGGCTGACATCGGAATCACCCTGATCGCT
GTGGACGAGGCTCACTGCATCTCCGAGTGGGGACACGACTTCCGCGACTCCTTCCGT
AAGCTGGGATCCTTGAAGACCGCTCTCCCTATGGTGCCTATCGTGGCCCTGACCGCC
ACTGCTTCCTCCTCCATCCGCGAGGACATCGTGCGTTGCCTGAACCTGCGCAACCCT
CAGATCACTTGCACCGGTTTCGACCGCCCTAACTTGTACCTCGAGGTGCGTCGCAAG
ACCGGTAACATCCTCCAGGACCTGCAGCCTTTCCTGGTCAAGACCTCCTCCCACTGG
GAATTTGAGGGCCCTACCATCATCTACTGCCCTTCCCGCAAGATGACCCAGCAAGTC
ACCGGCGAGCTGCGCAAGCTCAACCTCTCCTGCGGTACCTACCACGCTGGTATGTCC
TTCTCCACCCGCAAGGACATCCACCACCGCTTCGTCCGTGACGAAATCCAATGCGTC
ATCGCTACCATCGCTTTCGGAATGGGCATCAACAAGGCTGACATCCGCCAGGTGATC
CACTACGGCGCCCCCAAGGACATGGAATCCTACTACCAGGAAATCGGTCGCGCCGG
TCGCGACGGTCTGCAGTCTTCCTGTCACGTGCTGTGGGCCCCCGCTGACATCAACCT
GAACCGCCACCTGCTGACCGAAATCCGCAACGAGAAGTTCCGCCTGTACAAGCTCA
AGATGATGGCTAAGATGGAGAAGTACCTGCACTCCTCCCGCTGTCGCCGTCAGATCA
TCCTCTCCCACTTCGAGGACAAGCAAGTGCAAAAGGCTAGCCTGGGTATCATGGGC
ACCGAAAAGTGTTGTGACAACTGCCGCTCCCGCCTCGACCACTGCTACTCCATGGAC
GACAGCGAGGACACCTCCTGGGACTTCGGTCCTCAAGCTTTCAAGCTCTTGTCCGCT
GTGGACATCCTGGGCGAGAAGTTCGGTATCGGTCTCCCCATCCTCTTCCTGCGTGGT
AGCAACTCCCAACGCCTGGCTGACCAGTACCGCCGCCACTCCCTCTTCGGTACCGGT
AAGGACCAGACCGAGTCCTGGTGGAAGGCTTTCTCTCGCCAACTGATCACCGAAGG
TTTCCTGGTGGAGGTGTCCCGCTACAACAAGTTCATGAAGATCTGCGCTCTCACTAA
GAAGGGAAGGAACTGGCTGCACAAGGCTAACACTGAGTCCCAATCCCTCATCCTGC
AGGCTAACGAGGAGCTGTGCCCTAAGAAGTTCCTGCTGCCTTCCTCCAAGACCGTGT
CCTCCGGAACAAAGGAACACTGCTACAACCAAGTCCCTGTGGAGCTCTCCACCGAG
AAGAAGTCCAACCTGGAGAAGCTGTACAGCTACAAGCCTTGCGACAAGATCAGCTC
CGGTTCCAACATCAGCAAGAAGTCCATCATGGTGCAATCCCCTGAAAAGGCCTACTC
CAGCTCCCAACCTGTCATCTCCGCTCAAGAGCAAGAGACCCAGATCGTGCTGTACGG
TAAGCTGGTCGAAGCCCGCCAAAAGCACGCTAACAAGATGGACGTCCCTCCCGCTA
TCCTCGCCACCAACAAGATCCTCGTGGATATGGCTAAGATGCGCCCCACCACCGTCG
AGAACGTGAAGCGCATCGACGGTGTCTCCGAGGGTAAGGCCGCTATGCTGGCTCCT
CTGCTGGAAGTGATCAAGCACTTCTGCCAGACCAACTCCGTGCAGACCGACCTGTTC
AGTAGTGAGAACCTGTACTTCCAAGGCCACCATCATCATCATCATCACCACTAA
Protein sequence of WRN-BV08 [pFastBac1-WRN-(517-1235 L1074F)-TEV-8His]
(SEQ ID NO.: 4)
MNEGEEDDDKDFLWPAPNEEQVTCLKMYFGHSSFKPVQWKVIHSVLEERRDNVAVMA
TGYGKSLCFQYPPVYVGKIGLVISPLISLMEDQVLQLKMSNIPACFLGSAQSENVLTDIKL
GKYRIVYVTPEYCSGNMGLLQQLEADIGITLIAVDEAHCISEWGHDFRDSFRKLGSLKTA
LPMVPIVALTATASSSIREDIVRCLNLRNPQITCTGFDRPNLYLEVRRKTGNILQDLQPFL
VKTSSHWEFEGPTIIYCPSRKMTQQVTGELRKLNLSCGTYHAGMSFSTRKDIHHRFVRD
EIQCVIATIAFGMGINKADIRQVIHYGAPKDMESYYQEIGRAGRDGLQSSCHVLWAPADI
NLNRHLLTEIRNEKFRLYKLKMMAKMEKYLHSSRCRRQIILSHFEDKQVQKASLGIMGT
EKCCDNCRSRLDHCYSMDDSEDTSWDFGPQAFKLLSAVDILGEKFGIGLPILFLRGSNSQ
RLADQYRRHSLFGTGKDQTESWWKAFSRQLITEGFLVEVSRYNKFMKICALTKKGRNW
LHKANTESQSLILQANEELCPKKFLLPSSKTVSSGTKEHCYNQVPVELSTEKKSNLEKLY
SYKPCDKISSGSNISKKSIMVQSPEKAYSSSQPVISAQEQETQIVLYGKLVEARQKHANK
MDVPPAILATNKILVDMAKMRPTTVENVKRIDGVSEGKAAMLAPLLEVIKHFCQTNSV
QTDLFSSENLYFQGHHHHHHHH

Sf9 cells grown in SF900II media were infected with 1:200 WRN-BV08 P2 virus and incubated for protein expression for 72 h at 27° C. The WRN protein was purified using the following protocol. The cell pellets were thawed and resuspended in buffer A (50 mM Tris, pH 7.5, 500 mM NaCl, 1 mM TCEP, 10% Glycerol) supplemented with 0.5% CHAPS, 1 mM PMSF, 1 μg/mL Leupeptin, 1 μg/mL Pepstatin, and the Pierce Universal Nuclease and cocktail tablet. Cleared lysates were loaded onto a Ni Sepharose™ excel column and washed with buffer A and bound protein was eluted with buffer A supplemented with 300 mM imidazole. The eluted protein was dialyzed against buffer A and digested by His-tagged TEV (1:5 ratio) overnight at 4° C. ZnCl₂ was added into the sample at final 15 μM before loading onto a second Ni Sepharose™ excel column. Untagged WRN protein was eluted from the column with buffer A supplemented with 20 mM imidazole, dialyzed overnight into buffer B (50 mM Tris, pH 7.5, 1 mM TCEP, 10% Glycerol) supplemented with 150 mM NaCl and loaded onto a Heparin column. Proteins were eluted with a step gradient of buffer B supplemented with 150 mM, 200 mM, 300 mM and 500 mM NaCl. WRN containing fractions were pooled and concentrated prior to loading on to size exclusion chromatography using a HiLoad 16/600 Superdex™ 200 pg column (GE Healthcare) in buffer C (20 mM HEPES, pH 7.5, 250 mM NaCl, 0.25 mM TCEP, 2.5% Glycerol).

The resultant IC₅₀ results obtained for the tested compounds are shown below in Table 3. Compounds with an IC₅₀ less than or equal to 0.5 μM are designated as “A.” Compounds with an IC₅₀ greater than 0.5 μM and less than or equal to 5.0 μM are designated as “B.” Compounds with an IC₅₀ greater than 5.0 μM and less than or equal to 10.0 μM are designated as “C.” Compounds with an IC₅₀ greater than 10.0 μM and less than or equal to 50.0 μM are designated as “D.” Compounds with an IC₅₀ greater than 50.0 μM are designated as “E.”

TABLE 3

| Cmpd. No. | ADP-Glo_hWRN_IC50 [μM] |
|---|---|
| I-2 | A |
| I-3 | A |
| I-5 | A |
| I-6 | A |
| I-7 | A |
| I-8 | A |
| I-13 | A |
| I-16 | A |
| I-22 | A |
| I-36 | A |
| I-38 | A |
| I-48 | A |
| I-53 | A |
| I-50 | A |
| I-65 | A |
| I-81 | A |
| I-79 | A |
| I-103 | B |
| I-109 | B |
| II-1 | B |
| I-147 | B |
| I-233 | C |
| I-194 | C |
| I-269 | D |
| I-321 | D |
| I-566 | E |
| I-1 | A |
| I-12 | A |
| I-4 | A |
| I-10 | A |
| I-9 | A |
| I-11 | A |
| I-14 | A |
| I-19 | A |
| I-18 | A |
| I-15 | A |
| I-21 | A |
| I-17 | A |
| I-27 | A |
| I-24 | A |
| I-25 | A |
| I-20 | A |
| I-23 | A |
| I-40 | A |
| I-26 | A |
| I-28 | A |
| I-35 | A |
| I-32 | A |
| I-34 | A |
| I-30 | A |
| I-31 | A |
| I-37 | A |
| I-33 | A |
| I-42 | A |
| I-43 | A |
| I-29 | A |
| I-39 | A |
| I-45 | A |
| I-41 | A |
| I-46 | A |
| I-51 | A |
| I-60 | A |
| I-49 | A |
| I-58 | A |
| I-47 | A |
| I-56 | A |
| I-55 | A |
| I-52 | A |
| I-62 | A |
| I-44 | A |
| I-57 | A |
| I-70 | A |
| I-54 | A |
| I-59 | A |
| I-61 | A |
| I-68 | A |
| I-80 | A |
| I-67 | A |
| I-74 | A |
| I-63 | A |
| I-66 | A |
| I-64 | A |
| I-75 | A |
| I-72 | A |
| I-69 | A |
| I-73 | A |
| I-76 | A |
| I-71 | A |
| I-78 | A |
| I-82 | A |
| I-77 | A |
| I-85 | A |
| I-83 | A |
| I-92 | B |
| I-84 | B |
| I-87 | B |
| I-86 | B |
| I-93 | B |
| I-105 | B |
| I-91 | B |
| I-101 | B |
| I-97 | B |
| I-94 | B |
| I-95 | B |
| I-88 | B |
| I-96 | B |
| I-99 | B |
| I-90 | B |
| I-98 | B |
| I-110 | B |
| I-104 | B |
| I-89 | B |
| I-112 | B |
| I-102 | B |
| I-100 | B |
| I-107 | B |
| I-106 | B |
| I-117 | B |
| I-115 | B |
| I-108 | B |
| I-111 | B |
| I-114 | B |
| I-122 | B |
| I-119 | B |
| I-118 | B |
| I-127 | B |
| I-120 | B |
| I-123 | B |
| I-121 | B |
| I-136 | B |
| I-130 | B |
| I-113 | B |
| I-132 | B |
| I-126 | B |
| I-129 | B |
| I-124 | B |
| I-131 | B |
| I-141 | B |
| I-133 | B |
| I-125 | B |
| I-140 | B |
| I-134 | B |
| I-139 | B |
| I-144 | B |
| I-151 | B |
| I-142 | B |
| I-158 | B |
| I-137 | B |
| I-143 | B |
| I-138 | B |
| I-146 | B |
| I-145 | B |
| I-164 | B |
| I-116 | B |
| I-152 | B |
| I-157 | B |
| I-148 | B |
| I-149 | B |
| I-166 | B |
| I-153 | B |
| I-161 | B |
| I-154 | B |
| I-150 | B |
| I-156 | B |
| I-155 | B |
| I-162 | B |
| I-168 | B |
| I-169 | B |
| I-167 | B |
| I-163 | B |
| I-135 | B |
| I-160 | B |
| I-177 | B |
| I-170 | B |
| I-174 | B |
| I-181 | B |
| I-175 | B |
| I-173 | B |
| I-186 | B |
| I-159 | B |
| I-165 | B |
| I-182 | B |
| I-230 | B |
| I-128 | B |
| I-297 | B |
| I-180 | B |
| I-172 | B |
| I-179 | B |
| I-171 | B |
| I-178 | B |
| I-184 | B |
| I-183 | B |
| I-188 | B |
| I-191 | B |
| I-190 | B |
| I-187 | B |
| I-189 | B |
| II-2 | B |
| I-195 | B |
| I-176 | B |
| I-193 | B |
| I-185 | B |
| I-200 | B |
| I-238 | B |
| I-203 | B |
| I-201 | B |
| I-215 | B |
| I-202 | B |
| I-192 | B |
| I-199 | B |
| I-196 | B |
| I-209 | B |
| I-208 | B |
| I-204 | B |
| I-197 | B |
| I-217 | B |
| I-207 | B |
| I-198 | B |
| I-211 | B |
| I-206 | B |
| I-213 | B |
| I-205 | B |
| I-212 | B |
| I-221 | B |
| I-210 | B |
| I-218 | C |
| I-219 | C |
| I-227 | C |
| I-220 | C |
| I-223 | C |
| I-216 | C |
| I-226 | C |
| I-222 | C |
| I-224 | C |
| I-228 | C |
| I-225 | C |
| I-231 | C |
| I-236 | C |
| I-234 | C |
| I-273 | C |
| I-237 | C |
| I-229 | C |
| I-232 | C |
| I-245 | C |
| I-249 | C |
| I-243 | C |
| I-247 | C |
| I-239 | C |
| I-244 | C |
| I-241 | C |
| I-235 | C |
| I-254 | C |
| I-242 | C |
| I-240 | C |
| I-246 | C |
| I-290 | C |
| I-248 | C |
| I-260 | C |
| I-258 | C |
| I-262 | C |
| I-252 | C |
| I-255 | C |
| I-251 | C |
| I-263 | C |
| I-264 | C |
| I-261 | C |
| I-256 | C |
| I-257 | C |
| I-259 | C |
| I-285 | C |
| I-270 | C |
| I-265 | C |
| I-271 | C |
| I-277 | C |
| I-266 | C |
| I-250 | C |
| I-268 | C |
| I-276 | C |
| I-279 | C |
| I-287 | C |
| I-272 | C |
| I-274 | C |
| I-289 | D |
| I-280 | D |
| I-292 | D |
| I-278 | D |
| I-284 | D |
| I-293 | D |
| I-291 | D |
| I-281 | D |
| I-275 | D |
| I-253 | D |
| I-283 | D |
| I-288 | D |
| I-286 | D |
| I-282 | D |
| I-294 | D |
| I-296 | D |
| I-302 | D |
| I-295 | D |
| I-307 | D |
| I-298 | D |
| I-310 | D |
| I-301 | D |
| I-329 | D |
| I-303 | D |
| I-308 | D |
| II-3 | D |
| I-313 | D |
| I-306 | D |
| I-267 | D |
| I-318 | D |
| I-299 | D |
| I-305 | D |
| I-309 | D |
| I-300 | D |
| I-327 | D |
| I-304 | D |
| I-316 | D |
| I-314 | D |
| I-315 | D |
| I-325 | D |
| I-320 | D |
| I-322 | D |
| I-340 | D |
| I-331 | D |
| I-323 | D |
| I-317 | D |
| I-319 | D |
| II-4 | D |
| I-324 | D |
| I-335 | D |
| I-330 | D |
| I-338 | D |
| I-326 | D |
| I-339 | D |
| I-337 | D |
| I-328 | D |
| I-332 | D |
| I-343 | D |
| I-347 | D |
| I-334 | D |
| I-350 | D |
| I-348 | D |
| I-336 | D |
| I-349 | D |
| I-359 | D |
| I-345 | D |
| I-342 | D |
| I-341 | D |
| I-355 | D |
| I-353 | D |
| I-354 | D |
| I-351 | D |
| I-361 | D |
| I-352 | D |
| I-357 | D |
| I-360 | D |
| I-365 | D |
| I-356 | D |
| I-367 | D |
| I-363 | D |
| I-358 | D |
| I-368 | D |
| I-344 | D |
| I-364 | D |
| I-369 | D |
| I-469 | E |
| I-370 | E |
| I-371 | E |
| I-372 | E |
| I-373 | E |
| I-362 | E |
| I-374 | E |
| I-375 | E |
| I-376 | E |
| I-377 | E |
| I-378 | E |
| I-379 | E |
| I-380 | E |
| I-381 | E |
| I-382 | E |
| I-383 | E |
| I-384 | E |
| I-385 | E |
| I-386 | E |
| I-388 | E |
| I-389 | E |
| I-390 | E |
| I-391 | E |
| I-393 | E |
| I-394 | E |
| I-396 | E |
| I-397 | E |
| I-311 | E |
| I-399 | E |
| I-400 | E |
| I-312 | E |
| II-7 | E |
| I-401 | E |
| I-402 | E |
| I-403 | E |
| I-404 | E |
| I-405 | E |
| I-366 | E |
| I-406 | E |
| I-407 | E |
| I-408 | E |
| I-409 | E |
| I-333 | E |
| I-410 | E |
| I-412 | E |
| I-413 | E |
| I-414 | E |
| I-416 | E |
| I-417 | E |
| I-418 | E |
| I-419 | E |
| I-420 | E |
| I-421 | E |
| I-422 | E |
| I-423 | E |
| I-424 | E |
| I-425 | E |
| I-427 | E |
| I-428 | E |
| I-429 | E |
| I-430 | E |
| I-431 | E |
| I-432 | E |
| I-433 | E |
| I-434 | E |
| I-435 | E |
| I-436 | E |
| I-437 | E |
| I-438 | E |
| I-439 | E |
| I-440 | E |
| I-441 | E |
| I-442 | E |
| I-443 | E |
| I-444 | E |
| I-445 | E |
| I-446 | E |
| I-448 | E |
| I-448 | E |
| I-449 | E |
| I-450 | E |
| I-451 | E |
| I-452 | E |
| I-453 | E |
| I-454 | E |
| I-455 | E |
| I-456 | E |
| I-457 | E |
| I-458 | E |
| I-460 | E |
| I-462 | E |
| I-461 | E |
| I-463 | E |
| I-464 | E |
| I-466 | E |
| I-470 | E |
| I-467 | E |
| I-471 | E |
| I-479 | E |
| I-472 | E |
| I-477 | E |
| I-474 | E |
| I-475 | E |
| I-476 | E |
| I-465 | E |
| I-478 | E |
| I-481 | E |
| I-481 | E |
| I-482 | E |
| I-483 | E |
| I-485 | E |
| I-486 | E |
| I-487 | E |
| I-488 | E |
| I-490 | E |
| I-492 | E |
| I-493 | E |
| I-494 | E |
| I-495 | E |
| I-496 | E |
| I-497 | E |
| I-498 | E |
| I-499 | E |
| I-500 | E |
| I-501 | E |
| I-502 | E |
| I-503 | E |
| I-468 | E |
| I-506 | E |
| I-507 | E |
| I-508 | E |
| I-510 | E |
| I-511 | E |
| I-513 | E |
| I-514 | E |
| I-516 | E |
| I-519 | E |
| I-520 | E |
| I-521 | E |
| I-522 | E |
| I-524 | E |
| I-525 | E |
| I-526 | E |
| I-527 | E |
| I-484 | E |
| I-528 | E |
| I-529 | E |
| I-530 | E |
| I-532 | E |
| I-533 | E |
| I-534 | E |
| I-535 | E |
| I-536 | E |
| I-538 | E |
| I-539 | E |
| I-540 | E |
| I-541 | E |
| I-542 | E |
| I-543 | E |
| I-544 | E |
| I-545 | E |
| I-546 | E |
| I-547 | E |
| I-548 | E |
| I-550 | E |
| II-6 | E |
| I-551 | E |
| I-552 | E |
| I-553 | E |
| I-554 | E |
| I-555 | E |
| I-556 | E |
| I-557 | E |
| I-558 | E |
| I-559 | E |
| I-560 | E |
| I-561 | E |
| I-562 | E |
| I-563 | E |
| I-564 | E |
| I-565 | E |
| I-567 | E |
| I-568 | E |
| I-569 | E |
| I-570 | E |
| I-571 | E |
| I-572 | E |
| I-573 | E |
| I-574 | E |
| I-575 | E |
| I-577 | A |
| I-578 | D |
| I-579 | E |
| I-580 | E |
| I-581 | E |
| I-582 | E |
| I-583 | E |
| I-584 | E |
| I-585 | E |
| I-586 | E |
| I-587 | D |
| I-588 | D |
| I-589 | B |
| I-590 | C |
| I-591 | B |
| I-592 | D |
| I-593 | E |
| I-594 | B |
| I-595 | B |
| I-596 | D |
| I-597 | E |
| I-598 | A |
| I-599 | A |
| I-600 | B |
| I-601 | B |
| I-602 | A |
| I-603 | A |
| I-604 | B |
| I-605 | B |
| I-606 | A |
| I-607 | A |
| I-608 | D |
| I-609 | E |
| I-610 | E |
| I-611 | E |
| I-612 | A |
| I-613 | E |
| I-614 | E |
| I-615 | E |
| I-616 | E |
| I-617 | E |
| I-618 | E |
| I-619 | E |
| I-620 | E |
| I-621 | E |
| I-622 | E |
| I-623 | B |
| I-624 | B |
| I-625 | A |
| I-626 | A |
| I-627 | E |
| I-628 | A |
| I-629 | B |
| I-630 | E |
| I-631 | E |
| I-632 | A |
| I-633 | D |
| I-634 | C |
| I-635 | B |
| I-636 | D |
| I-637 | D |
| I-638 | A |
| I-639 | A |
| I-640 | E |
| I-641 | A |
| I-642 | E |
| I-643 | D |
| I-644 | B |
| I-645 | B |
| I-646 | C |
| I-647 | E |
| I-648 | C |
| I-649 | E |
| I-650 | E |
| I-651 | E |
| I-652 | E |
| I-653 | E |
| I-654 | E |
| I-655 | E |
| I-656 | E |
| I-657 | B |
| I-658 | E |
| I-659 | A |
| I-660 | A |
| I-661 | A |
| I-662 | A |
| I-663 | B |
| I-664 | A |
| I-665 | D |
| I-666 | C |
| I-667 | B |
| I-668 | C |
| I-669 | A |
| I-670 | B |
| I-671 | A |
| I-672 | C |
| I-673 | A |
| I-674 | C |
| I-675 | D |
| I-676 | A |
| I-677 | D |
| I-678 | E |
| I-679 | B |
| I-680 | E |
| I-681 | A |
| I-682 | A |
| I-683 | A |
| I-684 | B |
| I-685 | A |
| I-686 | B |
| I-687 | C |
| I-688 | A |
| I-689 | A |
| I-690 | B |
| I-691 | B |
| I-692 | D |
| I-693 | A |
| I-694 | A |
| I-695 | A |
| I-696 | E |
| I-697 | E |
| I-698 | B |
| I-699 | B |
| I-700 | C |
| I-701 | A |
| I-702 | B |
| I-703 | A |
| I-704 | A |
| I-705 | B |
| I-706 | B |
| I-707 | B |
| I-708 | C |
| I-709 | A |
| I-710 | E |
| I-711 | E |
| I-712 | B |
| I-713 | D |
| I-714 | E |
| I-715 | E |
| I-716 | E |
| I-717 | E |
| I-718 | A |
| I-719 | A |
| I-720 | B |
| I-721 | B |
| I-722 | E |
| I-723 | A |
| I-724 | A |
| I-725 | B |
| I-726 | A |
| I-727 | B |
| I-728 | B |
| I-729 | E |
| I-730 | A |
| I-731 | E |
| I-732 | A |
| I-733 | A |
| I-734 | E |
| I-735 | B |
| I-736 | E |
| I-737 | A |
| I-738 | E |
| I-739 | A |
| I-740 | B |
| I-741 | E |
| I-742 | B |
| I-743 | E |
| I-744 | B |
| I-745 | B |
| I-746 | A |
| I-747 | C |
| I-748 | A |
| I-749 | B |
| I-750 | A |
| I-751 | D |
| I-752 | C |
| I-753 | B |
| I-754 | B |
| I-755 | E |
| I-756 | E |
| I-757 | E |
| I-758 | B |
| I-759 | B |
| I-760 | A |
| I-761 | A |
| I-762 | B |
| I-763 | A |
| I-764 | A |
| I-765 | B |
| I-766 | C |
| I-767 | A |
| I-768 | A |
| I-769 | A |
| I-770 | B |
| I-771 | A |
| I-772 | A |
| I-773 | A |
| I-774 | D |
| I-775 | B |
| I-776 | A |
| I-777 | A |
| I-778 | B |
| I-779 | B |
| I-780 | B |
| I-781 | D |
| I-782 | A |
| I-783 | A |
| I-784 | C |
| I-785 | E |
| I-786 | B |
| I-787 | B |
| I-788 | C |
| I-789 | A |
| I-790 | B |
| I-791 | B |
| I-792 | B |
| I-793 | B |
| I-794 | B |
| I-795 | B |
| I-796 | B |
| I-797 | A |
| I-798 | A |
| I-799 | B |
| I-800 | A |
| I-801 | A |
| I-802 | B |
| I-803 | E |
| I-804 | A |
| I-805 | A |
| I-806 | B |
| I-807 | C |
| I-808 | B |
| I-809 | B |
| I-810 | B |
| I-811 | B |
| I-812 | A |
| I-813 | A |
| I-814 | B |
| I-815 | A |
| I-816 | E |
| I-817 | B |
| I-819 | D |
| I-820 | A |
| I-821 | B |
| I-822 | A |
| I-823 | E |
| I-824 | A |
| I-825 | A |
| I-826 | A |
| I-827 | A |
| I-828 | B |
| I-829 | A |
| I-830 | C |
| I-831 | B |
| I-832 | A |
| I-833 | D |
| I-834 | E |
| I-835 | C |

LENGTHY TABLES
The patent application contains a lengthy table section. A copy of the table is available in electronic form from the USPTO web site (https://seqdata.uspto.gov/docdetail?docId=US20260297070A1). An electronic copy of the table will also be available from the USPTO upon request and payment of the fee set forth in 37 CFR 1.19(b)(3).

We claim:

1. A compound, or pharmaceutically acceptable salt thereof, according to Formula I: 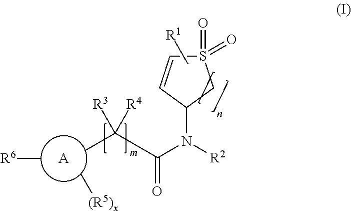
wherein
Ring A is a bivalent cyclic group selected from:
a 3-8 membered saturated or partially unsaturated monocyclic carbocyclylene,
phenylene,
a 4-8 membered saturated or partially unsaturated monocyclic heterocyclylene having 1-2 heteroatoms independently selected from nitrogen, oxygen, and sulfur,
a 5-6 membered monocyclic heteroarylene having 1-4 heteroatoms independently selected from nitrogen, oxygen, and sulfur,
an 8-10 membered bicyclic heteroarylene ring having 1-5 heteroatoms independently selected from nitrogen, oxygen, and sulfur,
a 7-12 membered saturated or partially unsaturated bicyclic heterocyclylene having 1-4 heteroatoms independently selected from nitrogen, oxygen, and sulfur,
a 5-12 membered saturated or partially unsaturated bicyclic carbocyclylene that is optionally bridged or spirocyclic, and
a 5-12 membered saturated or partially unsaturated bicyclic heterocyclylene that is optionally bridged or spirocyclic having 1-4 heteroatoms independently selected from nitrogen, oxygen, and sulfur;
R¹ is hydrogen, halogen, —CN, or an optionally substituted C₁₋₆ aliphatic group;
R² is a C₁₋₆ aliphatic group, a C₁₋₆ aliphatic-Cy′ group, or a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 7-12 membered saturated or partially unsaturated bicyclic heterocyclic ring having 1-4 heteroatoms independently selected from nitrogen, oxygen, and sulfur,
a 5-12 membered saturated or partially unsaturated bicyclic carbocyclic ring that is optionally bridged or spirocyclic, and
a 5-12 membered saturated or partially unsaturated bicyclic heterocyclic ring that is optionally bridged or spirocyclic having 1-4 heteroatoms independently selected from nitrogen, oxygen, and sulfur,
wherein R² is substituted with y instances of R^{A};
R³ and R⁴ are each independently selected at each occurrence from hydrogen, halogen, —CN, —NO₂, —OR, —SR, —NR₂, —NRC(O)R—S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group optionally substituted with —C(O)NR₂ or NR₂, a C₁₋₆ aliphatic-Cy″ group, or Cy″; wherein when m is 2 or 3, two adjacent instances of CR³R⁴ are optionally combined to forma carbon-carbon double bond of the formula —C(R⁴)═C(R⁴)—;
each instance of R⁵ is independently selected from hydrogen, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, a C₁₋₆ aliphatic group substituted with z instances of R^{B}, a C₁₋₆ aliphatic-Cy′″ group substituted with z instances of R^{B}, and Cy′″ substituted with z instances of R^{B}; or two instances of R⁵ on the same atom together form oxo; or two instances of R⁵ on adjacent atoms of Ring A, taken together with said adjacent atoms, form a phenyl ring fused to Ring A, wherein said phenyl ring is substituted with 0-4 independently selected halogens;
R⁶ is hydrogen, halogen, a C₁₋₆ aliphatic group, —SF₅, —OR, —SR, —S(O)₂NR₂, —NR₂, —S(O)₂R, —SiR₃, or a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic,
a 5-12 membered saturated or partially unsaturated bicyclic heterocyclic ring that is optionally bridged or spirocyclic having 1-4 heteroatoms independently selected from nitrogen, oxygen, and sulfur,
wherein the C₁₋₆ aliphatic group and cyclic group are substituted with z instances of R^{B};
each instance of R^{A} is independently selected from hydrogen; a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; —N(R)S(O)₂R; two instances of R^{A} on the same atom together form oxo; and a cyclic group selected from:
3-8 membered saturated or partially unsaturated monocyclic carbocyclic ring;
a 5-6 membered monocyclic heteroaromatic ring having 1-4 heteroatoms independently selected from nitrogen, oxygen, and sulfur; and
a 3-8 membered saturated or partially unsaturated monocyclic heterocyclic ring having 1-2 heteroatoms independently selected from nitrogen, oxygen, and sulfur,
wherein the cyclic group is optionally substituted with 1, 2, or 3 groups independently selected from —OR, —CR₂OR, and halogen;
each instance of R^{B} is independently selected from hydrogen; a C₁₋₆ aliphatic group optionally substituted with 1, 2, or 3 groups independently selected from halogen, —CN, —NR₂, and —OR; halogen; —CN; —NO₂; —OR; —SR; —NR₂; —S(O)₂R; —S(O)₂NR₂; —S(O)R; —S(O)NR₂; —C(O)R; —C(O)OR; —C(O)NR₂; —C(O)N(R)OR; —OC(O)R; —OC(O)NR₂; —N(R)C(O)OR; —N(R)C(O)R; —N(R)C(O)NR₂; —N(R)C(NR)NR₂; —N(R)S(O)₂NR₂; —N(R)S(O)₂R; phenyl; and two instances of R^{B} on the same atom together form oxo;
each of Cy′, Cy″, and Cy′″ is independently an optionally substituted cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic,
a 5-12 membered saturated or partially unsaturated bicyclic heterocyclic ring that is optionally bridged or spirocyclic having 1-4 heteroatoms independently selected from nitrogen, oxygen, and sulfur, and
an 8-10 membered bicyclic aromatic carbocyclic ring;
each R is independently hydrogen, —C(O)N(CH₃)₂, —C(O)₂CH₃, —C(O)₂C(CH₃)₃, —C(O)₂CH(CH₃)₂, —S(O)₂CH₃, an optionally substituted C₁₋₆ aliphatic group, an optionally substituted cyclic group selected from:
phenyl,
an optionally substituted 3-7 membered saturated or partially unsaturated carbocyclic ring,
an optionally substituted 3-7 membered saturated or partially unsaturated heterocyclic ring having 1-2 heteroatoms independently selected from nitrogen, oxygen, and sulfur,
or an optionally substituted 5-6 membered heteroaryl ring having 1-4 heteroatoms independently selected from nitrogen, oxygen, and sulfur; and/or
two R groups on the same atom are taken together with the same atom to form a cyclic group selected from:
an optionally substituted 4-7 membered saturated or partially unsaturated carbocycyl,
an optionally substituted 4-7 membered saturated or partially unsaturated heterocyclyl having 1-3 heteroatoms independently selected from nitrogen, oxygen, and sulfur, and
an optionally substituted 5-6 membered heteroaryl ring having 1-3 heteroatoms independently selected from nitrogen, oxygen, and sulfur;
x is 0, 1, 2, 3, or 4;
m is 0, 1, 2, or 3;
n is 1 or 2;
y is 0, 1, 2, or 3; and
z is 0, 1, 2, or 3.

2. The compound, or pharmaceutically acceptable salt thereof, of claim 1, wherein Ring A is:
phenylene, or
a 5-6 membered monocyclic heteroarylene having 1-4 heteroatoms independently selected from nitrogen, oxygen, and sulfur;
wherein said phenylene or 5-6 membered monocyclic heteroarylene is substituted with x instances of R⁵.

3. The compound, or pharmaceutically acceptable salt thereof, of claim 1, wherein 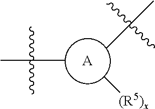
is selected from 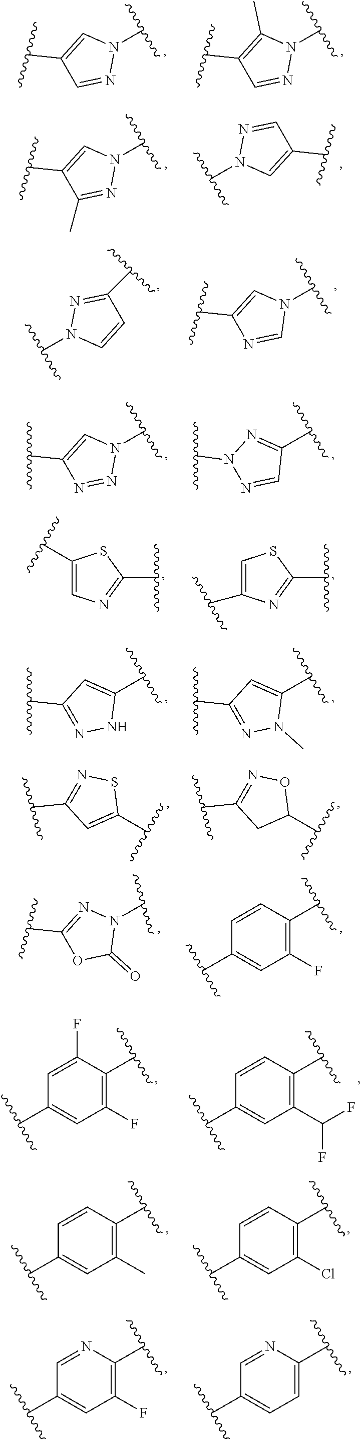 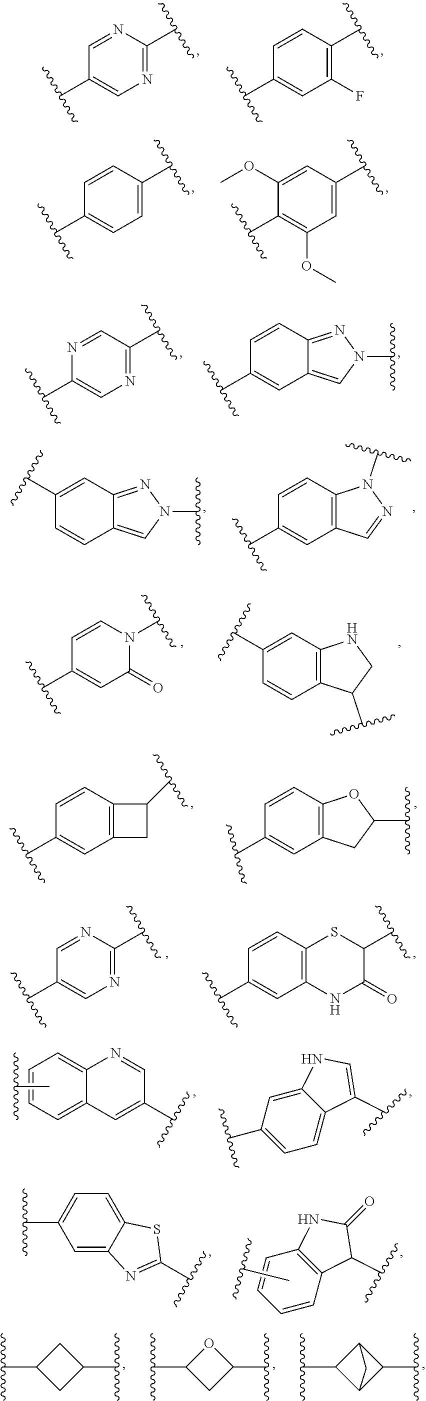 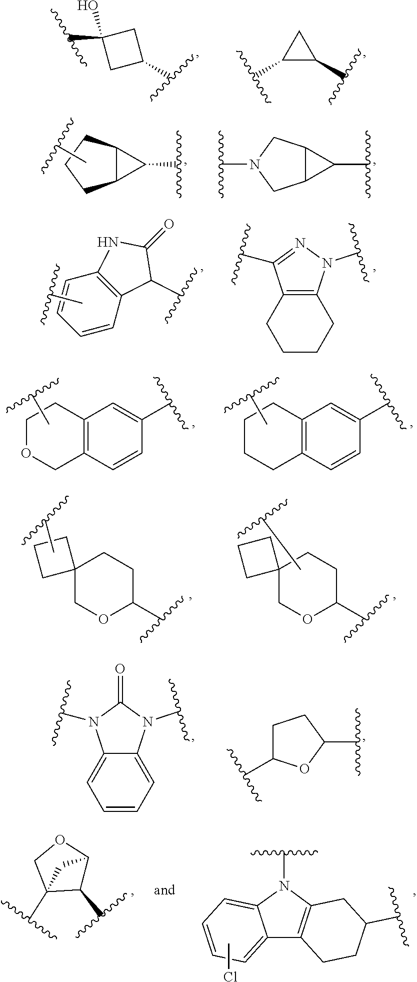
wherein the left-hand [symbol] is attached to R⁶.

4. The compound, or pharmaceutically acceptable salt thereof, of any one of claims 1-3, wherein R¹ is hydrogen.

5. The compound, or pharmaceutically acceptable salt thereof, of any one of claims 1-4, wherein R² is a C₁₋₆ aliphatic-Cy′ group substituted with y instances of R^{A}.

6. The compound, or pharmaceutically acceptable salt thereof, of any one of claims 1-5, wherein R² is —CH₂—Cy′.

7. The compound, or pharmaceutically acceptable salt thereof, of any one of claims 1-4, wherein R² is

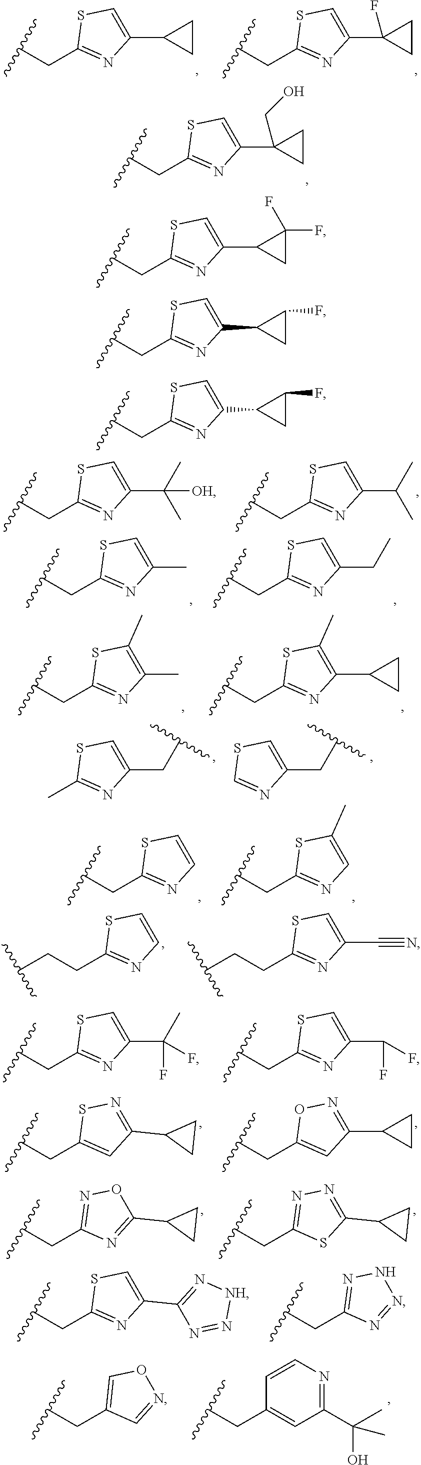

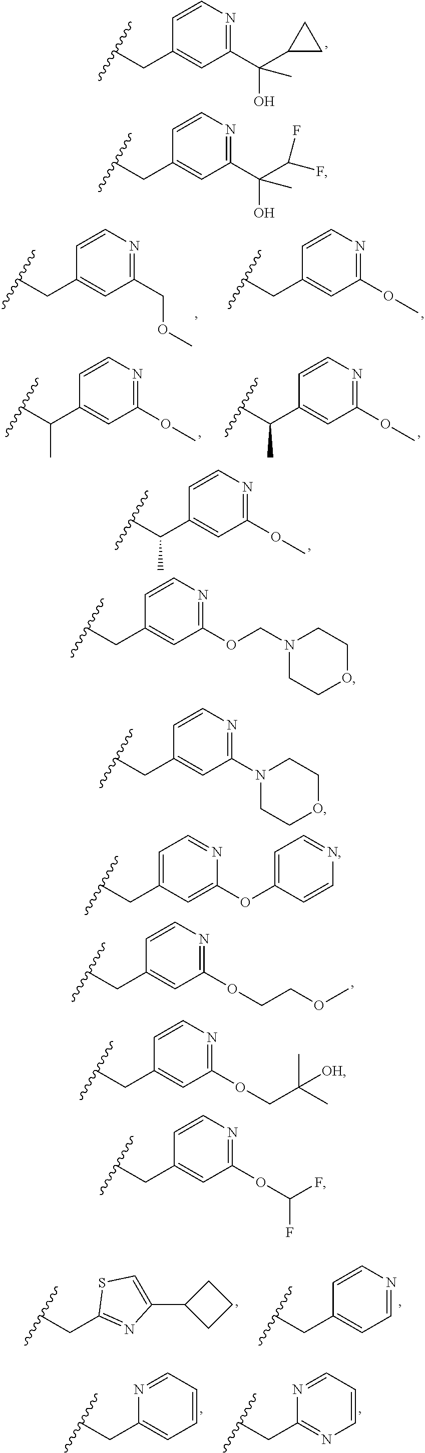

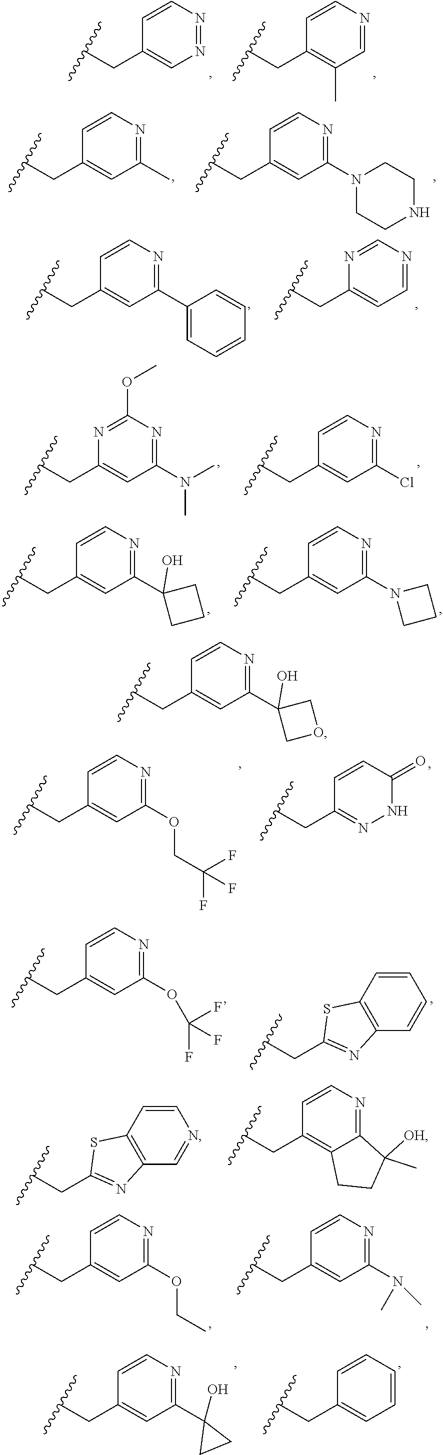

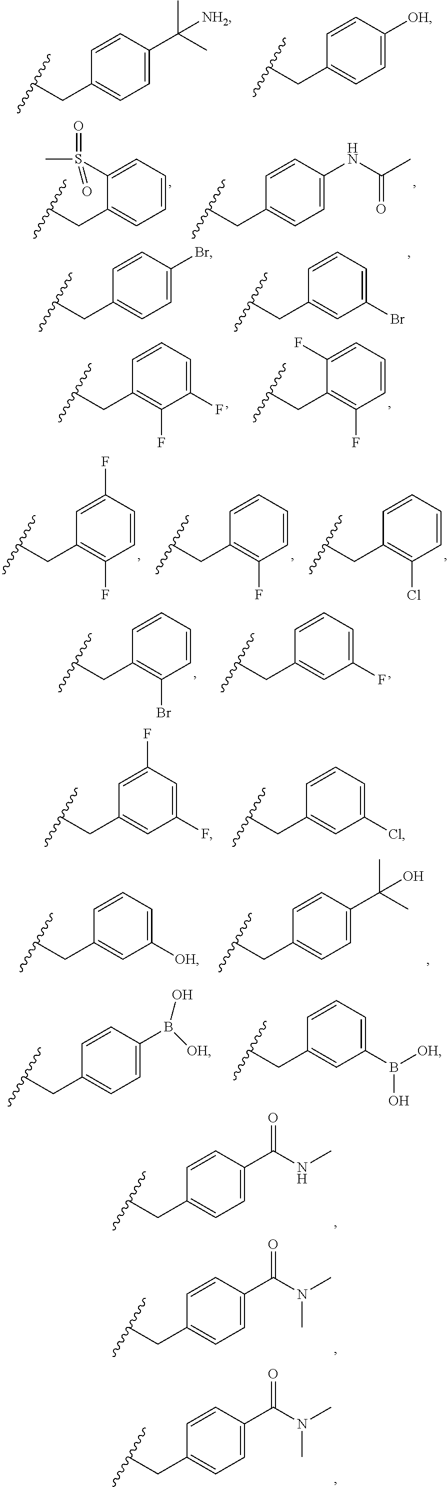

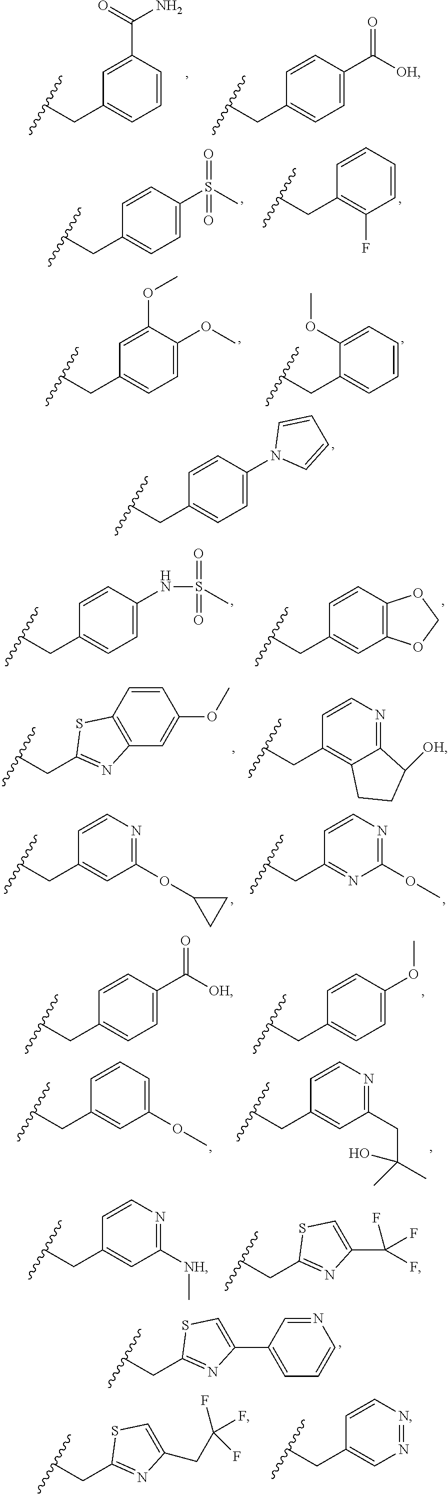

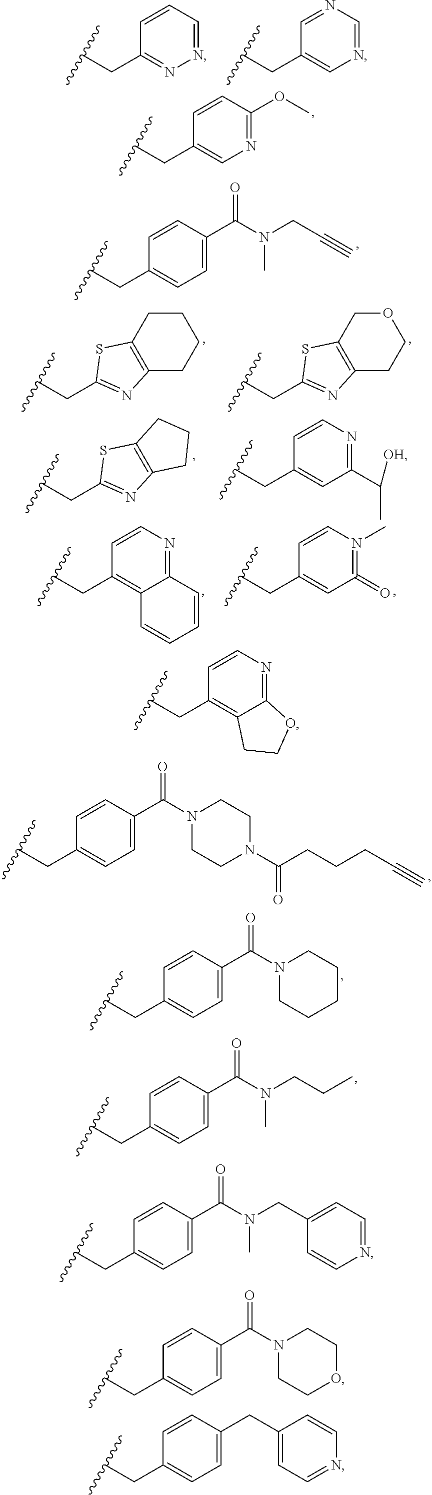

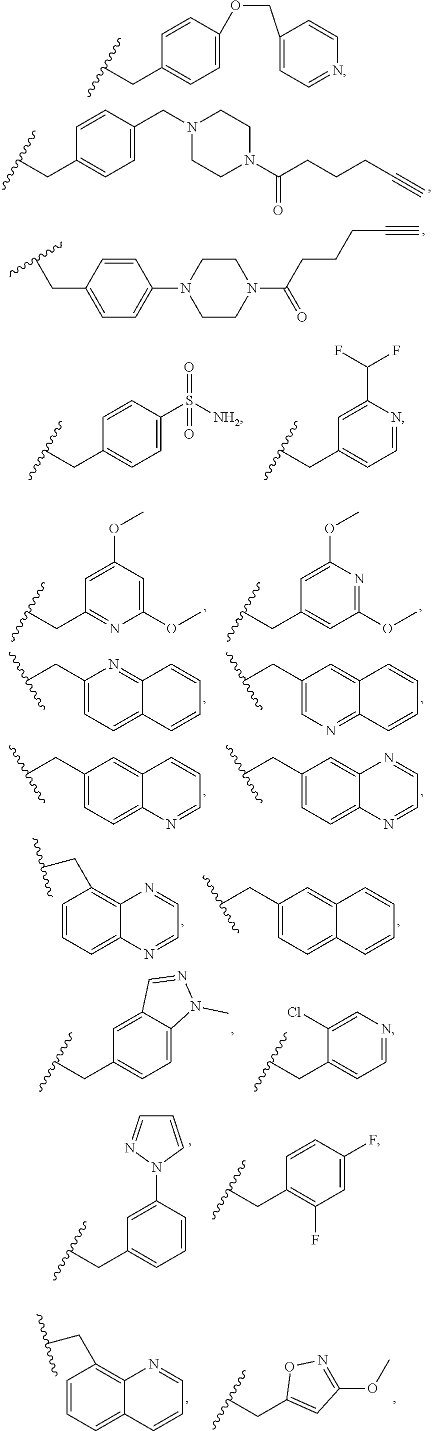

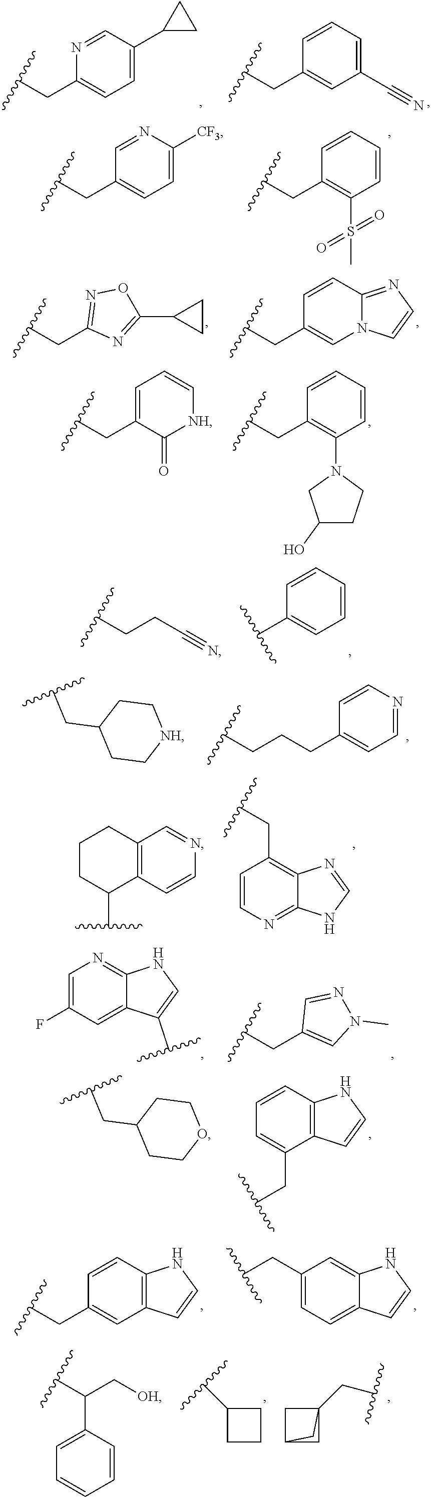

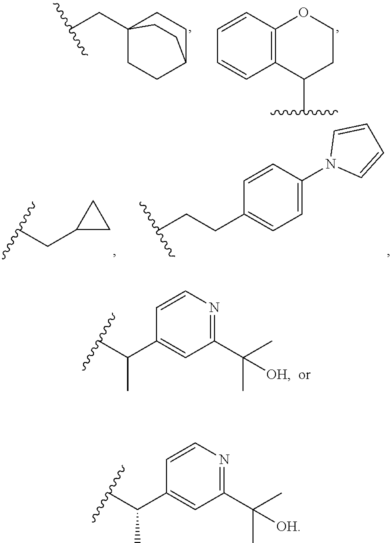

8. The compound, or pharmaceutically acceptable salt thereof, of any one of claims 1-7, wherein R³ is independently selected at each occurrence from halogen, a C₁₋₆ aliphatic group, —C(O)NR₂, —NR₂, and —OR.

9. The compound, or pharmaceutically acceptable salt thereof, of any one of claims 1-7, wherein R³ is independently selected at each occurrence from —CH₃, fluoro, —C(O)N(CH₃)₂, —C(O)N(CH₂CH₃)₂, 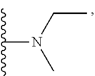 [SMILES: *N(C)CC]
—OCH₃, —OCH₂CH₃, —OCF₂CH₃, —OCH₂CH₂CH₃, and

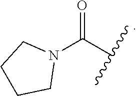 [SMILES: *C(=O)N1CCCC1]

10. The compound, or pharmaceutically acceptable salt thereof, of any one of claims 1-9, wherein R⁴ is hydrogen.

11. The compound, or pharmaceutically acceptable salt thereof, of any one of claims 1-7, wherein m is 1, R⁴ is hydrogen, and R³ is halogen, a C₁₋₆ aliphatic group, —C(O)NR₂, —NR₂, or —OR.

12. The compound, or pharmaceutically acceptable salt thereof, of any one of claims 1-7, wherein m is 2 or 3 and two adjacent instances of CR³R⁴ are optionally combined to form a carbon-carbon double bond of the formula —C(R⁴)═C(R⁴)—.

13. The compound, or pharmaceutically acceptable salt thereof, of claim 12, wherein m is 2 and the two adjacent instances of CR³R⁴ are combined to form a carbon-carbon double bond of the formula

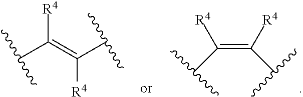

14. The compound, or pharmaceutically acceptable salt thereof, of any one of claims 1-7, wherein R⁴ and R³ are hydrogen.

15. The compound, or pharmaceutically acceptable salt thereof, of any one of claims 1-14, wherein each R⁵ is independently selected from halogen, —CN, —NO₂, —OR, —SR, —NR₂, —S(O)₂R, —S(O)₂NR₂, —S(O)R, —S(O)NR₂, —C(O)R, —C(O)OR, and —C(O)NR₂.

16. The compound, or pharmaceutically acceptable salt thereof, of any one of claims 1-15, wherein R⁵ is halogen and x is 1.

17. The compound, or pharmaceutically acceptable salt thereof, of any one of claims 1-16, wherein Ring A is 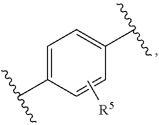
R⁵ is halogen, and x is 1.

18. The compound, or pharmaceutically acceptable salt thereof, of any one of claims 1-17, wherein R⁶ is halogen, a C₁₋₆ aliphatic group, —SF₅, —OR, —SR, —S(O)₂NR₂, —NR₂, —S(O)₂R, —SiR₃ or a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic,
a 5-12 membered saturated or partially unsaturated bicyclic heterocyclic ring that is optionally bridged or spirocyclic having 1-4 heteroatoms independently selected from nitrogen, oxygen, and sulfur,
wherein the C₁₋₆ aliphatic group and cyclic group are substituted with z instances of R^{B}.

19. The compound, or pharmaceutically acceptable salt thereof, of any one of claims 1-17, wherein R⁶ is halogen.

20. The compound, or pharmaceutically acceptable salt thereof, of any one of claims 1-17, wherein R⁶ is a C₁₋₆ aliphatic group, —SF₅, —OR, —SR, —S(O)₂NR₂, —S(O)₂R, or a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur,
a 5-6 membered monocyclic heteroaromatic ring having 1-4 heteroatoms independently selected from nitrogen, oxygen, and sulfur,
an 8-10 membered bicyclic heteroaromatic ring having 1-5 heteroatoms independently selected from nitrogen, oxygen, and sulfur,
a 5-12 membered saturated or partially unsaturated bicyclic carbocyclic ring that is optionally bridged or spirocyclic, a 5-12 membered saturated or partially unsaturated bicyclic heterocyclic ring that is optionally bridged or spirocyclic having 1-4 heteroatoms independently selected from nitrogen, oxygen, and sulfur,
wherein the cyclic group is substituted with z instances of R^{B}.

21. The compound, or pharmaceutically acceptable salt thereof, of any one of claims 1-17, wherein R⁶ is a cyclic group selected from:
a 3-8 membered saturated or partially unsaturated monocyclic carbocyclic ring,
phenyl,
a 3-8 membered saturated or partially unsaturated monocyclic heterocyclic ring having 1-2 heteroatoms independently selected from nitrogen, oxygen, and sulfur, and
a 5-6 membered monocyclic heteroaromatic ring having 1-4 heteroatoms independently selected from nitrogen, oxygen, and sulfur,
wherein the cyclic group is substituted with z instances of R^{B}.

22. The compound, or pharmaceutically acceptable salt thereof, of any one of claims 1-17, wherein R⁶ is phenyl substituted with z instances of R^{B}.

23. The compound, or pharmaceutically acceptable salt thereof, of any one of claims 1-17, wherein R⁶ is —SF₅.

24. The compound, or pharmaceutically acceptable salt thereof, of any one of claims 1-17, wherein Ring A is 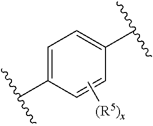
and R⁶ is fluoro-substituted phenyl.

25. The compound, or pharmaceutically acceptable salt thereof, of any one of claims 1-17, wherein Ring A is 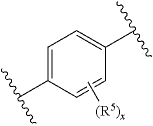
and R⁶ is —SF₅.

26. The compound, or pharmaceutically acceptable salt thereof, of any one of claims 1-17, wherein R⁶ is a 5-membered heteroaryl substituted with z instances of R^{B}.

27. The compound, or pharmaceutically acceptable salt thereof, of any one of claims 1-17, wherein R⁶ is isopropyl, sec-butyl, —CF₃, —SF₅, —CH₂CF₃, or —OCF₃.

28. The compound, or pharmaceutically acceptable salt thereof, of claim 1 or 2, wherein Ring A is bicyclic, m is greater than 0, and R⁶ is hydrogen.

29. The compound, or pharmaceutically acceptable salt thereof, of any one of claims 1-17, wherein R⁶ is —F, —Br, —Cl, —SF₅, —CF₃, —CH₃, —CH₂CF₃, —OCF₃, —OCH₃, —SCH₃,

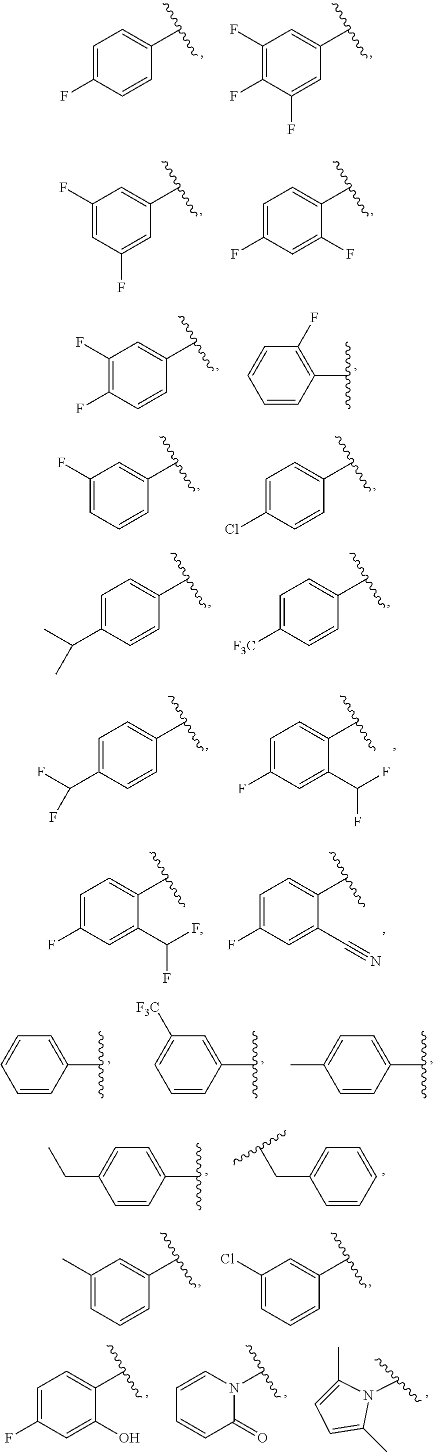

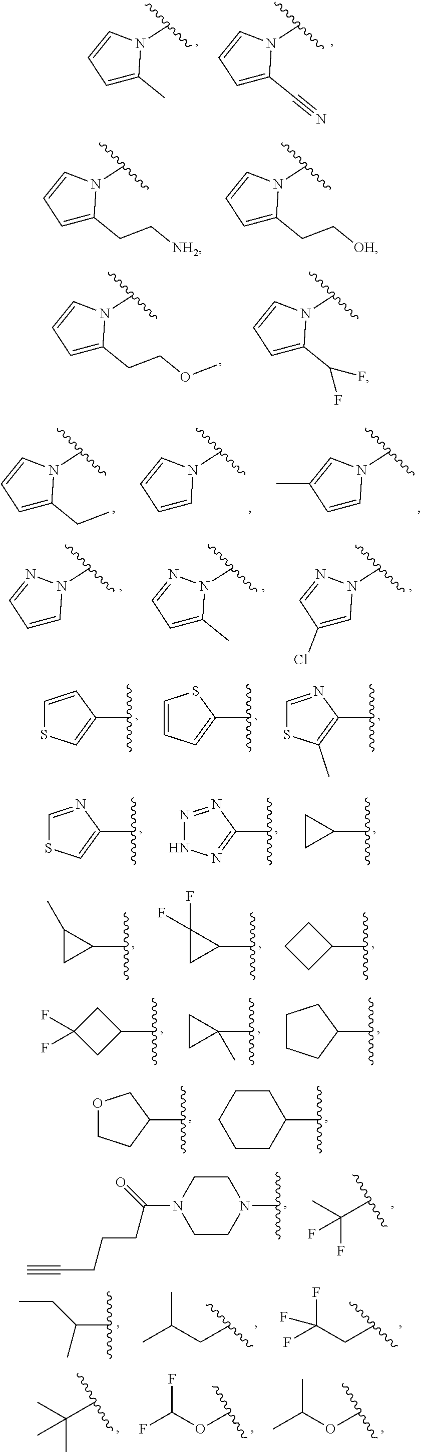

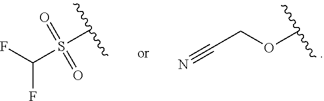 [SMILES: *S(=O)(=O)C(F)F ; *OCC#N]

30. The compound of claim 1 or 2, or pharmaceutically acceptable salt thereof, wherein R⁴ is hydrogen and R^{B} is independently selected at each occurrence from halogen or a C₁₋₆ aliphatic group substituted with 1, 2, or 3 groups independently selected from halogen, —CN, —OH, —SR, and —OR.

31. The compound of claim 5 or 6, or pharmaceutically acceptable salt thereof, wherein Cy′ is an optionally substituted 5-6 membered monocyclic heteroaromatic ring having 1-4 heteroatoms independently selected from nitrogen, oxygen, and sulfur.

32. The compound of any one of claims 1-10, or pharmaceutically acceptable salt thereof, wherein m is 0.

33. The compound of any one of claims 1-10, or pharmaceutically acceptable salt thereof, wherein m is 1.

34. The compound of claim 1, or a pharmaceutically acceptable salt thereof, of Formula II:

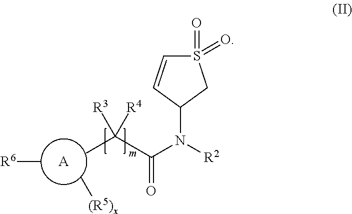

35. The compound of claim 1, or a pharmaceutically acceptable salt thereof, of Formula IIIa or IIb: 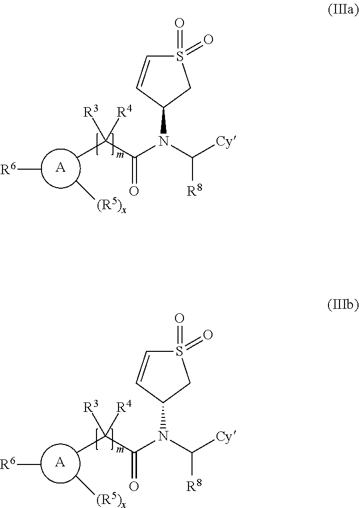
wherein R⁸ is hydrogen, optionally substituted C₁₋₆ aliphatic group, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂,—N(R)C(NR)NR₂, —N(R)S(O)₂NR₂, or —N(R)S(O)₂R.

36. The compound of claim 1, or a pharmaceutically acceptable salt thereof, of Formula IVa or IVb: 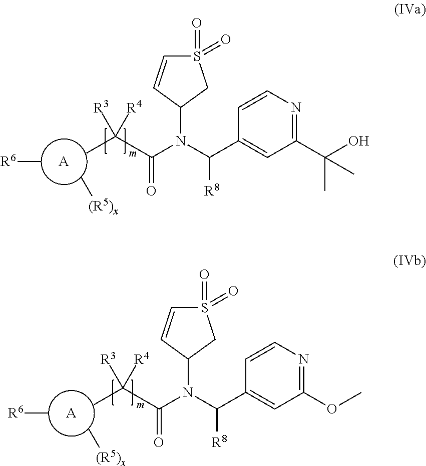
wherein R⁸ is hydrogen, optionally substituted C₁₋₆ aliphatic group, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂,—N(R)C(NR)NR₂, —N(R)S(O)₂NR₂, or —N(R)S(O)₂R.

37. The compound of claim 1, or a pharmaceutically acceptable salt thereof, of Formula Va or Vb: 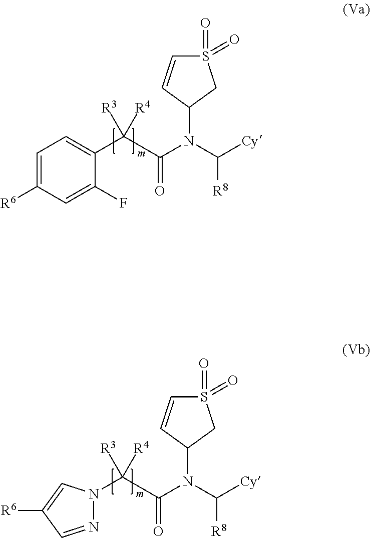
wherein R⁸ is hydrogen, optionally substituted C₁₋₆ aliphatic group, halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂,—N(R)C(NR)NR₂, —N(R)S(O)₂NR₂, or —N(R)S(O)₂R.

38. The compound of claim 37, or a pharmaceutically acceptable salt thereof, wherein m is 1.

39. A compound of claim 1, or a pharmaceutically acceptable salt thereof, wherein the compound is a compound of Table 1.

40. A compound selected from 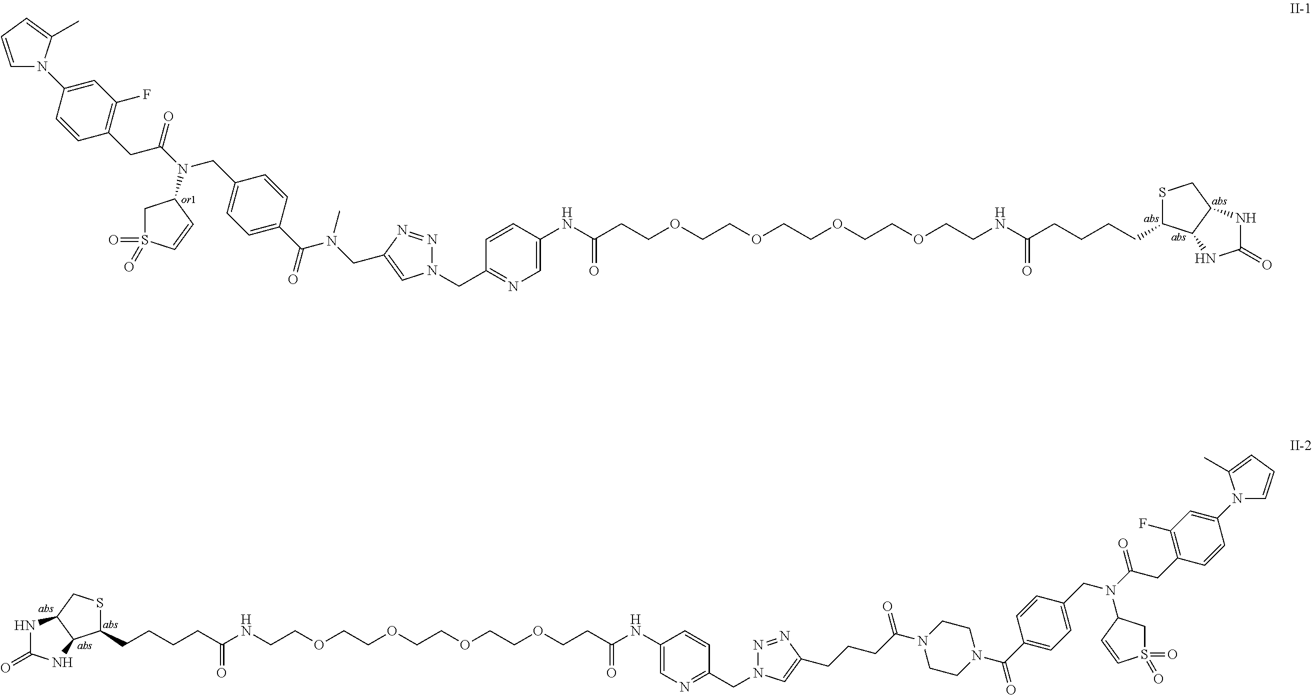 [SMILES: Cc1cccn1-c1ccc(CC(=O)N(Cc2ccc(C(=O)N(C)Cc3cn(Cc4ccc(NC(=O)CCOCCOCCOCCOCCNC(=O)CCCC[C@@H]5SC[C@@H]6NC(=O)N[C@@H]65)cn4)nn3)cc2)[C@@H]2C=CS(=O)(=O)C2)c(F)c1 ; Cc1cccn1-c1ccc(CC(=O)N(Cc2ccc(C(=O)N3CCN(C(=O)CCCc4cn(Cc5ccc(NC(=O)CCOCCOCCOCCOCCNC(=O)CCCC[C@@H]6SC[C@@H]7NC(=O)N[C@@H]76)cn5)nn4)CC3)cc2)C2C=CS(=O)(=O)C2)c(F)c1] 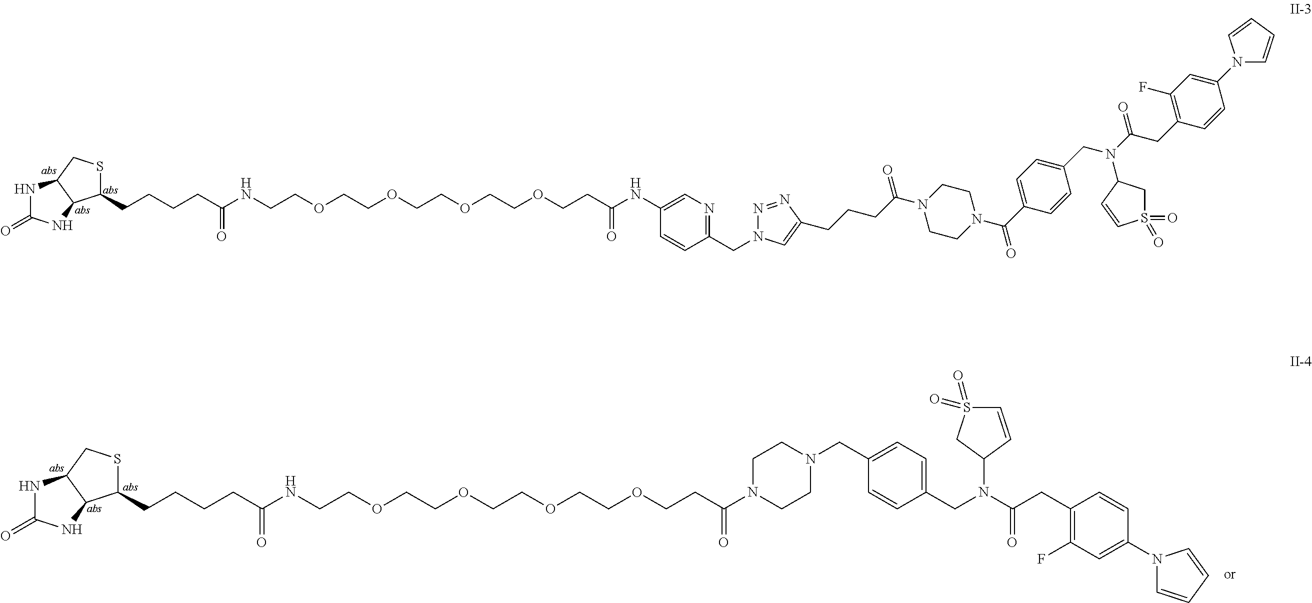 [SMILES: O=C(CCCC[C@@H]1SC[C@@H]2NC(=O)N[C@@H]21)NCCOCCOCCOCCOCCC(=O)Nc1ccc(Cn2cc(CCCC(=O)N3CCN(C(=O)c4ccc(CN(C(=O)Cc5ccc(-n6cccc6)cc5F)C5C=CS(=O)(=O)C5)cc4)CC3)nn2)nc1 ; O=C(CCCC[C@@H]1SC[C@@H]2NC(=O)N[C@@H]21)NCCOCCOCCOCCOCCC(=O)N1CCN(Cc2ccc(CN(C(=O)Cc3ccc(-n4cccc4)cc3F)C3C=CS(=O)(=O)C3)cc2)CC1] 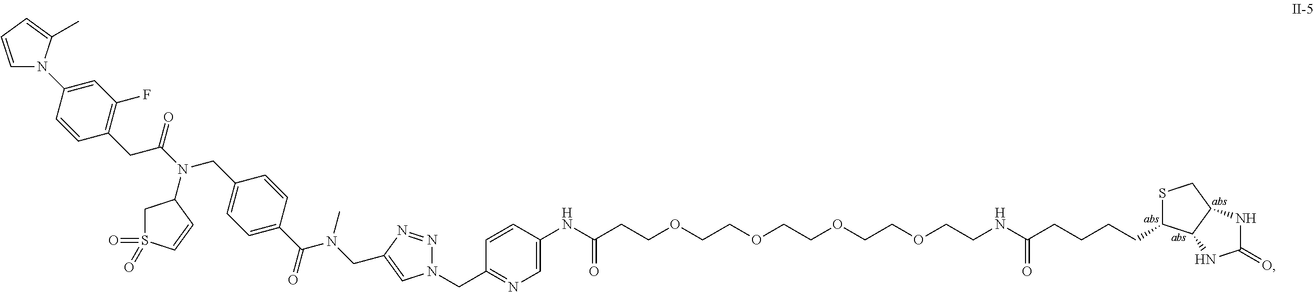 [SMILES: Cc1cccn1-c1ccc(CC(=O)N(Cc2ccc(C(=O)N(C)Cc3cn(Cc4ccc(NC(=O)CCOCCOCCOCCOCCNC(=O)CCCC[C@@H]5SC[C@@H]6NC(=O)N[C@@H]65)cn4)nn3)cc2)C2C=CS(=O)(=O)C2)c(F)c1]
or a pharmaceutically acceptable salt thereof.

41. A pharmaceutical composition comprising a compound or pharmaceutically acceptable salt thereof according to any one of claims 1-40, and one or more pharmaceutically acceptable carriers.

42. A method of treating cancer in a subject, wherein the cancer is characterized as microsatellite instability-high (MSI-H) or mismatch repair deficient (dMMR), comprising administering to the subject a therapeutically effective amount of a compound according to any one of claims 1-40, or a pharmaceutically acceptable salt thereof.

43. A method of modulating WRN activity in a subject, wherein the method comprises administering to the subject a therapeutically effective amount of the compound according to any one of claims 1-40, or a pharmaceutically acceptable salt thereof.

44. A method of inhibiting WRN in a subject, wherein the method comprises administering to the subject a therapeutically effective amount of the compound according to any one of claims 1-40, or a pharmaceutically acceptable salt thereof.

45. A method of treating a disorder or disease which can be treated by WRN inhibition in a subject, wherein the method comprises administering to the subject a therapeutically effective amount of the compound according to any one of claims 1-40, or a pharmaceutically acceptable salt thereof.

46. The method of claim 45, wherein the disorder or disease is a cancer characterized as microsatellite instability-high (MSI-H) or mismatch repair deficient (dMMR).

47. The method of claim 46, wherein the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer.
